(19) United States
(12) Patent Application Publication
(10) Pub. No.: US 2024/0336945 A1
(43) Pub. Date: Oct. 10, 2024

(54) TREM COMPOSITIONS AND USES THEREOF

(71) Applicant: Flagship Pioneering Innovations VI, LLC, Cambridge, MA (US)

(72) Inventors: David Arthur Berry, Newton, MA (US); Theonie Anastassiadis, Somerville, MA (US); Christine Elizabeth Hajdin, Boston, MA (US); Noubar Boghos Afeyan, Lexington, MA (US)

(21) Appl. No.: 18/292,098

(22) PCT Filed: Jul. 26, 2022
(86) PCT No.: PCT/US2022/038385

§ 371 Date: Jan. 26, 2024

Related U.S. Application Data
(60) Provisional application No. 63/225,841, filed on Jul. 26, 2021.

(51) Int. Cl. C12P 19/34; A61K 31/7105; C12N 15/10

(52) U.S. Cl. CPC C12P 19/34; A61K 31/7105; C12N 15/1003

(57) ABSTRACT

The invention relates generally to tRNA-based effector molecules and methods relating thereto.

### BACKGROUND

This application is a U.S. National Phase Application under 35 U.S.C. § 371 of International Application No. PCT/US2022/038385 filed Jul. 26, 2022, which claims priority to U.S. Provisional Application No. 63/225,841, which was filed on Jul. 26, 2021. The entire contents of each of the foregoing applications is hereby incorporated by reference.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Mar. 4 2024, is entitled “F2099-7005USNP_ST26.xml”, and is 828,923 bytes in size.

### SUMMARY

In an aspect, the disclosure provides a method of making a purified tRNA effector molecule (TREM) composition, e.g., a TREM pharmaceutical composition, comprising: providing a host cell, e.g., a fungal cell or cell line; an insect cell or cell line; or a plant, plant cell or cell line, comprising an exogenous nucleic acid, e.g., a DNA or RNA, encoding the TREM;
maintaining the host cell under conditions sufficient to express the TREM;
purifying the TREM from the host cell, e.g., according to a method described herein; and
formulating the purified TREM as a pharmaceutical composition, e.g., by combining the TREM with a pharmaceutical excipient,
thereby making the TREM composition.

In an embodiment, the host cell comprises a fungal cell or cell line. In an embodiment, the fungal cell or cell line is a fungal cell or cell line chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.

In an embodiment, the fungal cell or cell line is a Saccharomyces cell or cell line. In an embodiment, the fungal cell or cell line is a Saccharomyces cerevisiae fungal cell or cell line. In an embodiment, the fungal cell or cell line is a Schizosaccharomyces pombe fungal cell or cell line.

In an embodiment, the fungal cell or cell line is a Candida cylindracea fungal cell or cell line. In an embodiment, the fungal cell or cell line is a Candida albicans fungal cell or cell line.

In an embodiment, the fungal cell or cell line is a Neurospora crassa fungal cell or cell line.

In an embodiment, the fungal cell or cell line is a Pichia jadinii fungal cell or cell line.

In an embodiment, the host cell comprises an insect cell or cell line. In an embodiment, the insect cell or cell line is an insect cell or cell line chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.

In an embodiment, the insect cell is a Spodoptera frugiperda cell. In an embodiment, the Spodoptera frugiperda cell is an Sf9 cell.

In an embodiment, the insect cell is a Trichoplusia ni cell. In an embodiment, the insect cell is a H5 cell (High Five™, Invitrogen, Sorrento, CA).

In an embodiment, the host cell comprises a plant, plant cell or cell line. In an embodiment, the plant, plant cell or cell line is a monocotyledonous plant, cell or cell line. In an embodiment, the plant, plant cell or cell line is a dicotyledonous plant, cell or cell line.

In an embodiment, the plant, cell or cell line is a plant, cell or cell line chosen from: wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.

In an embodiment, the plant, cell or cell line is an Arabidopsis plant, cell or cell line. In an embodiment, the Arabidopsis plant, cell or cell line is an A. thaliana plant, cell or cell line.

In an embodiment, the nucleic acid comprises an RNA, which upon reverse transcription, results in a DNA which can be transcribed into the TREM.

In an embodiment, the nucleic acid comprises an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof.

In an embodiment, the nucleic acid comprises an RNA sequence comprising a consensus sequence, e.g., as provided herein, e.g., a consensus sequence of Formula I zzz, Formula II zzz, or Formula III zzz, wherein zzz indicates any of the twenty amino acids:Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In an embodiment, the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; or/and
(iii) separating a TREM from other RNA species in the small RNA preparation by affinity-based separation, e.g., sequence affinity-based separation.

In one aspect, the invention features a method of making a tRNA effector molecule (TREM) composition, comprising:
(a) providing a host cell, comprising exogenous nucleic acid, e.g., a DNA or RNA, encoding a TREM under conditions sufficient to express the TREM, and
(b) purifying the expressed TREM from the host cell culture to produce a TREM composition, thereby making a TREM composition.

In an embodiment, the TREM composition is a pharmaceutically acceptable composition.

In another aspect, the invention features a method of making a TREM composition, e.g., a pharmaceutical TREM composition, comprising:
a) providing a purified TREM composition, e.g., a purified TREM composition made by culturing a host cell, e.g., a fungal cell or cell line; an insect cell or cell line; or a plant, plant cell or cell line, comprising DNA or RNA encoding a TREM under conditions sufficient to express the TREM, and purifying the expressed TREM from the host cell culture to produce a purified TREM composition,
b) providing a value, e.g., by evaluating or testing, for a characteristic described herein (e.g., a characteristic related to identity (e.g., sequence), purity (e.g., process impurity such as TREM fragments, host cell protein or host cell DNA), activity (e.g., adaptor activity)),
c) optionally, formulating the purified TREM composition as a pharmaceutical drug product (e.g., combining the TREM composition with a pharmaceutical excipient) if it meets a reference criterion for the one or more characteristic,
thereby making the TREM composition.

In an embodiment, the host cell comprises a fungal cell or cell line. In an embodiment, the fungal host cell or cell line is a fungal cell or cell line chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.

In an embodiment, the fungal cell or cell line is a Saccharomyces cell or cell line. In an embodiment, the fungal cell or cell line is a Saccharomyces cerevisiae fungal cell or cell line. In an embodiment, the fungal cell or cell line is a Schizosaccharomyces pombe fungal cell or cell line.

In an embodiment, the fungal cell or cell line is a Candida cylindracea fungal cell or cell line. In an embodiment, the fungal cell or cell line is a Candida albicans fungal cell or cell line.

In an embodiment, the fungal cell or cell line is a Neurospora crassa fungal cell or cell line.

In an embodiment, the fungal cell or cell line is a Pichia jadinii fungal cell or cell line.

In an embodiment, the host cell comprises an insect cell or cell line. In an embodiment, the insect host cell or cell line is an insect cell or cell line chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.

In an embodiment, the insect cell is a Spodoptera frugiperda cell. In an embodiment, the Spodoptera frugiperda cell is an Sf9 cell.

In an embodiment, the insect cell is a Trichoplusia ni cell. In an embodiment, the insect cell is a H5 cell (High Five™, Invitrogen, Sorrento, CA).

In an embodiment, the host cell comprises a plant, plant cell or cell line. In an embodiment, the host plant, plant cell or cell line is a monocotyledonous plant, cell or cell line.

In an embodiment, the host plant, plant cell or cell line is a dicotyledonous plant, cell or cell line.

In an embodiment, the host plant, cell or cell line is a plant, cell or cell line chosen from: wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.

In an embodiment, the plant, cell or cell line is an Arabidopsis plant, cell or cell line. In an embodiment, the Arabidopsis plant, cell or cell line is an A. thaliana plant, cell or cell line.

In another aspect, the invention features a method of making a pharmaceutical TREM composition comprising:
combining
a) a TREM, e.g., a purified TREM composition, e.g., a TREM composition made by a method described herein; and
b) a pharmaceutically acceptable component, e.g., an excipient, thereby making a pharmaceutical TREM composition.

In another aspect, the present disclosure provides a composition comprising a purified tRNA effector molecule (TREM) (e.g., a purified TREM composition made according to a method described herein), comprising an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof.

In an aspect, the present disclosure provides a composition comprising a purified tRNA effector molecule (TREM) (e.g., a purified TREM composition made according to a method described herein), comprising an RNA sequence comprising a consensus sequence provided herein, e.g., a consensus sequence of Formula I zzz, Formula II zzz, or Formula III zzz, wherein zzz indicates any of the twenty amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In another aspect, the invention features a GMP-grade, recombinant TREM composition (e.g., a TREM composition made in compliance with cGMP, and/or in accordance with similar requirements) comprising an RNA sequence at least 80% (e.g., at least 85%, at least 90%, at least 95%, at least 97%, at least 98%, at least 99%) identical to an RNA encoded by a DNA sequence listed in Table 1, or a fragment or functional fragment thereof.

In another aspect, the invention features a GMP-grade, recombinant TREM composition (e.g., a TREM composition made in compliance with cGMP, and/or in accordance with similar requirements) comprising an RNA sequence comprising a consensus sequence provided herein.

In an aspect, the invention features a TREM comprising a consensus sequence provided herein.

In an aspect, the invention features a TREM comprising a consensus sequence of Formula I zzz, wherein zzz indicates any of the twenty amino acids and Formula I corresponds to all species.

In an aspect, the invention features a TREM comprising a consensus sequence of Formula II zzz, wherein zzz indicates any of the twenty amino acids and Formula II corresponds to mammals.

In an aspect, the invention features a TREM comprising a consensus sequence of Formula III zzz, wherein zzz indicates any of the twenty amino acids and Formula III corresponds to humans.

In an embodiment, ZZZ indicates any of the amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In an aspect, the invention features a GMP-grade, recombinant TREM composition comprising an RNA sequence comprising a consensus sequence provided herein.

In an embodiment of any of the TREM compositions or pharmaceutical TREM compositions provided herein, the composition comprises one or more, e.g., a plurality, of TREMs.

In an embodiment of any of the TREM compositions or pharmaceutical TREM compositions provided herein, the composition comprises at least 1, 2, 3, 4, 5, 6, 7, 8, 9 or 10 species of TREMs.

In an embodiment of any of the TREM compositions or pharmaceutical TREM compositions provided herein, the TREM composition (or an intermediate in the production of a TREM composition) comprises one or more of the following characteristics:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) fragments of less than 0.1%, 1%, 2%, 3%, 4%, 5%, 6%, 7%, 8%, 9% or 10%;
(vi) low levels or absence of endotoxins, e.g., a negative result as measured by the Limulus amebocyte lysate (LAL) test;
(vii) in-vitro translation activity, e.g., as measured by an assay described in Example 10;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>;
(x) viral contamination, e.g., the composition or preparation has an absence of, or an undetectable level of viral contamination; or
(xi) differential modification, e.g., comprising a modification characteristic of a fungal cell or cell line; an insect cell or cell line; or a plant, plant cell or cell line.

In another aspect, the invention features, a cell comprising an exogenous nucleic acid comprising:

a nucleic acid sequence, e.g., DNA or RNA, that encodes a TREM, wherein the nucleic acid sequence comprises:
(i) a control region sequence;
(ii) a sequence encoding a modified TREM;
(iii) a sequence encoding more than one TREM; or
(iv) a promoter sequence that comprises a Pol III recognition site, e.g., a U6 promoter, a 7 SK promoter or a H1 promoter, or a fragment thereof.

In an aspect, provided herein is a TREM made by a method described herein.

In another aspect, the disclosure provides a TREM comprising a modification characteristic of a fungal cell or cell line; an insect cell or cell line; or a plant, plant cell or cell line.

In an embodiment, the modification is not made in mammalian cells, or is made at a different site or at a different level as compared with a fungal cell or cell line; an insect cell or cell line; or a plant, plant cell or cell line In an embodiment, the modification is a modification listed in any one of Tables 2-4. In an embodiment, the modification is a modification listed in Table 2. In an embodiment, the formation of the modification is mediated by an enzyme listed in Table 2. In an embodiment, the modification is a modification listed in Table 3. In an embodiment, the modification is a modification listed in Table 4.

In another aspect, the disclosure provides a TREM comprising a modification characteristic of a fungal host cell or cell line.

In an embodiment, the modification is not made in mammalian cells, or is made at a different site or at a different level as compared with a fungal cell.

In an embodiment, the modification is a modification listed in Table 3.

In another aspect, the disclosure provides a TREM comprising a modification characteristic of an insect host cell or cell line.

In an embodiment, the modification is not made in mammalian cells, or is made at a different site or at a different level as compared with an insect cell.

In an embodiment, the modification is a modification listed in Table 4.

In another aspect, the disclosure provides a TREM comprising a modification characteristic of a plant host, plant cell or cell line.

In an embodiment, the modification is not made in mammalian cells, or is made at a different site or at a different level as compared with a plant, cell or cell line.

In an embodiment, the modification is a modification listed in Table 2. In an embodiment, the modification is added by an enzyme listed in Table 2.

In an aspect, the invention features a method of modulating a tRNA pool in a cell, e.g., a mammalian cell, comprising:

providing a TREM composition, e.g., a purified TREM composition, and contacting the cell with the TREM composition,

thereby modulating the tRNA pool in the cell,

wherein the TREM in the composition is made by a method described herein, e.g., by expression in a host cell, e.g., a fungal cell or cell line; an insect cell or cell line; or a plant, plant cell or cell line or the TREM in the composition comprises a modification charateritic of a fungal cell or cell line; an insect cell or cell line; or a plant, plant cell or cell line.

In an embodiment, the modification is not made in mammalian cells, or is made at a different site or at a different level than as compared with a fungal cell or cell line; an insect cell or cell line; or a plant, plant cell or cell line.

In another aspect, the invention features a method of delivering a TREM to a cell, tissue, or subject, e.g., a mammalian cell, tissue, or subject, comprising:

providing a cell, tissue, or subject, and contacting the cell, tissue, or subject, with a TREM composition, e.g., a pharmaceutical TREM composition comprising the TREM

wherein the TREM in the composition is made by a method described herein, e.g., by expression in a host cell, e.g., a fungal cell or cell line; an insect cell or cell line; or a plant, plant cell or cell line, or the TREM in the composition comprises a modification characteristic of a fungal cell or cell line; an insect cell or cell line; or a plant, plant cell or cell line.

In an embodiment, the modification is not made in mammalian cells, or is made at a different site or at a different level than as compared with a fungal (e.g., yeast), insect, or plant cell.

In an embodiment, the modification is a modification listed in any one of Tables 2-4. In an embodiment, the modification is a modification listed in Table 2. In an embodiment, the formation of the modification is mediated by an enzyme listed in Table 2. In an embodiment, the modification is a modification listed in Table 3. In an embodiment, the modification is a modification listed in Table 4.

In another aspect, the invention features a method of treating a subject, e.g., modulating the metabolism, e.g., the translational capacity of a cell, in a subject, comprising: providing, e.g., administering to the subject, an exogenous nucleic acid, e.g., a DNA or RNA, which encodes a TREM, thereby treating the subject.

In an embodiment of any of the methods disclosed herein, the TREM composition is made by:
providing a host cell, e.g., a fungal cell or cell line; an insect cell or cell line; or a plant, plant cell or cell line comprising an exogenous nucleic acid, e.g., a DNA or RNA, encoding the TREM;
maintaining the host cell under conditions sufficient to express the TREM; and/or purifying the TREM from the host cell, e.g., according to a method described herein.

In an embodiment, the host cell comprises a fungal cell or cell line. In an embodiment, the fungal host cell or cell line is a fungal cell or cell line chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.

In an embodiment, the fungal cell or cell line is a Saccharomyces cell or cell line. In an embodiment, the fungal cell or cell line is a Saccharomyces cerevisiae fungal cell or cell line. In an embodiment, the fungal cell or cell line is a Schizosaccharomyces pombe fungal cell or cell line.

In an embodiment, the fungal cell or cell line is a Candida cylindracea fungal cell or cell line. In an embodiment, the fungal cell or cell line is a Candida albicans fungal cell or cell line.

In an embodiment, the fungal cell or cell line is a Neurospora crassa fungal cell or cell line.

In an embodiment, the fungal cell or cell line is a Pichia jadinii fungal cell or cell line.

In an embodiment, the host cell comprises an insect cell or cell line. In an embodiment, the insect host cell or cell line is an insect cell or cell line chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.

In an embodiment, the insect cell is a Spodoptera frugiperda cell. In an embodiment, the Spodoptera frugiperda cell is an Sf9 cell.

In an embodiment, the insect cell is a Trichoplusia ni cell. In an embodiment, the insect cell is a H5 cell (High Five™, Invitrogen, Sorrento, CA).

In an embodiment, the host cell comprises a plant, plant cell or cell line. In an embodiment, the host plant, plant cell or cell line is a monocotyledonous plant, cell or cell line.

In an embodiment, the host plant, plant cell or cell line is a dicotyledonous plant, cell or cell line.

In an embodiment, the host plant, cell or cell line is a plant, cell or cell line chosen from: wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.

In an embodiment, the plant, cell or cell line is an Arabidopsis plant, cell or cell line. In an embodiment, the Arabidopsis plant, cell or cell line is an A. thaliana plant, cell or cell line.

In an embodiment of any of the methods disclosed herein, the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species by affinity-based separation, e.g., sequence affinity-based separation.

In an embodiment of any of the methods disclosed herein, the TREM comprises:
(i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
(ii) an RNA sequence comprising a consensus sequence provided herein.

In an aspect, the disclosure provides a method of making a purified tRNA effector molecule (TREM) composition, e.g., a TREM pharmaceutical composition, comprising: providing an insect host cell or cell line comprising an exogenous nucleic acid, e.g., a DNA or RNA, encoding the TREM;
maintaining the insect host cell or cell line under conditions sufficient to express the TREM;
purifying the TREM from the insect host cell or cell line, e.g., according to a method described herein; and
formulating the purified TREM as a pharmaceutical composition, e.g., by combining the TREM with a pharmaceutical excipient,
thereby making the TREM composition.

In an embodiment, the fungal host comprises an insect host cell or cell line. In an embodiment, the insect host cell or cell line is an insect cell or cell line chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.

In an embodiment, the insect cell is a Spodoptera frugiperda cell. In an embodiment, the Spodoptera frugiperda cell is an Sf9 cell.

In an embodiment, the insect cell is a Trichoplusia ni cell. In an embodiment, the insect cell is a H5 cell (High Five™, Invitrogen, Sorrento, CA).

In an embodiment, the purification step comprises one, two or all of the following steps, e.g., in the order recited:
(i) separating nucleic acids from protein to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity.

In an aspect, the disclosure provides a method of making a purified tRNA effector molecule (TREM) composition, e.g., a TREM pharmaceutical composition, comprising:
providing a fungal host cell comprising an exogenous nucleic acid, e.g., a DNA or RNA, encoding the TREM;
maintaining the fungal host cell under conditions sufficient to express the TREM;
purifying the TREM from the fungal host cell, e.g., according to a method described herein; and
formulating the purified TREM as a pharmaceutical composition, e.g., by combining the TREM with a pharmaceutical excipient,
thereby making the TREM composition.

In an embodiment, the fungal host comprises a fungal host cell or cell line. In an embodiment, the fungal host cell or cell line is a fungal cell or cell line chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.

In an embodiment, the fungal cell or cell line is a Saccharomyces cell or cell line. In an embodiment, the fungal cell or cell line is a Saccharomyces cerevisiae fungal cell or cell line. In an embodiment, the fungal cell or cell line is a Schizosaccharomyces pombe fungal cell or cell line.

In an embodiment, the fungal cell or cell line is a Candida cylindracea fungal cell or cell line. In an embodiment, the fungal cell or cell line is a Candida albicans fungal cell or cell line.

In an embodiment, the fungal cell or cell line is a Neurospora crassa fungal cell or cell line.

In an embodiment, the fungal cell or cell line is a Pichia jadinii fungal cell or cell line.

In an embodiment, the purification step comprises one, two or all of the following steps, e.g., in the order recited:
(i) separating nucleic acids from protein to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity.

In an aspect, the disclosure provides a method of making a purified tRNA effector molecule (TREM) composition, e.g., a TREM pharmaceutical composition, comprising:
providing a plant host, plant cell or cell line comprising an exogenous nucleic acid, e.g., a DNA or RNA, encoding the TREM;
maintaining the plant host, plant cell or cell line under conditions sufficient to express the TREM;
purifying the TREM from the plant host, plant cell or cell line, e.g., according to a method described herein; and
formulating the purified TREM as a pharmaceutical composition, e.g., by combining the TREM with a pharmaceutical excipient,
thereby making the TREM composition.

In an embodiment, the plant host comprises a plant, a plant cell or cell line. In an embodiment, the host plant, plant cell or cell line is a monocotyledonous plant, cell or cell line.

In an embodiment, the host plant, plant cell or cell line is a dicotyledonous plant, cell or cell line.

In an embodiment, the host plant, cell or cell line is a plant, cell or cell line chosen from: wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.

In an embodiment, the plant, cell or cell line is an Arabidopsis plant, cell or cell line. In an embodiment, the Arabidopsis plant, cell or cell line is an A. thaliana plant, cell or cell line.

In an embodiment, the purification step comprises one, two or all of the following steps, e.g., in the order recited:
(i) separating nucleic acids from protein to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity.

As disclosed herein tRNA-based effector molecules (TREMs) are complex molecules which can mediate a variety of cellular processes. Pharmaceutical TREM compositions can be administered to cells, tissues or subjects to modulate these functions, e.g., in vitro or in vivo. Disclosed herein are TREM compositions, preparations, methods of making TREM compositions and preparations, and methods of using TREM compositions and preparations.

Additional features of any of the aforesaid TREM compositions, preparations, methods of making TREM compositions and preparations, and methods of using TREM compositions and preparations include one or more of the following enumerated embodiments.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. A method of making a purified tRNA effector molecule (TREM) composition, e.g., a TREM pharmaceutical composition, comprising:
   providing a host cell e.g., a fungal cell or cell line (e.g., a yeast cell), an insect cell or cell line, or a plant, plant cell or cell line, comprising an exogenous nucleic acid, e.g., a DNA or RNA, encoding the TREM;
   maintaining the host cell under conditions sufficient to express the TREM;
   purifying the TREM from the host cell, e.g., according to a method described herein; and
   formulating the purified TREM as a pharmaceutical composition, e.g., by combining the TREM with a pharmaceutical excipient,
   thereby making the TREM composition.
2. A method of making a purified tRNA effector molecule (TREM) composition, e.g., a TREM pharmaceutical composition, comprising:
   providing an insect host cell comprising an exogenous nucleic acid, e.g., a DNA or RNA, encoding the TREM;
   maintaining the insect host cell under conditions sufficient to express the TREM;
   purifying the TREM from the insect host cell, e.g., according to a method described herein; and
   formulating the purified TREM as a pharmaceutical composition, e.g., by combining the TREM with a pharmaceutical excipient,
   thereby making the TREM composition.
3. The method of embodiment 1 or 2, wherein the insect host cell comprises an insect cell or cell line.
4. The method of embodiment 3, wherein the insect host cell is chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.
5. The method of embodiment 3 or 4, wherein the insect cell is a Spodoptera frugiperda cell, optionally wherein the Spodoptera frugiperda cell is an Sf9 cell.
6. The method of embodiment 3 or 4, wherein the insect cell is a Trichoplusia ni cell, optionally wherein the insect cell is a H5 cell (High Five™, Invitrogen, Sorrento, CA).
7. A method of making a purified tRNA effector molecule (TREM) composition, e.g., a TREM pharmaceutical composition, comprising:
   providing a fungal host cell comprising an exogenous nucleic acid, e.g., a DNA or RNA, encoding the TREM;
   maintaining the fungal host cell under conditions sufficient to express the TREM;
   purifying the TREM from the fungal host cell, e.g., according to a method described herein; and
   formulating the purified TREM as a pharmaceutical composition, e.g., by combining the TREM with a pharmaceutical excipient,
   thereby making the TREM composition.
8. The method of embodiment 1 or 7, wherein the fungal host cell comprises a fungal cell or cell line.
9. The method of embodiment 8, wherein the fungal cell or cell line is chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.
10. The method of embodiment 9, wherein the fungal cell or cell line is a Saccharomyces cell or cell line.
11. The method of embodiment 9, wherein the fungal cell or cell line is a Saccharomyces cerevisiae fungal cell or cell line.
12. The method of embodiment 9, wherein the fungal cell or cell line is a Schizosaccharomyces pombe fungal cell or cell line.
13. The method of embodiment 9, wherein the fungal cell or cell line is a Candida cylindracea fungal cell or cell line.
14. The method of embodiment 9, wherein the fungal cell or cell line is a Candida albicans fungal cell or cell line.
15. The method of embodiment 9, wherein the fungal cell or cell line is a Neurospora crassa fungal cell or cell line.
16. The method of embodiment 9, wherein the fungal cell or cell line is a Pichia jadinii fungal cell or cell line.
17. A method of making a purified tRNA effector molecule (TREM) composition, e.g., a TREM pharmaceutical composition, comprising:
   providing a plant host cell comprising an exogenous nucleic acid, e.g., a DNA or RNA, encoding the TREM;
   maintaining the plant host cell under conditions sufficient to express the TREM;
   purifying the TREM from the plant host cell, e.g., according to a method described herein; and
   formulating the purified TREM as a pharmaceutical composition, e.g., by combining the TREM with a pharmaceutical excipient,
   thereby making the TREM composition.
18. The method of embodiment 1 or 17, wherein the plant host cell comprises a plant, plant cell or cell line.
19. The method of embodiment 18, wherein the host plant, plant cell or cell line is a monocotyledonous plant, cell or cell line.
20. The method of embodiment 18, wherein the host plant, plant cell or cell line is a dicotyledonous plant, cell or cell line.
21. The method of embodiment 18, wherein the host plant, cell or cell line is a plant, cell or cell line chosen from: wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.
22. The method of embodiment 18, wherein the plant, cell or cell line is an Arabidopsis plant, cell or cell line.
23. The method of embodiment 18, wherein the Arabidopsis plant, cell or cell line is an A. thaliana plant, cell or cell line.
24. A method of making a tRNA effector molecule (TREM) composition, comprising:
   (a) providing a host cell e.g., a fungal cell (e.g., a yeast cell), an insect cell or a plant cell comprising exogenous nucleic acid, e.g., a DNA or RNA, encoding a TREM under conditions sufficient to express the TREM, and
   (b) purifying the expressed TREM from the host cell to produce a TREM composition, thereby making the TREM composition.
25. The method of embodiment 24, the TREM composition is formulated as a pharmaceutical composition, e.g., by combining the TREM with a pharmaceutical excipient.
26. A method of making a pharmaceutical TREM composition comprising:
   combining
   a) a TREM, e.g., a purified TREM composition, e.g., a TREM composition made by a method described herein; and
   b) a pharmaceutically acceptable component, e.g., an excipient, thereby making a pharmaceutical TREM composition.
27. The method of claim 26, wherein the TREM is purified from a host cell e.g., a fungal cell (e.g., a yeast cell), an insect cell or a plant cell, e.g., according to a method described herein.
28. A method of making a purified tRNA effector molecule (TREM) pharmaceutical composition, comprising:
   purifying the TREM from a host cell, e.g., a fungal cell (e.g., a yeast cell), an insect cell or a plant cell;
   formulating the purified TREM as a pharmaceutical composition, e.g., by combining the TREM with a pharmaceutical excipient,
   thereby making the TREM pharmaceutical composition.
29. The method of claim 27 or 28, wherein the host cell comprises an exogenous nucleic acid, e.g., a DNA or RNA, encoding the TREM.
30. The method of any one of embodiments 1-29, wherein the purification step comprises one, two or all of the following steps, e.g., in the order recited:
   (i) separating nucleic acids from protein to provide an RNA preparation;
   (ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation;
   (iii) separating a TREM from other RNA species in the small RNA preparation by affinity-based separation, e.g., sequence affinity.
31. The method of embodiment 30, comprising step (i).
32. The method of embodiment 30 or 31, comprising step (ii).
33. The method of any one of embodiments 30 to 32, comprising step (iii).
34. The method of any one of embodiments 30, or 32-33, comprising performing: step (i) before step (ii).
35. The method of any one of embodiments 30, or 32-33 comprising performing step (ii) before step (iii).
36. The method of any one of embodiments 30-35, wherein (i) comprises extracting the nucleic acids from protein.
37. The method of any one of embodiments 30-36, wherein (i) comprises a phenol/chloroform extraction.
38. The method of any one of embodiments 30-32 or 34-37, wherein (ii) comprises separating RNA of less than a first size class from RNA of a second, larger, size class.
39. The method of embodiment 38, wherein the first size class is less than 200 nt.
40. The method of any one of embodiments 30-39, wherein (ii) comprises performing a salt precipitation to enrich for RNA of less than 200 nt.
41. The method of embodiment 40, wherein the salt comprises LiCl.
42. The method of any one of embodiments 30-32 or 34-41, wherein (ii) further comprises performing a desalting or buffer exchange step.
43. The method of any one of embodiments 30 or 33-42, wherein (iii) comprises performing an affinity-based separation to enrich for a TREM.
44. The method of embodiment 43, wherein the affinity-based separation comprises a sequence based separation, e.g., using a probe comprising a sequence that binds to a TREM.
45. The method of any one of the preceding embodiments, wherein the TREM comprises a modification characteristic of a fungal (e.g., yeast), insect, or plant cell.
46. The method of embodiment 45, wherein the modification is not made in mammalian cells, or is made at a different site or at a different level as compared with a fungal (e.g., yeast), insect, or plant cell.
47. The method of embodiment 45 or 46, wherein the modification is chosen from a modification listed in any one of Tables 2-4.
48. A TREM comprising a modification characteristic of a fungal (e.g., yeast) cell or cell line e.g., as described herein.
49. The TREM of claim 48, wherein the modification is not made in mammalian cells, or is made at a different site or at a different level as compared with a fungal (e.g., yeast) cell or cell line.
50. The TREM of claim 49, wherein the modification is chosen from a modification provided in Table 3.
51. A TREM comprising a modification characteristic of an insect cell or cell line, e.g., as described herein.
52. The TREM of claim 51, wherein the modification is not made in mammalian cells, or is made at a different site or at a different level as compared with an insect or cell line.
53. The TREM of claim 52, wherein the modification is chosen from a modification provided in Table 2.
54. A TREM comprising a modification characteristic of a plant, plant cell or cell line e.g., as described herein.
55. The TREM of claim 54 wherein the modification is not made in mammalian cells, or is made at a different site or at a different level as compared with a plant, plant cell or cell line.
56. The TREM of claim 55, wherein the modification is chosen from a modification provided in Table 3.
57. The method of any one of embodiments 26-47, wherein the host cell comprises a host plant, cell or cell line.
58. The method of embodiment 57, wherein the host plant, plant cell or cell line is a monocotyledonous plant, cell or cell line.
59. The method of embodiment 57, wherein the host plant, plant cell or cell line is a dicotyledonous plant, cell or cell line.
60. The method of embodiment 57, wherein the host plant, cell or cell line is a plant, cell or cell line chosen from: wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.
61. The method of embodiment 57, wherein the plant, cell or cell line is an Arabidopsis plant, cell or cell line.
62. The method of embodiment 57, wherein the Arabidopsis plant, cell or cell line is an A. thaliana plant, cell or cell line.
63. The method of any one of embodiments 26-47, wherein the host cell comprises a fungal cell or cell line.
64. The method of embodiment 63, wherein the fungal cell or cell line is chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.
65. The method of embodiment 63, wherein the fungal cell or cell line is a Saccharomyces cell or cell line.
66. The method of embodiment 63, wherein the fungal cell or cell line is a Saccharomyces cerevisiae fungal cell or cell line.
67. The method of embodiment 63 wherein the fungal cell or cell line is a Schizosaccharomyces pombe fungal cell or cell line.
68. The method of embodiment 63, wherein the fungal cell or cell line is a Candida cylindracea fungal cell or cell line.
69. The method of embodiment 63, wherein the fungal cell or cell line is a Candida albicans fungal cell or cell line.
70. The method of embodiment 63, wherein the fungal cell or cell line is a Neurospora crassa fungal cell or cell line.
71. The method of embodiment 63, wherein the fungal cell or cell line is a Pichia jadinii fungal cell or cell line.
72. The method of any one of embodiments 26-47, wherein the host cell comprises an insect cell or cell line.
73. The method of embodiment 72 wherein the insect host cell is chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.
74. The method of embodiment 72, wherein the insect cell is a Spodoptera frugiperda cell, optionally wherein the Spodoptera frugiperda cell is an Sf9 cell.
75. The method of embodiment 72, wherein the insect cell is a Trichoplusia ni cell, optionally wherein the insect cell is a H5 cell (High Five™, Invitrogen, Sorrento, CA).
76. The method of any one of the preceding embodiments, wherein the TREM composition is a pharmaceutically acceptable composition.
77. The method of any one of the preceding embodiments, comprising introducing the exogenous DNA or RNA into the host cell.
78. The method of any one of the preceding embodiments, wherein the nucleic acid comprises a DNA, which upon transcription, expresses a TREM.
79. The method of any one of the preceding embodiments, wherein the nucleic acid comprises an RNA, which upon reverse transcription, results in a DNA which can be transcribed to provide the TREM.
80. The method of any one of the preceding embodiments, wherein the TREM mediates acceptance and incorporation of an amino acid.
81. The method of any one of the preceding embodiments, wherein the TREM comprises:
   (i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
   (ii) an RNA sequence comprising a consensus sequence provided herein.
82. The method of any one of the preceding embodiments, wherein the TREM composition comprises a TREM fragment, e.g., as described herein, optionally wherein the TREM fragment is produced in vivo, in the host cell.
83. The method of embodiment 82, wherein the TREM fragment is produced by fragmenting an expressed TREM after production of the TREM by the cell, e.g., a TREM produced by the host cell is fragmented after release or purification from the host cell, e.g., the TREM is fragmented ex vivo.
84. The method of any one of the preceding embodiments, wherein the method results in an increase, e.g., at least a 2.2, 2.5, 3, 4, 5, 6, 7, 8, 9, 10, or 20-fold increase in the production of total endogenous tRNA and TREM in the host cell, e.g., as compared with a reference cell, e.g., a similar cell but not engineered or modified to express a TREM.
85. The method of embodiment 84, wherein the method results in an increase in TREM production and/or tRNA production between 2.2 to 20-fold, between 2.2 to 15-fold, between 2.2 to 10-fold, between 2.2 to 9-fold, between 2.2 to 8-fold, between 2.2 to 7-fold, between 2.2 to 6-fold, between 2.2 to 5-fold, between 2.2 to 4-fold, between 2.2 to 3-fold, between 2.2 to 2.5-fold, between 2.5 to 20-fold, between 3 to 20-fold, between 4 to 20-fold, between 5 to 20-fold, between 6 to 20-fold, between 7 to 20-fold, between 8 to 20-fold, between 9 to 20-fold, between 10 to 20-fold, or between 15 to 20-fold.
86. The method of any one of the preceding embodiments, wherein the method results in a detectable level of TREM in the host cell, e.g., as measured by an assay described in any of Examples 1-4.
87. The method of any one of the preceding embodiments, wherein the host cell is capable of a post-transcriptional modification, of the TREM.
88. The method of any one of the preceding embodiments, wherein the host cell is capable of a post-transcriptional modification, of the TREM, e.g., a post-transcriptional modification selected from any one of Tables 2-4.
89. The method of any one of the preceding embodiments, wherein the host cell has been modified to modulate, e.g., increase, its ability to provide a post-transcriptional modification, of the TREM, e.g., a post-transcriptional modification selected from Table 2-4, e.g., the host cell has been modified to provide for, an increase, or decrease in, the expression of a gene, e.g., a gene encoding an enzyme from Table 2, or a gene encoding an enzyme having nuclease activity (e.g., endonuclease activity or ribonuclease activity), e.g., or one or more of Dicer, Angiogenin, RNaseA, RNaseP, RNaseZ, Rny1 or PrrC.
90. The method of any one of the preceding embodiments, comprising culturing the host cell in a medium that has an excess of nutrients, e.g., is not nutrient limiting.
91. The method of any one of the preceding embodiments, further comprising measuring one or more of the following characteristics of the TREM composition (or an intermediate in the production of a TREM composition):
   (i) purity of at least 30%, 40%, 50%, 60%, 70%, 80%, 85%, 90%, 91%, 92%, 93%, 94%, 95%, 96%, 97%, 98%, or 99%;
   (ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
   (iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (v) fragments of less than 0.1%, 1%, 2%, 3%, 4%, 5%, 6%, 7%, 8%, 9% or 10%;
   (vi) low levels or absence of endotoxins, e.g., a negative result as measured by the Limulus amebocyte lysate (LAL) test;
   (vii) in-vitro translation activity, e.g., as measured by an assay described in Example 10;
   (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
   (ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>;
   (x) viral contamination, e.g., the composition or preparation has an absence of or an undetectable level of viral contamination; or
   (xi) differential modification, e.g., comprising a modification characteristic of a fungal cell or cell line; an insect cell or cell line; or a plant, plant cell or cell line.
92. The method of embodiment 91, further comprising, comparing the measured value with a reference value or a standard.
93. The method of embodiment 92, further comprising, in response to the comparison, modulating the TREM composition to:
   (i) increase the purity of the TREM composition;
   (ii) decrease the amount of HCP in the composition;
   (iii) decrease the amount of DNA in the composition;
   (iv) decrease the amount of fragments in the composition;
   (v) decrease the amount of endotoxins in the composition;
   (vi) increase the in vitro translation activity of the composition;
   (vii) increase the TREM concentration of the composition; or
   (viii) increase the sterility of the composition.
94. A method of making a TREM composition, comprising:
   contacting a TREM containing a reaction mixture with a reagent, e.g., a capture reagent or a separation reagent, comprising a nucleic acid sequence complimentary with a TREM;
   thereby making a TREM composition.
95. The method of embodiment 94, further comprising, denaturing a TREM, e.g., prior to hybridization with the capture reagent.
96. The method of embodiment 94, further comprising, renaturing a TREM, e.g., after hybridization and/or release from the capture reagent.
97. The method of any of embodiments 94-96, further wherein a single capture reagent is used, e.g., to make a TREM composition, wherein at least 50%, 55%, 60%, 65%, 70%, 75%, 80%, 85%, 90%, or 95% of the TREMs have a sequence complimentary with the capture reagent.
98. The method of any of embodiments 94-96, further wherein a plurality of capture reagents are used, e.g., to make a TREM composition having a plurality of different TREMs.
99. The method of any of embodiments 94-97, wherein the TREM is made according to a method described herein, e.g., by expression in a host cell, e.g., a fungal cell, an insect cell or a plant cell.
100. A method of making a pharmaceutical composition, comprising:
   a) providing a purified TREM composition, e.g., a purified TREM composition made by culturing a host cell e.g., a fungal cell (e.g., a yeast cell), an insect cell or a plant cell, comprising DNA or RNA encoding a TREM under conditions sufficient to express the TREM, and purifying the expressed TREM from the host cell culture to produce a purified TREM composition,
   b) providing a value, e.g., by evaluating or testing, for one or more of the following characteristics of the purified TREM composition:
      (i) purity of at least 30%, 40%, 50%, 60%, 70%, 80%, 85%, 90%, 91%, 92%, 93%, 94%, 95%, 96%, 97%, 98%, or 99%;
      (ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
      (iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
      (iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
      (v) less than 0.1%, 0.5%, 1%, 2%, 3%, 4%, 5%, 6%, 7%, 8%, 9% or 10% TREM fragments relative to full length TREMs;
      (vi) low levels or absence of endotoxins, e.g., a negative result as measured by the Limulus amebocyte lysate (LAL) test;
      (vii) in-vitro translation activity, e.g., as measured by an assay described in Example 10;
      (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
      (ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>;
      (x) viral contamination, e.g., the composition or preparation has an absence of, or an undetectable level of viral contamination; or
      (xi) differential modification, e.g., comprising a modification characteristic of a fungal cell or cell line; an insect cell or cell line; or a plant, plant cell or cell line,
   c) optionally, formulating the purified TREM composition as a pharmaceutical drug product (e.g., combining the TREM composition with a pharmaceutical excipient) if it meets a reference criteria for the one or more characteristics, thereby making a pharmaceutical composition.
101. The method of embodiment 100, further comprising, comparing the measured value with a reference value or a standard.
102. The method of embodiment 101, further comprising, in response to the comparison, modulating the composition to:
   (i) increase the purity of the TREM composition;
   (ii) decrease the amount of HCP in the composition;
   (iii) decrease the amount of DNA in the composition;
   (iv) decrease the amount of fragments in the composition;
   (v) decrease the amount of endotoxins in the composition;
   (vi) increase the in vitro translation activity of the composition;
   (vii) increase the TREM concentration of the composition; or
   (viii) increase the sterility of the composition.
103. A composition comprising a purified tRNA effector molecule (TREM) (e.g., a purified TREM composition made according to a method described herein), comprising:
   (i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
   (ii) an RNA sequence comprising a consensus sequence provided herein, and optionally the RNA sequence is less than 100% identical to an RNA sequence encoded by a DNA sequence listed in Table 1.
104. A GMP-grade, recombinant TREM composition (e.g., a TREM composition made in compliance with cGMP, and/or in accordance with similar requirements) comprising:
   (i) an RNA sequence at least 80% ((e.g., at least 85%, at least 90%, at least 95%, at least 97%, at least 98%, at least 99%) identical to an RNA sequence encoded by a DNA sequence listed in Table 1, or a fragment or functional fragment thereof; or
   (ii) an RNA sequence comprising a consensus sequence provided herein, and optionally the RNA sequence is less than 100% identical to an RNA sequence encoded by a DNA sequence listed in Table 1.
105. A pharmaceutical tRNA effector molecule (TREM) composition, comprising
   (i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
   (ii) an RNA sequence comprising a consensus sequence provided herein, and optionally the RNA sequence is less than 100% identical to an RNA sequence encoded by a DNA sequence listed in Table 1.
106. The pharmaceutical TREM composition of claim 105, comprising a purified tRNA effector molecule (TREM) (e.g., a purified TREM composition made according to a method described herein).
107. The composition or pharmaceutical composition of any one of embodiments 103-106, wherein the TREM is made according to any one of embodiments 1-105.
108. A recombinant TREM composition of at least 0.5 g, 1 g, 2 g, 3 g, 4 g, 5 g, 6 g, 7 g, 8 g, 9 g, 10 g, 15 g, 20 g, 30 g, 40 g, 50 g, 100 g, 200 g, 300 g, 400 g or 500 g.
109. A recombinant TREM composition of between 0.5 g to 500 g, between 0.5 g to 400 g, between 0.5 g to 300 g, between 0.5 g to 200 g, between 0.5 g to 100 g, between 0.5 g to 50 g, between 0.5 g to 40 g, between 0.5 g to 30 g, between 0.5 g to 20 g, between 0.5 g to 10 g, between 0.5 g to 9 g, between 0.5 g to 8 g, between 0.5 g to 7 g, between 0.5 g to 6 g, between 0.5 g to 5 g, between 0.5 g to 4 g, between 0.5 g to 3 g, between 0.5 g to 2 g, between 0.5 g to 1 g, between 1 g to
500 g, between 2 g to 500 g, between 5 g to 500 g, between 10 g to 500 g, between 20 g to 500 g, between 30 g to 500 g, between 40 g to 500 g, between 50 g to 500 g, between 100 g to 500 g, between 200 g to 500 g, between 300 g to 500 g, or between 400 g to 500 g.
110. A TREM composition comprising a consensus sequence of Formula I zzz,
   R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein:
      R is a ribonucleotide residue;
      (i) zzz indicates any of the twenty amino acids;
      (ii) Formula I corresponds to all species; and
      (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
111. A TREM composition comprising a consensus sequence of Formula II zzz,
   R₀-R₁-R₂-R₃-R₄-R₅-R₆-R₇-R₈-R₉-R₁₀-R₁₁-R₁₂-R₁₃-R₁₄-R₁₅-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein:
      R is a ribonucleotide residue;
      (i) zzz indicates any of the twenty amino acids;
      (ii) Formula II corresponds to mammals; and
      (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3,x=4,x=5,x=6,x=7,x=8,x=9,x=10,x=11,x=12,x=13,x=14,x=15,x=16,x=17,x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
112. A TREM composition comprising a consensus sequence of Formula III zzz,
   R₀-R₁-R₂-R₃-R₄-R₅-R₆-R₇-R₈-R₉-R₁₀-R₁-R₁₂-R₁₃-R₁₄-R₁₅-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein:
      R is a ribonucleotide residue;
      (i) zzz indicates any of the twenty amino acids;
      (ii) Formula III corresponds to humans; and
      (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
113. The composition or pharmaceutical composition of any one of embodiments 105-112, wherein the composition comprises one or more, e.g., a plurality, of TREMs.
114. The composition or pharmaceutical composition of any one of embodiments 105-113, wherein the composition comprises one or more unique TREMs, e.g., one or more TREMs that comprise different anti-codon sequences.
115. The composition or pharmaceutical composition of any one of embodiments 105-114, wherein the composition comprises one or more unique TREMs, e.g., TREMs that recognize different codons.
116. The composition or pharmaceutical composition of any one of embodiments 105-115, wherein the TREM composition (or an intermediate in the production of a TREM composition) comprises one or more of the following characteristics:
   (i) purity of at least 30%, 40%, 50%, 60%, 70%, 80%, 85%, 90%, 91%, 92%, 93%, 94%, 95%, 96%, 97%, 98%, or 99%;
   (ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
   (iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (v) less than 0.1%, 0.5%, 1%, 2%, 3%, 4%, 5%, 6%, 7%, 8%, 9% or 10% TREM fragments relative to full length TREMs;
   (vi) low levels or absence of endotoxins, e.g., a negative result as measured by the Limulus amebocyte lysate (LAL) test;
   (vii) in-vitro translation activity, e.g., as measured by an assay described in Example 10; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
   (ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>; or
   (x) viral contamination, e.g., the composition or preparation has an absence of, or an undetectable level of viral contamination.
117. A method of modulating a tRNA pool in a cell, e.g., a mammalian cell, comprising:
   providing a purified TREM composition, e.g., as described herein, and contacting the cell with the TREM composition,
   thereby modulating the tRNA pool in the cell.
118. A method of contacting a cell, tissue, or subject, e.g., a mammalian cell, tissue, or subject, with a TREM, comprising
   contacting the cell, tissue or subject with a purified TREM composition, e.g., as described herein, thereby contacting a cell, tissue, or subject with the TREM.
119. A method of presenting a TREM to a cell, tissue, or subject, e.g., a mammalian cell, tissue, or subject, with a TREM, comprising
   contacting the cell, tissue or subject with a purified TREM composition, e.g., as described herein, thereby presenting the TREM to a cell, tissue, or subject.
120. A method of forming a TREM-contacted cell, tissue, or subject, e.g., TREM-contacted mammalian cell, tissue, or subject, comprising
   contacting the cell, tissue or subject with a purified TREM composition, e.g., as described herein, thereby forming a TREM-contacted cell, tissue, or subject.
121. A method of using a TREM comprising,
   contacting a cell, tissue or subject e.g., a mammalian cell, tissue, or subject, with a purified TREM composition, e.g., as described herein, thereby using the TREM.
122. A method of applying a TREM to a cell, tissue, or subject, e.g., a mammalian cell, tissue, or subject, comprising
   contacting the cell, tissue or subject with a purified TREM composition, e.g., as described herein, thereby applying a TREM to a cell, tissue, or subject.
123. A method of exposing a cell, tissue, or subject, e.g., a mammalian cell, tissue, or subject, to a TREM, comprising
   contacting the cell, tissue or subject with a purified TREM composition, e.g., as described herein, thereby exposing a cell, tissue, or subject to a TREM.
124. A method of forming an admixture of a TREM and a cell, tissue, or subject, e.g., a mammalian cell, tissue, or subject, comprising
   contacting the cell, tissue or subject with a TREM composition, e.g., as described herein, thereby forming an admixture of a TREM and a cell, tissue, or subject.
125. A method of delivering a TREM to a cell, tissue, or subject, e.g., a mammalian cell, tissue, or subject, comprising:
   providing a cell, tissue, or subject, and contacting the cell, tissue, or subject, with a TREM composition, e.g., a purified TREM composition, e.g., as described herein, e.g., a pharmaceutical TREM composition.
126. A method, e.g., an ex vivo method, of modulating the metabolism, e.g., the translational capacity of an organelle, e.g., of a mammalian cell, comprising:
   providing a preparation of an organelle, e.g., mitochondria or chloroplasts, and contacting the organelle with a TREM composition, e.g., as described herein.
127. A method of treating a subject, e.g., modulating the metabolism, e.g., the translational capacity of a cell, e.g., mammalian cell, in a subject, comprising:
   providing, e.g., administering to the subject, an exogenous nucleic acid, e.g., a DNA or RNA, which encodes a TREM, thereby treating the subject.
128. The method of any one of embodiments 117-127, wherein the TREM composition is made according to any one of embodiments 1-102, or the TREM comprises a composition provided in any one of embodiments 103-116.
129. The method of any one of embodiments 117-127, wherein the TREM composition is made by:
   providing a host cell e.g., a fungal cell (e.g., a yeast cell), an insect cell or a plant cell, comprising an exogenous nucleic acid, e.g., a DNA or RNA, encoding the TREM;
   maintaining the cell under conditions sufficient to express the TREM; and/or
   purifying the TREM from the host cell, e.g., according to a method described herein.
130. The method of embodiment 129, wherein the host cell comprises a host plant, cell or cell line.
131. The method of embodiment 130, wherein the host plant, plant cell or cell line is a monocotyledonous plant, cell or cell line.
132. The method of embodiment 130, wherein the host plant, plant cell or cell line is a dicotyledonous plant, cell or cell line.
133. The method of embodiment 130, wherein the host plant, cell or cell line is a plant, cell or cell line chosen from: wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.
134. The method of embodiment 130, wherein the plant, cell or cell line is an Arabidopsis plant, cell or cell line.
135. The method of embodiment 130, wherein the Arabidopsis plant, cell or cell line is an A. thaliana plant, cell or cell line.
136. The method of embodiment 129, wherein the host cell comprises a fungal cell or cell line.
137. The method of embodiment 136, wherein the fungal cell or cell line is chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.
138. The method of embodiment 136, wherein the fungal cell or cell line is a Saccharomyces cell or cell line.
139. The method of embodiment 136, wherein the fungal cell or cell line is a Saccharomyces 10 cerevisiae fungal cell or cell line.
140. The method of embodiment 136 wherein the fungal cell or cell line is a Schizosaccharomyces pombe fungal cell or cell line.
141. The method of embodiment 136, wherein the fungal cell or cell line is a Candida cylindracea fungal cell or cell line.
142. The method of embodiment 136, wherein the fungal cell or cell line is a Candida albicans fungal cell or cell line.
143. The method of embodiment 136, wherein the fungal cell or cell line is a Neurospora crassa fungal cell or cell line.
144. The method of embodiment 136, wherein the fungal cell or cell line is a Pichia jadinii fungal cell or cell line.
145. The method of embodiment 129, wherein the host cell comprises an insect cell or cell line.
146. The method of embodiment 145 wherein the insect host cell is chosen from Autographa 30 californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.
147. The method of embodiment 145, wherein the insect cell is a Spodoptera frugiperda cell, optionally wherein, the Spodoptera frugiperda cell is an Sf9 cell.
148. The method of embodiment 145, wherein the insect cell is a Trichoplusia ni cell, optionally wherein the insect cell is a H5 cell (High Five™, Invitrogen, Sorrento, CA).
149. The method of any one of embodiments 129-148, wherein the purification step comprises one, two or all of the following steps, e.g., in the order recited:
   (i) separating nucleic acids from cellular debris to provide an RNA preparation;
   (ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
   (iii) separating a TREM from other RNA species in the small RNA preparation by affinity-based separation, e.g., sequence affinity-based separation.
150. The method of any one of embodiments 117-149, wherein the TREM comprises:
   (i) an RNA sequence at least 80% (e.g., at least 85%, at least 90%, at least 95%, at least 97%, at least 98%, at least 99%) identical to an RNA sequence encoded by a DNA sequence listed in Table 1, or a fragment or functional fragment thereof; or
   (ii) an RNA sequence comprising a consensus sequence provided herein, and optionally the RNA sequence is less than 100% identical to an RNA sequence encoded by a DNA sequence listed in Table 1.
151. The method of any one of embodiments 117-150, wherein the method is an in vitro method, e.g., a mammalian cell or tissue, is contacted with the TREM composition in vitro.
152. The method of any one of embodiments 117-150, wherein the method is an ex vivo method, e.g., a cell or tissue, is contacted with the TREM composition ex vivo, and optionally, the contacted cell or tissue is introduced, e.g., administered, into a subject, e.g., the subject from which the cell or tissue came, or a different subject, optionally wherein the subject is a mammal.
153. The method of any one of embodiments 117-150, wherein the method is an in vivo method, e.g., a subject, or a tissue or cell of a subject, is contacted with the TREM composition in vivo, optionally wherein the subject is a mammal.
154. The method of any of embodiments 151-153, comprising contacting the TREM composition, e.g., a pharmaceutical TREM composition, with a cell, e.g., mammalian cell.
155. The method of any of embodiments 151-153, comprising contacting the TREM composition, e.g., a pharmaceutical TREM composition, with a tissue, e.g., mammalian tissue.
156. The method of any of embodiments 151-152 or 154, comprising administering the TREM composition, e.g., a pharmaceutical TREM composition, to a subject, e.g., mammal.
157. The method of any of embodiments 152 or 156, wherein the TREM composition is administered with a carrier or delivery agent, e.g., a liposome, a polymer (e.g., a polymer conjugate), a particle, a microsphere, microparticle, or a nanoparticle.
158. The method of any of embodiments 152 or 156, wherein the TREM composition is administered without a carrier or delivery agent.
159. The method of any of embodiments 151-158, wherein the cell is cancerous.
160. The method of any of embodiments 151-158, wherein the cell is noncancerous.
161. The method of any of embodiments 151-154 or 156-160, wherein the cell or tissue comprises:
   a muscle cell or tissue (e.g., a skeletal muscle cell or tissue, a smooth muscle cell or tissue, or a cardiac muscle cell or tissue),
   an epithelial cell or tissue;
   a connective cell or tissue (e.g., adipose cell or tissue, bone cell or tissue, or blood cell), or
   a nervous cell or tissue (e.g., a sensory neuron, a motor neuron, or an interneuron).
162. The method of any of embodiments 151-161, wherein the method comprises administering a cell that was contacted ex vivo or in vitro, with a TREM composition, to a subject.
163. A cell comprising a TREM made according to any one of embodiments 1-102.
164. A cell comprising a TREM of any one of embodiments 103-116.
165. A cell, e.g., a host cell, e.g., a fungal cell, an insect cell or a plant cell, comprising an exogenous nucleic acid comprising:
   a nucleic acid sequence, e.g., DNA or RNA, that encodes a TREM, wherein the nucleic acid sequence comprises:
      (i) a control region sequence;
      (ii) a sequence encoding a modified TREM;
      (iii) a sequence encoding more than one TREM; or
      (iv) a promoter sequence that comprises a Pol III recognition site, e.g., a U6 promoter, a 7 SK promoter or a H1 promoter, or a fragment thereof.
166. A reaction mixture comprising a TREM and a reagent, e.g., a capture reagent, or a separation reagent.
167. A bioreactor comprising a plurality of host cells described herein comprising exogenous DNA or RNA encoding a TREM.
168. The bioreactor of embodiment 167,
   (i) comprising at least 1×10⁷, 1×10⁸, 1×10⁹, 1×10¹⁰, 1×10¹¹, 1×10¹², 1×10¹³, or 1×10¹⁴ host cells;
   (ii) comprising between 100 mL and 100 liters of culture medium, e.g., at least 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, or 100 liters of culture medium;
   (iii) wherein the bioreactor is selected from a continuous flow bioreactor, a batch process bioreactor, a perfusion bioreactor, and a fed batch bioreactor; or
   (iv) wherein the bioreactor is held under conditions sufficient to express the TREM.
169. A method of evaluating a composition of TREM, e.g., a GMP-grade TREM (i.e., a TREM made in compliance with cGMP, and/or in accordance with similar requirements), comprising acquiring a value for one or more of the following characteristics of the purified TREM composition:
   (i) purity of at least 30%, 40%, 50%, 60%, 70%, 80%, 85%, 90%, 91%, 92%, 93%, 94%, 95%, 96%, 97%, 98%, or 99%;
   (ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
   (iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (v) less than 0.1%, 0.5%, 1%, 2%, 3%, 4%, 5%, 6%, 7%, 8%, 9% or 10% TREM fragments relative to full length TREMs;
   (vi) low levels or absence of endotoxins, e.g., a negative result as measured by the Limulus amebocyte lysate (LAL) test;
   (vii) in-vitro translation activity, e.g., as measured by an assay described in Example 10;
   (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
   (ix) sterility, e.g., the composition or preparation supports the growth of fewer than 100 viable microorganisms as tested under aseptic conditions, the composition or preparation meets the standard of USP<71>, and/or the composition or preparation meets the standard of USP<85> as described by cGMP guidelines for sterile drug products produced by aseptic processing;
   (x) viral contamination, e.g., the composition or preparation has an absence of, or an undetectable level of viral contamination; or
   (xi) differential modification, e.g., comprising a modification characteristic of a fungal cell or cell line; an insect cell or cell line; or a plant, plant cell or cell line.
170. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the nucleic acid sequence comprises a promoter sequence, e.g., a promoter sequence described herein, e.g., a promoter sequence that is active, e.g., expressed, in the host cell, e.g., the fungal host cell, insect host cell or plant host cell.
171. The method, composition or pharmaceutical composition, cell, reaction mixture, bioreactor, or master cell bank of embodiment 170, wherein the nucleic acid sequence comprises a promoter sequence that comprises an RNA polymerase III (Pol III) recognition site, e.g., a Pol III binding site, e.g., a U6 promoter sequence or fragment thereof.
172. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM enhances:
   (a) the stability of a product, e.g., a protein, and/or
   (b) ribosome occupancy of a product.
173. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167 wherein the TREM:
   modulates ribosome occupancy;
   modulates protein translation or stability;
   modulates mRNA stability;
   modulates protein folding or structure;
   modulates protein transduction or compartmentalization;
   modulates codon usage;
   modulates cell fate; or
   modulates a signaling pathway, e.g., a cellular signaling pathway.
174. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM comprises cognate adaptor function, and wherein the TREM mediates acceptance and incorporation of an amino acid associated in nature with the anti-codon of the TREM in the initiation or elongation of a peptide chain.
175. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM comprises non-cognate adaptor function, and wherein the TREM mediates acceptance and incorporation of an amino acid, e.g., a non-cognate amino acid, other than the amino acid associated in nature with the anti-codon of the TREM, in the initiation or elongation of a peptide chain, and the non-cognate amino acid residue is, e.g., a desired residue, e.g., a residue that does not mediate a disorder or unwanted trait, e.g., a wild type residue.
176. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM comprises an anti-codon sequence which is complimentary with a codon which
   specifies a first amino acid residue, e.g., an unwanted or undesired codon, e.g., a codon associated with a disorder or unwanted trait, e.g., a mutant codon, and
   the TREM mediates incorporation of a second amino acid residue, e.g., a desired codon, e.g., an amino acid not associated with a disorder or unwanted trait, e.g., a wild type amino acid.
177. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM comprises an RNA sequence at least 80% (e.g., at least 85%, at least 90%, at least 95%, at least 97%, at least 98%, at least 99%) identical to an RNA sequence of a tRNA which occurs naturally.
178. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM comprises an RNA sequence at least 80% (e.g., at least 85%, at least 90%, at least 95%, at least 97%, at least 98%, at least 99%) identical to an RNA encoded by a DNA sequence listed in Table 1, or a fragment or functional fragment thereof.
179. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM comprises:
   an RNA sequence encoded by a DNA sequence listed in Table 1, or a fragment thereof.
180. The method of any one of embodiments 1-102, the composition of any one of embodiments 103-116, the method of any one of embodiments 117-162, the cell of any one of embodiments 163-165, the reaction mixture of embodiment 166 or the bioreactor of embodiment 167, wherein the TREM comprises
   an RNA sequence at least XX % identical to an RNA sequence encoded by a DNA sequence listed in Table 1, or a fragment thereof, wherein XX is selected from 80, 85, 90, 95, 96, 97, 98, or 99.
181. The method, composition or pharmaceutical composition, cell, reaction mixture, or the bioreactor of embodiment 180, wherein XX is 80.
182. The method, composition or pharmaceutical composition, cell, reaction mixture, or the bioreactor of embodiment 180, wherein XX is 85.
183. The method, composition or pharmaceutical composition, cell, reaction mixture, or the bioreactor of embodiment 180, wherein XX is 90.
184. The method, composition or pharmaceutical composition, cell, reaction mixture, or the bioreactor of embodiment 180, wherein XX is 95.
185. The method, composition or pharmaceutical composition, cell, reaction mixture, or the bioreactor of embodiment 180, wherein XX is 97.
186. The method, composition or pharmaceutical composition, cell, reaction mixture, or the bioreactor of embodiment 180, wherein XX is 98.
187. The method, composition or pharmaceutical composition, cell, reaction mixture, or the bioreactor of embodiment 180, wherein XX is 99.
188. The method, composition or pharmaceutical composition, cell, reaction mixture, or the bioreactor of any one of embodiments 180-187, wherein the DNA sequence is SEQ ID NO:1 or a fragment thereof, or SEQ ID NO:2 or a fragment thereof, or SEQ ID NO: 3 or a fragment thereof, or SEQ ID NO:4 or a fragment thereof, or SEQ ID NO: 5 or a fragment thereof, or SEQ ID NO: 6 or a fragment thereof, or SEQ ID NO: 7 or a fragment thereof, or SEQ ID NO:8 or a fragment thereof, or SEQ ID NO: 9 or a fragment thereof, or SEQ ID NO:10 or a fragment thereof, or SEQ ID NO: 11 or a fragment thereof, or SEQ ID NO:12 or a fragment thereof, or SEQ ID NO: 13 or a fragment thereof, or SEQ ID NO: 14 or a fragment thereof, or SEQ ID NO: 15 or a fragment thereof, or SEQ ID NO: 16 or a fragment thereof, or SEQ ID NO: 17 or a fragment thereof, or SEQ ID NO: 18 or a fragment thereof, or SEQ ID NO: 19 or a fragment thereof, or SEQ ID NO: 20 or a fragment thereof, or SEQ ID NO: 21 or a fragment thereof, or SEQ ID NO: 22 or a fragment thereof, or SEQ ID NO: 23 or a fragment thereof, or SEQ ID NO: 24 or a fragment thereof, or SEQ ID NO: 25 or a fragment thereof, or SEQ ID NO: 26 or a fragment thereof, or SEQ ID NO: 27 or a fragment thereof, or SEQ ID NO: 28 or a fragment thereof, or SEQ ID NO: 29 or a fragment thereof, or SEQ ID NO: 30 or a fragment thereof, or SEQ ID NO: 31 or a fragment thereof, or SEQ ID NO: 32 or a fragment thereof, or SEQ ID NO: 33 or a fragment thereof, or SEQ ID NO: 34 or a fragment thereof, or SEQ ID NO: 35 or a fragment thereof, or SEQ ID NO: 36 or a fragment thereof, or SEQ ID NO: 37 or a fragment thereof, or SEQ ID NO: 38 or a fragment thereof, or SEQ ID NO: 39 or a fragment thereof, or SEQ ID NO: 40 or a fragment thereof, or SEQ ID NO: 41 or a fragment thereof, or SEQ ID NO: 42 or a fragment thereof, or SEQ ID NO: 43 or a fragment thereof, or SEQ ID NO: 44 or a fragment thereof, or SEQ ID NO: 45 or a fragment thereof, or SEQ ID NO: 46 or a fragment thereof, or SEQ ID NO: 47 or a fragment thereof, or SEQ ID NO: 48 or a fragment thereof, or SEQ ID NO: 49 or a fragment thereof, or SEQ ID NO: 50 or a fragment thereof, or SEQ ID NO: 51 or a fragment thereof, or SEQ ID NO: 52 or a fragment thereof, or SEQ ID NO: 53 or a fragment thereof, or SEQ ID NO: 54 or a fragment thereof, or SEQ ID NO: 55 or a fragment thereof, or SEQ ID NO: 56 or a fragment thereof, or SEQ ID NO: 57 or a fragment thereof, or SEQ ID NO: 58 or a fragment thereof, or SEQ ID NO: 59 or a fragment thereof, or SEQ ID NO: 60 or a fragment thereof, or SEQ ID NO: 61 or a fragment thereof, or SEQ ID NO: 62 or a fragment thereof, or SEQ ID NO: 63 or a fragment thereof, or SEQ ID NO: 64 or a fragment thereof, or SEQ ID NO: 65 or a fragment thereof, or SEQ ID NO: 66 or a fragment thereof, or SEQ ID NO: 67 or a fragment thereof, or SEQ ID NO: 68 or a fragment thereof, or SEQ ID NO: 69 or a fragment thereof, or SEQ ID NO: 70 or a fragment thereof,} or SEQ ID NO: 71 or a fragment thereof, or SEQ ID NO: 72 or a fragment thereof, or SEQ ID NO: 73 or a fragment thereof, or SEQ ID NO: 74 or a fragment thereof, or SEQ ID NO: 75 or a fragment thereof, or SEQ ID NO: 76 or a fragment thereof, or SEQ ID NO: 77 or a fragment thereof, or SEQ ID NO: 78 or a fragment thereof, or SEQ ID NO: 79 or a fragment thereof, or SEQ ID NO: 80 or a fragment thereof, or SEQ ID NO: 81 or a fragment thereof, or SEQ ID NO: 82 or a fragment thereof, or SEQ ID NO: 83 or a fragment thereof, or SEQ ID NO: 84 or a fragment thereof, or SEQ ID NO: 85 or a fragment thereof, or SEQ ID NO: 86 or a fragment thereof, or SEQ ID NO: 87 or a fragment thereof, or SEQ ID NO: 88 or a fragment thereof, or SEQ ID NO: 89 or a fragment thereof, or SEQ ID NO: 90 or a fragment thereof, or SEQ ID NO: 91 or a fragment thereof, or SEQ ID NO: 92 or a fragment thereof, or SEQ ID NO: 93 or a fragment thereof, or SEQ ID NO: 94 or a fragment thereof, or SEQ ID NO: 95 or a fragment thereof, or SEQ ID NO: 96 or a fragment thereof, or SEQ ID NO: 97 or a fragment thereof, or SEQ ID NO: 98 or a fragment thereof, or SEQ ID NO: 99 or a fragment thereof, or SEQ ID NO: 100 or a fragment thereof, or SEQ ID NO: 101 or a fragment thereof, or SEQ ID NO: 102 or a fragment thereof, or SEQ ID NO: 103 or a fragment thereof, or SEQ ID NO: 104 or a fragment thereof, or SEQ ID NO: 105 or a fragment thereof, or SEQ ID NO: 106 or a fragment thereof, or SEQ ID NO: 107 or a fragment thereof, or SEQ ID NO: 108 or a fragment thereof, or SEQ ID NO:109 or a fragment thereof, or SEQ ID NO: 110 or a fragment thereof, or SEQ ID NO: 111 or a fragment thereof, or SEQ ID NO: 112 or a fragment thereof, or SEQ ID NO: 113 or a fragment thereof, or SEQ ID NO: 114 or a fragment thereof, or SEQ ID NO: 115 or a fragment thereof, or SEQ ID NO: 116 or a fragment thereof, or SEQ ID NO: 117 or a fragment thereof, or SEQ ID NO: 118 or a fragment thereof, or SEQ ID NO: 119 or a fragment thereof, or SEQ ID NO: 120 or a fragment thereof, or SEQ ID NO: 121 or a fragment thereof, or SEQ ID NO: 122 or a fragment thereof, or SEQ ID NO: 123 or a fragment thereof, or SEQ ID NO: 124 or a fragment thereof, or SEQ ID NO: 125 or a fragment thereof, or SEQ ID NO: 126 or a fragment thereof, or SEQ ID NO: 127 or a fragment thereof, or SEQ ID NO: 128 or a fragment thereof, or SEQ ID NO: 129 or a fragment thereof, or SEQ ID NO: 130 or a fragment thereof, or SEQ ID NO: 131 or a fragment thereof, or SEQ ID NO: 132 or a fragment thereof, or SEQ ID NO: 133 or a fragment thereof, or SEQ ID NO: 134 or a fragment thereof, or SEQ ID NO: 135 or a fragment thereof, or SEQ ID NO:136 or a fragment thereof, or SEQ ID NO: 137 or a fragment thereof, or SEQ ID NO: 138 or a fragment thereof, or SEQ ID NO: 139 or a fragment thereof, or SEQ ID NO: 140 or a fragment thereof, or SEQ ID NO: 141 or a fragment thereof, or SEQ ID NO: 142 or a fragment thereof, or SEQ ID NO: 143 or a fragment thereof, or SEQ ID NO: 144 or a fragment thereof, or SEQ ID NO: 145 or a fragment thereof, or SEQ ID NO: 146 or a fragment thereof, or SEQ ID NO: 147 or a fragment thereof, or SEQ ID NO: 148 or a fragment thereof, or SEQ ID NO: 149 or a fragment thereof, or SEQ ID NO: 150 or a fragment thereof, or SEQ ID NO: 151 or a fragment thereof, or SEQ ID NO: 152 or a fragment thereof, or SEQ ID NO: 153 or a fragment thereof, or SEQ ID NO: 154 or a fragment thereof, or SEQ ID NO: 155 or a fragment thereof, or SEQ ID NO: 156 or a fragment thereof, or SEQ ID NO: 157 or a fragment thereof, or SEQ ID NO: 158 or a fragment thereof, or SEQ ID NO: 159 or a fragment thereof, or SEQ ID NO: 160 or a fragment thereof, or SEQ ID NO: 161 or a fragment thereof, or SEQ ID NO: 162 or a fragment thereof, or SEQ ID NO: 163 or a fragment thereof, or SEQ ID NO: 164 or a fragment thereof, or SEQ ID NO: 165 or a fragment thereof, or SEQ ID NO: 166 or a fragment thereof, or SEQ ID NO: 167 or a fragment thereof, or SEQ ID NO: 168 or a fragment thereof, or SEQ ID NO: 169 or a fragment thereof, or SEQ ID NO: 170 or a fragment thereof, or SEQ ID NO: 171 or a fragment thereof, or SEQ ID NO: 172 or a fragment thereof, or SEQ ID NO: 173 or a fragment thereof, or SEQ ID NO: 174 or a fragment thereof, or SEQ ID NO: 175 or a fragment thereof, or SEQ ID NO: 176 or a fragment thereof, or SEQ ID NO: 177 or a fragment thereof, or SEQ ID NO: 178 or a fragment thereof, or SEQ ID NO: 179 or a fragment thereof, or SEQ ID NO: 180 or a fragment thereof, or SEQ ID NO: 181 or a fragment thereof, or SEQ ID NO: 182 or a fragment thereof, or SEQ ID NO: 183 or a fragment thereof, or SEQ ID NO: 184 or a fragment thereof, or SEQ ID NO: 185 or a fragment thereof, or SEQ ID NO: 186 or a fragment thereof, or SEQ ID NO: 187 or a fragment thereof, or SEQ ID NO: 188 or a fragment thereof, or SEQ ID NO: 189 or a fragment thereof, or SEQ ID NO: 190 or a fragment thereof, or SEQ ID NO: 191 or a fragment thereof, or SEQ ID NO: 192 or a fragment thereof, or SEQ ID NO: 193 or a fragment thereof, or SEQ ID NO: 194 or a fragment thereof, or SEQ ID NO: 195 or a fragment thereof, or SEQ ID NO: 196 or a fragment thereof, or SEQ ID NO: 197 or a fragment thereof, or SEQ ID NO: 198 or a fragment thereof, or SEQ ID NO: 199 or a fragment thereof, or SEQ ID NO: 200 or a fragment thereof, or SEQ ID NO: 201 or a fragment thereof, or SEQ ID NO: 202 or a fragment thereof, or SEQ ID NO: 203 or a fragment thereof, or SEQ ID NO: 204 or a fragment thereof, or SEQ ID NO: 205 or a fragment thereof, or SEQ ID NO: 206 or a fragment thereof, or SEQ ID NO: 207 or a fragment thereof, or SEQ ID NO: 208 or a fragment thereof, or SEQ ID NO: 209 or a fragment thereof, or SEQ ID NO: 210 or a fragment thereof, or SEQ ID NO: 211 or a fragment thereof, or SEQ ID NO: 212 or a fragment thereof, or SEQ ID NO: 213 or a fragment thereof, or SEQ ID NO: 214 or a fragment thereof, or SEQ ID NO: 215 or a fragment thereof, or SEQ ID NO: 216 or a fragment thereof, or SEQ ID NO: 217 or a fragment thereof, or SEQ ID NO: 218 or a fragment thereof, or SEQ ID NO: 219 or a fragment thereof, or SEQ ID NO: 220 or a fragment thereof, or SEQ ID NO: 221 or a fragment thereof, or SEQ ID NO: 222 or a fragment thereof, or SEQ ID NO: 223 or a fragment thereof, or SEQ ID NO: 224 or a fragment thereof, or SEQ ID NO: 225 or a fragment thereof, or SEQ ID NO: 226 or a fragment thereof, or SEQ ID NO: 227 or a fragment thereof, or SEQ ID NO: 228 or a fragment thereof, or SEQ ID NO: 229 or a fragment thereof, or SEQ ID NO: 230 or a fragment thereof, or SEQ ID NO: 231 or a fragment thereof, or SEQ ID NO: 232 or a fragment thereof, or SEQ ID NO: 233 or a fragment thereof, or SEQ ID NO: 234 or a fragment thereof, or SEQ ID NO: 235 or a fragment thereof, or SEQ ID NO: 236 or a fragment thereof, or SEQ ID NO: 237 or a fragment thereof, or SEQ ID NO: 238 or a fragment thereof, or SEQ ID NO: 239 or a fragment thereof, or SEQ ID NO: 240 or a fragment thereof, or SEQ ID NO: 241 or a fragment thereof, or SEQ ID NO: 242 or a fragment thereof, or SEQ ID NO: 243 or a fragment thereof, or SEQ ID NO: 244 or a fragment thereof, or SEQ ID NO: 245 or a fragment thereof, or SEQ ID NO: 246 or a fragment thereof, or SEQ ID NO: 247 or a fragment thereof, or SEQ ID NO: 248 or a fragment thereof, or SEQ ID NO: 249 or a fragment thereof, or SEQ ID NO: 250 or a fragment thereof, or SEQ ID NO: 251 or a fragment thereof, or SEQ ID NO: 252 or a fragment thereof, or SEQ ID NO: 253 or a fragment thereof, or SEQ ID NO: 254 or a fragment thereof, or SEQ ID NO: 255 or a fragment thereof, or SEQ ID NO: 256 or a fragment thereof, or SEQ ID NO: 257 or a fragment thereof, or SEQ ID NO: 258 or a fragment thereof, or SEQ ID NO: 259 or a fragment thereof, or SEQ ID NO: 260 or a fragment thereof, or SEQ ID NO: 261 or a fragment thereof, or SEQ ID NO: 262 or a fragment thereof, or SEQ ID NO: 263 or a fragment thereof, or SEQ ID NO: 264 or a fragment thereof, or SEQ ID NO: 265 or a fragment thereof, or SEQ ID NO: 266 or a fragment thereof, or SEQ ID NO: 267 or a fragment thereof, or SEQ ID NO: 268 or a fragment thereof, or SEQ ID NO: 269 or a fragment thereof, or SEQ ID NO: 270 or a fragment thereof, or SEQ ID NO: 271 or a fragment thereof, or SEQ ID NO: 272 or a fragment thereof, or SEQ ID NO: 273 or a fragment thereof, or SEQ ID NO: 274 or a fragment thereof, or SEQ ID NO: 275 or a fragment thereof, or SEQ ID NO: 276 or a fragment thereof, or SEQ ID NO: 277 or a fragment thereof, or SEQ ID NO: 278 or a fragment thereof, or SEQ ID NO: 279 or a fragment thereof, or SEQ ID NO: 280 or a fragment thereof, or SEQ ID NO: 281 or a fragment thereof, or SEQ ID NO: 282 or a fragment thereof, or SEQ ID NO: 283 or a fragment thereof, or SEQ ID NO: 284 or a fragment thereof, or SEQ ID NO: 285 or a fragment thereof, or SEQ ID NO: 286 or a fragment thereof, or SEQ ID NO: 287 or a fragment thereof, or SEQ ID NO: 288 or a fragment thereof, or SEQ ID NO: 289 or a fragment thereof, or SEQ ID NO: 290 or a fragment thereof, or SEQ ID NO: 291 or a fragment thereof, or SEQ ID NO: 292 or a fragment thereof, or SEQ ID NO: 293 or a fragment thereof, or SEQ ID NO: 294 or a fragment thereof, or SEQ ID NO: 295 or a fragment thereof, or SEQ ID NO: 296 or a fragment thereof, or SEQ ID NO: 297 or a fragment thereof, or SEQ ID NO: 298 or a fragment thereof, or SEQ ID NO: 299 or a fragment thereof, or SEQ ID NO: 300 or a fragment thereof, or SEQ ID NO: 301 or a fragment thereof, or SEQ ID NO: 302 or a fragment thereof, or SEQ ID NO: 303 or a fragment thereof, or SEQ ID NO: 304 or a fragment thereof, or SEQ ID NO: 305 or a fragment thereof, or SEQ ID NO: 306 or a fragment thereof, or SEQ ID NO: 307 or a fragment thereof, or SEQ ID NO: 308 or a fragment thereof, or SEQ ID NO: 309 or a fragment thereof, or SEQ ID NO: 310 or a fragment thereof, or SEQ ID NO: 311 or a fragment thereof, or SEQ ID NO: 312 or a fragment thereof, or SEQ ID NO: 313 or a fragment thereof, or SEQ ID NO: 314 or a fragment thereof, or SEQ ID NO: 315 or a fragment thereof, or SEQ ID NO: 316 or a fragment thereof, or SEQ ID NO: 317 or a fragment thereof, or SEQ ID NO: 318 or a fragment thereof, or SEQ ID NO: 319 or a fragment thereof, or SEQ ID NO: 320 or a fragment thereof, or SEQ ID NO: 321 or a fragment thereof, or SEQ ID NO: 322 or a fragment thereof, or SEQ ID NO: 323 or a fragment thereof, or SEQ ID NO: 324 or a fragment thereof, or SEQ ID NO: 325 or a fragment thereof, or SEQ ID NO: 326 or a fragment thereof, or SEQ ID NO: 327 or a fragment thereof, or SEQ ID NO: 328 or a fragment thereof, or SEQ ID NO: 329 or a fragment thereof, or SEQ ID NO: 330 or a fragment thereof, or SEQ ID NO: 331 or a fragment thereof, or SEQ ID NO: 332 or a fragment thereof, or SEQ ID NO: 333 or a fragment thereof, or SEQ ID NO: 334 or a fragment thereof, or SEQ ID NO: 335 or a fragment thereof, or SEQ ID NO: 336 or a fragment thereof, or SEQ ID NO: 337 or a fragment thereof, or SEQ ID NO: 338 or a fragment thereof, or SEQ ID NO: 339 or a fragment thereof, or SEQ ID NO: 340 or a fragment thereof, or SEQ ID NO: 341 or a fragment thereof, or SEQ ID NO: 342 or a fragment thereof, or SEQ ID NO: 343 or a fragment thereof, or SEQ ID NO: 344 or a fragment thereof, or SEQ ID NO: 345 or a fragment thereof, or SEQ ID NO: 346 or a fragment thereof, or SEQ ID NO: 347 or a fragment thereof, or SEQ ID NO: 348 or a fragment thereof, or SEQ ID NO: 349 or a fragment thereof, or SEQ ID NO: 350 or a fragment thereof, or SEQ ID NO: 351 or a fragment thereof, or SEQ ID NO: 352 or a fragment thereof, or SEQ ID NO: 353 or a fragment thereof, or SEQ ID NO: 354 or a fragment thereof, or SEQ ID NO: 355 or a fragment thereof, or SEQ ID NO: 356 or a fragment thereof, or SEQ ID NO: 357 or a fragment thereof, or SEQ ID NO: 358 or a fragment thereof, or SEQ ID NO: 359 or a fragment thereof, or SEQ ID NO: 360 or a fragment thereof, or SEQ ID NO: 361 or a fragment thereof, or SEQ ID NO: 362 or a fragment thereof, or SEQ ID NO: 363 or a fragment thereof, or SEQ ID NO: 364 or a fragment thereof, or SEQ ID NO: 365 or a fragment thereof, or SEQ ID NO: 366 or a fragment thereof, or SEQ ID NO: 367 or a fragment thereof, or SEQ ID NO: 368 or a fragment thereof, or SEQ ID NO: 369 or a fragment thereof, or SEQ ID NO: 370 or a fragment thereof, or SEQ ID NO: 371 or a fragment thereof, or SEQ ID NO: 372 or a fragment thereof, or SEQ ID NO: 373 or a fragment thereof, or SEQ ID NO: 374 or a fragment thereof, or SEQ ID NO: 375 or a fragment thereof, or SEQ ID NO: 376 or a fragment thereof, or SEQ ID NO: 377 or a fragment thereof, or SEQ ID NO: 378 or a fragment thereof, or SEQ ID NO: 379 or a fragment thereof, or SEQ ID NO: 380 or a fragment thereof, or SEQ ID NO: 381 or a fragment thereof, or SEQ ID NO: 382 or a fragment thereof, or SEQ ID NO: 383 or a fragment thereof, or SEQ ID NO: 384 or a fragment thereof, or SEQ ID NO: 385 or a fragment thereof, or SEQ ID NO: 386 or a fragment thereof, or SEQ ID NO: 387 or a fragment thereof, or SEQ ID NO: 388 or a fragment thereof, or SEQ ID NO: 389 or a fragment thereof, or SEQ ID NO: 390 or a fragment thereof, or SEQ ID NO: 391 or a fragment thereof, or SEQ ID NO: 392 or a fragment thereof, or SEQ ID NO: 393 or a fragment thereof, or SEQ ID NO: 394 or a fragment thereof, or SEQ ID NO: 395 or a fragment thereof, or SEQ ID NO: 396 or a fragment thereof, or SEQ ID NO: 397 or a fragment thereof, or SEQ ID NO: 398 or a fragment thereof, or SEQ ID NO: 399 or a fragment thereof, or SEQ ID NO: 400 or a fragment thereof, or SEQ ID NO: 401 or a fragment thereof, or SEQ ID NO: 402 or a fragment thereof, or SEQ ID NO: 403 or a fragment thereof, or SEQ ID NO: 404 or a fragment thereof, or SEQ ID NO: 405 or a fragment thereof, or SEQ ID NO: 406 or a fragment thereof, or SEQ ID NO: 407 or a fragment thereof, or SEQ ID NO: 408 or a fragment thereof, or SEQ ID NO: 409 or a fragment thereof, or SEQ ID NO: 410 or a fragment thereof, or SEQ ID NO: 411 or a fragment thereof, or SEQ ID NO: 412 or a fragment thereof, or SEQ ID NO: 413 or a fragment thereof, or SEQ ID NO: 414 or a fragment thereof, or SEQ ID NO: 415 or a fragment thereof, or SEQ ID NO: 416 or a fragment thereof, or SEQ ID NO: 417 or a fragment thereof, or SEQ ID NO: 418 or a fragment thereof, or SEQ ID NO: 419 or a fragment thereof, or SEQ ID NO: 420 or a fragment thereof, or SEQ ID NO: 421 or a fragment thereof, or SEQ ID NO: 422 or a fragment thereof, or SEQ ID NO: 423 or a fragment thereof, or SEQ ID NO: 424 or a fragment thereof, or SEQ ID NO: 425 or a fragment thereof, or SEQ ID NO: 426 or a fragment thereof, or SEQ ID NO: 427 or a fragment thereof, or SEQ ID NO:428 or a fragment thereof, or SEQ ID NO: 429 or a fragment thereof, or SEQ ID NO: 430 or a fragment thereof, or SEQ ID NO: 431 or a fragment thereof, or SEQ ID NO: 432 or a fragment thereof, or SEQ ID NO: 433 or a fragment thereof, or SEQ ID NO: 434 or a fragment thereof, or SEQ ID NO: 435 or a fragment thereof, or SEQ ID NO: 436 or a fragment thereof, or SEQ ID NO: 437 or a fragment thereof, or SEQ ID NO: 438 or a fragment thereof, or SEQ ID NO: 439 or a fragment thereof, or SEQ ID NO: 440 or a fragment thereof, or SEQ ID NO: 441 or a fragment thereof, or SEQ ID NO: 442 or a fragment thereof, or SEQ ID NO: 443 or a fragment thereof, or SEQ ID NO: 444 or a fragment thereof, or SEQ ID NO: 445 or a fragment thereof, or SEQ ID NO: 446 or a fragment thereof, or SEQ ID NO: 447 or a fragment thereof, or SEQ ID NO: 448 or a fragment thereof, or SEQ ID NO: 449 or a fragment thereof, or SEQ ID NO: 450 or a fragment thereof, or SEQ ID NO: 451 or a fragment thereof,
   optionally wherein, a fragment comprises one or more, but not all, of: a Linker 1 region, an AStD stem region; a Linker 2 region; a stem-loop region, e.g., a D arm Region; a Linker 3 Region; a stem-loop region, e.g., an AC arm region; a variable region; a stem-loop region, e.g., a T arm Region; and a Linker 4 region, e.g., as these regions are described herein.
189. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM comprises a property selected from the following (e.g., in a TREM having a structure R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇1-R₇₂, wherein R is a ribonucleotide residue):
   a) under physiological conditions residue R₀ forms a linker region, e.g., a Linker 1 region;
   b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;
   c) under physiological conditions residues R₈-R₉ forms a linker region, e.g., a Linker 2 region;
   d) under physiological conditions residues-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;
   e) under physiological conditions residue-R₂₉ forms a linker region, e.g., a Linker 3 Region;
   f) under physiological conditions residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;
   g) under physiological conditions residue-[R₄₇]ₓ comprises a variable region;
   h) under physiological conditions residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or
   i) under physiological conditions residue R₇₂ forms a linker region, e.g., a Linker 4 region.
190. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM comprises a consensus sequence provided herein.
191. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM comprises a consensus sequence of Formula I zzz, wherein zzz indicates any of the twenty amino acids and Formula I corresponds to all species.
192. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, or the method of evaluating of embodiment 138 or 139-171, wherein the TREM comprises a consensus sequence of Formula II zzz, wherein zzz indicates any of the twenty amino acids and Formula II corresponds to mammals.
193. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM comprises a consensus sequence of Formula III zzz, wherein zzz indicates any of the twenty amino acids and Formula III corresponds to humans.
194. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM comprises a variable region at position R₄₇.
195. The method, composition or pharmaceutical composition, cell, reaction mixture, or the bioreactor, of embodiment 194, wherein the variable region is 1-271 residues in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 residues).
196. The method, composition or pharmaceutical composition, cell, reaction mixture, or the bioreactor of embodiment 194 or 195, wherein the variable region the variable region comprises any one, all or a combination of Adenine, Cytosine, Guanine or Uracil.
197. The method, composition or pharmaceutical composition, cell, reaction mixture, or the bioreactor of any one of embodiments 194-196, wherein the variable region comprises a ribonucleic acid (RNA) sequence encoded by a deoxyribonucleic acid (DNA) sequence disclosed in Table 5.
198. The method, composition or pharmaceutical composition, cell, reaction mixture, or the bioreactor of any one of embodiments 194-196, wherein the variable region comprises a ribonucleic acid (RNA) sequence encoded by a deoxyribonucleic acid (DNA) sequence of any one of SEQ ID NOs: 452-561 disclosed in Table 5.
199. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM recognizes a stop codon.
200. The composition or pharmaceutical composition, cell, reaction mixture, or the bioreactor of embodiment 199, wherein the TREM mediates acceptance and incorporation of an amino acid.
201. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM does not recognize a stop codon.
202. The composition or pharmaceutical composition, cell, reaction mixture, or the bioreactor of embodiment 201, wherein the TREM mediates acceptance and incorporation of an amino acid.
203. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM is formulated as a lyophilized TREM composition.
204. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM is formulated as a liquid TREM composition.
205. The method of any one of embodiments 1-102 or 170-188, the composition of any one of embodiments 103-116 or 170-188, the method of any one of embodiments 117-162 or 170-188, the cell of any one of embodiments 163-165 or 170-188, the reaction mixture of any one of embodiments 166 or 170-188 or the bioreactor of any one of embodiments 167 or 170-188, wherein the TREM is formulated as a frozen TREM composition.
206. The method, composition or pharmaceutical composition, cell, reaction mixture, or the bioreactor of any one of embodiments 1-205, wherein the TREM comprises an RNA sequence encoded by the DNA sequence of SEQ ID NO: 1, or a fragment thereof.
207. The method, composition or pharmaceutical composition, cell, reaction mixture, or the bioreactor of any one of embodiments 1-205, wherein the TREM comprises an RNA sequence encoded by the DNA sequence of SEQ ID NO: 2, or a fragment thereof.
208. The method, composition or pharmaceutical composition, cell, reaction mixture, or the bioreactor of any one of embodiments 1-205, wherein the TREM comprises an RNA sequence encoded by the DNA sequence of SEQ ID NO: 3, or a fragment thereof.
209. The method, composition or pharmaceutical composition, cell, reaction mixture, or the bioreactor of any one of embodiments 1-205, wherein the TREM comprises an RNA sequence encoded by the DNA sequence of SEQ ID NO: 4, or a fragment thereof.
210. The method, composition or pharmaceutical composition, cell, reaction mixture, or the bioreactor of any one of embodiments 1-205, wherein the TREM comprises an RNA sequence encoded by the DNA sequence of SEQ ID NO: 5, or a fragment thereof.
211. The method, composition or pharmaceutical composition, cell, reaction mixture, or the bioreactor of any one of embodiments 1-205, wherein the TREM comprises an RNA sequence encoded by the DNA sequence of SEQ ID NO: 6, or a fragment thereof.
212. The method, composition or pharmaceutical composition, cell, reaction mixture, or the bioreactor of any one of embodiments 1-205, wherein the TREM comprises an RNA sequence encoded by the DNA sequence of SEQ ID NO: 7, or a fragment thereof.
213. The method, composition or pharmaceutical composition, cell, reaction mixture, or the bioreactor of any one of embodiments 1-205, wherein the TREM comprises an RNA sequence encoded by the DNA sequence of SEQ ID NO: 8, or a fragment thereof.
214. The method, composition or pharmaceutical composition, cell, reaction mixture, or the bioreactor of any one of embodiments 1-205, wherein the TREM comprises an RNA sequence encoded by the DNA sequence of SEQ ID NO: 9, or a fragment thereof.
215. The method, composition or pharmaceutical composition, cell, reaction mixture, or the bioreactor of any one of embodiments 1-205, wherein the TREM comprises an RNA sequence encoded by the DNA sequence of SEQ ID NO: 10, or a fragment thereof.
216. The method, composition or pharmaceutical composition, cell, reaction mixture, or the bioreactor of any one of embodiments 1-205, wherein the TREM comprises an RNA sequence encoded by the DNA sequence of SEQ ID NO: 11, or a fragment thereof.
217. The method, composition or pharmaceutical composition, cell, reaction mixture, or the bioreactor of any one of embodiments 1-205, wherein the TREM comprises an RNA sequence encoded by the DNA sequence of SEQ ID NO: 12, or a fragment thereof.
218. The method, composition or pharmaceutical composition, cell, reaction mixture, or the bioreactor of any one of embodiments 1-205, wherein the TREM comprises an RNA sequence encoded by the DNA sequence of SEQ ID NO: 13, or a fragment thereof.
219. The method, composition or pharmaceutical composition, cell, reaction mixture, or the bioreactor of any one of embodiments 1-205, wherein the TREM comprises an RNA sequence encoded by the DNA sequence of SEQ ID NO:14, or a fragment thereof.
220. The method, composition or pharmaceutical composition, cell, reaction mixture, or the bioreactor of any one of embodiments 1-205, wherein the TREM comprises an RNA sequence encoded by the DNA sequence of SEQ ID NO: 15, or a fragment thereof.
221. The method, composition or pharmaceutical composition, cell, reaction mixture, or the bioreactor of any one of embodiments 1-205, wherein the TREM comprises an RNA sequence encoded by the DNA sequence of SEQ ID NO: 16, or a fragment thereof.
222. The method, composition or pharmaceutical composition, cell, reaction mixture, or the bioreactor of any one of embodiments 1-205, wherein the TREM comprises an RNA sequence encoded by the DNA sequence of SEQ ID NO: 17, or a fragment thereof.
223. The method, composition or pharmaceutical composition, cell, reaction mixture, or the bioreactor of any one of embodiments 1-205, wherein the TREM comprises an RNA sequence encoded by the DNA sequence of SEQ ID NO: 18, or a fragment thereof.
224. The method, composition or pharmaceutical composition, cell, reaction mixture, or the bioreactor of any one of embodiments 1-205, wherein the TREM comprises an RNA sequence encoded by the DNA sequence of SEQ ID NO:19, or a fragment thereof.
225. The method, composition or pharmaceutical composition, cell, reaction mixture, or the bioreactor of any one of embodiments 1-205, wherein the TREM comprises an RNA sequence encoded by the DNA sequence of SEQ ID NO: 20, or a fragment thereof.
226. The method, composition or pharmaceutical composition, cell, reaction mixture, or the bioreactor of any one of embodiments 1-205, wherein the TREM comprises an RNA sequence encoded by the DNA sequence of SEQ ID NO: 21, or a fragment thereof.
227. The method, composition or pharmaceutical composition, cell, reaction mixture, or the bioreactor of any one of embodiments 1-205, wherein the TREM comprises an RNA sequence encoded by the DNA sequence of SEQ ID NO: 22, or a fragment thereof.
228. The method, composition or pharmaceutical composition, cell, reaction mixture, or the bioreactor of any one of embodiments 1-205, wherein the TREM comprises an RNA sequence encoded by the DNA sequence of SEQ ID NO: 23, or a fragment thereof.
229. The method, composition or pharmaceutical composition, cell, reaction mixture, or the bioreactor of any one of embodiments 1-205, wherein the TREM comprises an RNA sequence encoded by the DNA sequence of SEQ ID NO: 24, or a fragment thereof.
230. The method, composition or pharmaceutical composition, cell, reaction mixture, or the bioreactor of any one of embodiments 1-205, wherein the TREM comprises an RNA sequence encoded by the DNA sequence of SEQ ID NO: 25, or a fragment thereof.
231. The method, composition or pharmaceutical composition, cell, reaction mixture, or the bioreactor of any one of embodiments 1-205, wherein the TREM comprises an RNA sequence encoded by the DNA sequence of SEQ ID NO: 26, or a fragment thereof.
232. The method, composition or pharmaceutical composition, cell, reaction mixture, or the bioreactor of any one of embodiments 1-205, wherein the TREM comprises an RNA sequence encoded by the DNA sequence of SEQ ID NO: 27, or a fragment thereof.
233. The method, composition or pharmaceutical composition, cell, reaction mixture, or the bioreactor of any one of embodiments 1-205, wherein the TREM comprises an RNA sequence encoded by the DNA sequence of SEQ ID NO: 28, or a fragment thereof.
234. The method, composition or pharmaceutical composition, cell, reaction mixture, or the bioreactor of any one of embodiments 1-205, wherein the TREM comprises an RNA sequence encoded by the DNA sequence of SEQ ID NO: 29, or a fragment thereof.
235. The method, composition or pharmaceutical composition, cell, reaction mixture, or the bioreactor of any one of embodiments 1-205, wherein the TREM comprises an RNA sequence encoded by the DNA sequence of SEQ ID NO: 30, or a fragment thereof.
236. The method, composition or pharmaceutical composition, cell, reaction mixture, or the bioreactor of any one of embodiments 1-205, wherein the TREM comprises an RNA sequence encoded by the DNA sequence of SEQ ID NO: 31, or a fragment thereof.
237. The method, composition or pharmaceutical composition, cell, reaction mixture, or the bioreactor of any one of embodiments 1-205, wherein the TREM comprises an RNA sequence encoded by the DNA sequence of SEQ ID NO: 32, or a fragment thereof.
238. The method, composition or pharmaceutical composition, cell, reaction mixture, or the bioreactor of any one of embodiments 1-205, wherein the TREM comprises an RNA sequence encoded by the DNA sequence of SEQ ID NO: 33, or a fragment thereof.
239. The method, composition or pharmaceutical composition, cell, reaction mixture, or the bioreactor of any one of embodiments 1-205, wherein the TREM comprises an RNA sequence encoded by the DNA sequence of SEQ ID NO: 34, or a fragment thereof.
240. The method, composition or pharmaceutical composition, cell, reaction mixture, or the bioreactor of any one of embodiments 1-205, wherein the TREM comprises an RNA sequence encoded by the DNA sequence of SEQ ID NO: 35, or a fragment thereof.
241. The method, composition or pharmaceutical composition, cell, reaction mixture, or the bioreactor of any one of embodiments 1-205, wherein the TREM comprises an RNA sequence encoded by the DNA sequence of SEQ ID NO: 36, or a fragment thereof.
242. The method, composition or pharmaceutical composition, cell, reaction mixture, or the bioreactor of any one of embodiments 1-205, wherein the TREM comprises an RNA sequence encoded by the DNA sequence of SEQ ID NO: 37, or a fragment thereof.
243. The method, composition or pharmaceutical composition, cell, reaction mixture, or the bioreactor of any one of embodiments 1-205, wherein the TREM comprises an RNA sequence encoded by the DNA sequence of SEQ ID NO: 38, or a fragment thereof.
244. The method, composition or pharmaceutical composition, cell, reaction mixture, or the bioreactor of any one of embodiments 1-205, wherein the TREM comprises an RNA sequence encoded by the DNA sequence of SEQ ID NO: 39, or a fragment thereof.
245. The method, composition or pharmaceutical composition, cell, reaction mixture, or the bioreactor of any one of embodiments 1-205, wherein the TREM comprises an RNA sequence encoded by the DNA sequence of SEQ ID NO: 40, or a fragment thereof.
246. The method, composition or pharmaceutical composition, cell, reaction mixture, or the bioreactor of any one of embodiments 1-205, wherein the TREM comprises an RNA sequence encoded by the DNA sequence of SEQ ID NO: 41, or a fragment thereof.
247. The method, composition or pharmaceutical composition, cell, reaction mixture, or the bioreactor of any one of embodiments 1-205, wherein the TREM comprises an RNA sequence encoded by the DNA sequence of SEQ ID NO: 42, or a fragment thereof.
248. The method, composition or pharmaceutical composition, cell, reaction mixture, or the bioreactor of any one of embodiments 1-205, wherein the TREM comprises an RNA sequence encoded by the DNA sequence of SEQ ID NO: 43, or a fragment thereof.
249. The method, composition or pharmaceutical composition, cell, reaction mixture, or the bioreactor of any one of embodiments 1-205, wherein the TREM comprises an RNA sequence encoded by the DNA sequence of SEQ ID NO: 44, or a fragment thereof.
250. The method, composition or pharmaceutical composition, cell, reaction mixture, or the bioreactor of any one of embodiments 1-205, wherein the TREM comprises an RNA sequence encoded by the DNA sequence of SEQ ID NO: 45, or a fragment thereof.
251. The method, composition or pharmaceutical composition, cell, reaction mixture, or the bioreactor of any one of embodiments 1-205, wherein the TREM comprises an RNA sequence encoded by the DNA sequence of SEQ ID NO: 46, or a fragment thereof.
252. The method, composition or pharmaceutical composition, cell, reaction mixture, or the bioreactor of any one of embodiments 1-205, wherein the TREM comprises an RNA sequence encoded by the DNA sequence of SEQ ID NO: 47, or a fragment thereof.
253. The method, composition or pharmaceutical composition, cell, reaction mixture, or the bioreactor of any one of embodiments 1-205, wherein the TREM comprises an RNA sequence encoded by the DNA sequence of SEQ ID NO: 48, or a fragment thereof.
254. The method, composition or pharmaceutical composition, cell, reaction mixture, or the bioreactor of any one of embodiments 1-205, wherein the TREM comprises an RNA sequence encoded by the DNA sequence of SEQ ID NO: 49, or a fragment thereof.
255. The method, composition or pharmaceutical composition, cell, reaction mixture, or the bioreactor of any one of embodiments 1-205, wherein the TREM comprises an RNA sequence encoded by the DNA sequence of SEQ ID NO: 50, or a fragment thereof.
256. The method, composition or pharmaceutical composition, cell, reaction mixture, or the bioreactor of any one of embodiments 1-205, wherein the TREM comprises an RNA sequence encoded by the DNA sequence of SEQ ID NO: 51, or a fragment thereof.
257. The method, composition or pharmaceutical composition, cell, reaction mixture, or the bioreactor of any one of embodiments 1-205, wherein the TREM comprises an RNA sequence encoded by the DNA sequence of SEQ ID NO: 52, or a fragment thereof.
258. The method, composition or pharmaceutical composition, cell, reaction mixture, or the bioreactor of any one of embodiments 1-205, wherein the TREM comprises an RNA sequence encoded by the DNA sequence of SEQ ID NO: 53, or a fragment thereof.
259. The method, composition or pharmaceutical composition, cell, reaction mixture, or the bioreactor of any one of embodiments 1-205, wherein the TREM comprises an RNA sequence encoded by the DNA sequence of SEQ ID NO: 54, or a fragment thereof.
260. The method, composition or pharmaceutical composition, cell, reaction mixture, or the bioreactor of any one of embodiments 1-205, wherein the TREM comprises an RNA sequence encoded by the DNA sequence of SEQ ID NO: 55, or a fragment thereof.
261. The method, composition or pharmaceutical composition, cell, reaction mixture, or the bioreactor of any one of embodiments 1-205, wherein the TREM comprises an RNA sequence encoded by the DNA sequence of SEQ ID NO: 56, or a fragment thereof.
262. The method, composition or pharmaceutical composition, cell, reaction mixture, or the bioreactor of any one of embodiments 1-205, wherein the TREM comprises an RNA sequence encoded by the DNA sequence of SEQ ID NO: 57, or a fragment thereof.
263. The method, composition or pharmaceutical composition, cell, reaction mixture, or the bioreactor of any one of embodiments 1-205, wherein the TREM comprises an RNA sequence encoded by the DNA sequence of SEQ ID NO: 58, or a fragment thereof.
264. The method, composition or pharmaceutical composition, cell, reaction mixture, or the bioreactor of any one of embodiments 1-205, wherein the TREM comprises an RNA sequence encoded by the DNA sequence of SEQ ID NO: 59, or a fragment thereof.
265. The method, composition or pharmaceutical composition, cell, reaction mixture, or the bioreactor of any one of embodiments 1-205, wherein the TREM comprises an RNA sequence encoded by the DNA sequence of SEQ ID NO: 60, or a fragment thereof.
266. The method, composition or pharmaceutical composition, cell, reaction mixture, or the bioreactor of any one of embodiments 1-205, wherein the TREM comprises an RNA sequence encoded by the DNA sequence of SEQ ID NO: 61, or a fragment thereof.
267. The method, composition or pharmaceutical composition, cell, reaction mixture, or the bioreactor of any one of embodiments 1-205, wherein the TREM comprises an RNA sequence encoded by the DNA sequence of SEQ ID NO: 62, or a fragment thereof.
268. The method, composition or pharmaceutical composition, cell, reaction mixture, or the bioreactor of any one of embodiments 1-205, wherein the TREM comprises an RNA sequence encoded by the DNA sequence of SEQ ID NO: 63, or a fragment thereof.
269. The method, composition or pharmaceutical composition, cell, reaction mixture, or the bioreactor of any one of embodiments 1-205, wherein the TREM comprises an RNA sequence encoded by the DNA sequence of SEQ ID NO: 64, or a fragment thereof.
270. The method, composition or pharmaceutical composition, cell, reaction mixture, or the bioreactor of any one of embodiments 1-205, wherein the TREM comprises an RNA sequence encoded by the DNA sequence of SEQ ID NO: 65, or a fragment thereof.
271. The method, composition or pharmaceutical composition, cell, reaction mixture, or the bioreactor of any one of embodiments 1-205, wherein the TREM comprises an RNA sequence encoded by the DNA sequence of SEQ ID NO: 66, or a fragment thereof.
272. The method, composition or pharmaceutical composition, cell, reaction mixture, or the bioreactor of any one of embodiments 1-205, wherein the TREM comprises an RNA sequence encoded by the DNA sequence of SEQ ID NO: 67, or a fragment thereof.
273. The method, composition or pharmaceutical composition, cell, reaction mixture, or the bioreactor of any one of embodiments 1-205, wherein the TREM comprises an RNA sequence encoded by the DNA sequence of SEQ ID NO: 68, or a fragment thereof.
274. The method, composition or pharmaceutical composition, cell, reaction mixture, or the bioreactor of any one of embodiments 1-205, wherein the TREM comprises an RNA sequence encoded by the DNA sequence of SEQ ID NO: 69, or a fragment thereof.
275. The method, composition or pharmaceutical composition, cell, reaction mixture, or the bioreactor of any one of embodiments 1-205, wherein the TREM comprises an RNA sequence encoded by the DNA sequence of SEQ ID NO: 70, or a fragment thereof.
276. The method, composition or pharmaceutical composition, cell, reaction mixture, or the bioreactor of any one of embodiments 1-205, wherein the TREM comprises an RNA sequence encoded by the DNA sequence of SEQ ID NO: 71, or a fragment thereof.
277. The method, composition or pharmaceutical composition, cell, reaction mixture, or the bioreactor of any one of embodiments 1-205, wherein the TREM comprises an RNA sequence encoded by the DNA sequence of SEQ ID NO: 72, or a fragment thereof.
278. The method, composition or pharmaceutical composition, cell, reaction mixture, or the bioreactor of any one of embodiments 1-205, wherein the TREM comprises an RNA sequence encoded by the DNA sequence of SEQ ID NO: 73, or a fragment thereof.
279. The method, composition or pharmaceutical composition, cell, reaction mixture, or the bioreactor of any one of embodiments 1-205, wherein the TREM comprises an RNA sequence encoded by the DNA sequence of SEQ ID NO: 74, or a fragment thereof.
280. The method, composition or pharmaceutical composition, cell, reaction mixture, or the bioreactor of any one of embodiments 1-205, wherein the TREM comprises an RNA sequence encoded by the DNA sequence of SEQ ID NO: 75, or a fragment thereof.
281. The method, composition or pharmaceutical composition, cell, reaction mixture, or the bioreactor of any one of embodiments 1-205, wherein the TREM comprises an RNA sequence encoded by the DNA sequence of SEQ ID NO: 76, or a fragment thereof.
282. The method, composition or pharmaceutical composition, cell, reaction mixture, or the bioreactor of any one of embodiments 1-205, wherein the TREM comprises an RNA sequence encoded by the DNA sequence of SEQ ID NO: 77, or a fragment thereof.
283. The method, composition or pharmaceutical composition, cell, reaction mixture, or the bioreactor of any one of embodiments 1-205, wherein the TREM comprises an RNA sequence encoded by the DNA sequence of SEQ ID NO: 78, or a fragment thereof.
284. The method, composition or pharmaceutical composition, cell, reaction mixture, or the bioreactor of any one of embodiments 1-205, wherein the TREM comprises an RNA sequence encoded by the DNA sequence of SEQ ID NO: 79, or a fragment thereof.
285. The method, composition or pharmaceutical composition, cell, reaction mixture, or the bioreactor of any one of embodiments 1-205, wherein the TREM comprises an RNA sequence encoded by the DNA sequence of SEQ ID NO: 80, or a fragment thereof.
286. The method, composition or pharmaceutical composition, cell, reaction mixture, or the bioreactor of any one of embodiments 1-205, wherein the TREM comprises an RNA sequence encoded by the DNA sequence of SEQ ID NO: 81, or a fragment thereof.
287. The method, composition or pharmaceutical composition, cell, reaction mixture, or the bioreactor of any one of embodiments 1-205, wherein the TREM comprises an RNA sequence encoded by the DNA sequence of SEQ ID NO: 82, or a fragment thereof.
288. The method, composition or pharmaceutical composition, cell, reaction mixture, or the bioreactor of any one of embodiments 1-205, wherein the TREM comprises an RNA sequence encoded by the DNA sequence of SEQ ID NO: 83, or a fragment thereof.
289. The method, composition or pharmaceutical composition, cell, reaction mixture, or the bioreactor of any one of embodiments 1-205, wherein the TREM comprises an RNA sequence encoded by the DNA sequence of SEQ ID NO: 84, or a fragment thereof.
290. The method, composition or pharmaceutical composition, cell, reaction mixture, or the bioreactor of any one of embodiments 1-205, wherein the TREM comprises an RNA sequence encoded by the DNA sequence of SEQ ID NO: 85, or a fragment thereof.
291. The method, composition or pharmaceutical composition, cell, reaction mixture, or the bioreactor of any one of embodiments 1-205, wherein the TREM comprises an RNA sequence encoded by the DNA sequence of SEQ ID NO:86, or a fragment thereof.
292. The method, composition or pharmaceutical composition, cell, reaction mixture, or the bioreactor of any one of embodiments 1-205, wherein the TREM comprises an RNA sequence encoded by the DNA sequence of SEQ ID NO: 87, or a fragment thereof.
293. The method, composition or pharmaceutical composition, cell, reaction mixture, or the bioreactor of any one of embodiments 1-205, wherein the TREM comprises an RNA sequence encoded by the DNA sequence of SEQ ID NO: 88, or a fragment thereof.
294. The method, composition or pharmaceutical composition, cell, reaction mixture, or the bioreactor of any one of embodiments 1-205, wherein the TREM comprises an RNA sequence encoded by the DNA sequence of SEQ ID NO: 89, or a fragment thereof.
295. The method, composition or pharmaceutical composition, cell, reaction mixture, or the bioreactor of any one of embodiments 1-205, wherein the TREM comprises an RNA sequence encoded by the DNA sequence of SEQ ID NO: 90, or a fragment thereof.
296. The method, composition or pharmaceutical composition, cell, reaction mixture, or the bioreactor of any one of embodiments 1-205, wherein the TREM comprises an RNA sequence encoded by the DNA sequence of SEQ ID NO: 91, or a fragment thereof.
297. The method, composition or pharmaceutical composition, cell, reaction mixture, or the bioreactor of any one of embodiments 1-205, wherein the TREM comprises an RNA sequence encoded by the DNA sequence of SEQ ID NO: 92, or a fragment thereof.
298. The method, composition or pharmaceutical composition, cell, reaction mixture, or the bioreactor of any one of embodiments 1-205, wherein the TREM comprises an RNA sequence encoded by the DNA sequence of SEQ ID NO: 93, or a fragment thereof.
299. The method, composition or pharmaceutical composition, cell, reaction mixture, or the bioreactor of any one of embodiments 1-205, wherein the TREM comprises an RNA sequence encoded by the DNA sequence of SEQ ID NO: 94, or a fragment thereof.
300. The method, composition or pharmaceutical composition, cell, reaction mixture, or the bioreactor of any one of embodiments 1-205, wherein the TREM comprises an RNA sequence encoded by the DNA sequence of SEQ ID NO: 95, or a fragment thereof.
301. The method, composition or pharmaceutical composition, cell, reaction mixture, or the bioreactor of any one of embodiments 1-205, wherein the TREM comprises an RNA sequence encoded by the DNA sequence of SEQ ID NO: 96, or a fragment thereof.
302. The method, composition or pharmaceutical composition, cell, reaction mixture, or the bioreactor of any one of embodiments 1-205, wherein the TREM comprises an RNA sequence encoded by the DNA sequence of SEQ ID NO: 97, or a fragment thereof.
303. The method, composition or pharmaceutical composition, cell, reaction mixture, or the bioreactor of any one of embodiments 1-205, wherein the TREM comprises an RNA sequence encoded by the DNA sequence of SEQ ID NO: 98, or a fragment thereof.
304. The method, composition or pharmaceutical composition, cell, reaction mixture, or the bioreactor of any one of embodiments 1-205, wherein the TREM comprises an RNA sequence encoded by the DNA sequence of SEQ ID NO: 99, or a fragment thereof.
305. The method, composition or pharmaceutical composition, cell, reaction mixture, or the bioreactor of any one of embodiments 1-205, wherein the TREM comprises an RNA sequence encoded by the DNA sequence of SEQ ID NO: 100, or a fragment thereof.
306. The method, composition or pharmaceutical composition, cell, reaction mixture, or the bioreactor of any one of embodiments 1-205, wherein the TREM comprises an RNA sequence encoded by the DNA sequence of SEQ ID NO: 101, or a fragment thereof.
307. The method, composition or pharmaceutical composition, cell, reaction mixture, or the bioreactor of any one of embodiments 1-205, wherein the TREM comprises an RNA sequence encoded by the DNA sequence of SEQ ID NO: 102, or a fragment thereof.
308. The method, composition or pharmaceutical composition, cell, reaction mixture, or the bioreactor of any one of embodiments 1-205, wherein the TREM comprises an RNA sequence encoded by the DNA sequence of SEQ ID NO: 103, or a fragment thereof.
309. The method, composition or pharmaceutical composition, cell, reaction mixture, or the bioreactor of any one of embodiments 1-205, wherein the TREM comprises an RNA sequence encoded by the DNA sequence of SEQ ID NO: 104, or a fragment thereof.
310. The method, composition or pharmaceutical composition, cell, reaction mixture, or the bioreactor of any one of embodiments 1-205, wherein the TREM comprises an RNA sequence encoded by the DNA sequence of SEQ ID NO: 105, or a fragment thereof.
311. The method, composition or pharmaceutical composition, cell, reaction mixture, or the bioreactor of any one of embodiments 1-205, wherein the TREM comprises an RNA sequence encoded by the DNA sequence of SEQ ID NO:106, or a fragment thereof.
312. The method, composition or pharmaceutical composition, cell, reaction mixture, or the bioreactor of any one of embodiments 1-205, wherein the TREM comprises an RNA sequence encoded by the DNA sequence of SEQ ID NO:107, or a fragment thereof.
313. The method, composition or pharmaceutical composition, cell, reaction mixture, or the bioreactor of any one of embodiments 1-205, wherein the TREM comprises an RNA sequence encoded by the DNA sequence of SEQ ID NO:108, or a fragment thereof.
314. The method, composition or pharmaceutical composition, cell, reaction mixture, or the bioreactor of any one of embodiments 1-205, wherein the TREM comprises an RNA sequence encoded by the DNA sequence of SEQ ID NO:109, or a fragment thereof.
315. The method, composition or pharmaceutical composition, cell, reaction mixture, or the bioreactor of any one of embodiments 1-205, wherein the TREM comprises an RNA sequence encoded by the DNA sequence of SEQ ID NO:110, or a fragment thereof.
316. The method, composition or pharmaceutical composition, cell, reaction mixture, or the bioreactor of any one of embodiments 1-205, wherein the TREM comprises an RNA sequence encoded by the DNA sequence of SEQ ID NO:111, or a fragment thereof.
317. The method, composition or pharmaceutical composition, cell, reaction mixture, or the bioreactor of any one of embodiments 1-205, wherein the TREM comprises an RNA sequence encoded by the DNA sequence of SEQ ID NO:112, or a fragment thereof.
318. The method, composition or pharmaceutical composition, cell, reaction mixture, or the bioreactor of any one of embodiments 1-205, wherein the TREM comprises an RNA sequence encoded by the DNA sequence of SEQ ID NO:113, or a fragment thereof.
319. The method, composition or pharmaceutical composition, cell, reaction mixture, or the bioreactor of any one of embodiments 1-205, wherein the TREM comprises an RNA sequence encoded by the DNA sequence of SEQ ID NO:114, or a fragment thereof.
320. The method, composition or pharmaceutical composition, cell, reaction mixture, or the bioreactor of any one of embodiments 1-205, wherein the TREM comprises an RNA sequence encoded by the DNA sequence of SEQ ID NO:115, or a fragment thereof.
321. The method, composition or pharmaceutical composition, cell, reaction mixture, or the bioreactor of any one of embodiments 1-205, wherein the TREM comprises an RNA sequence encoded by the DNA sequence of SEQ ID NO:116, or a fragment thereof.
322. The method, composition or pharmaceutical composition, cell, reaction mixture, or the bioreactor of any one of embodiments 1-205, wherein the TREM comprises an RNA sequence encoded by the DNA sequence of SEQ ID NO:117, or a fragment thereof.
323. The method, composition or pharmaceutical composition, cell, reaction mixture, or the bioreactor of any one of embodiments 1-205, wherein the TREM comprises an RNA sequence encoded by the DNA sequence of SEQ ID NO:118, or a fragment thereof.
324. The method, composition or pharmaceutical composition, cell, reaction mixture, or the bioreactor of any one of embodiments 1-205, wherein the TREM comprises an RNA sequence encoded by the DNA sequence of SEQ ID NO:119, or a fragment thereof.
325. The method, composition or pharmaceutical composition, cell, reaction mixture, or the bioreactor of any one of embodiments 1-205, wherein the TREM comprises an RNA sequence encoded by the DNA sequence of SEQ ID NO:120, or a fragment thereof.
326. The method, composition or pharmaceutical composition, cell, reaction mixture, or the bioreactor of any one of embodiments 1-205, wherein the TREM comprises an RNA sequence encoded by the DNA sequence of SEQ ID NO:121, or a fragment thereof.
327. The method, composition or pharmaceutical composition, cell, reaction mixture, or the bioreactor of any one of embodiments 1-205, wherein the TREM comprises an RNA sequence encoded by the DNA sequence of SEQ ID NO:122, or a fragment thereof.
328. The method, composition or pharmaceutical composition, cell, reaction mixture, or the bioreactor of any one of embodiments 1-205, wherein the TREM comprises an RNA sequence encoded by the DNA sequence of SEQ ID NO:123, or a fragment thereof.
329. The method, composition or pharmaceutical composition, cell, reaction mixture, or the bioreactor of any one of embodiments 1-205, wherein the TREM comprises an RNA sequence encoded by the DNA sequence of SEQ ID NO:124, or a fragment thereof.
330. The method, composition or pharmaceutical composition, cell, reaction mixture, or the bioreactor of any one of embodiments 1-205, wherein the TREM comprises an RNA sequence encoded by the DNA sequence of SEQ ID NO:125, or a fragment thereof.
331. The method, composition or pharmaceutical composition, cell, reaction mixture, or the bioreactor of any one of embodiments 1-205, wherein the TREM comprises an RNA sequence encoded by the DNA sequence of SEQ ID NO:126, or a fragment thereof.
332. The method, composition or pharmaceutical composition, cell, reaction mixture, or the bioreactor of any one of embodiments 1-205, wherein the TREM comprises an RNA sequence encoded by the DNA sequence of SEQ ID NO:127, or a fragment thereof.
333. The method, composition or pharmaceutical composition, cell, reaction mixture, or the bioreactor of any one of embodiments 1-205, wherein the TREM comprises an RNA sequence encoded by the DNA sequence of SEQ ID NO:128, or a fragment thereof.
334. The method, composition or pharmaceutical composition, cell, reaction mixture, or the bioreactor of any one of embodiments 1-205, wherein the TREM comprises an RNA sequence encoded by the DNA sequence of SEQ ID NO:129, or a fragment thereof.
335. The method, composition or pharmaceutical composition, cell, reaction mixture, or the bioreactor of any one of embodiments 1-205, wherein the TREM comprises an RNA sequence encoded by the DNA sequence of SEQ ID NO:130, or a fragment thereof.
336. The method, composition or pharmaceutical composition, cell, reaction mixture, or the bioreactor of any one of embodiments 1-205, wherein the TREM comprises an RNA sequence encoded by the DNA sequence of SEQ ID NO:131, or a fragment thereof.
337. The method, composition or pharmaceutical composition, cell, reaction mixture, or the bioreactor of any one of embodiments 1-205, wherein the TREM comprises an RNA sequence encoded by the DNA sequence of SEQ ID NO:132, or a fragment thereof.
338. The method, composition or pharmaceutical composition, cell, reaction mixture, or the bioreactor of any one of embodiments 1-205, wherein the TREM comprises an RNA sequence encoded by the DNA sequence of SEQ ID NO:133, or a fragment thereof.
339. The method, composition or pharmaceutical composition, cell, reaction mixture, or the bioreactor of any one of embodiments 1-205, wherein the TREM comprises an RNA sequence encoded by the DNA sequence of SEQ ID NO:134, or a fragment thereof.
340. The method, composition or pharmaceutical composition, cell, reaction mixture, or the bioreactor of any one of embodiments 1-205, wherein the TREM comprises an RNA sequence encoded by the DNA sequence of SEQ ID NO:135, or a fragment thereof.
341. The method, composition or pharmaceutical composition, cell, reaction mixture, or the bioreactor of any one of embodiments 1-205, wherein the TREM comprises an RNA sequence encoded by the DNA sequence of SEQ ID NO:136, or a fragment thereof.
342. The method, composition or pharmaceutical composition, cell, reaction mixture, or the bioreactor of any one of embodiments 1-205, wherein the TREM comprises an RNA sequence encoded by the DNA sequence of SEQ ID NO:137, or a fragment thereof.
343. The method, composition or pharmaceutical composition, cell, reaction mixture, or the bioreactor of any one of embodiments 1-205, wherein the TREM comprises an RNA sequence encoded by the DNA sequence of SEQ ID NO:138, or a fragment thereof.
344. The method, composition or pharmaceutical composition, cell, reaction mixture, or the bioreactor of any one of embodiments 1-205, wherein the TREM comprises an RNA sequence encoded by the DNA sequence of SEQ ID NO:139, or a fragment thereof.
345. The method, composition or pharmaceutical composition, cell, reaction mixture, or the bioreactor of any one of embodiments 1-205, wherein the TREM comprises an RNA sequence encoded by the DNA sequence of SEQ ID NO:140, or a fragment thereof.
346. The method, composition or pharmaceutical composition, cell, reaction mixture, or the bioreactor of any one of embodiments 1-205, wherein the TREM comprises an RNA sequence encoded by the DNA sequence of SEQ ID NO:141, or a fragment thereof.
347. The method, composition or pharmaceutical composition, cell, reaction mixture, or the bioreactor of any one of embodiments 1-205, wherein the TREM comprises an RNA sequence encoded by the DNA sequence of SEQ ID NO:142, or a fragment thereof.
348. The method, composition or pharmaceutical composition, cell, reaction mixture, or the bioreactor of any one of embodiments 1-205, wherein the TREM comprises an RNA sequence encoded by the DNA sequence of SEQ ID NO:143, or a fragment thereof.
349. The method, composition or pharmaceutical composition, cell, reaction mixture, or the bioreactor of any one of embodiments 1-205, wherein the TREM comprises an RNA sequence encoded by the DNA sequence of SEQ ID NO:144, or a fragment thereof.
350. The method, composition or pharmaceutical composition, cell, reaction mixture, or the bioreactor of any one of embodiments 1-205, wherein the TREM comprises an RNA sequence encoded by the DNA sequence of SEQ ID NO:145, or a fragment thereof.
351. The method, composition or pharmaceutical composition, cell, reaction mixture, or the bioreactor of any one of embodiments 1-205, wherein the TREM comprises an RNA sequence encoded by the DNA sequence of SEQ ID NO:146, or a fragment thereof.
352. The method, composition or pharmaceutical composition, cell, reaction mixture, or the bioreactor of any one of embodiments 1-205, wherein the TREM comprises an RNA sequence encoded by the DNA sequence of SEQ ID NO:147, or a fragment thereof.
353. The method, composition or pharmaceutical composition, cell, reaction mixture, or the bioreactor of any one of embodiments 1-205, wherein the TREM comprises an RNA sequence encoded by the DNA sequence of SEQ ID NO:148, or a fragment thereof.
354. The method, composition or pharmaceutical composition, cell, reaction mixture, or the bioreactor of any one of embodiments 1-205, wherein the TREM comprises an RNA sequence encoded by the DNA sequence of SEQ ID NO:149, or a fragment thereof.
355. The method, composition or pharmaceutical composition, cell, reaction mixture, or the bioreactor of any one of embodiments 1-205, wherein the TREM comprises an RNA sequence encoded by the DNA sequence of SEQ ID NO:150, or a fragment thereof.
356. The method, composition or pharmaceutical composition, cell, reaction mixture, or the bioreactor of any one of embodiments 1-205, wherein the TREM comprises an RNA sequence encoded by the DNA sequence of SEQ ID NO:151, or a fragment thereof.
357. The method, composition or pharmaceutical composition, cell, reaction mixture, or the bioreactor of any one of embodiments 1-205, wherein the TREM comprises an RNA sequence encoded by the DNA sequence of SEQ ID NO:152, or a fragment thereof.
358. The method, composition or pharmaceutical composition, cell, reaction mixture, or the bioreactor of any one of embodiments 1-205, wherein the TREM comprises an RNA sequence encoded by the DNA sequence of SEQ ID NO:153, or a fragment thereof.
359. The method, composition or pharmaceutical composition, cell, reaction mixture, or the bioreactor of any one of embodiments 1-205, wherein the TREM comprises an RNA sequence encoded by the DNA sequence of SEQ ID NO:154, or a fragment thereof.
360. The method, composition or pharmaceutical composition, cell, reaction mixture, or the bioreactor of any one of embodiments 1-205, wherein the TREM comprises an RNA sequence encoded by the DNA sequence of SEQ ID NO:155, or a fragment thereof.
361. The method, composition or pharmaceutical composition, cell, reaction mixture, or the bioreactor of any one of embodiments 1-205, wherein the TREM comprises an RNA sequence encoded by the DNA sequence of SEQ ID NO:156, or a fragment thereof.
362. The method, composition or pharmaceutical composition, cell, reaction mixture, or the bioreactor of any one of embodiments 1-205, wherein the TREM comprises an RNA sequence encoded by the DNA sequence of SEQ ID NO:157, or a fragment thereof.
363. The method, composition or pharmaceutical composition, cell, reaction mixture, or the bioreactor of any one of embodiments 1-205, wherein the TREM comprises an RNA sequence encoded by the DNA sequence of SEQ ID NO:158, or a fragment thereof.
364. The method, composition or pharmaceutical composition, cell, reaction mixture, or the bioreactor of any one of embodiments 1-205, wherein the TREM comprises an RNA sequence encoded by the DNA sequence of SEQ ID NO:159, or a fragment thereof.
365. The method, composition or pharmaceutical composition, cell, reaction mixture, or the bioreactor of any one of embodiments 1-205, wherein the TREM comprises an RNA sequence encoded by the DNA sequence of SEQ ID NO:160, or a fragment thereof.
366. The method, composition or pharmaceutical composition, cell, reaction mixture, or the bioreactor of any one of embodiments 1-205, wherein the TREM comprises an RNA sequence encoded by the DNA sequence of SEQ ID NO:161, or a fragment thereof.
367. The method, composition or pharmaceutical composition, cell, reaction mixture, or the bioreactor of any one of embodiments 1-205, wherein the TREM comprises an RNA sequence encoded by the DNA sequence of SEQ ID NO:162, or a fragment thereof.
368. The method, composition or pharmaceutical composition, cell, reaction mixture, or the bioreactor of any one of embodiments 1-205, wherein the TREM comprises an RNA sequence encoded by the DNA sequence of SEQ ID NO:163, or a fragment thereof.
369. The method, composition or pharmaceutical composition, cell, reaction mixture, or the bioreactor of any one of embodiments 1-205, wherein the TREM comprises an RNA sequence encoded by the DNA sequence of SEQ ID NO:164, or a fragment thereof.
370. The method, composition or pharmaceutical composition, cell, reaction mixture, or the bioreactor of any one of embodiments 1-205, wherein the TREM comprises an RNA sequence encoded by the DNA sequence of SEQ ID NO:165, or a fragment thereof.
371. The method, composition or pharmaceutical composition, cell, reaction mixture, or the bioreactor of any one of embodiments 1-205, wherein the TREM comprises an RNA sequence encoded by the DNA sequence of SEQ ID NO:166, or a fragment thereof.
372. The method, composition or pharmaceutical composition, cell, reaction mixture, or the bioreactor of any one of embodiments 1-205, wherein the TREM comprises an RNA sequence encoded by the DNA sequence of SEQ ID NO:167, or a fragment thereof.
373. The method, composition or pharmaceutical composition, cell, reaction mixture, or the bioreactor of any one of embodiments 1-205, wherein the TREM comprises an RNA sequence encoded by the DNA sequence of SEQ ID NO:168, or a fragment thereof.
374. The method, composition or pharmaceutical composition, cell, reaction mixture, or the bioreactor of any one of embodiments 1-205, wherein the TREM comprises an RNA sequence encoded by the DNA sequence of SEQ ID NO:169, or a fragment thereof.
375. The method, composition or pharmaceutical composition, cell, reaction mixture, or the bioreactor of any one of embodiments 1-205, wherein the TREM comprises an RNA sequence encoded by the DNA sequence of SEQ ID NO:170, or a fragment thereof.
376. The method, composition or pharmaceutical composition, cell, reaction mixture, or the bioreactor of any one of embodiments 1-205, wherein the TREM comprises an RNA sequence encoded by the DNA sequence of SEQ ID NO:171, or a fragment thereof.
377. The method, composition or pharmaceutical composition, cell, reaction mixture, or the bioreactor of any one of embodiments 1-205, wherein the TREM comprises an RNA sequence encoded by the DNA sequence of SEQ ID NO:172, or a fragment thereof.
378. The method, composition or pharmaceutical composition, cell, reaction mixture, or the bioreactor of any one of embodiments 1-205, wherein the TREM comprises an RNA sequence encoded by the DNA sequence of SEQ ID NO:173, or a fragment thereof.
379. The method, composition or pharmaceutical composition, cell, reaction mixture, or the bioreactor of any one of embodiments 1-205, wherein the TREM comprises an RNA sequence encoded by the DNA sequence of SEQ ID NO:174, or a fragment thereof.
380. The method, composition or pharmaceutical composition, cell, reaction mixture, or the bioreactor of any one of embodiments 1-205, wherein the TREM comprises an RNA sequence encoded by the DNA sequence of SEQ ID NO:175, or a fragment thereof.
381. The method, composition or pharmaceutical composition, cell, reaction mixture, or the bioreactor of any one of embodiments 1-205, wherein the TREM comprises an RNA sequence encoded by the DNA sequence of SEQ ID NO:176, or a fragment thereof.
382. The method, composition or pharmaceutical composition, cell, reaction mixture, or the bioreactor of any one of embodiments 1-205, wherein the TREM comprises an RNA sequence encoded by the DNA sequence of SEQ ID NO:177, or a fragment thereof.
383. The method, composition or pharmaceutical composition, cell, reaction mixture, or the bioreactor of any one of embodiments 1-205, wherein the TREM comprises an RNA sequence encoded by the DNA sequence of SEQ ID NO:178, or a fragment thereof.
384. The method, composition or pharmaceutical composition, cell, reaction mixture, or the bioreactor of any one of embodiments 1-205, wherein the TREM comprises an RNA sequence encoded by the DNA sequence of SEQ ID NO:179, or a fragment thereof.
385. The method, composition or pharmaceutical composition, cell, reaction mixture, or the bioreactor of any one of embodiments 1-205, wherein the TREM comprises an RNA sequence encoded by the DNA sequence of SEQ ID NO:180, or a fragment thereof.
386. The method, composition or pharmaceutical composition, cell, reaction mixture, or the bioreactor of any one of embodiments 1-205, wherein the TREM comprises an RNA sequence encoded by the DNA sequence of SEQ ID NO:181, or a fragment thereof.
387. The method, composition or pharmaceutical composition, cell, reaction mixture, or the bioreactor of any one of embodiments 1-205, wherein the TREM comprises an RNA sequence encoded by the DNA sequence of SEQ ID NO:182, or a fragment thereof.
388. The method, composition or pharmaceutical composition, cell, reaction mixture, or the bioreactor of any one of embodiments 1-205, wherein the TREM comprises an RNA sequence encoded by the DNA sequence of SEQ ID NO:183, or a fragment thereof.
389. The method, composition or pharmaceutical composition, cell, reaction mixture, or the bioreactor of any one of embodiments 1-205, wherein the TREM comprises an RNA sequence encoded by the DNA sequence of SEQ ID NO:184, or a fragment thereof.
390. The method, composition or pharmaceutical composition, cell, reaction mixture, or the bioreactor of any one of embodiments 1-205, wherein the TREM comprises an RNA sequence encoded by the DNA sequence of SEQ ID NO:185, or a fragment thereof.
391. The method, composition or pharmaceutical composition, cell, reaction mixture, or the bioreactor of any one of embodiments 1-205, wherein the TREM comprises an RNA sequence encoded by the DNA sequence of SEQ ID NO:186, or a fragment thereof.
392. The method, composition or pharmaceutical composition, cell, reaction mixture, or the bioreactor of any one of embodiments 1-205, wherein the TREM comprises an RNA sequence encoded by the DNA sequence of SEQ ID NO:187, or a fragment thereof.
393. The method, composition or pharmaceutical composition, cell, reaction mixture, or the bioreactor of any one of embodiments 1-205, wherein the TREM comprises an RNA sequence encoded by the DNA sequence of SEQ ID NO:188, or a fragment thereof.
394. The method, composition or pharmaceutical composition, cell, reaction mixture, or the bioreactor of any one of embodiments 1-205, wherein the TREM comprises an RNA sequence encoded by the DNA sequence of SEQ ID NO:189, or a fragment thereof.
395. The method, composition or pharmaceutical composition, cell, reaction mixture, or the bioreactor of any one of embodiments 1-205, wherein the TREM comprises an RNA sequence encoded by the DNA sequence of SEQ ID NO:190, or a fragment thereof.
396. The method, composition or pharmaceutical composition, cell, reaction mixture, or the bioreactor of any one of embodiments 1-205, wherein the TREM comprises an RNA sequence encoded by the DNA sequence of SEQ ID NO:191, or a fragment thereof.
397. The method, composition or pharmaceutical composition, cell, reaction mixture, or the bioreactor of any one of embodiments 1-205, wherein the TREM comprises an RNA sequence encoded by the DNA sequence of SEQ ID NO:192, or a fragment thereof.
398. The method, composition or pharmaceutical composition, cell, reaction mixture, or the bioreactor of any one of embodiments 1-205, wherein the TREM comprises an RNA sequence encoded by the DNA sequence of SEQ ID NO:193, or a fragment thereof.
399. The method, composition or pharmaceutical composition, cell, reaction mixture, or the bioreactor of any one of embodiments 1-205, wherein the TREM comprises an RNA sequence encoded by the DNA sequence of SEQ ID NO:194, or a fragment thereof.
400. The method, composition or pharmaceutical composition, cell, reaction mixture, or the bioreactor of any one of embodiments 1-205, wherein the TREM comprises an RNA sequence encoded by the DNA sequence of SEQ ID NO:195, or a fragment thereof.
401. The method, composition or pharmaceutical composition, cell, reaction mixture, or the bioreactor of any one of embodiments 1-205, wherein the TREM comprises an RNA sequence encoded by the DNA sequence of SEQ ID NO:196, or a fragment thereof.
402. The method, composition or pharmaceutical composition, cell, reaction mixture, or the bioreactor of any one of embodiments 1-205, wherein the TREM comprises an RNA sequence encoded by the DNA sequence of SEQ ID NO:197, or a fragment thereof.
403. The method, composition or pharmaceutical composition, cell, reaction mixture, or the bioreactor of any one of embodiments 1-205, wherein the TREM comprises an RNA sequence encoded by the DNA sequence of SEQ ID NO:198, or a fragment thereof.
404. The method, composition or pharmaceutical composition, cell, reaction mixture, or the bioreactor of any one of embodiments 1-205, wherein the TREM comprises an RNA sequence encoded by the DNA sequence of SEQ ID NO:199, or a fragment thereof.
405. The method, composition or pharmaceutical composition, cell, reaction mixture, or the bioreactor of any one of embodiments 1-205, wherein the TREM comprises an RNA sequence encoded by the DNA sequence of SEQ ID NO:200, or a fragment thereof.
406. The method, composition or pharmaceutical composition, cell, reaction mixture, or the bioreactor of any one of embodiments 1-205, wherein the TREM comprises an RNA sequence encoded by the DNA sequence of SEQ ID NO:201, or a fragment thereof.
407. The method, composition or pharmaceutical composition, cell, reaction mixture, or the bioreactor of any one of embodiments 1-205, wherein the TREM comprises an RNA sequence encoded by the DNA sequence of SEQ ID NO:202, or a fragment thereof.
408. The method, composition or pharmaceutical composition, cell, reaction mixture, or the bioreactor of any one of embodiments 1-205, wherein the TREM comprises an RNA sequence encoded by the DNA sequence of SEQ ID NO:203, or a fragment thereof.
409. The method, composition or pharmaceutical composition, cell, reaction mixture, or the bioreactor of any one of embodiments 1-205, wherein the TREM comprises an RNA sequence encoded by the DNA sequence of SEQ ID NO:204, or a fragment thereof.
410. The method, composition or pharmaceutical composition, cell, reaction mixture, or the bioreactor of any one of embodiments 1-205, wherein the TREM comprises an RNA sequence encoded by the DNA sequence of SEQ ID NO:205, or a fragment thereof.
411. The method, composition or pharmaceutical composition, cell, reaction mixture, or the bioreactor of any one of embodiments 1-205, wherein the TREM comprises an RNA sequence encoded by the DNA sequence of SEQ ID NO:206, or a fragment thereof.
412. The method, composition or pharmaceutical composition, cell, reaction mixture, or the bioreactor of any one of embodiments 1-205, wherein the TREM comprises an RNA sequence encoded by the DNA sequence of SEQ ID NO:207, or a fragment thereof.
413. The method, composition or pharmaceutical composition, cell, reaction mixture, or the bioreactor of any one of embodiments 1-205, wherein the TREM comprises an RNA sequence encoded by the DNA sequence of SEQ ID NO:208, or a fragment thereof.
414. The method, composition or pharmaceutical composition, cell, reaction mixture, or the bioreactor of any one of embodiments 1-205, wherein the TREM comprises an RNA sequence encoded by the DNA sequence of SEQ ID NO:209, or a fragment thereof.
415. The method, composition or pharmaceutical composition, cell, reaction mixture, or the bioreactor of any one of embodiments 1-205, wherein the TREM comprises an RNA sequence encoded by the DNA sequence of SEQ ID NO:210, or a fragment thereof.
416. The method, composition or pharmaceutical composition, cell, reaction mixture, or the bioreactor of any one of embodiments 1-205, wherein the TREM comprises an RNA sequence encoded by the DNA sequence of SEQ ID NO:211, or a fragment thereof.
417. The method, composition or pharmaceutical composition, cell, reaction mixture, or the bioreactor of any one of embodiments 1-205, wherein the TREM comprises an RNA sequence encoded by the DNA sequence of SEQ ID NO:212, or a fragment thereof.
418. The method, composition or pharmaceutical composition, cell, reaction mixture, or the bioreactor of any one of embodiments 1-205, wherein the TREM comprises an RNA sequence encoded by the DNA sequence of SEQ ID NO:213, or a fragment thereof.
419. The method, composition or pharmaceutical composition, cell, reaction mixture, or the bioreactor of any one of embodiments 1-205, wherein the TREM comprises an RNA sequence encoded by the DNA sequence of SEQ ID NO:214, or a fragment thereof.
420. The method, composition or pharmaceutical composition, cell, reaction mixture, or the bioreactor of any one of embodiments 1-205, wherein the TREM comprises an RNA sequence encoded by the DNA sequence of SEQ ID NO:215, or a fragment thereof.
421. The method, composition or pharmaceutical composition, cell, reaction mixture, or the bioreactor of any one of embodiments 1-205, wherein the TREM comprises an RNA sequence encoded by the DNA sequence of SEQ ID NO:216, or a fragment thereof.
422. The method, composition or pharmaceutical composition, cell, reaction mixture, or the bioreactor of any one of embodiments 1-205, wherein the TREM comprises an RNA sequence encoded by the DNA sequence of SEQ ID NO:217, or a fragment thereof.
423. The method, composition or pharmaceutical composition, cell, reaction mixture, or the bioreactor of any one of embodiments 1-205, wherein the TREM comprises an RNA sequence encoded by the DNA sequence of SEQ ID NO:218, or a fragment thereof.
424. The method, composition or pharmaceutical composition, cell, reaction mixture, or the bioreactor of any one of embodiments 1-205, wherein the TREM comprises an RNA sequence encoded by the DNA sequence of SEQ ID NO:219, or a fragment thereof.
425. The method, composition or pharmaceutical composition, cell, reaction mixture, or the bioreactor of any one of embodiments 1-205, wherein the TREM comprises an RNA sequence encoded by the DNA sequence of SEQ ID NO:220, or a fragment thereof.
426. The method, composition or pharmaceutical composition, cell, reaction mixture, or the bioreactor of any one of embodiments 1-205, wherein the TREM comprises an RNA sequence encoded by the DNA sequence of SEQ ID NO:221, or a fragment thereof.
427. The method, composition or pharmaceutical composition, cell, reaction mixture, or the bioreactor of any one of embodiments 1-205, wherein the TREM comprises an RNA sequence encoded by the DNA sequence of SEQ ID NO:222, or a fragment thereof.
428. The method, composition or pharmaceutical composition, cell, reaction mixture, or the bioreactor of any one of embodiments 1-205, wherein the TREM comprises an RNA sequence encoded by the DNA sequence of SEQ ID NO:223, or a fragment thereof.
429. The method, composition or pharmaceutical composition, cell, reaction mixture, or the bioreactor of any one of embodiments 1-205, wherein the TREM comprises an RNA sequence encoded by the DNA sequence of SEQ ID NO:224, or a fragment thereof.
430. The method, composition or pharmaceutical composition, cell, reaction mixture, or the bioreactor of any one of embodiments 1-205, wherein the TREM comprises an RNA sequence encoded by the DNA sequence of SEQ ID NO:225, or a fragment thereof.
431. The method, composition or pharmaceutical composition, cell, reaction mixture, or the bioreactor of any one of embodiments 1-205, wherein the TREM comprises an RNA sequence encoded by the DNA sequence of SEQ ID NO:226, or a fragment thereof.
432. The method, composition or pharmaceutical composition, cell, reaction mixture, or the bioreactor of any one of embodiments 1-205, wherein the TREM comprises an RNA sequence encoded by the DNA sequence of SEQ ID NO:227, or a fragment thereof.
433. The method, composition or pharmaceutical composition, cell, reaction mixture, or the bioreactor of any one of embodiments 1-205, wherein the TREM comprises an RNA sequence encoded by the DNA sequence of SEQ ID NO:228, or a fragment thereof.
434. The method, composition or pharmaceutical composition, cell, reaction mixture, or the bioreactor of any one of embodiments 1-205, wherein the TREM comprises an RNA sequence encoded by the DNA sequence of SEQ ID NO:229, or a fragment thereof.
435. The method, composition or pharmaceutical composition, cell, reaction mixture, or the bioreactor of any one of embodiments 1-205, wherein the TREM comprises an RNA sequence encoded by the DNA sequence of SEQ ID NO:230, or a fragment thereof.
436. The method, composition or pharmaceutical composition, cell, reaction mixture, or the bioreactor of any one of embodiments 1-205, wherein the TREM comprises an RNA sequence encoded by the DNA sequence of SEQ ID NO:231, or a fragment thereof.
437. The method, composition or pharmaceutical composition, cell, reaction mixture, or the bioreactor of any one of embodiments 1-205, wherein the TREM comprises an RNA sequence encoded by the DNA sequence of SEQ ID NO:232, or a fragment thereof.
438. The method, composition or pharmaceutical composition, cell, reaction mixture, or the bioreactor of any one of embodiments 1-205, wherein the TREM comprises an RNA sequence encoded by the DNA sequence of SEQ ID NO:233, or a fragment thereof.
439. The method, composition or pharmaceutical composition, cell, reaction mixture, or the bioreactor of any one of embodiments 1-205, wherein the TREM comprises an RNA sequence encoded by the DNA sequence of SEQ ID NO:234, or a fragment thereof.
440. The method, composition or pharmaceutical composition, cell, reaction mixture, or the bioreactor of any one of embodiments 1-205, wherein the TREM comprises an RNA sequence encoded by the DNA sequence of SEQ ID NO:235, or a fragment thereof.
441. The method, composition or pharmaceutical composition, cell, reaction mixture, or the bioreactor of any one of embodiments 1-205, wherein the TREM comprises an RNA sequence encoded by the DNA sequence of SEQ ID NO:236, or a fragment thereof.
442. The method, composition or pharmaceutical composition, cell, reaction mixture, or the bioreactor of any one of embodiments 1-205, wherein the TREM comprises an RNA sequence encoded by the DNA sequence of SEQ ID NO:237, or a fragment thereof.
443. The method, composition or pharmaceutical composition, cell, reaction mixture, or the bioreactor of any one of embodiments 1-205, wherein the TREM comprises an RNA sequence encoded by the DNA sequence of SEQ ID NO:238, or a fragment thereof.
444. The method, composition or pharmaceutical composition, cell, reaction mixture, or the bioreactor of any one of embodiments 1-205, wherein the TREM comprises an RNA sequence encoded by the DNA sequence of SEQ ID NO:239, or a fragment thereof.
445. The method, composition or pharmaceutical composition, cell, reaction mixture, or the bioreactor of any one of embodiments 1-205, wherein the TREM comprises an RNA sequence encoded by the DNA sequence of SEQ ID NO:240, or a fragment thereof.
446. The method, composition or pharmaceutical composition, cell, reaction mixture, or the bioreactor of any one of embodiments 1-205, wherein the TREM comprises an RNA sequence encoded by the DNA sequence of SEQ ID NO:241, or a fragment thereof.
447. The method, composition or pharmaceutical composition, cell, reaction mixture, or the bioreactor of any one of embodiments 1-205, wherein the TREM comprises an RNA sequence encoded by the DNA sequence of SEQ ID NO:242, or a fragment thereof.
448. The method, composition or pharmaceutical composition, cell, reaction mixture, or the bioreactor of any one of embodiments 1-205, wherein the TREM comprises an RNA sequence encoded by the DNA sequence of SEQ ID NO:243, or a fragment thereof.
449. The method, composition or pharmaceutical composition, cell, reaction mixture, or the bioreactor of any one of embodiments 1-205, wherein the TREM comprises an RNA sequence encoded by the DNA sequence of SEQ ID NO:244, or a fragment thereof.
450. The method, composition or pharmaceutical composition, cell, reaction mixture, or the bioreactor of any one of embodiments 1-205, wherein the TREM comprises an RNA sequence encoded by the DNA sequence of SEQ ID NO:245, or a fragment thereof.
451. The method, composition or pharmaceutical composition, cell, reaction mixture, or the bioreactor of any one of embodiments 1-205, wherein the TREM comprises an RNA sequence encoded by the DNA sequence of SEQ ID NO:246, or a fragment thereof.
452. The method, composition or pharmaceutical composition, cell, reaction mixture, or the bioreactor of any one of embodiments 1-205, wherein the TREM comprises an RNA sequence encoded by the DNA sequence of SEQ ID NO:247, or a fragment thereof.
453. The method, composition or pharmaceutical composition, cell, reaction mixture, or the bioreactor of any one of embodiments 1-205, wherein the TREM comprises an RNA sequence encoded by the DNA sequence of SEQ ID NO:248, or a fragment thereof.
454. The method, composition or pharmaceutical composition, cell, reaction mixture, or the bioreactor of any one of embodiments 1-205, wherein the TREM comprises an RNA sequence encoded by the DNA sequence of SEQ ID NO:249, or a fragment thereof.
455. The method, composition or pharmaceutical composition, cell, reaction mixture, or the bioreactor of any one of embodiments 1-205, wherein the TREM comprises an RNA sequence encoded by the DNA sequence of SEQ ID NO:250, or a fragment thereof.
456. The method, composition or pharmaceutical composition, cell, reaction mixture, or the bioreactor of any one of embodiments 1-205, wherein the TREM comprises an RNA sequence encoded by the DNA sequence of SEQ ID NO:251, or a fragment thereof.
457. The method, composition or pharmaceutical composition, cell, reaction mixture, or the bioreactor of any one of embodiments 1-205, wherein the TREM comprises an RNA sequence encoded by the DNA sequence of SEQ ID NO:252, or a fragment thereof.
458. The method, composition or pharmaceutical composition, cell, reaction mixture, or the bioreactor of any one of embodiments 1-205, wherein the TREM comprises an RNA sequence encoded by the DNA sequence of SEQ ID NO:253, or a fragment thereof.
459. The method, composition or pharmaceutical composition, cell, reaction mixture, or the bioreactor of any one of embodiments 1-205, wherein the TREM comprises an RNA sequence encoded by the DNA sequence of SEQ ID NO:254, or a fragment thereof.
460. The method, composition or pharmaceutical composition, cell, reaction mixture, or the bioreactor of any one of embodiments 1-205, wherein the TREM comprises an RNA sequence encoded by the DNA sequence of SEQ ID NO:255, or a fragment thereof.
461. The method, composition or pharmaceutical composition, cell, reaction mixture, or the bioreactor of any one of embodiments 1-205, wherein the TREM comprises an RNA sequence encoded by the DNA sequence of SEQ ID NO:256, or a fragment thereof.
462. The method, composition or pharmaceutical composition, cell, reaction mixture, or the bioreactor of any one of embodiments 1-205, wherein the TREM comprises an RNA sequence encoded by the DNA sequence of SEQ ID NO:257, or a fragment thereof.
463. The method, composition or pharmaceutical composition, cell, reaction mixture, or the bioreactor of any one of embodiments 1-205, wherein the TREM comprises an RNA sequence encoded by the DNA sequence of SEQ ID NO:258, or a fragment thereof.
464. The method, composition or pharmaceutical composition, cell, reaction mixture, or the bioreactor of any one of embodiments 1-205, wherein the TREM comprises an RNA sequence encoded by the DNA sequence of SEQ ID NO:259, or a fragment thereof.
465. The method, composition or pharmaceutical composition, cell, reaction mixture, or the bioreactor of any one of embodiments 1-205, wherein the TREM comprises an RNA sequence encoded by the DNA sequence of SEQ ID NO:260, or a fragment thereof.
466. The method, composition or pharmaceutical composition, cell, reaction mixture, or the bioreactor of any one of embodiments 1-205, wherein the TREM comprises an RNA sequence encoded by the DNA sequence of SEQ ID NO:261, or a fragment thereof.
467. The method, composition or pharmaceutical composition, cell, reaction mixture, or the bioreactor of any one of embodiments 1-205, wherein the TREM comprises an RNA sequence encoded by the DNA sequence of SEQ ID NO:262, or a fragment thereof.
468. The method, composition or pharmaceutical composition, cell, reaction mixture, or the bioreactor of any one of embodiments 1-205, wherein the TREM comprises an RNA sequence encoded by the DNA sequence of SEQ ID NO:263, or a fragment thereof.
469. The method, composition or pharmaceutical composition, cell, reaction mixture, or the bioreactor of any one of embodiments 1-205, wherein the TREM comprises an RNA sequence encoded by the DNA sequence of SEQ ID NO:264, or a fragment thereof.
470. The method, composition or pharmaceutical composition, cell, reaction mixture, or the bioreactor of any one of embodiments 1-205, wherein the TREM comprises an RNA sequence encoded by the DNA sequence of SEQ ID NO:265, or a fragment thereof.
471. The method, composition or pharmaceutical composition, cell, reaction mixture, or the bioreactor of any one of embodiments 1-205, wherein the TREM comprises an RNA sequence encoded by the DNA sequence of SEQ ID NO:266, or a fragment thereof.
472. The method, composition or pharmaceutical composition, cell, reaction mixture, or the bioreactor of any one of embodiments 1-205, wherein the TREM comprises an RNA sequence encoded by the DNA sequence of SEQ ID NO:267, or a fragment thereof.
473. The method, composition or pharmaceutical composition, cell, reaction mixture, or the bioreactor of any one of embodiments 1-205, wherein the TREM comprises an RNA sequence encoded by the DNA sequence of SEQ ID NO:268, or a fragment thereof.
474. The method, composition or pharmaceutical composition, cell, reaction mixture, or the bioreactor of any one of embodiments 1-205, wherein the TREM comprises an RNA sequence encoded by the DNA sequence of SEQ ID NO:269, or a fragment thereof.
475. The method, composition or pharmaceutical composition, cell, reaction mixture, or the bioreactor of any one of embodiments 1-205, wherein the TREM comprises an RNA sequence encoded by the DNA sequence of SEQ ID NO:270, or a fragment thereof.
476. The method, composition or pharmaceutical composition, cell, reaction mixture, or the bioreactor of any one of embodiments 1-205, wherein the TREM comprises an RNA sequence encoded by the DNA sequence of SEQ ID NO:271, or a fragment thereof.
477. The method, composition or pharmaceutical composition, cell, reaction mixture, or the bioreactor of any one of embodiments 1-205, wherein the TREM comprises an RNA sequence encoded by the DNA sequence of SEQ ID NO:272, or a fragment thereof.
478. The method, composition or pharmaceutical composition, cell, reaction mixture, or the bioreactor of any one of embodiments 1-205, wherein the TREM comprises an RNA sequence encoded by the DNA sequence of SEQ ID NO:273, or a fragment thereof.
479. The method, composition or pharmaceutical composition, cell, reaction mixture, or the bioreactor of any one of embodiments 1-205, wherein the TREM comprises an RNA sequence encoded by the DNA sequence of SEQ ID NO:274, or a fragment thereof.
480. The method, composition or pharmaceutical composition, cell, reaction mixture, or the bioreactor of any one of embodiments 1-205, wherein the TREM comprises an RNA sequence encoded by the DNA sequence of SEQ ID NO:275, or a fragment thereof.
481. The method, composition or pharmaceutical composition, cell, reaction mixture, or the bioreactor of any one of embodiments 1-205, wherein the TREM comprises an RNA sequence encoded by the DNA sequence of SEQ ID NO:276, or a fragment thereof.
482. The method, composition or pharmaceutical composition, cell, reaction mixture, or the bioreactor of any one of embodiments 1-205, wherein the TREM comprises an RNA sequence encoded by the DNA sequence of SEQ ID NO:277, or a fragment thereof.
483. The method, composition or pharmaceutical composition, cell, reaction mixture, or the bioreactor of any one of embodiments 1-205, wherein the TREM comprises an RNA sequence encoded by the DNA sequence of SEQ ID NO:278, or a fragment thereof.
484. The method, composition or pharmaceutical composition, cell, reaction mixture, or the bioreactor of any one of embodiments 1-205, wherein the TREM comprises an RNA sequence encoded by the DNA sequence of SEQ ID NO:279, or a fragment thereof.
485. The method, composition or pharmaceutical composition, cell, reaction mixture, or the bioreactor of any one of embodiments 1-205, wherein the TREM comprises an RNA sequence encoded by the DNA sequence of SEQ ID NO:280, or a fragment thereof.
486. The method, composition or pharmaceutical composition, cell, reaction mixture, or the bioreactor of any one of embodiments 1-205, wherein the TREM comprises an RNA sequence encoded by the DNA sequence of SEQ ID NO:281, or a fragment thereof.
487. The method, composition or pharmaceutical composition, cell, reaction mixture, or the bioreactor of any one of embodiments 1-205, wherein the TREM comprises an RNA sequence encoded by the DNA sequence of SEQ ID NO:282, or a fragment thereof.
488. The method, composition or pharmaceutical composition, cell, reaction mixture, or the bioreactor of any one of embodiments 1-205, wherein the TREM comprises an RNA sequence encoded by the DNA sequence of SEQ ID NO:283, or a fragment thereof.
489. The method, composition or pharmaceutical composition, cell, reaction mixture, or the bioreactor of any one of embodiments 1-205, wherein the TREM comprises an RNA sequence encoded by the DNA sequence of SEQ ID NO:284, or a fragment thereof.
490. The method, composition or pharmaceutical composition, cell, reaction mixture, or the bioreactor of any one of embodiments 1-205, wherein the TREM comprises an RNA sequence encoded by the DNA sequence of SEQ ID NO:285, or a fragment thereof.
491. The method, composition or pharmaceutical composition, cell, reaction mixture, or the bioreactor of any one of embodiments 1-205, wherein the TREM comprises an RNA sequence encoded by the DNA sequence of SEQ ID NO:286, or a fragment thereof.
492. The method, composition or pharmaceutical composition, cell, reaction mixture, or the bioreactor of any one of embodiments 1-205, wherein the TREM comprises an RNA sequence encoded by the DNA sequence of SEQ ID NO:287, or a fragment thereof.
493. The method, composition or pharmaceutical composition, cell, reaction mixture, or the bioreactor of any one of embodiments 1-205, wherein the TREM comprises an RNA sequence encoded by the DNA sequence of SEQ ID NO:288, or a fragment thereof.
494. The method, composition or pharmaceutical composition, cell, reaction mixture, or the bioreactor of any one of embodiments 1-205, wherein the TREM comprises an RNA sequence encoded by the DNA sequence of SEQ ID NO:289, or a fragment thereof.
495. The method, composition or pharmaceutical composition, cell, reaction mixture, or the bioreactor of any one of embodiments 1-205, wherein the TREM comprises an RNA sequence encoded by the DNA sequence of SEQ ID NO:290, or a fragment thereof.
496. The method, composition or pharmaceutical composition, cell, reaction mixture, or the bioreactor of any one of embodiments 1-205, wherein the TREM comprises an RNA sequence encoded by the DNA sequence of SEQ ID NO:291, or a fragment thereof.
497. The method, composition or pharmaceutical composition, cell, reaction mixture, or the bioreactor of any one of embodiments 1-205, wherein the TREM comprises an RNA sequence encoded by the DNA sequence of SEQ ID NO:292, or a fragment thereof.
498. The method, composition or pharmaceutical composition, cell, reaction mixture, or the bioreactor of any one of embodiments 1-205, wherein the TREM comprises an RNA sequence encoded by the DNA sequence of SEQ ID NO:293, or a fragment thereof.
499. The method, composition or pharmaceutical composition, cell, reaction mixture, or the bioreactor of any one of embodiments 1-205, wherein the TREM comprises an RNA sequence encoded by the DNA sequence of SEQ ID NO:294, or a fragment thereof.
500. The method, composition or pharmaceutical composition, cell, reaction mixture, or the bioreactor of any one of embodiments 1-205, wherein the TREM comprises an RNA sequence encoded by the DNA sequence of SEQ ID NO:295, or a fragment thereof.
501. The method, composition or pharmaceutical composition, cell, reaction mixture, or the bioreactor of any one of embodiments 1-205, wherein the TREM comprises an RNA sequence encoded by the DNA sequence of SEQ ID NO:296, or a fragment thereof.
502. The method, composition or pharmaceutical composition, cell, reaction mixture, or the bioreactor of any one of embodiments 1-205, wherein the TREM comprises an RNA sequence encoded by the DNA sequence of SEQ ID NO:297, or a fragment thereof.
503. The method, composition or pharmaceutical composition, cell, reaction mixture, or the bioreactor of any one of embodiments 1-205, wherein the TREM comprises an RNA sequence encoded by the DNA sequence of SEQ ID NO:298, or a fragment thereof.
504. The method, composition or pharmaceutical composition, cell, reaction mixture, or the bioreactor of any one of embodiments 1-205, wherein the TREM comprises an RNA sequence encoded by the DNA sequence of SEQ ID NO:299, or a fragment thereof.
505. The method, composition or pharmaceutical composition, cell, reaction mixture, or the bioreactor of any one of embodiments 1-205, wherein the TREM comprises an RNA sequence encoded by the DNA sequence of SEQ ID NO:300, or a fragment thereof.
506. The method, composition or pharmaceutical composition, cell, reaction mixture, or the bioreactor of any one of embodiments 1-205, wherein the TREM comprises an RNA sequence encoded by the DNA sequence of SEQ ID NO:301, or a fragment thereof.
507. The method, composition or pharmaceutical composition, cell, reaction mixture, or the bioreactor of any one of embodiments 1-205, wherein the TREM comprises an RNA sequence encoded by the DNA sequence of SEQ ID NO:302, or a fragment thereof.
508. The method, composition or pharmaceutical composition, cell, reaction mixture, or the bioreactor of any one of embodiments 1-205, wherein the TREM comprises an RNA sequence encoded by the DNA sequence of SEQ ID NO:303, or a fragment thereof.
509. The method, composition or pharmaceutical composition, cell, reaction mixture, or the bioreactor of any one of embodiments 1-205, wherein the TREM comprises an RNA sequence encoded by the DNA sequence of SEQ ID NO:304, or a fragment thereof.
510. The method, composition or pharmaceutical composition, cell, reaction mixture, or the bioreactor of any one of embodiments 1-205, wherein the TREM comprises an RNA sequence encoded by the DNA sequence of SEQ ID NO:305, or a fragment thereof.
511. The method, composition or pharmaceutical composition, cell, reaction mixture, or the bioreactor of any one of embodiments 1-205, wherein the TREM comprises an RNA sequence encoded by the DNA sequence of SEQ ID NO:306, or a fragment thereof.
512. The method, composition or pharmaceutical composition, cell, reaction mixture, or the bioreactor of any one of embodiments 1-205, wherein the TREM comprises an RNA sequence encoded by the DNA sequence of SEQ ID NO:307, or a fragment thereof.
513. The method, composition or pharmaceutical composition, cell, reaction mixture, or the bioreactor of any one of embodiments 1-205, wherein the TREM comprises an RNA sequence encoded by the DNA sequence of SEQ ID NO:308, or a fragment thereof. 514. The method, composition or pharmaceutical composition, cell, reaction mixture, or the bioreactor of any one of embodiments 1-205, wherein the TREM comprises an RNA sequence encoded by the DNA sequence of SEQ ID NO:309, or a fragment thereof.
515. The method, composition or pharmaceutical composition, cell, reaction mixture, or the bioreactor of any one of embodiments 1-205, wherein the TREM comprises an RNA sequence encoded by the DNA sequence of SEQ ID NO:310, or a fragment thereof.
516. The method, composition or pharmaceutical composition, cell, reaction mixture, or the bioreactor of any one of embodiments 1-205, wherein the TREM comprises an RNA sequence encoded by the DNA sequence of SEQ ID NO:311, or a fragment thereof.
517. The method, composition or pharmaceutical composition, cell, reaction mixture, or the bioreactor of any one of embodiments 1-205, wherein the TREM comprises an RNA sequence encoded by the DNA sequence of SEQ ID NO:312, or a fragment thereof.
518. The method, composition or pharmaceutical composition, cell, reaction mixture, or the bioreactor of any one of embodiments 1-205, wherein the TREM comprises an RNA sequence encoded by the DNA sequence of SEQ ID NO:313, or a fragment thereof.
519. The method, composition or pharmaceutical composition, cell, reaction mixture, or the bioreactor of any one of embodiments 1-205, wherein the TREM comprises an RNA sequence encoded by the DNA sequence of SEQ ID NO:314, or a fragment thereof.
520. The method, composition or pharmaceutical composition, cell, reaction mixture, or the bioreactor of any one of embodiments 1-205, wherein the TREM comprises an RNA sequence encoded by the DNA sequence of SEQ ID NO:315, or a fragment thereof.
521. The method, composition or pharmaceutical composition, cell, reaction mixture, or the bioreactor of any one of embodiments 1-205, wherein the TREM comprises an RNA sequence encoded by the DNA sequence of SEQ ID NO:316, or a fragment thereof. 522. The method, composition or pharmaceutical composition, cell, reaction mixture, or the bioreactor of any one of embodiments 1-205, wherein the TREM comprises an RNA sequence encoded by the DNA sequence of SEQ ID NO:317, or a fragment thereof.
523. The method, composition or pharmaceutical composition, cell, reaction mixture, or the bioreactor of any one of embodiments 1-205, wherein the TREM comprises an RNA sequence encoded by the DNA sequence of SEQ ID NO:318, or a fragment thereof.
524. The method, composition or pharmaceutical composition, cell, reaction mixture, or the bioreactor of any one of embodiments 1-205, wherein the TREM comprises an RNA sequence encoded by the DNA sequence of SEQ ID NO:319, or a fragment thereof.
525. The method, composition or pharmaceutical composition, cell, reaction mixture, or the bioreactor of any one of embodiments 1-205, wherein the TREM comprises an RNA sequence encoded by the DNA sequence of SEQ ID NO:320, or a fragment thereof.
526. The method, composition or pharmaceutical composition, cell, reaction mixture, or the bioreactor of any one of embodiments 1-205, wherein the TREM comprises an RNA sequence encoded by the DNA sequence of SEQ ID NO:321, or a fragment thereof.
527. The method, composition or pharmaceutical composition, cell, reaction mixture, or the bioreactor of any one of embodiments 1-205, wherein the TREM comprises an RNA sequence encoded by the DNA sequence of SEQ ID NO:322, or a fragment thereof.
528. The method, composition or pharmaceutical composition, cell, reaction mixture, or the bioreactor of any one of embodiments 1-205, wherein the TREM comprises an RNA sequence encoded by the DNA sequence of SEQ ID NO:323, or a fragment thereof.
529. The method, composition or pharmaceutical composition, cell, reaction mixture, or the bioreactor of any one of embodiments 1-205, wherein the TREM comprises an RNA sequence encoded by the DNA sequence of SEQ ID NO:324, or a fragment thereof. 530. The method, composition or pharmaceutical composition, cell, reaction mixture, or the bioreactor of any one of embodiments 1-205, wherein the TREM comprises an RNA sequence encoded by the DNA sequence of SEQ ID NO:325, or a fragment thereof.
531. The method, composition or pharmaceutical composition, cell, reaction mixture, or the bioreactor of any one of embodiments 1-205, wherein the TREM comprises an RNA sequence encoded by the DNA sequence of SEQ ID NO:326, or a fragment thereof.
532. The method, composition or pharmaceutical composition, cell, reaction mixture, or the bioreactor of any one of embodiments 1-205, wherein the TREM comprises an RNA sequence encoded by the DNA sequence of SEQ ID NO:327, or a fragment thereof.
533. The method, composition or pharmaceutical composition, cell, reaction mixture, or the bioreactor of any one of embodiments 1-205, wherein the TREM comprises an RNA sequence encoded by the DNA sequence of SEQ ID NO:328, or a fragment thereof.
534. The method, composition or pharmaceutical composition, cell, reaction mixture, or the bioreactor of any one of embodiments 1-205, wherein the TREM comprises an RNA sequence encoded by the DNA sequence of SEQ ID NO:329, or a fragment thereof.
535. The method, composition or pharmaceutical composition, cell, reaction mixture, or the bioreactor of any one of embodiments 1-205, wherein the TREM comprises an RNA sequence encoded by the DNA sequence of SEQ ID NO:330, or a fragment thereof.
536. The method, composition or pharmaceutical composition, cell, reaction mixture, or the bioreactor of any one of embodiments 1-205, wherein the TREM comprises an RNA sequence encoded by the DNA sequence of SEQ ID NO:331, or a fragment thereof.
537. The method, composition or pharmaceutical composition, cell, reaction mixture, or the bioreactor of any one of embodiments 1-205, wherein the TREM comprises an RNA sequence encoded by the DNA sequence of SEQ ID NO:332, or a fragment thereof.
538. The method, composition or pharmaceutical composition, cell, reaction mixture, or the bioreactor of any one of embodiments 1-205, wherein the TREM comprises an RNA sequence encoded by the DNA sequence of SEQ ID NO:333, or a fragment thereof.
539. The method, composition or pharmaceutical composition, cell, reaction mixture, or the bioreactor of any one of embodiments 1-205, wherein the TREM comprises an RNA sequence encoded by the DNA sequence of SEQ ID NO:334, or a fragment thereof.
540. The method, composition or pharmaceutical composition, cell, reaction mixture, or the bioreactor of any one of embodiments 1-205, wherein the TREM comprises an RNA sequence encoded by the DNA sequence of SEQ ID NO:335, or a fragment thereof.
541. The method, composition or pharmaceutical composition, cell, reaction mixture, or the bioreactor of any one of embodiments 1-205, wherein the TREM comprises an RNA sequence encoded by the DNA sequence of SEQ ID NO:336, or a fragment thereof.
542. The method, composition or pharmaceutical composition, cell, reaction mixture, or the bioreactor of any one of embodiments 1-205, wherein the TREM comprises an RNA sequence encoded by the DNA sequence of SEQ ID NO:337, or a fragment thereof.
543. The method, composition or pharmaceutical composition, cell, reaction mixture, or the bioreactor of any one of embodiments 1-205, wherein the TREM comprises an RNA sequence encoded by the DNA sequence of SEQ ID NO:338, or a fragment thereof.
544. The method, composition or pharmaceutical composition, cell, reaction mixture, or the bioreactor of any one of embodiments 1-205, wherein the TREM comprises an RNA sequence encoded by the DNA sequence of SEQ ID NO:339, or a fragment thereof.
545. The method, composition or pharmaceutical composition, cell, reaction mixture, or the bioreactor of any one of embodiments 1-205, wherein the TREM comprises an RNA sequence encoded by the DNA sequence of SEQ ID NO:340, or a fragment thereof.
546. The method, composition or pharmaceutical composition, cell, reaction mixture, or the bioreactor of any one of embodiments 1-205, wherein the TREM comprises an RNA sequence encoded by the DNA sequence of SEQ ID NO:341, or a fragment thereof.
547. The method, composition or pharmaceutical composition, cell, reaction mixture, or the bioreactor of any one of embodiments 1-205, wherein the TREM comprises an RNA sequence encoded by the DNA sequence of SEQ ID NO:342, or a fragment thereof.
548. The method, composition or pharmaceutical composition, cell, reaction mixture, or the bioreactor of any one of embodiments 1-205, wherein the TREM comprises an RNA sequence encoded by the DNA sequence of SEQ ID NO:343, or a fragment thereof.
549. The method, composition or pharmaceutical composition, cell, reaction mixture, or the bioreactor of any one of embodiments 1-205, wherein the TREM comprises an RNA sequence encoded by the DNA sequence of SEQ ID NO:344, or a fragment thereof.
550. The method, composition or pharmaceutical composition, cell, reaction mixture, or the bioreactor of any one of embodiments 1-205, wherein the TREM comprises an RNA sequence encoded by the DNA sequence of SEQ ID NO:345, or a fragment thereof.
551. The method, composition or pharmaceutical composition, cell, reaction mixture, or the bioreactor of any one of embodiments 1-205, wherein the TREM comprises an RNA sequence encoded by the DNA sequence of SEQ ID NO:346, or a fragment thereof.
552. The method, composition or pharmaceutical composition, cell, reaction mixture, or the bioreactor of any one of embodiments 1-205, wherein the TREM comprises an RNA sequence encoded by the DNA sequence of SEQ ID NO:347, or a fragment thereof.
553. The method, composition or pharmaceutical composition, cell, reaction mixture, or the bioreactor of any one of embodiments 1-205, wherein the TREM comprises an RNA sequence encoded by the DNA sequence of SEQ ID NO:348, or a fragment thereof.
554. The method, composition or pharmaceutical composition, cell, reaction mixture, or the bioreactor of any one of embodiments 1-205, wherein the TREM comprises an RNA sequence encoded by the DNA sequence of SEQ ID NO:349, or a fragment thereof.
555. The method, composition or pharmaceutical composition, cell, reaction mixture, or the bioreactor of any one of embodiments 1-205, wherein the TREM comprises an RNA sequence encoded by the DNA sequence of SEQ ID NO:350, or a fragment thereof.
556. The method, composition or pharmaceutical composition, cell, reaction mixture, or the bioreactor of any one of embodiments 1-205, wherein the TREM comprises an RNA sequence encoded by the DNA sequence of SEQ ID NO:351, or a fragment thereof.
557. The method, composition or pharmaceutical composition, cell, reaction mixture, or the bioreactor of any one of embodiments 1-205, wherein the TREM comprises an RNA sequence encoded by the DNA sequence of SEQ ID NO:352, or a fragment thereof.
558. The method, composition or pharmaceutical composition, cell, reaction mixture, or the bioreactor of any one of embodiments 1-205, wherein the TREM comprises an RNA sequence encoded by the DNA sequence of SEQ ID NO:353, or a fragment thereof.
559. The method, composition or pharmaceutical composition, cell, reaction mixture, or the bioreactor of any one of embodiments 1-205, wherein the TREM comprises an RNA sequence encoded by the DNA sequence of SEQ ID NO:354, or a fragment thereof.
560. The method, composition or pharmaceutical composition, cell, reaction mixture, or the bioreactor of any one of embodiments 1-205, wherein the TREM comprises an RNA sequence encoded by the DNA sequence of SEQ ID NO:355, or a fragment thereof.
561. The method, composition or pharmaceutical composition, cell, reaction mixture, or the bioreactor of any one of embodiments 1-205, wherein the TREM comprises an RNA sequence encoded by the DNA sequence of SEQ ID NO:356, or a fragment thereof.
562. The method, composition or pharmaceutical composition, cell, reaction mixture, or the bioreactor of any one of embodiments 1-205, wherein the TREM comprises an RNA sequence encoded by the DNA sequence of SEQ ID NO:357, or a fragment thereof.
563. The method, composition or pharmaceutical composition, cell, reaction mixture, or the bioreactor of any one of embodiments 1-205, wherein the TREM comprises an RNA sequence encoded by the DNA sequence of SEQ ID NO:358, or a fragment thereof.
564. The method, composition or pharmaceutical composition, cell, reaction mixture, or the bioreactor of any one of embodiments 1-205, wherein the TREM comprises an RNA sequence encoded by the DNA sequence of SEQ ID NO:359, or a fragment thereof.
565. The method, composition or pharmaceutical composition, cell, reaction mixture, or the bioreactor of any one of embodiments 1-205, wherein the TREM comprises an RNA sequence encoded by the DNA sequence of SEQ ID NO:360, or a fragment thereof.
566. The method, composition or pharmaceutical composition, cell, reaction mixture, or the bioreactor of any one of embodiments 1-205, wherein the TREM comprises an RNA sequence encoded by the DNA sequence of SEQ ID NO:361, or a fragment thereof.
567. The method, composition or pharmaceutical composition, cell, reaction mixture, or the bioreactor of any one of embodiments 1-205, wherein the TREM comprises an RNA sequence encoded by the DNA sequence of SEQ ID NO:362, or a fragment thereof.
568. The method, composition or pharmaceutical composition, cell, reaction mixture, or the bioreactor of any one of embodiments 1-205, wherein the TREM comprises an RNA sequence encoded by the DNA sequence of SEQ ID NO:363, or a fragment thereof.
569. The method, composition or pharmaceutical composition, cell, reaction mixture, or the bioreactor of any one of embodiments 1-205, wherein the TREM comprises an RNA sequence encoded by the DNA sequence of SEQ ID NO:364, or a fragment thereof.
570. The method, composition or pharmaceutical composition, cell, reaction mixture, or the bioreactor of any one of embodiments 1-205, wherein the TREM comprises an RNA sequence encoded by the DNA sequence of SEQ ID NO:365, or a fragment thereof.
571. The method, composition or pharmaceutical composition, cell, reaction mixture, or the bioreactor of any one of embodiments 1-205, wherein the TREM comprises an RNA sequence encoded by the DNA sequence of SEQ ID NO:366, or a fragment thereof.
572. The method, composition or pharmaceutical composition, cell, reaction mixture, or the bioreactor of any one of embodiments 1-205, wherein the TREM comprises an RNA sequence encoded by the DNA sequence of SEQ ID NO:367, or a fragment thereof.
573. The method, composition or pharmaceutical composition, cell, reaction mixture, or the bioreactor of any one of embodiments 1-205, wherein the TREM comprises an RNA sequence encoded by the DNA sequence of SEQ ID NO:368, or a fragment thereof.
574. The method, composition or pharmaceutical composition, cell, reaction mixture, or the bioreactor of any one of embodiments 1-205, wherein the TREM comprises an RNA sequence encoded by the DNA sequence of SEQ ID NO:369, or a fragment thereof.
575. The method, composition or pharmaceutical composition, cell, reaction mixture, or the bioreactor of any one of embodiments 1-205, wherein the TREM comprises an RNA sequence encoded by the DNA sequence of SEQ ID NO:370, or a fragment thereof.
576. The method, composition or pharmaceutical composition, cell, reaction mixture, or the bioreactor of any one of embodiments 1-205, wherein the TREM comprises an RNA sequence encoded by the DNA sequence of SEQ ID NO:371, or a fragment thereof.
577. The method, composition or pharmaceutical composition, cell, reaction mixture, or the bioreactor of any one of embodiments 1-205, wherein the TREM comprises an RNA sequence encoded by the DNA sequence of SEQ ID NO:372, or a fragment thereof.
578. The method, composition or pharmaceutical composition, cell, reaction mixture, or the bioreactor of any one of embodiments 1-205, wherein the TREM comprises an RNA sequence encoded by the DNA sequence of SEQ ID NO:373, or a fragment thereof.
579. The method, composition or pharmaceutical composition, cell, reaction mixture, or the bioreactor of any one of embodiments 1-205, wherein the TREM comprises an RNA sequence encoded by the DNA sequence of SEQ ID NO:374, or a fragment thereof.
580. The method, composition or pharmaceutical composition, cell, reaction mixture, or the bioreactor of any one of embodiments 1-205, wherein the TREM comprises an RNA sequence encoded by the DNA sequence of SEQ ID NO:375, or a fragment thereof.
581. The method, composition or pharmaceutical composition, cell, reaction mixture, or the bioreactor of any one of embodiments 1-205, wherein the TREM comprises an RNA sequence encoded by the DNA sequence of SEQ ID NO:376, or a fragment thereof.
582. The method, composition or pharmaceutical composition, cell, reaction mixture, or the bioreactor of any one of embodiments 1-205, wherein the TREM comprises an RNA sequence encoded by the DNA sequence of SEQ ID NO:377, or a fragment thereof.
583. The method, composition or pharmaceutical composition, cell, reaction mixture, or the bioreactor of any one of embodiments 1-205, wherein the TREM comprises an RNA sequence encoded by the DNA sequence of SEQ ID NO:378, or a fragment thereof.
584. The method, composition or pharmaceutical composition, cell, reaction mixture, or the bioreactor of any one of embodiments 1-205, wherein the TREM comprises an RNA sequence encoded by the DNA sequence of SEQ ID NO:379, or a fragment thereof.
585. The method, composition or pharmaceutical composition, cell, reaction mixture, or the bioreactor of any one of embodiments 1-205, wherein the TREM comprises an RNA sequence encoded by the DNA sequence of SEQ ID NO:380, or a fragment thereof.
586. The method, composition or pharmaceutical composition, cell, reaction mixture, or the bioreactor of any one of embodiments 1-205, wherein the TREM comprises an RNA sequence encoded by the DNA sequence of SEQ ID NO:381, or a fragment thereof.
587. The method, composition or pharmaceutical composition, cell, reaction mixture, or the bioreactor of any one of embodiments 1-205, wherein the TREM comprises an RNA sequence encoded by the DNA sequence of SEQ ID NO:382, or a fragment thereof.
588. The method, composition or pharmaceutical composition, cell, reaction mixture, or the bioreactor of any one of embodiments 1-205, wherein the TREM comprises an RNA sequence encoded by the DNA sequence of SEQ ID NO:383, or a fragment thereof.
589. The method, composition or pharmaceutical composition, cell, reaction mixture, or the bioreactor of any one of embodiments 1-205, wherein the TREM comprises an RNA sequence encoded by the DNA sequence of SEQ ID NO:384, or a fragment thereof.
590. The method, composition or pharmaceutical composition, cell, reaction mixture, or the bioreactor of any one of embodiments 1-205, wherein the TREM comprises an RNA sequence encoded by the DNA sequence of SEQ ID NO:385, or a fragment thereof.
591. The method, composition or pharmaceutical composition, cell, reaction mixture, or the bioreactor of any one of embodiments 1-205, wherein the TREM comprises an RNA sequence encoded by the DNA sequence of SEQ ID NO:386, or a fragment thereof.
592. The method, composition or pharmaceutical composition, cell, reaction mixture, or the bioreactor of any one of embodiments 1-205, wherein the TREM comprises an RNA sequence encoded by the DNA sequence of SEQ ID NO:387, or a fragment thereof.
593. The method, composition or pharmaceutical composition, cell, reaction mixture, or the bioreactor of any one of embodiments 1-205, wherein the TREM comprises an RNA sequence encoded by the DNA sequence of SEQ ID NO:388, or a fragment thereof.
594. The method, composition or pharmaceutical composition, cell, reaction mixture, or the bioreactor of any one of embodiments 1-205, wherein the TREM comprises an RNA sequence encoded by the DNA sequence of SEQ ID NO:389, or a fragment thereof.
595. The method, composition or pharmaceutical composition, cell, reaction mixture, or the bioreactor of any one of embodiments 1-205, wherein the TREM comprises an RNA sequence encoded by the DNA sequence of SEQ ID NO:390, or a fragment thereof.
596. The method, composition or pharmaceutical composition, cell, reaction mixture, or the bioreactor of any one of embodiments 1-205, wherein the TREM comprises an RNA sequence encoded by the DNA sequence of SEQ ID NO:391, or a fragment thereof.
597. The method, composition or pharmaceutical composition, cell, reaction mixture, or the bioreactor of any one of embodiments 1-205, wherein the TREM comprises an RNA sequence encoded by the DNA sequence of SEQ ID NO:392, or a fragment thereof.
598. The method, composition or pharmaceutical composition, cell, reaction mixture, or the bioreactor of any one of embodiments 1-205, wherein the TREM comprises an RNA sequence encoded by the DNA sequence of SEQ ID NO:393, or a fragment thereof.
599. The method, composition or pharmaceutical composition, cell, reaction mixture, or the bioreactor of any one of embodiments 1-205, wherein the TREM comprises an RNA sequence encoded by the DNA sequence of SEQ ID NO:394, or a fragment thereof.
600. The method, composition or pharmaceutical composition, cell, reaction mixture, or the bioreactor of any one of embodiments 1-205, wherein the TREM comprises an RNA sequence encoded by the DNA sequence of SEQ ID NO:395, or a fragment thereof.
601. The method, composition or pharmaceutical composition, cell, reaction mixture, or the bioreactor of any one of embodiments 1-205, wherein the TREM comprises an RNA sequence encoded by the DNA sequence of SEQ ID NO:396, or a fragment thereof.
602. The method, composition or pharmaceutical composition, cell, reaction mixture, or the bioreactor of any one of embodiments 1-205, wherein the TREM comprises an RNA sequence encoded by the DNA sequence of SEQ ID NO:397, or a fragment thereof.
603. The method, composition or pharmaceutical composition, cell, reaction mixture, or the bioreactor of any one of embodiments 1-205, wherein the TREM comprises an RNA sequence encoded by the DNA sequence of SEQ ID NO:398, or a fragment thereof.
604. The method, composition or pharmaceutical composition, cell, reaction mixture, or the bioreactor of any one of embodiments 1-205, wherein the TREM comprises an RNA sequence encoded by the DNA sequence of SEQ ID NO:399, or a fragment thereof.
605. The method, composition or pharmaceutical composition, cell, reaction mixture, or the bioreactor of any one of embodiments 1-205, wherein the TREM comprises an RNA sequence encoded by the DNA sequence of SEQ ID NO:400, or a fragment thereof.
606. The method, composition or pharmaceutical composition, cell, reaction mixture, or the bioreactor of any one of embodiments 1-205, wherein the TREM comprises an RNA sequence encoded by the DNA sequence of SEQ ID NO:401, or a fragment thereof.
607. The method, composition or pharmaceutical composition, cell, reaction mixture, or the bioreactor of any one of embodiments 1-205, wherein the TREM comprises an RNA sequence encoded by the DNA sequence of SEQ ID NO:402, or a fragment thereof.
608. The method, composition or pharmaceutical composition, cell, reaction mixture, or the bioreactor of any one of embodiments 1-205, wherein the TREM comprises an RNA sequence encoded by the DNA sequence of SEQ ID NO: 403, or a fragment thereof.
609. The method, composition or pharmaceutical composition, cell, reaction mixture, or the bioreactor of any one of embodiments 1-205, wherein the TREM comprises an RNA sequence encoded by the DNA sequence of SEQ ID NO: 404, or a fragment thereof.
610. The method, composition or pharmaceutical composition, cell, reaction mixture, or the bioreactor of any one of embodiments 1-205, wherein the TREM comprises an RNA sequence encoded by the DNA sequence of SEQ ID NO: 405, or a fragment thereof.
611. The method, composition or pharmaceutical composition, cell, reaction mixture, or the bioreactor of any one of embodiments 1-205, wherein the TREM comprises an RNA sequence encoded by the DNA sequence of SEQ ID NO: 406, or a fragment thereof.
612. The method, composition or pharmaceutical composition, cell, reaction mixture, or the bioreactor of any one of embodiments 1-205, wherein the TREM comprises an RNA sequence encoded by the DNA sequence of SEQ ID NO: 407, or a fragment thereof.
613. The method, composition or pharmaceutical composition, cell, reaction mixture, or the bioreactor of any one of embodiments 1-205, wherein the TREM comprises an RNA sequence encoded by the DNA sequence of SEQ ID NO: 408, or a fragment thereof.
614. The method, composition or pharmaceutical composition, cell, reaction mixture, or the bioreactor of any one of embodiments 1-205, wherein the TREM comprises an RNA sequence encoded by the DNA sequence of SEQ ID NO: 409, or a fragment thereof.
615. The method, composition or pharmaceutical composition, cell, reaction mixture, or the bioreactor of any one of embodiments 1-205, wherein the TREM comprises an RNA sequence encoded by the DNA sequence of SEQ ID NO: 410, or a fragment thereof.
616. The method, composition or pharmaceutical composition, cell, reaction mixture, or the bioreactor of any one of embodiments 1-205, wherein the TREM comprises an RNA sequence encoded by the DNA sequence of SEQ ID NO: 411, or a fragment thereof.
617. The method, composition or pharmaceutical composition, cell, reaction mixture, or the bioreactor of any one of embodiments 1-205, wherein the TREM comprises an RNA sequence encoded by the DNA sequence of SEQ ID NO: 412, or a fragment thereof.
618. The method, composition or pharmaceutical composition, cell, reaction mixture, or the bioreactor of any one of embodiments 1-205, wherein the TREM comprises an RNA sequence encoded by the DNA sequence of SEQ ID NO: 413, or a fragment thereof.
619. The method, composition or pharmaceutical composition, cell, reaction mixture, or the bioreactor of any one of embodiments 1-205, wherein the TREM comprises an RNA sequence encoded by the DNA sequence of SEQ ID NO: 414, or a fragment thereof.
620. The method, composition or pharmaceutical composition, cell, reaction mixture, or the bioreactor of any one of embodiments 1-205, wherein the TREM comprises an RNA sequence encoded by the DNA sequence of SEQ ID NO: 415, or a fragment thereof.
621. The method, composition or pharmaceutical composition, cell, reaction mixture, or the bioreactor of any one of embodiments 1-205, wherein the TREM comprises an RNA sequence encoded by the DNA sequence of SEQ ID NO: 416, or a fragment thereof.
622. The method, composition or pharmaceutical composition, cell, reaction mixture, or the bioreactor of any one of embodiments 1-205, wherein the TREM comprises an RNA sequence encoded by the DNA sequence of SEQ ID NO: 417, or a fragment thereof.
623. The method, composition or pharmaceutical composition, cell, reaction mixture, or the bioreactor of any one of embodiments 1-205, wherein the TREM comprises an RNA sequence encoded by the DNA sequence of SEQ ID NO: 418, or a fragment thereof.
624. The method, composition or pharmaceutical composition, cell, reaction mixture, or the bioreactor of any one of embodiments 1-205, wherein the TREM comprises an RNA sequence encoded by the DNA sequence of SEQ ID NO: 419, or a fragment thereof.
625. The method, composition or pharmaceutical composition, cell, reaction mixture, or the bioreactor of any one of embodiments 1-205, wherein the TREM comprises an RNA sequence encoded by the DNA sequence of SEQ ID NO: 420, or a fragment thereof.
626. The method, composition or pharmaceutical composition, cell, reaction mixture, or the bioreactor of any one of embodiments 1-205, wherein the TREM comprises an RNA sequence encoded by the DNA sequence of SEQ ID NO: 421, or a fragment thereof.
627. The method, composition or pharmaceutical composition, cell, reaction mixture, or the bioreactor of any one of embodiments 1-205, wherein the TREM comprises an RNA sequence encoded by the DNA sequence of SEQ ID NO: 422, or a fragment thereof.
628. The method, composition or pharmaceutical composition, cell, reaction mixture, or the bioreactor of any one of embodiments 1-205, wherein the TREM comprises an RNA sequence encoded by the DNA sequence of SEQ ID NO: 423, or a fragment thereof.
629. The method, composition or pharmaceutical composition, cell, reaction mixture, or the bioreactor of any one of embodiments 1-205, wherein the TREM comprises an RNA sequence encoded by the DNA sequence of SEQ ID NO: 424, or a fragment thereof.
630. The method, composition or pharmaceutical composition, cell, reaction mixture, or the bioreactor of any one of embodiments 1-205, wherein the TREM comprises an RNA sequence encoded by the DNA sequence of SEQ ID NO: 425, or a fragment thereof.
631. The method, composition or pharmaceutical composition, cell, reaction mixture, or the bioreactor of any one of embodiments 1-205, wherein the TREM comprises an RNA sequence encoded by the DNA sequence of SEQ ID NO: 426, or a fragment thereof.
632. The method, composition or pharmaceutical composition, cell, reaction mixture, or the bioreactor of any one of embodiments 1-205, wherein the TREM comprises an RNA sequence encoded by the DNA sequence of SEQ ID NO: 427, or a fragment thereof.
633. The method, composition or pharmaceutical composition, cell, reaction mixture, or the bioreactor of any one of embodiments 1-205, wherein the TREM comprises an RNA sequence encoded by the DNA sequence of SEQ ID NO: 428, or a fragment thereof.
634. The method, composition or pharmaceutical composition, cell, reaction mixture, or the bioreactor of any one of embodiments 1-205, wherein the TREM comprises an RNA sequence encoded by the DNA sequence of SEQ ID NO: 429, or a fragment thereof.
635. The method, composition or pharmaceutical composition, cell, reaction mixture, or the bioreactor of any one of embodiments 1-205, wherein the TREM comprises an RNA sequence encoded by the DNA sequence of SEQ ID NO: 430, or a fragment thereof.
636. The method, composition or pharmaceutical composition, cell, reaction mixture, or the bioreactor of any one of embodiments 1-205, wherein the TREM comprises an RNA sequence encoded by the DNA sequence of SEQ ID NO: 431, or a fragment thereof.
637. The method, composition or pharmaceutical composition, cell, reaction mixture, or the bioreactor of any one of embodiments 1-205, wherein the TREM comprises an RNA sequence encoded by the DNA sequence of SEQ ID NO: 432, or a fragment thereof.
638. The method, composition or pharmaceutical composition, cell, reaction mixture, or the bioreactor of any one of embodiments 1-205, wherein the TREM comprises an RNA sequence encoded by the DNA sequence of SEQ ID NO: 433, or a fragment thereof.
639. The method, composition or pharmaceutical composition, cell, reaction mixture, or the bioreactor of any one of embodiments 1-205, wherein the TREM comprises an RNA sequence encoded by the DNA sequence of SEQ ID NO: 434, or a fragment thereof.
640. The method, composition or pharmaceutical composition, cell, reaction mixture, or the bioreactor of any one of embodiments 1-205, wherein the TREM comprises an RNA sequence encoded by the DNA sequence of SEQ ID NO: 435, or a fragment thereof.
641. The method, composition or pharmaceutical composition, cell, reaction mixture, or the bioreactor of any one of embodiments 1-205, wherein the TREM comprises an RNA sequence encoded by the DNA sequence of SEQ ID NO: 436, or a fragment thereof.
642. The method, composition or pharmaceutical composition, cell, reaction mixture, or the bioreactor of any one of embodiments 1-205, wherein the TREM comprises an RNA sequence encoded by the DNA sequence of SEQ ID NO: 437, or a fragment thereof.
643. The method, composition or pharmaceutical composition, cell, reaction mixture, or the bioreactor of any one of embodiments 1-205, wherein the TREM comprises an RNA sequence encoded by the DNA sequence of SEQ ID NO: 438, or a fragment thereof.
644. The method, composition or pharmaceutical composition, cell, reaction mixture, or the bioreactor of any one of embodiments 1-205, wherein the TREM comprises an RNA sequence encoded by the DNA sequence of SEQ ID NO: 439, or a fragment thereof.
645. The method, composition or pharmaceutical composition, cell, reaction mixture, or the bioreactor of any one of embodiments 1-205, wherein the TREM comprises an RNA sequence encoded by the DNA sequence of SEQ ID NO: 440, or a fragment thereof.
646. The method, composition or pharmaceutical composition, cell, reaction mixture, or the bioreactor of any one of embodiments 1-205, wherein the TREM comprises an RNA sequence encoded by the DNA sequence of SEQ ID NO: 441, or a fragment thereof.
647. The method, composition or pharmaceutical composition, cell, reaction mixture, or the bioreactor of any one of embodiments 1-205, wherein the TREM comprises an RNA sequence encoded by the DNA sequence of SEQ ID NO: 442, or a fragment thereof.
648. The method, composition or pharmaceutical composition, cell, reaction mixture, or the bioreactor of any one of embodiments 1-205, wherein the TREM comprises an RNA sequence encoded by the DNA sequence of SEQ ID NO: 443, or a fragment thereof.
649. The method, composition or pharmaceutical composition, cell, reaction mixture, or the bioreactor of any one of embodiments 1-205, wherein the TREM comprises an RNA sequence encoded by the DNA sequence of SEQ ID NO: 444, or a fragment thereof.
650. The method, composition or pharmaceutical composition, cell, reaction mixture, or the bioreactor of any one of embodiments 1-205, wherein the TREM comprises an RNA sequence encoded by the DNA sequence of SEQ ID NO: 445, or a fragment thereof.
651. The method, composition or pharmaceutical composition, cell, reaction mixture, or the bioreactor of any one of embodiments 1-205, wherein the TREM comprises an RNA sequence encoded by the DNA sequence of SEQ ID NO: 446, or a fragment thereof.
652. The method, composition or pharmaceutical composition, cell, reaction mixture, or the bioreactor of any one of embodiments 1-205, wherein the TREM comprises an RNA sequence encoded by the DNA sequence of SEQ ID NO: 447, or a fragment thereof.
653. The method, composition or pharmaceutical composition, cell, reaction mixture, or the bioreactor of any one of embodiments 1-205, wherein the TREM comprises an RNA sequence encoded by the DNA sequence of SEQ ID NO: 448, or a fragment thereof.
654. The method, composition or pharmaceutical composition, cell, reaction mixture, or the bioreactor of any one of embodiments 1-205, wherein the TREM comprises an RNA sequence encoded by the DNA sequence of SEQ ID NO: 449, or a fragment thereof.
655. The method, composition or pharmaceutical composition, cell, reaction mixture, or the bioreactor of any one of embodiments 1-205, wherein the TREM comprises an RNA sequence encoded by the DNA sequence of SEQ ID NO: 450, or a fragment thereof.
656. The method, composition or pharmaceutical composition, cell, reaction mixture, or the bioreactor of any one of embodiments 1-205, wherein the TREM comprises an RNA sequence encoded by the DNA sequence of SEQ ID NO: 451, or a fragment thereof.
657. The method, composition or pharmaceutical composition, cell, reaction mixture, or the bioreactor of any one of embodiments 1-205, wherein the TREM comprises an RNA sequence encoded by the DNA sequence of SEQ ID NO: 562, or a fragment thereof.
658. The method, composition or pharmaceutical composition, cell, reaction mixture, or the bioreactor of any one of embodiments 1-205, wherein the TREM comprises an RNA sequence encoded by the DNA sequence of SEQ ID NO: 563, or a fragment thereof.
659. The method, composition or pharmaceutical composition, cell, reaction mixture, or the bioreactor of any one of embodiments 1-205, wherein the TREM comprises an RNA sequence encoded by the DNA sequence of SEQ ID NO: 564, or a fragment thereof.
660. The method, composition or pharmaceutical composition, cell, reaction mixture, or the bioreactor of any one of embodiments 1-205, wherein the TREM comprises an RNA sequence encoded by the DNA sequence of SEQ ID NO: 565, or a fragment thereof.
661. The method, composition or pharmaceutical composition, cell, reaction mixture, or the bioreactor of any one of embodiments 1-205, wherein the TREM comprises an RNA sequence encoded by the DNA sequence of SEQ ID NO: 566, or a fragment thereof.
662. The method, composition or pharmaceutical composition, cell, reaction mixture, or the bioreactor of any one of embodiments 1-205, wherein the TREM comprises an RNA sequence encoded by the DNA sequence of SEQ ID NO: 567, or a fragment thereof.
663. The method, composition or pharmaceutical composition, cell, reaction mixture, or the bioreactor of any one of embodiments 1-205, wherein the TREM comprises an RNA sequence encoded by the DNA sequence of SEQ ID NO: 568, or a fragment thereof.
664. The method, composition or pharmaceutical composition, cell, reaction mixture, or the bioreactor of any one of embodiments 1-205, wherein the TREM comprises an RNA sequence encoded by the DNA sequence of SEQ ID NO: 569, or a fragment thereof.
665. The method, composition or pharmaceutical composition, cell, reaction mixture, or the bioreactor of any one of embodiments 1-205, wherein the TREM comprises an RNA sequence encoded by the DNA sequence of SEQ ID NO: 570, or a fragment thereof.
666. The method, composition or pharmaceutical composition, cell, reaction mixture, or the bioreactor of any one of embodiments 1-205, wherein the TREM comprises an RNA sequence encoded by the DNA sequence of SEQ ID NO: 571 or a fragment thereof.
667. The method, composition or pharmaceutical composition, cell, reaction mixture, or the bioreactor of any one of embodiments 1-205, wherein the TREM comprises an RNA sequence encoded by the DNA sequence of SEQ ID NO: 572, or a fragment thereof.
668. The method, composition or pharmaceutical composition, cell, reaction mixture, or the bioreactor of any one of embodiments 1-205, wherein the TREM comprises an RNA sequence encoded by the DNA sequence of SEQ ID NO: 573, or a fragment thereof.
669. The method, composition or pharmaceutical composition, cell, reaction mixture, or the bioreactor of any one of embodiments 1-205, wherein the TREM comprises an RNA sequence encoded by the DNA sequence of SEQ ID NO: 574, or a fragment thereof.
670. The method, composition or pharmaceutical composition, cell, reaction mixture, or the bioreactor of any one of embodiments 1-205, wherein the TREM comprises an RNA sequence encoded by the DNA sequence of SEQ ID NO: 575, or a fragment thereof.
671. The method, composition or pharmaceutical composition, cell, reaction mixture, or the bioreactor of any one of embodiments 1-205, wherein the TREM comprises an RNA sequence encoded by the DNA sequence of SEQ ID NO: 576, or a fragment thereof.
672. The method, composition or pharmaceutical composition, cell, reaction mixture, or the bioreactor of any one of embodiments 1-205, wherein the TREM comprises an RNA sequence encoded by the DNA sequence of SEQ ID NO: 577, or a fragment thereof.
673. The method, composition or pharmaceutical composition, cell, reaction mixture, or the bioreactor of any one of embodiments 1-205, wherein the TREM comprises an RNA sequence encoded by the DNA sequence of SEQ ID NO: 578, or a fragment thereof.
674. The method, composition or pharmaceutical composition, cell, reaction mixture, or the bioreactor of any one of embodiments 1-205, wherein the TREM comprises an RNA sequence encoded by the DNA sequence of SEQ ID NO: 579, or a fragment thereof.
675. The method, composition or pharmaceutical composition, cell, reaction mixture, or the bioreactor of any one of embodiments 1-205, wherein the TREM comprises an RNA sequence encoded by the DNA sequence of SEQ ID NO: 580, or a fragment thereof.
676. The method, composition or pharmaceutical composition, cell, reaction mixture, or the bioreactor of any one of embodiments 1-205, wherein the TREM comprises an RNA sequence encoded by the DNA sequence of SEQ ID NO: 581, or a fragment thereof.
677. The method, composition or pharmaceutical composition, cell, reaction mixture, or the bioreactor of any one of embodiments 1-205, wherein the TREM comprises an RNA sequence encoded by the DNA sequence of SEQ ID NO: 582, or a fragment thereof.
678. The method, composition or pharmaceutical composition, cell, reaction mixture, or the bioreactor of any one of embodiments 1-205, wherein the TREM comprises an RNA sequence encoded by the DNA sequence of SEQ ID NO: 583, or a fragment thereof.
679. The method, composition or pharmaceutical composition, cell, reaction mixture, or the bioreactor of any one of embodiments 1-205, wherein the TREM comprises an RNA sequence encoded by the DNA sequence of SEQ ID NO: 584, or a fragment thereof.
680. The method, composition or pharmaceutical composition, cell, reaction mixture, or the bioreactor of any one of embodiments 1-205, wherein the TREM comprises an RNA sequence encoded by the DNA sequence of SEQ ID NO: 585, or a fragment thereof.
681. The method, composition or pharmaceutical composition, cell, reaction mixture, or the bioreactor of any one of embodiments 1-205, wherein the TREM comprises an RNA sequence encoded by the DNA sequence of SEQ ID NO: 586, or a fragment thereof.
682. The method, composition or pharmaceutical composition, cell, reaction mixture, or the bioreactor of any one of embodiments 1-205, wherein the TREM comprises an RNA sequence encoded by the DNA sequence of SEQ ID NO: 587, or a fragment thereof.
683. The method, composition or pharmaceutical composition, cell, reaction mixture, or the bioreactor of any one of embodiments 1-205, wherein the TREM comprises an RNA sequence encoded by the DNA sequence of SEQ ID NO: 588, or a fragment thereof.
684. The method, composition or pharmaceutical composition, cell, reaction mixture, or the bioreactor of any one of embodiments 1-205, wherein the TREM comprises an RNA sequence encoded by the DNA sequence of SEQ ID NO: 589, or a fragment thereof.
685. The method, composition or pharmaceutical composition, cell, reaction mixture, or the bioreactor of any one of embodiments 1-205, wherein the TREM comprises an RNA sequence encoded by the DNA sequence of SEQ ID NO: 590, or a fragment thereof.
686. The method, composition or pharmaceutical composition, cell, reaction mixture, or the bioreactor of any one of embodiments 1-205, wherein the TREM comprises an RNA sequence encoded by the DNA sequence of SEQ ID NO: 591, or a fragment thereof.
687. The method, composition or pharmaceutical composition, cell, reaction mixture, or the bioreactor of any one of embodiments 1-205, wherein the TREM comprises an RNA sequence encoded by the DNA sequence of SEQ ID NO: 592, or a fragment thereof.
688. The method, composition or pharmaceutical composition, cell, reaction mixture, or the bioreactor of any one of embodiments 1-205, wherein the TREM comprises an RNA sequence encoded by the DNA sequence of SEQ ID NO: 593, or a fragment thereof.
689. The method, composition or pharmaceutical composition, cell, reaction mixture, or the bioreactor of any one of embodiments 1-205, wherein the TREM comprises an RNA sequence encoded by the DNA sequence of SEQ ID NO: 594, or a fragment thereof.
690. The method, composition or pharmaceutical composition, cell, reaction mixture, or the bioreactor of any one of embodiments 1-205, wherein the TREM comprises an RNA sequence encoded by the DNA sequence of SEQ ID NO: 595, or a fragment thereof.
691. The method, composition or pharmaceutical composition, cell, reaction mixture, or the bioreactor of any one of embodiments 1-205, wherein the TREM comprises an RNA sequence encoded by the DNA sequence of SEQ ID NO: 596, or a fragment thereof.
692. The method, composition or pharmaceutical composition, cell, reaction mixture, or the bioreactor of any one of embodiments 1-205, wherein the TREM comprises an RNA sequence encoded by the DNA sequence of SEQ ID NO: 597, or a fragment thereof.
693. The method, composition or pharmaceutical composition, cell, reaction mixture, or the bioreactor of any one of embodiments 1-205, wherein the TREM comprises an RNA sequence encoded by the DNA sequence of SEQ ID NO: 598, or a fragment thereof.
694. The method, composition or pharmaceutical composition, cell, reaction mixture, or the bioreactor of any one of embodiments 1-205, wherein the TREM comprises an RNA sequence encoded by the DNA sequence of SEQ ID NO: 599, or a fragment thereof.
695. The method, composition or pharmaceutical composition, cell, reaction mixture, or the bioreactor of any one of embodiments 1-205, wherein the TREM comprises an RNA sequence encoded by the DNA sequence of SEQ ID NO: 600, or a fragment thereof.
696. The method, composition or pharmaceutical composition, cell, reaction mixture, or the bioreactor of any one of embodiments 1-205, wherein the TREM comprises an RNA sequence encoded by the DNA sequence of SEQ ID NO: 601, or a fragment thereof.
697. The method, composition or pharmaceutical composition, cell, reaction mixture, or the bioreactor of any one of embodiments 1-205, wherein the TREM comprises an RNA sequence encoded by the DNA sequence of SEQ ID NO: 602, or a fragment thereof.
698. The method, composition or pharmaceutical composition, cell, reaction mixture, or the bioreactor of any one of embodiments 1-205, wherein the TREM comprises an RNA sequence encoded by the DNA sequence of SEQ ID NO: 603, or a fragment thereof.
699. The method, composition or pharmaceutical composition, cell, reaction mixture, or the bioreactor of any one of embodiments 1-205, wherein the TREM comprises an RNA sequence encoded by the DNA sequence of SEQ ID NO: 604, or a fragment thereof.
700. The method, composition or pharmaceutical composition, cell, reaction mixture, or the bioreactor of any one of embodiments 1-205, wherein the TREM comprises an RNA sequence encoded by the DNA sequence of SEQ ID NO: 605, or a fragment thereof.
701. The method, composition or pharmaceutical composition, cell, reaction mixture, or the bioreactor of any one of embodiments 1-205, wherein the TREM comprises an RNA sequence encoded by the DNA sequence of SEQ ID NO: 606, or a fragment thereof.
702. The method, composition or pharmaceutical composition, cell, reaction mixture, or the bioreactor of any one of embodiments 1-205, wherein the TREM comprises an RNA sequence encoded by the DNA sequence of SEQ ID NO: 607, or a fragment thereof.
703. The method, composition or pharmaceutical composition, cell, reaction mixture, or the bioreactor of any one of embodiments 1-205, wherein the TREM comprises an RNA sequence encoded by the DNA sequence of SEQ ID NO: 608, or a fragment thereof.
704. The method, composition or pharmaceutical composition, cell, reaction mixture, or the bioreactor of any one of embodiments 1-205, wherein the TREM comprises an RNA sequence encoded by the DNA sequence of SEQ ID NO: 609, or a fragment thereof.
705. The method, composition or pharmaceutical composition, cell, reaction mixture, or the bioreactor of any one of embodiments 1-205, wherein the TREM comprises an RNA sequence encoded by the DNA sequence of SEQ ID NO:610, or a fragment thereof.
706. The method, composition or pharmaceutical composition, cell, reaction mixture, or the bioreactor of any one of embodiments 1-205, wherein the TREM comprises an RNA sequence encoded by the DNA sequence of SEQ ID NO: 611, or a fragment thereof.
707. The method, composition or pharmaceutical composition, cell, reaction mixture, or the bioreactor of any one of embodiments 1-205, wherein the TREM comprises an RNA sequence encoded by the DNA sequence of SEQ ID NO:612, or a fragment thereof.
708. The method, composition or pharmaceutical composition, cell, reaction mixture, or the bioreactor of any one of embodiments 1-205, wherein the TREM comprises an RNA sequence encoded by the DNA sequence of SEQ ID NO: 613, or a fragment thereof.
709. The method, composition or pharmaceutical composition, cell, reaction mixture, or the bioreactor of any one of embodiments 1-205, wherein the TREM comprises an RNA sequence encoded by the DNA sequence of SEQ ID NO: 614, or a fragment thereof.
710. The method, composition or pharmaceutical composition, cell, reaction mixture, or the bioreactor of any one of embodiments 1-205, wherein the TREM comprises an RNA sequence encoded by the DNA sequence of SEQ ID NO: 615, or a fragment thereof.
711. The method, composition or pharmaceutical composition, cell, reaction mixture, or the bioreactor of any one of embodiments 1-205, wherein the TREM comprises an RNA sequence encoded by the DNA sequence of SEQ ID NO: 616, or a fragment thereof.
712. The method, composition or pharmaceutical composition, cell, reaction mixture, or the bioreactor of any one of embodiments 1-205, wherein the TREM comprises an RNA sequence encoded by the DNA sequence of SEQ ID NO: 617, or a fragment thereof.
713. The method, composition or pharmaceutical composition, cell, reaction mixture, or the bioreactor of any one of embodiments 1-205, wherein the TREM comprises an RNA sequence encoded by the DNA sequence of SEQ ID NO: 618, or a fragment thereof.
714. The method, composition or pharmaceutical composition, cell, reaction mixture, or the bioreactor of any one of embodiments 1-205, wherein the TREM comprises an RNA sequence encoded by the DNA sequence of SEQ ID NO: 619, or a fragment thereof.
715. The method, composition or pharmaceutical composition, cell, reaction mixture, or the bioreactor of any one of embodiments 1-205, wherein the TREM comprises an RNA sequence encoded by the DNA sequence of SEQ ID NO: 620, or a fragment thereof.
716. The method, composition or pharmaceutical composition, cell, reaction mixture, or the bioreactor of any one of embodiments 1-205, wherein the TREM comprises an RNA sequence encoded by the DNA sequence of SEQ ID NO: 621, or a fragment thereof.
717. The method, composition or pharmaceutical composition, cell, reaction mixture, bioreactor, or master cell bank of any one of embodiments 206-716, wherein, a fragment comprises one or more, but not all, of: a Linker 1 region, an AStD stem region; a Linker 2 region; a stem-loop region, e.g., a D arm Region; a Linker 3-Region; a stem-loop region, e.g., an AC arm region; a variable region; a stem-loop region, e.g., a T arm Region; and a Linker 4 region, e.g., as these regions are described herein.

Other features, objects, and advantages of the invention will be apparent from the description and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### DETAILED DESCRIPTION OF CERTAIN EMBODIMENTS

The present disclosure features tRNA-based effector molecules (TREMs) and methods relating thereto. As disclosed herein tRNA-based effector molecules (TREMs) are complex molecules which can mediate a variety of cellular processes. Pharmaceutical TREM compositions can be administered to a cell, a tissue, or to a subject (e.g., a mammalian cell, tissue or subject) to modulate these functions.

### Definitions

A “cognate adaptor function TREM,” as that term is used herein, refers to a TREM which mediates initiation or elongation with the AA (the cognate AA) associated in nature with the anti-codon of the TREM.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

“Differentially modified,” as that term is used herein, refers to a TREM having a modification which is made by a fungal cell or cell line; an insect cell or cell line; or a plant, plant cell or cell line (e.g., a fungal cell or cell line; an insect cell or cell line; or a plant, plant cell or cell line in which the TREM was made). In an embodiment, the modification differs, in terms of presence, location, or prevalence, from what is seen if the same TREM is made in a reference cell, e.g., mammalian cell, e.g., human cell, e.g., a human primary or transformed cell line, or a rodent cell, e.g., a primary or transformed cell line. In an embodiment, the differentially modified TREM is to be administered to a subject, e.g., a human, and the reference cell is a cell from the same species as the subject, e.g., the reference cell is a cell type believed to be a target cell in the subject. In embodiments, the reference cell is a primary or secondary cell line or culture. In embodiments, the reference cell is a cell from a subject having a disease or disorder. In embodiments, the reference cell is a cell from a tissue, e.g., a tissue from a subject having a disease or disorder. Examples of such modifications include, for fungal cells modifications provided in Table 3; for insect cells modifications provided in Table 2; and for plant cells modifications provided in Table 4.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:
(a) differs by at least one nucleotide or one post transcriptional modification from the closest sequence tRNA in a reference cell, e.g., a cell into which the exogenous nucleic acid is introduced;
(b) has been introduced into a cell other than the cell in which it was transcribed;
(c) is present in a cell other than one in which it naturally occurs;
(d) has a differential modification;
(e) was made in a fungal (e.g., yeast), insect, or plant cell; or
(f) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(f).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of Adenine, a Cytosine is replaced by a residue other than an analog of Cytosine, a Guanine is replaced by a residue other than an analog of Guanine, and a Uracil is replaced by a residue other than an analog of Uracil.

An analog refers to any possible derivative of the ribonucleotides, A, G, C or U. In an embodiment, a sequence having a derivative of any one of ribonucleotides A, G, C or U is a non-naturally occurring sequence.

An “oncogene,” as that term is used herein, refers to a gene that modulates one or more cellular processes including: cell fate determination, cell survival and genome maintenance. In an embodiment, an oncogene provides a selective growth advantage to the cell in which it is present, e.g., deregulated, e.g., genetically deregulated (e.g., mutated or amplified) or epigenetically deregulated. Exemplary oncogenes include, Myc (e.g., c-Myc, N-Myc or L-Myc), c-Jun, Wnt, or RAS.

A “pharmaceutical TREM composition,” as that term is used herein, refers to a TREM composition that is suitable for pharmaceutical use. Typically, a pharmaceutical TREM composition comprises a pharmaceutical excipient. In an embodiment the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a post-transcriptional modification listed in Table 2.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “synthetic TREM,” as that term is used herein, refers to a TREM which was synthesized other than in a cell having an endogenous nucleic acid encoding the TREM, e.g., by cell-free solid phase synthesis. A synthetic TREM can have the same, or a different, sequence, set of post-transcriptional modifications, or tertiary structure, as a native tRNA.

A “tRNA”, as that term is used herein, refers to a naturally occurring transfer ribonucleic acid in its native state.

A “tRNA-based effector molecule” or “TREM,” as that term is used herein, refers to an RNA molecule comprising a structure or property from (a)-(v) below, which is a recombinant TREM, a synthetic TREM, or a TREM which was made in a fungal, e.g., yeast, insect, or plant cell. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:
(a′) an optional linker region of a consensus sequence provided in the “Consensus Sequence” section, e.g., a Linker 1 region;
(a) an amino acid attachment domain that binds an amino acid, e.g., an acceptor stem domain (AStD), wherein an AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the AStD has at least 75, 80, 85, 85, 90, 95, or 100% identity with a naturally occurring AStD, e.g., an AStD encoded by a nucleic acid in Table 1. In an embodiment, the TREM can comprise a fragment or analog of an AStD, e.g., an AStD encoded by a nucleic acid in Table 1, which fragment in embodiments has AStD activity and in other embodiments does not have AStD activity. (One of ordinary skill can determine the relevant corresponding sequence for any of the domains, stems, loops, or other sequence features mentioned herein from a sequence encoded by a nucleic acid in Table 1. E.g., one of ordinary skill can determine the sequence which corresponds to an AStD from a tRNA sequence encoded by a nucleic acid in Table 1.) In an embodiment the AStD falls under the corresponding sequence of a consensus sequence provided in the “Consensus Sequence” section, or differs from the consensus sequence by no more than 1, 2, 5, or 10 positions;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula I zzz, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II zzz, wherein ZZZ indicates any of the twenty amino acids;

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula III zzz, wherein ZZZ indicates any of the twenty amino acids;
(a′-1) a linker comprising residues R₈-R₉ of a consensus sequence provided in the “Consensus Sequence” section, e.g., a Linker 2 region;
(b) a dihydrouridine hairpin domain (DHD), wherein a DHD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 1. In an embodiment, the TREM can comprise a fragment or analog of a DHD, e.g., a DHD encoded by a nucleic acid in Table 1, which fragment in embodiments has DHD activity and in other embodiments does not have DHD activity.

In an embodiment the DHD falls under the corresponding sequence of a consensus sequence provided in the “Consensus Sequence” section, or differs from the consensus sequence by no more than 1, 2, 5, or 10 positions;

In an embodiment, the DHD comprises residues R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈ of Formula I zzz, wherein ZZZ indicates any of the twenty amino acids;

In an embodiment, the DHD comprises residues R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈ of Formula II zzz, wherein ZZZ indicates any of the twenty amino acids;

In an embodiment, the DHD comprises residues R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈ of Formula III zzz, wherein ZZZ indicates any of the twenty amino acids;
(b′-1) a linker comprising residue R₂₉ of a consensus sequence provided in the “Consensus Sequence” section, e.g., a Linker 3 region;
(c) an anticodon that binds a respective codon in an mRNA, e.g., an anticodon hairpin domain (ACHD), wherein an ACHD comprises sufficient sequence, e.g., an anticodon triplet, to mediate, e.g., when present in an otherwise wildtype tRNA, pairing (with or without wobble) with a codon; In an embodiment the ACHD has at least 75, 80, 85, 85, 90, 95, or 100% identity with a naturally occurring ACHD, e.g., an ACHD encoded by a nucleic acid in Table 1. In an embodiment, the TREM can comprise a fragment or analog of an ACHD, e.g., an ACHD encoded by a nucleic acid in Table 1, which fragment in embodiments has ACHD activity and in other embodiments does not have ACHD activity.

In an embodiment the ACHD falls under the corresponding sequence of a consensus sequence provided in the “Consensus Sequence” section, or differs from the consensus sequence by no more than 1, 2, 5, or 10 positions;

In an embodiment, the ACHD comprises residues-R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄-R₄₆ of Formula I zzz, wherein ZZZ indicates any of the twenty amino acids;

In an embodiment, the ACHD comprises residues-R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄-R₄₆ of Formula II zzz, wherein ZZZ indicates any of the twenty amino acids; In an embodiment, the ACHD comprises residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III zzz, wherein ZZZ indicates any of the twenty amino acids;
(d) a variable loop domain (VLD), wherein a VLD comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 1. In an embodiment, the TREM can comprise a fragment or analog of a VLD, e.g., a VLD encoded by a nucleic acid in Table 1, which fragment in embodiments has VLD activity and in other embodiments does not have VLD activity.

In an embodiment the VLD falls under the corresponding sequence of a consensus sequence provided in the “Consensus Sequence” section.

In an embodiment, the VLD comprises residue-[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271);
(e) a thymine hairpin domain (THD), wherein a THD comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 1. In an embodiment, the TREM can comprise a fragment or analog of a THD, e.g., a THD encoded by a nucleic acid in Table 1, which fragment in embodiments has THD activity and in other embodiments does not have THD activity.

In an embodiment the THD falls under the corresponding sequence of a consensus sequence provided in the “Consensus Sequence” section, or differs from the consensus sequence by no more than 1, 2, 5, or 10 positions;

In an embodiment, the THD comprises residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I zzz, wherein ZZZ indicates any of the twenty amino acids;

In an embodiment, the THD comprises residues-R₄₈-R₄₉-R₅₀-R₅₁-R₅₂-R₅₃-R₅₄-R₅₅-R₅₆-R₅₇-R₅₅-R₅₉-R₆₀-R₆₁-R₆₂-R₆₃-R₆₄ of Formula II zzz, wherein ZZZ indicates any of the twenty amino acids;

In an embodiment, the THD comprises residues-R₄₈-R₄₉-R₅₀-R₅₁-R₅₂-R₅₃-R₅₄-R₅₅-R₅₆-R₅₇-R₅₅-R₅₉-R₆₀-R₆₁-R₆₂-R₆₃-R₆₄ of Formula III zzz, wherein ZZZ indicates any of the twenty amino acids;
(e′ 1) a linker comprising residue R₇₂ of a consensus sequence provided in the “Consensus Sequence” section, e.g., a Linker 4 region;
(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 1. In an embodiment, the TREM can comprise a fragment or analog of a stem or loop structure, e.g., a stem or loop structure encoded by a nucleic acid in Table 1, which fragment in embodiments has activity of a stem or loop structure, and in other embodiments does not have activity of a stem or loop structure;
(g) a tertiary structure, e.g., an L-shaped tertiary structure;
(h) adaptor function, i.e., the TREM mediates acceptance of an amino acid, e.g., its cognate amino acid and transfer of the AA in the initiation or elongation of a polypeptide chain;
(i) cognate adaptor function wherein the TREM mediates acceptance and incorporation of an amino acid (e.g., cognate amino acid) associated in nature with the anti-codon of the TREM to initiate or elongate a polypeptide chain;
(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;
(k) a regulatory function, e.g., an epigenetic function (e.g., gene silencing function or signaling pathway modulation function), cell fate modulation function, mRNA stability 5 modulation function, protein stability modulation function, protein transduction modulation function, or protein compartmentalization function;
(l) a structure which allows for ribosome binding;
(m) a post-transcriptional modification, e.g., it comprises one or more modifications from Table 2, e.g., 1, 2, 3, 4, 5, 6, 7, 8, 9, 10, 11, 12, 13, 14, or 15 modifications listed in Table 2;
(n) the ability to inhibit a functional property of a tRNA, e.g., any of properties (h)-(k) possessed by a tRNA;
(o) the ability to modulate cell fate;
(p) the ability to modulate ribosome occupancy;
(q) the ability to modulate protein translation;
(r) the ability to modulate mRNA stability;
(s) the ability to modulate protein folding and structure;
(t) the ability to modulate protein transduction or compartmentalization;
(u) the ability to modulate protein stability; or
(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:
(i) an amino acid attachment domain that binds an amino acid (e.g., an AStD, as described in (a) herein; and
(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 1, or a fragment or functional fragment thereof. In an embodiment, a TREM comprises an RNA sequence encoded by a DNA sequence listed in Table 1, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 1, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 1, or a fragment or a functional fragment thereof. In an embodiment, a TREM comprises a TREM domain, e.g., a domain described herein, comprising an RNA sequence encoded by DNA sequence listed in Table 1, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 1, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′ halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM composition,” as that term is used herein, refers to a composition comprising a plurality of TREMs. A TREM composition can comprise one or more species of TREMs. In an embodiment, the composition comprises only a single species of TREM. In an embodiment, the TREM composition comprises a first TREM species and a second TREM species. In an embodiment, the TREM composition comprises X TREM species, wherein X=2, 3, 4, 5, 6, 7, 8, 9, or 10. In an embodiment, the TREM has at least 70, 75, 80, 85, 90, or 95, or has 100%, identity with a sequence encoded by a nucleic acid in Table 1. A TREM composition can comprise one or more species of TREMs. In an embodiment, the TREM composition is purified from cell culture. In an embodiment the cell culture from which the TREM is purified comprises at least 1×10⁷ host cells, 1×10⁸ host cells, 1×10⁹ host cells, 1×10¹⁰ host cells, 1×10¹¹ host cells, 1×10¹² host cells, 1×10¹³ host cells, or 1×10¹⁴ host cells. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM.

A “tumor suppressor,” as that term is used herein, refers to a gene that modulates one or more cellular processes including: cell fate determination, cell survival and genome maintenance. In an embodiment, a tumor suppressor provides a selective growth advantage to the cell in which it is deregulated, e.g., genetically deregulated (e.g., mutated or deleted) or epigenetically deregulated. Exemplary tumor suppressors include p53 or Rb.

### Host Cells

A host cell is a cell (e.g., a cultured cell) that can be used for expression and/or purification of a TREM. In an embodiment, a host cell comprises a fungal cell, e.g., fungal cell or cell line, an insect cell, e.g., insect cell or cell line, or a plant cell, e.g., plant cell or cell line.

In an embodiment, a host cell is a cell that can be maintained under conditions that allow for expression of a TREM.

### Method of Culturing Host Cell

A host cell can be cultured in a medium that promotes growth, e.g., proliferation or hyperproliferation of the host cell. A host cell can be cultured in a suitable media, e.g., media suitable for the culture of a fungal cell or cell line, an insect cell or cell line, or a plant cell or cell line. In an embodiment, a host cell is cultured in media that has an excess of nutrients, e.g., is not nutrient limiting. A host cell can be cultured in a medium comprising or supplemented with one or a combination of growth factors, cytokines or hormones.

A host cell can also be cultured under conditions that induce stress, e.g., cellular stress, osmotic stress, translational stress, or oncogenic stress. In an embodiment, a host cell expressing a TREM, cultured under conditions that induce stress (e.g., as described herein) results in a fragment of the TREM, e.g., as described herein.

A host cell can be cultured under nutrient limiting conditions, e.g., the host cell is cultured in media that has a limited amount of one or more nutrients. Examples of nutrients that can be limiting are amino acids, lipids, carbohydrates, hormones, growth factors or vitamins. In an embodiment, a host cell expressing a TREM, cultured in media that has a limited amount of one or more nutrients, e.g., the media is nutrient starved, results in a fragment of the TREM, e.g., as described herein. In an embodiment, a host cell expressing a TREM, cultured in media that has a limited amount of one or more nutrients, e.g., the media is nutrient starved, results in a TREM that is uncharged (e.g. a uTREM).

A host cell can be cultured in suspension or as a monolayer. Cell culture vessels include a cell culture dish, plate or flask. Exemplary cell culture vessels include 35 mm, 60 mm, 100 mm, or 150 mm dishes, multi-well plates (e.g., 6-well, 12-well, 24-well, 48-well or 96 well plates), or T-25, T-75 or T-160 flasks.

Host cell cultures can be performed in a cell culture vessel or a bioreactor. In an embodiment, a bioreactor refers to a culture vessel with a capacity of at least 1 L (e.g., at least 5 L, at least 10 L, at least 50 L, at least 100 L, at least 500 L, or at least 1000 L) that allows for culturing, propagating, cultivating, maintaining, or storing of a host cell, e.g., a fungal cell or cell line; an insect cell or cell line; or a plant, plant cell or celline. In an embodiment, a bioreactor is maintained under controlled conditions (e.g., one or more of controlled sterility, mixing rate, temperature, light, oxygen supply, and/or nutrient medium).

In an embodiment, when a plant, plant cell or plant cell line is used, a bioreactor may contain entire plants or plant parts (e.g., may comprise a hydroponic system) or plant cells (e.g., may contain a plant cell culture). A bioreactor may contain any suitable substrate for plant, plant part, or plant cell growth, a liquid, solid, semi-solid, or gel substrate.

A variety of bioreactors may be used to culture a host cell, e.g., as described herein. Such a bioreactor may include a vessel that may be a closed or open system having several possible shapes, such as a vat, tank, flask, tube, jar, or bag. The vessel may be composed of a suitable material (e.g., glass, plastic, or metal). In some embodiments, the vessel may be reusable, e.g., an Eppendorf BioFio® 120 vessel. Reusable vessels may be sterilized between uses by, for example, autoclaving or the use of heated steam. Alternatively, the vessel may be single-use (e.g., CELL-tainer® Single-use Bioreactor Bag, WAVE® Bioreactor System 200, Flexsafe® R M Bag). The vessel may be a fermenter.

A bioreactor can be, e.g., a continuous flow batch bioreactor, a perfusion bioreactor, a batch process bioreactor or a fed batch bioreactor. A bioreactor can be maintained under conditions sufficient to express the TREM. The culture conditions can be modulated to optimize yield, purity or structure of the TREM. In an embodiment, a bioreactor comprises at least 1×10⁷, 1×10⁸, 1×10⁹, 1×10¹⁰, 1×10¹¹, 1×10¹², 1×10¹³, or 1×10¹⁴ host cells. In an embodiment, a bioreactor comprises between 1×10⁷ to 1×10¹⁴ host cells; between 1×10⁷ to 0.5×10¹⁴ host cells; between 1×10⁷ to 1×10¹³ host cells; between 1×10⁷ to 0.5×10¹³ host cells; between 1×10⁷ to 1×10¹² host cells; between 1×10⁷ to 0.5×10¹² host cells; between 1×10⁷ to 1×10¹¹ host cells; between 1×10⁷ to 0.5×10¹¹ host cells; between 1×10⁷ to 1×10¹⁰ host cells; between 1×10⁷ to 0.5×10¹⁰ host cells; between 1×10⁷ to 1×10⁹ host cells; between 1×10⁷ to 0.5×10⁹ host cells; between 1×10⁷ to 1×10⁸ host cells; between 1×10⁷ to 0.5×10⁸ host cells; between 0.5×10⁸ to 1×10¹⁴ host cells; between 1×10⁸ to 1×10¹⁴ host cells; between 0.5×10⁹ to 1×10¹⁴ host cells; between 1×10⁹ to 1×10¹⁴ host cells; between 0.5×10¹⁰ to 1×10¹⁴ host cells; between 1×10¹⁰ to 1×10¹⁴ host cells; between 0.5×10¹¹ to 1×10¹⁴ host cells; between 1×10¹¹ to 1×10¹⁴ host cells; between 0.5×10¹² to 1×10¹⁴ host cells; between 1×10¹² to 1×10¹⁴ host cells; between 0.5×10¹³ to 1×10¹⁴ host cells; between 1×10¹³ to 1×10¹⁴ host cells; or between 0.5×10¹³ to 1×10¹⁴ host cells.

In an embodiment, a bioreactor comprises at least 1×10⁵ host cells/mL, 2×10⁵ host cells/mL, 3×10⁵ host cells/mL, 4×10⁵ host cells/mL, 5×10⁵ host cells/mL, 6×10⁵ host cells/mL, 7×10⁵ host cells/mL, 8×10⁵ host cells/mL, 9×10⁵ host cells/mL, 1×10⁶ host cells/mL, 2×10⁶ host cells/mL, 3×10⁶ host cells/mL, 4×10⁶ host cells/mL, 5×10⁶ host cells/mL, 6×10⁶ host cells/mL, 7×10⁶ host cells/mL, 8×10⁶ host cells/mL, 9×10⁶ host cells/mL, 1×10⁷ host cells/mL, 2×10⁷ host cells/mL, 3×10⁷ host cells/mL, 4×10⁷ host cells/mL, 5×10⁷ host cells/mL, 6×10⁷ host cells/mL, 7×10⁷ host cells/mL, 8×10⁷ host cells/mL, 9×10⁷ host cells/mL, 1×10⁸ host cell/mL, 2×10⁸ host cells/mL, 3×10⁸ host cells/mL, 4×10⁸ host cells/mL, 5×10⁸ host cells/mL, 6×10⁸ host cells/mL, 7×10⁸ host cells/mL, 8×10⁸ host cells/mL, 9×10⁸ host cells/mL, or 1×10⁹ host cells/mL. In an embodiment, a bioreactor comprises between 1×10⁵ host cells/mL to 1×10⁹ host cells/mL, between 5×10⁵ host cells/mL to 1×10⁹ host cells/mL, between 1×10⁶ host cells/mL to 1×10⁹ host cells/mL; between 5×10⁶ host cells/mL to 1×10⁹ host cells/mL, between 1×10⁷ host cells/mL to 1×10⁹ host cells/mL, between 5×10⁷ host cells/mL to 1×10⁹ host cells/mL, between 1×10⁸ host cells/mL to 1×10⁹ host cells/mL, between 5×10⁸ host cells/mL to 1×10⁹ host cells/mL, between 1×10⁵ host cells/mL to 5×10⁸ host cells/mL, between 1×10⁵ host cells/mL to 1×10⁸ host cells/mL, between 1×10⁵ host cells/mL to 5×10⁷ host cells/mL, between 1×10⁵ host cells/mL to 1×10⁷ host cells/mL, between 1×10⁵ host cells/mL to 5×10⁶ host cells/mL, between 1×10⁵ host cells/mL to 1×10⁶ host cells/mL, or between 1×10⁵ host cells/mL to 5×10⁵ host cells/mL.

In an embodiment, a batch process bioreactor comprises 1×10⁶ to 1×10⁷ host cells/ml.

In an embodiment, a batch process bioreactor with a 100 mL volume comprises 1×10⁸ to 1×10⁹ host cells.

In an embodiment, a batch process bioreactor with a 100 L volume comprises 1×10¹¹ to 1×10¹² host cells.

In an embodiment, a fed batch bioreactor comprises 1×10⁷ to 3×10⁷ host cells/ml.

In an embodiment, a fed batch bioreactor with a 100 mL volume comprises 1×10⁹ to 3×10⁹ host cells.

In an embodiment, a fed batch bioreactor with a 100 L volume comprises 1×10¹² to 3×10¹² host cells.

In an embodiment, a perfusion bioreactor comprises 1×10⁸ host cells/ml.

In an embodiment, a perfusion bioreactor with a 100 mL volume comprises 1×10¹⁰ host cells.

In an embodiment, a perfusion bioreactor with a 100 L volume comprises 1×10¹³ host cells.

In an embodiment, a bioreactor is maintained under conditions that promote growth of the host cell, e.g., at a temperature (e.g., 37° C.) and gas concentration (e.g., 5% CO₂) that is permissive for growth of the host cell.

For example, in some aspects, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and C02 levels, maintenance of pH level, agitation (e.g., stirring), and/or cleaning/sterilizing. Exemplary bioreactor units, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. Any suitable bioreactor diameter can be used.

In an embodiment, the bioreactor can have a volume between about 100 mL and about 100 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters. Additionally, suitable reactors can be multi-use, single-use, disposable, or non-disposable and can be formed of any suitable material including metal alloys such as stainless steel (e.g., 316 L or any other suitable stainless steel) and Inconel, plastics, and/or glass. In some embodiments, suitable reactors can be round, e.g., cylindrical. In some embodiments, suitable reactors can be square, e.g., rectangular. Square reactors may in some cases provide benefits over round reactors such as ease of use (e.g., loading and setup by skilled persons), greater mixing and homogeneity of reactor contents, and lower floor footprint.

### Method of Modifying Host Cells

A host cell can be modified to optimize the production of a TREM, e.g., to have optimized TREM yield, purity, structure (e.g., folding), or stability. In an embodiment, a host cell can be modified (e.g., using a method described herein), to increase or decrease the expression of a desired molecule, e.g., gene, which optimizes production of the TREM, e.g., optimizes yield, purity, structure or stability of the TREM. In an embodiment, a host cell can be epigenetically modified, e.g., using a method described herein, to increase or decrease the expression of a desired gene, which optimizes production of the TREM.

In an embodiment, a host cell can be modified by: transformation (e.g., as described herein); transfection (e.g., transient transfection or stable transfection); transduction (e.g., viral transduction, e.g., lentiviral, adenoviral or retroviral transduction); electroporation; lipid-based delivery of an agent (e.g., liposomes), nanoparticle based delivery of an agent; or other methods known in the art.

In an embodiment, a host cell can be modified to increase the expression of, e.g., overexpress, a desired molecule, e.g., a gene (e.g., an oncogene, or a gene involved in tRNA or TREM modulation. Exemplary methods of increasing the expression of a gene include: (a) contacting the host cell with a nucleic acid (e.g., DNA, or RNA) encoding the gene; (b) contacting the host cell with a peptide that expresses the target protein; (c) contacting the host cell with a molecule (e.g., a small RNA (e.g., a micro RNA, or a small interfering RNA) or a low molecular weight compound) that modulates, e.g., increases the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of a negative regulator of the target gene. In an embodiment, a nucleic acid encoding the gene, or a plasmid containing a nucleic acid encoding the gene can be introduced into the host cell by transfection or electroporation. In an embodiment, a nucleic acid encoding a gene can be introduced into the host cell by contacting the host cell with a virus (e.g., a lentivirus, adenovirus or retrovirus) expressing the gene.

In an embodiment, a host cell can be modified to decrease the expression of, e.g., minimize the expression, of a desired molecule, e.g., a gene (e.g., a gene involved in tRNA or TREM modulation). Exemplary methods of decreasing the expression of a gene include: (a) contacting the host cell with a nucleic acid (e.g., DNA, or RNA) encoding an inhibitor of the gene (e.g., a dominant negative variant or a negative regulator of the gene or protein encoded by the gene); (b) contacting the host cell with a peptide that inhibits the target protein; (c) contacting the host cell with a molecule (e.g., a small RNA (e.g., a micro RNA, or a small interfering RNA) or a low molecular weight compound) that modulates, e.g., inhibits the expression of the target gene; or (d) contacting the host cell with a gene editing moiety (e.g., a zinc finger nuclease (ZFN) or a Cas9/CRISPR molecule) that inhibits (e.g., mutates or knocks-out) the expression of the target gene. In an embodiment, a nucleic acid encoding an inhibitor of the gene, or a plasmid containing a nucleic acid encoding an inhibitor of the gene can be introduced into the host cell by transfection or electroporation. In an embodiment, a nucleic acid encoding an inhibitor of the gene can be introduced into the host cell by contacting the host cell with a virus (e.g., a lentivirus, adenovirus or retrovirus) expressing the inhibitor of the gene.

Fungal host cells In an embodiment, a host cell is a fungal cell or cell line that can be used for expression and/or purification of a TREM. In an embodiment, a fungal host cell or cell line can be maintained under conditions that allow for expression of a TREM.

A fungal cell or cell line described herein includes fungal cells or cell lines from species that reproduce asexually (anamorphic) or sexually (teleomorphic). Fungal cells or cell lines can exist in unicellular form or may be able to form pseudohyphae (strings of connected budding cells). Fungal cell or cell lines may be haploid and/or diploid.

A fungal cell or cell line can be cultured using methods known in the art, e.g., as described in Non-Conventional Yeasts in Genetics, Biochemistry and Biotechnology: Practical Protocols (K. Wolf, K. D. Breunig, G. Barth, Eds., Springer-Verlag, Berlin, Germany, 2003), Yeasts in Natural and Artificial Habitats (J. F. T. Spencer, D. M. Spencer, Eds., Springer-Verlag, Berlin, Germany, 1997), and/or Yeast Biotechnology: Diversity and Applications (T. Satyanarayana, G. Kunze, Eds., Springer, 2009). Any appropriate media suitable for culturing a fungal cell or cell line can be used. For example, a fungal cell or cell line can be cultured in YPD medium, YPG medium or YPAD medium. Synthetic minimal media or synthetic complete media which include yeast nitrogen base can also be used.

A fungal cell or cell line can be modified using methods known in the art. For example, a fungal cell or cell line can be modified by transformation, e.g., transfer of a nucleic acid molecule into the fungal cell or cell line. For example, the nucleic acid molecule may be one that replicates autonomously, or that integrates into the genome of the host cell or that exists transiently in the host cell without replicating or integrating. Non-limiting examples of nucleic acid molecules suitable for transformation include vectors and linear DNA molecules. The nucleic acid molecule can include a promoter for controlling gene expression. A variety of promoters derived from yeasts and other eukaryotes can be used. The expression vector can also include a terminator region. Methods of transformation for fungal cells or cell lines are known in the art and include transfection methods, conjugation methods, protoplast methods, electroporation methods, lipofection methods, and lithium acetate methods. Exemplary methods of transforming fungal cells is described in Example 1.

Recombinant yeast methods are also disclosed in Molecular Cloning, 3rd Edition and Current Protocols in Molecular Biology, the entire contents of which are hereby incorporated by reference.

Exemplary fungal cell or cell lines are chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, and Pachysolen. In an embodiment, the fungal cell or cell line is a Saccharomyces cell or cell line. In an embodiment, the fungal cell or cell line is a Saccharomyces cerevisiae fungal cell or cell line. In an embodiment, the fungal cell or cell line is a Schizosaccharomyces pombe fungal cell or cell line. In an embodiment, the fungal cell or cell line is a Candida cylindracea fungal cell or cell line. In an embodiment, the fungal cell or cell line is a Candida albicans fungal cell or cell line. In an embodiment, the fungal cell or cell line is a Neurospora crassa fungal cell or cell line. In an embodiment, the fungal cell or cell line is a Pichia jadinii fungal cell or cell line.

In some embodiments, the host cells are filamentous fungal host cells. The term “filamentous fungi” refers to all filamentous forms of the subdivision Eumycotina (See, 10 Alexopoulos, C. J. (1962), INTRODUCTORY MYCOLOGY, Wiley, New York).

These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolism is obligatory aerobic. In the present invention, the filamentous fungal parent cell may 15 be a cell of a species of, but not limited to, Trichoderma, (e.g., Trichoderma reesei, the asexual morph of Hypocrea jecorina, previously classified as T. longibrachiatum, Trichoderma viride, Trichoderma koningii, Trichoderma harzianum) (Sheir-Neirs et al., (1984) Appl. Microbiol. Biotechnol 20:46 “53; ATCC No. 56765 and ATCC No. 26921); Penicillium sp., Humicola sp. (e.g., H. insolens, H, lanuginosa and H. grisea); Chrysosporium sp. (e.g., C. lucknowense), Gliocladium sp., Aspergillus sp. (e.g., A. 20 oryzae, A. niger, A sojae, A. japonicus, A. nidulans, and A. awamori) (Ward et al., (1993) Appl. Microbiol. Biotechnol. 39:738 “743 and Goedegebuur et al., (2002) Genet 41:89-98), Fusarium sp., (e.g. F. roseum, F. graminum F. cereal is, F. oxysporum and F. venenatum), Neurospora sp., (N. crassa), Hypocrea sp., Mucor sp., (M miehei), Rhizopus sp. and Emericella sp. (See also, Innis et al., (1985) Sci. 228:21-26). The term “Trichoderma” or “Trichoderma sp.” or “Trichoderma spp.” refer to any fungal 25 genus previously or currently classified as Trichoderma.

Additonal exemplary fungal cells and methods of modifying and culturing the same are disclosed in US20160281097A1 and US2020190540A1 the entire contents of each of which are hereby incorporated by reference.

### Insect Host Cells

In an embodiment, a host cell is an insect cell or cell line that can be used for expression and/or purification of a TREM. In an embodiment, an insect host cell or cell line can be maintained under conditions that allow for expression of a TREM.

An insect cell or cell line can be cultured using methods known in the art. Any appropriate media can be used to culture an insect cell or cell line. For example, Express Five SFM media or Sf-900 II SFM media can be used to culture insect cells. As another example, IPL-41 medium (JRH Biosciences, Inc.) containing 10% fetal calf serum (Hyclone Laboratories, Inc.) as described in U.S. Pat. No. 5,759,809 can be used. As yet another example, insect cells can be cultured in media containing fish serum, as described in U.S. Pat. No. 5,498,540, incorporated herein by reference.

An insect cell or cell line can be modified using methods known in the art. For example, an insect cell or cell line can be modified by introduction of a vector to the insect cell or cell line.

In an embodiment, the vector can be introduced transiently. In an embodiment, the vector can be introduced premanently. The vectors can be introduced by any method known in the art, for example by chemical treatment of the cells, electroporation, or infection. In an embodiment, the vector is a baculovirus, a viral vector, or a plasmid. An exemplary method of using a baculovirus system for expression in insect cells is provided in Example 4.

Insect cell baculovirus systems can be used to introduce a nucleic acid of interest into an insect cell or cell line. For example, the polyhedrin promoter can be used to control expression of the nucleic acid. Additional promoters and/or enhancers can be used to further control expression of the nucleic acid molecule. Baculovirus transfection of insect cells and additional baculovirus promoters and enhances are described in U.S. Pat. Nos. 5,759,809; 6,342,216 the entire contencts of each of which are incorporated herein by reference.

Exemplary insect cells or cell lines include cells or cell lines from Autographa californica, Aedes aegypti, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, Lepidopteran insect family, or Drosophila melanogaster. Non-limiting examples of insect cell lines include, for exmapl,e Sf21, Sf9, High Five (BT1-TN-5B1-4), BT1-Ea88, Tn-368, mb0507, Tn mg-1, and Tn Ap2, among others. In an embodiment, the insect cell is a Spodoptera frugiperda cell. In an embodiment, the Spodoptera frugiperda cell is an Sf9 cell. In an embodiment, the Sf9 cell is a Sf-9S cell line.

In an embodiment, the insect cell is a Trichoplusia ni cell. In an embodiment, the insect cell is a H5 cell (High Five™, Invitrogen, Sorrento, CA).

### Plant Host and Cells

In an embodiment, a plant host includes a whole plant (e.g., whole seedlings or whole adult plants), plant organs, plant parts, plant tissues, seeds, plant cells, seeds, cell lines and progeny of the same. In an embodiment, a plant cell includes cells that are a cell of a whole plant or a portion of a plant, or a cell of a plant tissue, or a cell line that can be used for expression and/or purification of a TREM. In an embodiment, plant tissue includes differentiated and undifferentiated tissues of plants, including, but not limited to, roots, shoots, leaves, pollen, seeds, tumor tissue and various forms of cells in culture, such as single cells, protoplasts, embryos and callus tissue. Plant cells include, without limitation, cells from seeds, suspension cultures, embryos, meristematic regions, callus tissue, cambial tissue, hypocotyl, leaves, roots, radicles, shoots, gametophytes, sporophytes, pollen, and microspores. Plant cells also include single-celled plants, e.g., single-celled plastid-containing organisms such as algae. Plant parts include differentiated and undifferentiated tissues including, but not limited to the following: roots, radicles, flowers, pistils, stamens, stems, hypocotyls, cambial tissue, shoots, leaves, pollen, seeds, fruit, harvested produce, tumor tissue, sap (e.g., xylem sap and phloem sap), and various forms of cells and culture (e.g., single cells, protoplasts, embryos, and callus tissue).

In an embodiment, a plant, plant cell or cell line can be maintained under conditions that allow for expression of a TREM.

A plant, plant cell or cell line can be modified using methods known in the art. For example, an insect cell or cell line can be modified by transformation with a nucleic acid molecule, e.g., an expression vector. The expression vector can contain one or more sequences for stably replicating the vector in a plant cell, either episomally, or as part of an endogenous plant chromosome. Sequences for facilitating integration into a plant chromosome can also be included. The vector can include a promoter which allows expression in, e.g., the leaf, stem, root, floral and/or seed tissue. For examples, the Arabidopsis Actin 2 promoter, the OCS(MAS) promoter and various forms thereof, the CaMV 35S, and figwort mosaic virus 34S promoter can be used. As another example, the ubiquitin promoter can be used in transgenic plants (e.g., sunflower (Binet et al., Plant Science 79: 87-94 (1991); and maize (Christensen et al., Plant Molec. Biol. 12, 619-632 (1989)). Further useful promoters are the U2 and U5 snRNA promoters from maize (Brown et al., Nucleic Acids Res. 17, 8991 (1989)) and the promoter from alcohol dehydrogenase (Dennis et al., Nucleic Acids Res. 12, 3983 (1984)). Additional promoters that can be used include promoters disclosed in U.S. Pat. No. 9,040,774 in the section titled “Promoter,” the entire contents of which is hereby incorporated by reference. For example, such promoters include: an opaline synthase promoter isolated from T-DNA of Agrobacterium; a cauliflower mosaic virus 35S promoter; enhanced promoter elements or chimeric promoter elements such as an enhanced cauliflower mosaic virus (CaMV) 35S promoter linked to an enhancer element (an intron from heat shock protein 70 of Zea mays); root specific promoters such as those disclosed in U.S. Pat. Nos. 5,837,848; 6,437,217; 6,426,446; a maize L3 oleosin promoter disclosed in U.S. Pat. No. 6,433,252; a promoter for a plant nuclear gene encoding a plastid-localized aldolase disclosed in U.S. Patent Application Publication 2004/0216189; cold-inducible promoters disclosed in U.S. Pat. No. 6,084,089; salt-inducible promoters disclosed in U.S. Pat. No. 6,140,078; light-inducible promoters disclosed in U.S. Pat. No. 6,294,714; pathogen-inducible promoters disclosed in U.S. Pat. No. 6,252,138; and water deficit-inducible promoters disclosed in U.S. Patent Application Publication 2004/0123347 A1.

Additionally, plant vascular- or phloem-specific promoters of include a rolC or rolA promoter of Agrobacterium rhizogenes, a promoter of a Agrobacterium tumefaciens T-DNA gene 5, the rice sucrose synthase RSs1 gene promoter, a Commelina yellow mottle badnavirus promoter, a coconut foliar decay virus promoter, a rice tungro bacilliform virus promoter, the promoter of a pea glutamine synthase GS3A gene, a invCDl11 and invCD141 promoters of a potato invertase genes, a promoter isolated from Arabidopsis shown to have phloem-specific expression in tobacco by Kertbundit et al. (1991) Proc. Natl. Acad. Sci. USA., 88:5212-5216, a VAHOXI promoter region, a pea cell wall invertase gene promoter, an acid invertase gene promoter from carrot, a promoter of a sulfate transporter gene Sultrl; 3, a promoter of a plant sucrose synthase gene, and a promoter of a plant sucrose transporter gene.

In some embodiments, the plant host cells are cells from a monocot plant (e.g., corn, wheat and sorghum) or cells from a dicot plant (e.g., soybean).

All of the above-described patents and patent publications disclosing promoters and their use, especially in recombinant DNA constructs functional in plants are incorporated herein by reference.

Methods of plant cell transformation with vectors are known in the art. For example, vacuum-infiltration, dipping, microinjection, chemical treatment, microprojectile bombardment or biolistics can be used to transform a plant cell or cell line. As another example, viral vectors can be used to introduce a nucleic acid molecule into a plant cell or cell line. Exemplary methods of transforming plants are provided in Examples 2-3.

As another example, transformation of a plant can be performed by dipping developing floral tissues into an Agrobacterium solution. This step can be done with or without subjecting the small plants (35 days old or so) to a vacuum during the dipping stage. After a few weeks of the floral dip, the plants set seed which can be harvested and screened for transgenic plants that contain a gene of interest. See, e.g., Clough and Bent, Plant J. 16: 735-43 (1998). Additional plant specific promoters and methods of transforming plants are disclosed in International Application WO 2003/012035, the entire contents of which are hereby incorporated by reference.

In an embodiment, the plant cell or cell line is transformed with a gene to increase the utility of the plants as a source for large-scale production. In an embodiment, such genes includes genes which make plant cells or cell lines resistant to diseases and insects, and/or genes which encode proteins providing antifungal, antibacterial or antiviral activity.

A plant, plant cell or cell line, e.g., a modified plant, can be cultured using methods known in the art. In an embodiment, plant culture comprise a plant or a plurality of plants, plant parts, plant cells, or plant tissue that is propagated in or on a medium, e.g., a liquid, gaseous, gel, semi-solid, or solid medium. Plant culture includes, but is not limited to, culture of naturally occurring plants, plant parts, plant cells, or plant tissue or genetically modified plants, plant parts, plant cells, or plant tissues. Plant cultures can be classified, for example, as unorganized cultures (e.g., plant cell cultures such as callus, suspension, or protoplast cultures) or organized cultures (such as root, seedling, embryo, or entire plant cultures) depending on the tissue source and the level of differentiation of the cultured plant material.

The plant culture may be a hydroponic culture. As used herein, the term“hydroponic” refers to a hydrated growth system for a plant or plant part (e.g., a plant root) that does not include a natural soil. Such hydroponic growth systems include, e.g., a plant growth system comprising a liquid or semi-liquid (e.g., aqueous), gel, semi-solid, or hydrated solid culture medium. Hydroponic cultures may include aquaponic, hydroculture, or aquaculture growth systems.

Additional plant culture methods, systems and media are disclosed in International Application WO 2020/041784, the entire contents of which are hereby incorporated by reference.

A host plant, plant cell or cell line described herein includes a monocotyledonous (monocot) plant, cell or cell line; or a dicotyledonous (dicot) plant, cell or cell line. Exemplary monocotyledonous plants include: wheat (e.g., Triticum aestivum), rice, maize (e.g., Zea mays), or barley (e.g., Hordeum vulgare). Exemplary dicotyledonous plants include tobacco plant (e.g., Nicotiana rustica or Nicotiana tabacum), Arabidopsis (e.g., A. thaliana), lupins (e.g., Lupinus albus), bean (e.g., Phaseolus vulgaris), pea (e.g., Pisum sativum), potato (e.g., Solanum tuberosum), or spinach (e.g., Spinacia oleracea).

In an embodiment, the host plant, cell or cell line is chosen from: wheat (e.g., Triticum aestivum), rice, maize (e.g., Zea mays), barley (e.g., Hordeum vulgare), tobacco plant (e.g., Nicotiana rustica or Nicotiana tabacum), Arabidopsis, lupins (e.g., Lupinus albus), beans (e.g., Phaseolus vulgaris), peas (e.g., Pisum sativum), potato (e.g., Solanum tuberosum), spinach (e.g., Spinacia oleracea), or an Arabidopsis plant, cell or cell line.

In an embodiment, the host plant, cell, or cell line is chosen from: Brassica, Nicotiana, Solanum, Lycopersicon, Daucus, Hordeum, Triticum, and Oryza.

In an embodiment, the plant, cell or cell line is an Arabidopsis thaliana plant, cell or cell line. Arabidopsis strains are commercially available and can be obtained, for example, from The Arabidopsis Biological Resource Center (ABRC). In an embodiment, the host plant, cell or cell line is an Arabidopsis plant, cell or cell line.

### Trem

A “tRNA-based effector molecule” or “TREM” refers to an RNA molecule comprising one or more of the properties described herein. A TREM can be charged with an amino acid, e.g., a cognate amino acid; charged with a non-cognate amino acid (e.g., a mischarged TREM (mTREM); or not charged with an amino acid, e.g., an uncharged TREM (uTREM).

In an embodiment, a TREM comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM comprises at least 30 consecutive nucleotides of an RNA sequence encoded by a DNA sequence disclosed in Table 1, e.g., at least 30 consecutive nucleotides of an RNA sequence encoded by any one of SEQ ID NOs: 1-451 disclosed in Table 1. In an embodiment, a TREM comprises at least 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises at least 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

TABLE 1
List of tRNA sequences

| SEQ | | |
|---|---|---|
| ID | | |
| NO | tRNA name | tRNA sequence |
| 1 | Ala_AGC_chr6:28763741-28763812 (−) | GGGGGTATAGCTCAGTGGTAGAGCGCGTGCTTAGCATGCACGAGGTCC |
| | | TGGGTTCGATCCCCAGTACCTCCA |
| 2 | Ala_AGC_chr6:26687485-26687557 (+) | GGGGAATTAGCTCAAGTGGTAGAGCGCTTGCTTAGCACGCAAGAGGTA |
| | | GTGGGATCGATGCCCACATTCTCCA |
| 3 | Ala_AGC_chr6:26572092-26572164 (−) | GGGGAATTAGCTCAAATGGTAGAGCGCTCGCTTAGCATGCGAGAGGTA |
| | | GCGGGATCGATGCCCGCATTCTCCA |
| 4 | Ala_AGC_chr6:26682715-26682787 (+) | GGGGAATTAGCTCAAGTGGTAGAGCGCTTGCTTAGCATGCAAGAGGTA |
| | | GTGGGATCGATGCCCACATTCTCCA |
| 5 | Ala_AGC_chr6:26705606-26705678 (+) | GGGGAATTAGCTCAAGCGGTAGAGCGCTTGCTTAGCATGCAAGAGGTA |
| | | GTGGGATCGATGCCCACATTCTCCA |
| 6 | Ala_AGC_chr6:26673590-26673662 (+) | GGGGAATTAGCTCAAGTGGTAGAGCGCTTGCTTAGCATGCAAGAGGTA |
| | | GTGGGATCAATGCCCACATTCTCCA |
| 7 | Ala_AGC_chr14:89445442-89445514 (+) | GGGGAATTAGCTCAAGTGGTAGAGCGCTCGCTTAGCATGCGAGAGGTA |
| | | GTGGGATCGATGCCCGCATTCTCCA |
| 8 | Ala_AGC_chr6:58196623-58196695 (−) | GGGGAATTAGCCCAAGTGGTAGAGCGCTTGCTTAGCATGCAAGAGGTA |
| | | GTGGGATCGATGCCCACATTCTCCA |
| 9 | Ala_AGC_chr6:28806221-28806292 (−) | GGGGGTGTAGCTCAGTGGTAGAGCGCGTGCTTAGCATGCACGAGGCCC |
| | | CGGGTTCAATCCCCGGCACCTCCA |
| 10 | Ala_AGC_chr6:28574933-28575004 (+) | GGGGGTGTAGCTCAGTGGTAGAGCGCGTGCTTAGCATGTACGAGGTCC |
| | | CGGGTTCAATCCCCGGCACCTCCA |
| 11 | Ala_AGC_chr6:28626014-28626085 (−) | GGGGATGTAGCTCAGTGGTAGAGCGCATGCTTAGCATGCATGAGGTCC |
| | | CGGGTTCGATCCCCAGCATCTCCA |
| 12 | Ala_AGC_chr6:28678366-28678437 (+) | GGGGGTGTAGCTCAGTGGTAGAGCGCGTGCTTAGCATGCACGAGGCCC |
| | | TGGGTTCAATCCCCAGCACCTCCA |
| 13 | Ala_AGC_chr6:28779849-28779920 (−) | GGGGGTATAGCTCAGCGGTAGAGCGCGTGCTTAGCATGCACGAGGTCC |
| | | TGGGTTCAATCCCCAATACCTCCA |
| 14 | Ala_AGC_chr6:28687481-28687552 (+) | GGGGGTGTAGCTCAGTGGTAGAGCGCGTGCTTAGCATGCACGAGGCCC |
| | | CGGGTTCAATCCCTGGCACCTCCA |
| 15 | Ala_AGC_chr2:27274082-27274154 (+) | GGGGGATTAGCTCAAATGGTAGAGCGCTCGCTTAGCATGCGAGAGGTA |
| | | GCGGGATCGATGCCCGCATCCTCCA |
| 16 | Ala_AGC_chr6:26730737-26730809 (+) | GGGGAATTAGCTCAGGCGGTAGAGCGCTCGCTTAGCATGCGAGAGGTA |
| | | GCGGGATCGACGCCCGCATTCTCCA |
| 17 | Ala_CGC_chr6:26553731-26553802 (+) | GGGGATGTAGCTCAGTGGTAGAGCGCATGCTTCGCATGTATGAGGTCC |
| | | CGGGTTCGATCCCCGGCATCTCCA |
| 18 | Ala_CGC_chr6:28641613-28641684 (−) | GGGGATGTAGCTCAGTGGTAGAGCGCATGCTTCGCATGTATGAGGCCC |
| | | CGGGTTCGATCCCCGGCATCTCCA |
| 19 | Ala_CGC_chr2:157257281-157257352 (+) | GGGGATGTAGCTCAGTGGTAGAGCGCGCGCTTCGCATGTGTGAGGTCC |
| | | CGGGTTCAATCCCCGGCATCTCCA |
| 20 | Ala_CGC_chr6:28697092-28697163 (+) | GGGGGTGTAGCTCAGTGGTAGAGCGCGTGCTTCGCATGTACGAGGCCC |
| | | CGGGTTCGACCCCCGGCTCCTCCA |
| 21 | Ala_TGC_chr6:28757547-28757618 (−) | GGGGGTGTAGCTCAGTGGTAGAGCGCATGCTTTGCATGTATGAGGTCC |
| | | CGGGTTCGATCCCCGGCACCTCCA |
| 22 | Ala_TGC_chr6:28611222-28611293 (+) | GGGGATGTAGCTCAGTGGTAGAGCGCATGCTTTGCATGTATGAGGTCC |
| | | CGGGTTCGATCCCCGGCATCTCCA |
| 23 | Ala_TGC_chr5:180633868-180633939 (+) | GGGGATGTAGCTCAGTGGTAGAGCGCATGCTTTGCATGTATGAGGCCC |
| | | CGGGTTCGATCCCCGGCATCTCCA |
| 24 | Ala_TGC_chr12:125424512-125424583 (+) | GGGGATGTAGCTCAGTGGTAGAGCGCATGCTTTGCACGTATGAGGCCC |
| | | CGGGTTCAATCCCCGGCATCTCCA |
| 25 | Ala_TGC_chr6:28785012-28785083 (−) | GGGGGTGTAGCTCAGTGGTAGAGCGCATGCTTTGCATGTATGAGGCCT |
| | | CGGGTTCGATCCCCGACACCTCCA |
| 26 | Ala_TGC_chr6:28726141-28726212 (−) | GGGGGTGTAGCTCAGTGGTAGAGCACATGCTTTGCATGTGTGAGGCCC |
| | | CGGGTTCGATCCCCGGCACCTCCA |
| 27 | Ala_TGC_chr6:28770577-28770647 (−) | GGGGGTGTAGCTCAGTGGTAGAGCGCATGCTTTGCATGTATGAGGCCT |
| | | CGGTTCGATCCCCGACACCTCCA |
| 28 | Arg_ACG_chr6:26328368-26328440 (+) | GGGCCAGTGGCGCAATGGATAACGCGTCTGACTACGGATCAGAAGATT |
| | | CCAGGTTCGACTCCTGGCTGGCTCG |
| 29 | Arg_ACG_chr3:45730491-45730563 (−) | GGGCCAGTGGCGCAATGGATAACGCGTCTGACTACGGATCAGAAGATT |
| | | CTAGGTTCGACTCCTGGCTGGCTCG |
| 30 | Arg_CCG_chr6:28710729-28710801 (−) | GGCCGCGTGGCCTAATGGATAAGGCGTCTGATTCCGGATCAGAAGATT |
| | | GAGGGTTCGAGTCCCTTCGTGGTCG |
| 31 | Arg_CCG_chr17:66016013-66016085 (−) | GACCCAGTGGCCTAATGGATAAGGCATCAGCCTCCGGAGCTGGGGATT |
| | | GTGGGTTCGAGTCCCATCTGGGTCG |
| 32 | Arg_CCT_chr17:73030001-73030073 (+) | GCCCCAGTGGCCTAATGGATAAGGCACTGGCCTCCTAAGCCAGGGATT |
| | | GTGGGTTCGAGTCCCACCTGGGGTA |
| 33 | Arg_CCT_chr17:73030526-73030598 (−) | GCCCCAGTGGCCTAATGGATAAGGCACTGGCCTCCTAAGCCAGGGATT |
| | | GTGGGTTCGAGTCCCACCTGGGGTG |
| 34 | Arg_CCT_chr16:3202901-3202973 (+) | GCCCCGGTGGCCTAATGGATAAGGCATTGGCCTCCTAAGCCAGGGATT |
| | | GTGGGTTCGAGTCCCACCCGGGGTA |
| 35 | Arg_CCT_chr7:139025446-139025518 (+) | GCCCCAGTGGCCTAATGGATAAGGCATTGGCCTCCTAAGCCAGGGATT |
| | | GTGGGTTCGAGTCCCATCTGGGGTG |
| 36 | Arg_CCT_chr16:3243918-3243990 (+) | GCCCCAGTGGCCTGATGGATAAGGTACTGGCCTCCTAAGCCAGGGATT |
| | | GTGGGTTCGAGTTCCACCTGGGGTA |
| 37 | Arg_TCG_chr15:89878304-89878376 (+) | GGCCGCGTGGCCTAATGGATAAGGCGTCTGACTTCGGATCAGAAGATT |
| | | GCAGGTTCGAGTCCTGCCGCGGTCG |
| 38 | Arg_TCG_chr6:26323046-26323118 (+) | GACCACGTGGCCTAATGGATAAGGCGTCTGACTTCGGATCAGAAGATT |
| | | GAGGGTTCGAATCCCTCCGTGGTTA |
| 39 | Arg_TCG_chr17:73031208-73031280 (+) | GACCGCGTGGCCTAATGGATAAGGCGTCTGACTTCGGATCAGAAGATT |
| | | GAGGGTTCGAGTCCCTTCGTGGTCG |
| 40 | Arg_TCG_chr6:26299905-26299977 (+) | GACCACGTGGCCTAATGGATAAGGCGTCTGACTTCGGATCAGAAGATT |
| | | GAGGGTTCGAATCCCTTCGTGGTTA |
| 41 | Arg_TCG_chr6:28510891-28510963 (−) | GACCACGTGGCCTAATGGATAAGGCGTCTGACTTCGGATCAGAAGATT |
| | | GAGGGTTCGAATCCCTTCGTGGTTG |
| 42 | Arg_TCG_chr9:112960803-112960875 (+) | GGCCGTGTGGCCTAATGGATAAGGCGTCTGACTTCGGATCAAAAGATT |
| | | GCAGGTTTGAGTTCTGCCACGGTCG |
| 43 | Arg_TCT_chr1:94313129-94313213 (+) | GGCTCCGTGGCGCAATGGATAGCGCATTGGACTTCTAGAGGCTGAAGG |
| | | CATTCAAAGGTTCCGGGTTCGAGTCCCGGCGGAGTCG |
| 44 | Arg_TCT_chr17:8024243-8024330 (+) | GGCTCTGTGGCGCAATGGATAGCGCATTGGACTTCTAGTGACGAATAG |
| | | AGCAATTCAAAGGTTGTGGGTTCGAATCCCACCAGAGTCG |
| 45 | Arg_TCT_chr9:131102355-131102445 (−) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCG |
| 46 | Arg_TCT_chr11:59318767-59318852 (+) | GGCTCTGTGGCGCAATGGATAGCGCATTGGACTTCTAGATAGTTAGAG |
| | | AAATTCAAAGGTTGTGGGTTCGAGTCCCACCAGAGTCG |
| 47 | Arg_TCT_chr1:159111401-159111474 (−) | GTCTCTGTGGCGCAATGGACGAGCGCGCTGGACTTCTAATCCAGAGGT |
| | | TCCGGGTTCGAGTCCCGGCAGAGATG |
| 48 | Arg_TCT_chr6:27529963-27530049 (+) | GGCTCTGTGGCGCAATGGATAGCGCATTGGACTTCTAGCCTAAATCAA |
| | | GAGATTCAAAGGTTGCGGGTTCGAGTCCCTCCAGAGTCG |
| 49 | Asn_GTT_chr1:161510031-161510104 (+) | GTCTCTGTGGCGCAATCGGTTAGCGCGTTCGGCTGTTAACCGAAAGGT |
| | | TGGTGGTTCGATCCCACCCAGGGACG |
| 50 | Asn_GTT_chr1:143879832-143879905 (−) | GTCTCTGTGGCGCAATCGGCTAGCGCGTTTGGCTGTTAACTAAAAGGT |
| | | TGGCGGTTCGAACCCACCCAGAGGCG |
| 51 | Asn_GTT_chr1:144301611-144301684 (+) | GTCTCTGTGGTGCAATCGGTTAGCGCGTTCCGCTGTTAACCGAAAGCT |
| | | GTGTGGTTCGAGCCCACCCAGGGATG |
| 52 | Asn_GTT_chr1:149326272-149326345 (−) | GTCTCTGTGGCGCAATCGGCTAGCGCGTTTGGCTGTTAACTAAAAAGT |
| | | TGGTGGTTCGAACACACCCAGAGGCG |
| 53 | Asn_GTT_chr1:148248115-148248188 (+) | GTCTCTGTGGCGCAATCGGTTAGCGCGTTCGGCTGTTAACCGAAAGGT |
| | | TGGTGGTTCGAGCCCACCCAGGGACG |
| 54 | Asn_GTT_chr1:148598314-148598387 (−) | GTCTCTGTGGCGCAATCGGTTAGCGCATTCGGCTGTTAACCGAAAGGT |
| | | TGGTGGTTCGAGCCCACCCAGGGACG |
| 55 | Asn_GTT_chr1:17216172-17216245 (+) | GTCTCTGTGGCGCAATCGGTTAGCGCGTTCGGCTGTTAACCGAAAGAT |
| | | TGGTGGTTCGAGCCCACCCAGGGACG |
| 56 | Asn_GTT_chr1:16847080-16847153 (−) | GTCTCTGTGGCGCAATCGGTTAGCGCGTTCGGCTGTTAACTGAAAGGT |
| | | TGGTGGTTCGAGCCCACCCAGGGACG |
| 57 | Asn_GTT_chr1:149230570-149230643 (−) | GTCTCTGTGGCGCAATGGGTTAGCGCGTTCGGCTGTTAACCGAAAGGT |
| | | TGGTGGTTCGAGCCCATCCAGGGACG |
| 58 | Asn_GTT_chr1:148000805-148000878 (+) | GTCTCTGTGGCGTAGTCGGTTAGCGCGTTCGGCTGTTAACCGAAAAGT |
| | | TGGTGGTTCGAGCCCACCCAGGAACG |
| 59 | Asn_GTT_chr1:149711798-149711871 (−) | GTCTCTGTGGCGCAATCGGCTAGCGCGTTTGGCTGTTAACTAAAAGGT |
| | | TGGTGGTTCGAACCCACCCAGAGGCG |
| 60 | Asn_GTT_chr1:145979034-145979107 (−) | GTCTCTGTGGCGCAATCGGTTAGCGCGTTCGGCTGTTAACTGAAAGGT |
| | | TAGTGGTTCGAGCCCACCCGGGGACG |
| 61 | Asp_GTC_chr12:98897281-98897352 (+) | TCCTCGTTAGTATAGTGGTTAGTATCCCCGCCTGTCACGCGGGAGACC |
| | | GGGGTTCAATTCCCCGACGGGGAG |
| 62 | Asp_GTC_chr1:161410615-161410686 (−) | TCCTCGTTAGTATAGTGGTGAGTATCCCCGCCTGTCACGCGGGAGACC |
| | | GGGGTTCGATTCCCCGACGGGGAG |
| 63 | Asp_GTC_chr6:27551236-27551307 (−) | TCCTCGTTAGTATAGTGGTGAGTGTCCCCGTCTGTCACGCGGGAGACC |
| | | GGGGTTCGATTCCCCGACGGGGAG |
| 64 | Cys_GCA_chr7:149007281-149007352 (+) | GGGGGCATAGCTCAGTGGTAGAGCATTTGACTGCAGATCAAGAGGTCC |
| | | CTGGTTCAAATCCAGGTGCCCCCT |
| 65 | Cys_GCA_chr7: 149074601-149074672 (−) | GGGGGTATAGCTCAGGGGTAGAGCATTTGACTGCAGATCAAGAGGTCC |
| | | CTGGTTCAAATCCAGGTGCCCCCC |
| 66 | Cys_GCA_chr7:149112229-149112300 (−) | GGGGGTATAGCTTAGCGGTAGAGCATTTGACTGCAGATCAAGAGGTCC |
| | | CCGGTTCAAATCCGGGTGCCCCCT |
| 67 | Cys_GCA_chr7: 149344046-149344117 (−) | GGGGGTATAGCTTAGGGGTAGAGCATTTGACTGCAGATCAAAAGGTCC |
| | | CTGGTTCAAATCCAGGTGCCCCTT |
| 68 | Cys_GCA_chr7:149052766-149052837 (−) | GGGGGTATAGCTCAGGGGTAGAGCATTTGACTGCAGATCAAGAGGTCC |
| | | CCAGTTCAAATCTGGGTGCCCCCT |
| 69 | Cys_GCA_chr17:37017937-37018008 (−) | GGGGGTATAGCTCAGGGGTAGAGCATTTGACTGCAGATCAAGAAGTCC |
| | | CCGGTTCAAATCCGGGTGCCCCCT |
| 70 | Cys_GCA_chr7:149281816-149281887 (+) | GGGGGTATAGCTCAGGGGTAGAGCATTTGACTGCAGATCAAGAGGTCT |
| | | CTGGTTCAAATCCAGGTGCCCCCT |
| 71 | Cys_GCA_chr7:149243631-149243702 (+) | GGGGGTATAGCTCAGGGGTAGAGCACTTGACTGCAGATCAAGAAGTCC |
| | | TTGGTTCAAATCCAGGTGCCCCCT |
| 72 | Cys_GCA_chr7:149388272-149388343 (−) | GGGGATATAGCTCAGGGGTAGAGCATTTGACTGCAGATCAAGAGGTCC |
| | | CCGGTTCAAATCCGGGTGCCCCCC |
| 73 | Cys_GCA_chr7:149072850-149072921 (−) | GGGGGTATAGTTCAGGGGTAGAGCATTTGACTGCAGATCAAGAGGTCC |
| | | CTGGTTCAAATCCAGGTGCCCCCT |
| 74 | Cys_GCA_chr7:149310156-149310227 (−) | GGGGGTATAGCTCAGGGGTAGAGCATTTGACTGCAAATCAAGAGGTCC |
| | | CTGATTCAAATCCAGGTGCCCCCT |
| 75 | Cys_GCA_chr4:124430005-124430076 (−) | GGGGGTATAGCTCAGTGGTAGAGCATTTGACTGCAGATCAAGAGGTCC |
| | | CCGGTTCAAATCCGGGTGCCCCCT |
| 76 | Cys_GCA_chr7:149295046-149295117 (+) | GGGCGTATAGCTCAGGGGTAGAGCATTTGACTGCAGATCAAGAGGTCC |
| | | CCAGTTCAAATCTGGGTGCCCCCT |
| 77 | Cys_GCA_chr7:149361915-149361986 (+) | GGGGGTATAGCTCACAGGTAGAGCATTTGACTGCAGATCAAGAGGTCC |
| | | CCGGTTCAAATCTGGGTGCCCCCT |
| 78 | Cys_GCA_chr7:149253802-149253871 (+) | GGGCGTATAGCTCAGGGGTAGAGCATTTGACTGCAGATCAAGAGGTCC |
| | | CCAGTTCAAATCTGGGTGCCCA |
| 79 | Cys_GCA_chr7:149292305-149292376 (−) | GGGGGTATAGCTCACAGGTAGAGCATTTGACTGCAGATCAAGAGGTCC |
| | | CCGGTTCAAATCCGGTTACTCCCT |
| 80 | Cys_GCA_chr7:149286164-149286235 (−) | GGGGGTATAGCTCAGGGGTAGAGCACTTGACTGCAGATCAAGAGGTCC |
| | | CTGGTTCAAATCCAGGTGCCCCCT |
| 81 | Cys_GCA_chr17:37025545-37025616 (−) | GGGGGTATAGCTCAGTGGTAGAGCATTTGACTGCAGATCAAGAGGTCC |
| | | CTGGTTCAAATCCGGGTGCCCCCT |
| 82 | Cys_GCA_chr15:80036997-80037069 (+) | GGGGGTATAGCTCAGTGGGTAGAGCATTTGACTGCAGATCAAGAGGTC |
| | | CCCGGTTCAAATCCGGGTGCCCCCT |
| 83 | Cys_GCA_chr3:131947944-131948015 (−) | GGGGGTGTAGCTCAGTGGTAGAGCATTTGACTGCAGATCAAGAGGTCC |
| | | CTGGTTCAAATCCAGGTGCCCCCT |
| 84 | Cys_GCA_chr1:93981834-93981906 (−) | GGGGGTATAGCTCAGGTGGTAGAGCATTTGACTGCAGATCAAGAGGTC |
| | | CCCGGTTCAAATCCGGGTGCCCCCT |
| 85 | Cys_GCA_chr14:73429679-73429750 (+) | GGGGGTATAGCTCAGGGGTAGAGCATTTGACTGCAGATCAAGAGGTCC |
| | | CCGGTTCAAATCCGGGTGCCCCCT |
| 86 | Cys_GCA_chr3:131950642-131950713 (−) | GGGGGTATAGCTCAGGGGTAGAGCATTTGACTGCAGATCAAGAGGTCC |
| | | CTGGTTCAAATCCAGGTGCCCCCT |
| 87 | Gln_CTG_chr6:18836402-18836473 (+) | GGTTCCATGGTGTAATGGTTAGCACTCTGGACTCTGAATCCAGCGATC |
| | | CGAGTTCAAATCTCGGTGGAACCT |
| 88 | Gln_CTG_chr6:27515531-27515602 (−) | GGTTCCATGGTGTAATGGTTAGCACTCTGGACTCTGAATCCAGCGATC |
| | | CGAGTTCAAGTCTCGGTGGAACCT |
| 89 | Gln_CTG_chr1:145963304-145963375 (+) | GGTTCCATGGTGTAATGGTGAGCACTCTGGACTCTGAATCCAGCGATC |
| | | CGAGTTCGAGTCTCGGTGGAACCT |
| 90 | Gln_CTG_chr1:147737382-147737453 (−) | GGTTCCATGGTGTAATGGTAAGCACTCTGGACTCTGAATCCAGCGATC |
| | | CGAGTTCGAGTCTCGGTGGAACCT |
| 91 | Gln_CTG_chr6:27263212-27263283 (+) | GGTTCCATGGTGTAATGGTTAGCACTCTGGACTCTGAATCCGGTAATC |
| | | CGAGTTCAAATCTCGGTGGAACCT |
| 92 | Gln_CTG_chr6:27759135-27759206 (−) | GGCCCCATGGTGTAATGGTCAGCACTCTGGACTCTGAATCCAGCGATC |
| | | CGAGTTCAAATCTCGGTGGGACCC |
| 93 | Gln_CTG_chr1:147800937-147801008 (+) | GGTTCCATGGTGTAATGGTAAGCACTCTGGACTCTGAATCCAGCCATC |
| | | TGAGTTCGAGTCTCTGTGGAACCT |
| 94 | Gln_TTG_chr17:47269890-47269961 (+) | GGTCCCATGGTGTAATGGTTAGCACTCTGGACTTTGAATCCAGCGATC |
| | | CGAGTTCAAATCTCGGTGGGACCT |
| 95 | Gln_TTG_chr6:28557156-28557227 (+) | GGTCCCATGGTGTAATGGTTAGCACTCTGGACTTTGAATCCAGCAATC |
| | | CGAGTTCGAATCTCGGTGGGACCT |
| 96 | Gln_TTG_chr6:26311424-26311495 (−) | GGCCCCATGGTGTAATGGTTAGCACTCTGGACTTTGAATCCAGCGATC |
| | | CGAGTTCAAATCTCGGTGGGACCT |
| 97 | Gln_TTG_chr6:145503859-145503930 (+) | GGTCCCATGGTGTAATGGTTAGCACTCTGGGCTTTGAATCCAGCAATC |
| | | CGAGTTCGAATCTTGGTGGGACCT |
| 98 | Glu_CTC_chr1:145399233-145399304 (−) | TCCCTGGTGGTCTAGTGGTTAGGATTCGGCGCTCTCACCGCCGCGGCC |
| | | CGGGTTCGATTCCCGGTCAGGGAA |
| 99 | Glu_CTC_chr1:249168447-249168518 (+) | TCCCTGGTGGTCTAGTGGTTAGGATTCGGCGCTCTCACCGCCGCGGCC |
| | | CGGGTTCGATTCCCGGTCAGGAAA |
| 100 | Glu_TTC_chr2:131094701-131094772 (−) | TCCCATATGGTCTAGCGGTTAGGATTCCTGGTTTTCACCCAGGTGGCC |
| | | CGGGTTCGACTCCCGGTATGGGAA |
| 101 | Glu_TTC_chr13:45492062-45492133 (−) | TCCCACATGGTCTAGCGGTTAGGATTCCTGGTTTTCACCCAGGCGGCC |
| | | CGGGTTCGACTCCCGGTGTGGGAA |
| 102 | Glu_TTC_chr1:17199078-17199149 (+) | TCCCTGGTGGTCTAGTGGCTAGGATTCGGCGCTTTCACCGCCGCGGCC |
| | | CGGGTTCGATTCCCGGCCAGGGAA |
| 103 | Glu_TTC_chr1:16861774-16861845 (−) | TCCCTGGTGGTCTAGTGGCTAGGATTCGGCGCTTTCACCGCCGCGGCC |
| | | CGGGTTCGATTCCCGGTCAGGGAA |
| 104 | Gly_CCC_chr1:16872434-16872504 (−) | GCATTGGTGGTTCAGTGGTAGAATTCTCGCCTCCCACGCGGGAGACCC |
| | | GGGTTCAATTCCCGGCCAATGCA |
| 105 | Gly_CCC_chr2:70476123-70476193 (−) | GCGCCGCTGGTGTAGTGGTATCATGCAAGATTCCCATTCTTGCGACCC |
| | | GGGTTCGATTCCCGGGCGGCGCA |
| 106 | Gly_CCC_chr17: 19764175-19764245 (+) | GCATTGGTGGTTCAATGGTAGAATTCTCGCCTCCCACGCAGGAGACCC |
| | | AGGTTCGATTCCTGGCCAATGCA |
| 107 | Gly_GCC_chr1:161413094-161413164 (+) | GCATGGGTGGTTCAGTGGTAGAATTCTCGCCTGCCACGCGGGAGGCCC |
| | | GGGTTCGATTCCCGGCCCATGCA |
| 108 | Gly_GCC_chr1: 161493637-161493707 (−) | GCATTGGTGGTTCAGTGGTAGAATTCTCGCCTGCCACGCGGGAGGCCC |
| | | GGGTTCGATTCCCGGCCAATGCA |
| 109 | Gly_GCC_chr16:70812114-70812184 (−) | GCATTGGTGGTTCAGTGGTAGAATTCTCGCCTGCCACGCGGGAGGCCC |
| | | GGGTTTGATTCCCGGCCAGTGCA |
| 110 | Gly_GCC_chr1:161450356-161450426 (+) | GCATAGGTGGTTCAGTGGTAGAATTCTTGCCTGCCACGCAGGAGGCCC |
| | | AGGTTTGATTCCTGGCCCATGCA |
| 111 | Gly_GCC_chr16:70822597-70822667 (+) | GCATTGGTGGTTCAGTGGTAGAATTCTCGCCTGCCATGCGGGCGGCCG |
| | | GGCTTCGATTCCTGGCCAATGCA |
| 112 | Gly_TCC_chr19:4724082-4724153 (+) | GCGTTGGTGGTATAGTGGTTAGCATAGCTGCCTTCCAAGCAGTTGACC |
| | | CGGGTTCGATTCCCGGCCAACGCA |
| 113 | Gly_TCC_chr1:145397864-145397935 (−) | GCGTTGGTGGTATAGTGGTGAGCATAGCTGCCTTCCAAGCAGTTGACC |
| | | CGGGTTCGATTCCCGGCCAACGCA |
| 114 | Gly_TCC_chr17:8124866-8124937 (+) | GCGTTGGTGGTATAGTGGTAAGCATAGCTGCCTTCCAAGCAGTTGACC |
| | | CGGGTTCGATTCCCGGCCAACGCA |
| 115 | Gly_TCC_chr1:161409961-161410032 (−) | GCGTTGGTGGTATAGTGGTGAGCATAGTTGCCTTCCAAGCAGTTGACC |
| | | CGGGCTCGATTCCCGCCCAACGCA |
| 116 | His_GTG_chr1:145396881-145396952 (−) | GCCGTGATCGTATAGTGGTTAGTACTCTGCGTTGTGGCCGCAGCAACC |
| | | TCGGTTCGAATCCGAGTCACGGCA |
| 117 | His_GTG_chr1:149155828-149155899 (−) | GCCATGATCGTATAGTGGTTAGTACTCTGCGCTGTGGCCGCAGCAACC |
| | | TCGGTTCGAATCCGAGTCACGGCA |
| 118 | Ile_AAT_chr6:58149254-58149327 (+) | GGCCGGTTAGCTCAGTTGGTTAGAGCGTGGCGCTAATAACGCCAAGGT |
| | | CGCGGGTTCGATCCCCGTACGGGCCA |
| 119 | Ile_AAT_chr6:27655967-27656040 (+) | GGCCGGTTAGCTCAGTTGGTTAGAGCGTGGTGCTAATAACGCCAAGGT |
| | | CGCGGGTTCGATCCCCGTACTGGCCA |
| 120 | Ile_AAT_chr6:27242990-27243063 (−) | GGCTGGTTAGCTCAGTTGGTTAGAGCGTGGTGCTAATAACGCCAAGGT |
| | | CGCGGGTTCGATCCCCGTACTGGCCA |
| 121 | Ile_AAT_chr17:8130309-8130382 (−) | GGCCGGTTAGCTCAGTTGGTTAGAGCGTGGTGCTAATAACGCCAAGGT |
| | | CGCGGGTTCGAACCCCGTACGGGCCA |
| 122 | Ile_AAT_chr6:26554350-26554423 (+) | GGCCGGTTAGCTCAGTTGGTTAGAGCGTGGTGCTAATAACGCCAAGGT |
| | | CGCGGGTTCGATCCCCGTACGGGCCA |
| 123 | Ile_AAT_chr6:26745255-26745328 (−) | GGCCGGTTAGCTCAGTTGGTTAGAGCGTGGTGCTAATAACGCTAAGGT |
| | | CGCGGGTTCGATCCCCGTACTGGCCA |
| 124 | Ile_AAT_chr6:26721221-26721294 (−) | GGCCGGTTAGCTCAGTTGGTCAGAGCGTGGTGCTAATAACGCCAAGGT |
| | | CGCGGGTTCGATCCCCGTACGGGCCA |
| 125 | Ile_AAT_chr6:27636362-27636435 (+) | GGCCGGTTAGCTCAGTCGGCTAGAGCGTGGTGCTAATAACGCCAAGGT |
| | | CGCGGGTTCGATCCCCGTACGGGCCA |
| 126 | Ile_AAT_chr6:27241739-27241812 (+) | GGCTGGTTAGTTCAGTTGGTTAGAGCGTGGTGCTAATAACGCCAAGGT |
| | | CGTGGGTTCGATCCCCATATCGGCCA |
| 127 | Ile_GAT_chrX:3756418-3756491 (−) | GGCCGGTTAGCTCAGTTGGTAAGAGCGTGGTGCTGATAACACCAAGGT |
| | | CGCGGGCTCGACTCCCGCACCGGCCA |
| 128 | Ile_TAT_chr19:39902808-39902900 (−) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCA |
| 129 | Ile_TAT_chr2:43037676-43037768 (+) | GCTCCAGTGGCGCAATCGGTTAGCGCGCGGTACTTATACAGCAGTACA |
| | | TGCAGAGCAATGCCGAGGTTGTGAGTTCGAGCCTCACCTGGAGCA |
| 130 | Ile_TAT_chr6:26988125-26988218 (+) | GCTCCAGTGGCGCAATCGGTTAGCGCGCGGTACTTATATGGCAGTATG |
| | | TGTGCGAGTGATGCCGAGGTTGTGAGTTCGAGCCTCACCTGGAGCA |
| 131 | Ile_TAT_chr6:27599200-27599293 (+) | GCTCCAGTGGCGCAATCGGTTAGCGCGCGGTACTTATACAACAGTATA |
| | | TGTGCGGGTGATGCCGAGGTTGTGAGTTCGAGCCTCACCTGGAGCA |
| 132 | Ile_TAT_chr6:28505367-28505460 (+) | GCTCCAGTGGCGCAATCGGTTAGCGCGCGGTACTTATAAGACAGTGCA |
| | | CCTGTGAGCAATGCCGAGGTTGTGAGTTCAAGCCTCACCTGGAGCA |
| 133 | Leu_AAG_chr5:180524474-180524555 (−) | GGTAGCGTGGCCGAGCGGTCTAAGGCGCTGGATTAAGGCTCCAGTCTC |
| | | TTCGGAGGCGTGGGTTCGAATCCCACCGCTGCCA |
| 134 | Leu_AAG_chr5:180614701-180614782 (+) | GGTAGCGTGGCCGAGCGGTCTAAGGCGCTGGATTAAGGCTCCAGTCTC |
| | | TTCGGGGGCGTGGGTTCGAATCCCACCGCTGCCA |
| 135 | Leu_AAG_chr6:28956779-28956860 (+) | GGTAGCGTGGCCGAGCGGTCTAAGGCGCTGGATTAAGGCTCCAGTCTC |
| | | TTCGGGGGCGTGGGTTCAAATCCCACCGCTGCCA |
| 136 | Leu_AAG_chr6:28446400-28446481 (−) | GGTAGCGTGGCCGAGTGGTCTAAGACGCTGGATTAAGGCTCCAGTCTC |
| | | TTCGGGGGCGTGGGTTTGAATCCCACCGCTGCCA |
| 137 | Leu_CAA_chr6:28864000-28864105 (−) | GTCAGGATGGCCGAGTGGTCTAAGGCGCCAGACTCAAGCTAAGCTTCC |
| | | TCCGCGGTGGGGATTCTGGTCTCCAATGGAGGCGTGGGTTCGAATCCC |
| 138 | Leu_CAA_chr6:28908830-28908934 (+) | GTCAGGATGGCCGAGTGGTCTAAGGCGCCAGACTCAAGCTTGGCTTCC |
| | | TCGTGTTGAGGATTCTGGTCTCCAATGGAGGCGTGGGTTCGAATCCCA |
| 139 | Leu_CAA_chr6:27573417-27573524 (−) | GTCAGGATGGCCGAGTGGTCTAAGGCGCCAGACTCAAGCTTACTGCTT |
| | | CCTGTGTTCGGGTCTTCTGGTCTCCGTATGGAGGCGTGGGTTCGAATC |
| | | C |
| 140 | Leu_CAA_chr6:27570348-27570454 (−) | GTCAGGATGGCCGAGTGGTCTAAGGCGCCAGACTCAAGTTGCTACTTC |
| | | CCAGGTTTGGGGCTTCTGGTCTCCGCATGGAGGCGTGGGTTCGAATCC |
| 141 | Leu_CAA_chr1:249168054-249168159 (+) | GTCAGGATGGCCGAGTGGTCTAAGGCGCCAGACTCAAGGTAAGCACCT |
| | | TGCCTGCGGGCTTTCTGGTCTCCGGATGGAGGCGTGGGTTCGAATCCC |
| 142 | Leu_CAA_chr11:9296790-9296863 (+) | GCCTCCTTAGTGCAGTAGGTAGCGCATCAGTCTCAAAATCTGAATGGT |
| | | CCTGAGTTCAAGCCTCAGAGGGGGCA |
| 143 | Leu_CAA_chr1:161581736-161581819 (−) | GTCAGGATGGCCGAGCAGTCTTAAGGCGCTGCGTTCAAATCGCACCCT |
| | | CCGCTGGAGGCGTGGGTTCGAATCCCACTTTTGACA |
| 144 | Leu_CAG_chr1:161411323-161411405 (+) | GTCAGGATGGCCGAGCGGTCTAAGGCGCTGCGTTCAGGTCGCAGTCTC |
| | | CCCTGGAGGCGTGGGTTCGAATCCCACTCCTGACA |
| 145 | Leu_CAG_chr16:57333863-57333945 (+) | GTCAGGATGGCCGAGCGGTCTAAGGCGCTGCGTTCAGGTCGCAGTCTC |
| | | CCCTGGAGGCGTGGGTTCGAATCCCACTTCTGACA |
| 146 | Leu_TAA_chr6:144537684-144537766 (+) | ACCAGGATGGCCGAGTGGTTAAGGCGTTGGACTTAAGATCCAATGGAC |
| | | ATATGTCCGCGTGGGTTCGAACCCCACTCCTGGTA |
| 147 | Leu_TAA_chr6:27688898-27688980 (−) | ACCGGGATGGCCGAGTGGTTAAGGCGTTGGACTTAAGATCCAATGGGC |
| | | TGGTGCCCGCGTGGGTTCGAACCCCACTCTCGGTA |
| 148 | Leu_TAA_chr11:59319228-59319310 (+) | ACCAGAATGGCCGAGTGGTTAAGGCGTTGGACTTAAGATCCAATGGAT |
| | | TCATATCCGCGTGGGTTCGAACCCCACTTCTGGTA |
| 149 | Leu_TAA_chr6:27198334-27198416 (−) | ACCGGGATGGCTGAGTGGTTAAGGCGTTGGACTTAAGATCCAATGGAC |
| | | AGGTGTCCGCGTGGGTTCGAGCCCCACTCCCGGTA |
| 150 | Leu_TAG_chr17:8023632-8023713 (−) | GGTAGCGTGGCCGAGCGGTCTAAGGCGCTGGATTTAGGCTCCAGTCTC |
| | | TTCGGAGGCGTGGGTTCGAATCCCACCGCTGCCA |
| 151 | Leu_TAG_chr14:21093529-21093610 (+) | GGTAGTGTGGCCGAGCGGTCTAAGGCGCTGGATTTAGGCTCCAGTCTC |
| | | TTCGGGGGCGTGGGTTCGAATCCCACCACTGCCA |
| 152 | Leu_TAG_chr16:22207032-22207113 (−) | GGTAGCGTGGCCGAGTGGTCTAAGGCGCTGGATTTAGGCTCCAGTCAT |
| | | TTCGATGGCGTGGGTTCGAATCCCACCGCTGCCA |
| 153 | Lys_CTT_chr14:58706613-58706685 (−) | GCCCGGCTAGCTCAGTCGGTAGAGCATGGGACTCTTAATCCCAGGGTC |
| | | GTGGGTTCGAGCCCCACGTTGGGCG |
| 154 | Lys_CTT_chr19:36066750-36066822 (+) | GCCCAGCTAGCTCAGTCGGTAGAGCATAAGACTCTTAATCTCAGGGTT |
| | | GTGGATTCGTGCCCCATGCTGGGTG |
| 155 | Lys_CTT_chr19:52425393-52425466 (−) | GCAGCTAGCTCAGTCGGTAGAGCATGAGACTCTTAATCTCAGGGTCAT |
| | | GGGTTCGTGCCCCATGTTGGGTGCCA |
| 156 | Lys_CTT_chr1:145395522-145395594 (−) | GCCCGGCTAGCTCAGTCGGTAGAGCATGAGACTCTTAATCTCAGGGTC |
| | | GTGGGTTCGAGCCCCACGTTGGGCG |
| 157 | Lys_CTT_chr16:3207406-3207478 (−) | GCCCGGCTAGCTCAGTCGGTAGAGCATGAGACCCTTAATCTCAGGGTC |
| | | GTGGGTTCGAGCCCCACGTTGGGCG |
| 158 | Lys_CTT_chr16:3241501-3241573 (+) | GCCCGGCTAGCTCAGTCGGTAGAGCATGGGACTCTTAATCTCAGGGTC |
| | | GTGGGTTCGAGCCCCACGTTGGGCG |
| 159 | Lys_CTT_chr16:3230555-3230627 (−) | GCCCGGCTAGCTCAGTCGATAGAGCATGAGACTCTTAATCTCAGGGTC |
| | | GTGGGTTCGAGCCGCACGTTGGGCG |
| 160 | Lys_CTT_chr1:55423542-55423614 (−) | GCCCAGCTAGCTCAGTCGGTAGAGCATGAGACTCTTAATCTCAGGGTC |
| | | ATGGGTTTGAGCCCCACGTTTGGTG |
| 161 | Lys_CTT_chr16:3214939-3215011 (+) | GCCTGGCTAGCTCAGTCGGCAAAGCATGAGACTCTTAATCTCAGGGTC |
| | | GTGGGCTCGAGCTCCATGTTGGGCG |
| 162 | Lys_CTT_chr5:26198539-26198611 (−) | GCCCGACTACCTCAGTCGGTGGAGCATGGGACTCTTCATCCCAGGGTT |
| | | GTGGGTTCGAGCCCCACATTGGGCA |
| 163 | Lys_TTT_chr16:73512216-73512288 (−) | GCCTGGATAGCTCAGTTGGTAGAGCATCAGACTTTTAATCTGAGGGTC |
| | | CAGGGTTCAAGTCCCTGTTCAGGCA |
| 164 | Lys_TTT_chr12:27843306-27843378 (+) | ACCCAGATAGCTCAGTCAGTAGAGCATCAGACTTTTAATCTGAGGGTC |
| | | CAAGGTTCATGTCCCTTTTTGGGTG |
| 165 | Lys_TTT_chr11:122430655-122430727 (+) | GCCTGGATAGCTCAGTTGGTAGAGCATCAGACTTTTAATCTGAGGGTC |
| | | CAGGGTTCAAGTCCCTGTTCAGGCG |
| 166 | Lys_TTT_chr1:204475655-204475727 (+) | GCCCGGATAGCTCAGTCGGTAGAGCATCAGACTTTTAATCTGAGGGTC |
| | | CAGGGTTCAAGTCCCTGTTCGGGCG |
| 167 | Lys_TTT_chr6:27559593-27559665 (−) | GCCTGGATAGCTCAGTCGGTAGAGCATCAGACTTTTAATCTGAGGGTC |
| | | CAGGGTTCAAGTCCCTGTTCAGGCG |
| 168 | Lys_TTT_chr11:59323902-59323974 (+) | GCCCGGATAGCTCAGTCGGTAGAGCATCAGACTTTTAATCTGAGGGTC |
| | | CGGGGTTCAAGTCCCTGTTCGGGCG |
| 169 | Lys_TTT_chr6:27302769-27302841 (−) | GCCTGGGTAGCTCAGTCGGTAGAGCATCAGACTTTTAATCTGAGGGTC |
| | | CAGGGTTCAAGTCCCTGTCCAGGCG |
| 170 | Lys_TTT_chr6:28715521-28715593 (+) | GCCTGGATAGCTCAGTTGGTAGAACATCAGACTTTTAATCTGACGGTG |
| | | CAGGGTTCAAGTCCCTGTTCAGGCG |
| 171 | Met_CAT_chr8:124169470-124169542 (−) | GCCTCGTTAGCGCAGTAGGTAGCGCGTCAGTCTCATAATCTGAAGGTC |
| | | GTGAGTTCGATCCTCACACGGGGCA |
| 172 | Met_CAT_chr16:71460396-71460468 (+) | GCCCTCTTAGCGCAGTGGGCAGCGCGTCAGTCTCATAATCTGAAGGTC |
| | | CTGAGTTCGAGCCTCAGAGAGGGCA |
| 173 | Met_CAT_chr6:28912352-28912424 (+) | GCCTCCTTAGCGCAGTAGGCAGCGCGTCAGTCTCATAATCTGAAGGTC |
| | | CTGAGTTCGAACCTCAGAGGGGGCA |
| 174 | Met_CAT_chr6:26735574-26735646 (−) | GCCCTCTTAGCGCAGCGGGCAGCGCGTCAGTCTCATAATCTGAAGGTC |
| | | CTGAGTTCGAGCCTCAGAGAGGGCA |
| 175 | Met_CAT_chr6:26701712-26701784 (+) | GCCCTCTTAGCGCAGCTGGCAGCGCGTCAGTCTCATAATCTGAAGGTC |
| | | CTGAGTTCAAGCCTCAGAGAGGGCA |
| 176 | Met_CAT_chr16:87417628-87417700 (−) | GCCTCGTTAGCGCAGTAGGCAGCGCGTCAGTCTCATAATCTGAAGGTC |
| | | GTGAGTTCGAGCCTCACACGGGGCA |
| 177 | Met_CAT_chr6:58168492-58168564 (−) | GCCCTCTTAGTGCAGCTGGCAGCGCGTCAGTTTCATAATCTGAAAGTC |
| | | CTGAGTTCAAGCCTCAGAGAGGGCA |
| 178 | Phe_GAA_chr6:28758499-28758571 (−) | GCCGAAATAGCTCAGTTGGGAGAGCGTTAGACTGAAGATCTAAAGGTC |
| | | CCTGGTTCGATCCCGGGTTTCGGCA |
| 179 | Phe_GAA_chr11:59333853-59333925 (−) | GCCGAAATAGCTCAGTTGGGAGAGCGTTAGACTGAAGATCTAAAGGTC |
| | | CCTGGTTCAATCCCGGGTTTCGGCA |
| 180 | Phe_GAA_chr6:28775610-28775682 (−) | GCCGAGATAGCTCAGTTGGGAGAGCGTTAGACTGAAGATCTAAAGGTC |
| | | CCTGGTTCAATCCCGGGTTTCGGCA |
| 181 | Phe_GAA_chr6:28791093-28791166 (−) | GCCGAAATAGCTCAGTTGGGAGAGCGTTAGACCGAAGATCTTAAAGGT |
| | | CCCTGGTTCAATCCCGGGTTTCGGCA |
| 182 | Phe_GAA_chr6:28731374-28731447 (−) | GCTGAAATAGCTCAGTTGGGAGAGCGTTAGACTGAAGATCTTAAAGTT |
| | | CCCTGGTTCAACCCTGGGTTTCAGCC |
| 183 | Pro_AGG_chr16:3241989-3242060 (+) | GGCTCGTTGGTCTAGGGGTATGATTCTCGCTTAGGATGCGAGAGGTCC |
| | | CGGGTTCAAATCCCGGACGAGCCC |
| 184 | Pro_AGG_chr1:167684725-167684796 (−) | GGCTCGTTGGTCTAGGGGTATGATTCTCGCTTAGGGTGCGAGAGGTCC |
| | | CGGGTTCAAATCCCGGACGAGCCC |
| 185 | Pro_CGG_chr1:167683962-167684033 (+) | GGCTCGTTGGTCTAGGGGTATGATTCTCGCTTCGGGTGCGAGAGGTCC |
| | | CGGGTTCAAATCCCGGACGAGCCC |
| 186 | Pro_CGG_chr6:27059521-27059592 (+) | GGCTCGTTGGTCTAGGGGTATGATTCTCGCTTCGGGTGTGAGAGGTCC |
| | | CGGGTTCAAATCCCGGACGAGCCC |
| 187 | Pro_TGG_chr14:21101165-21101236 (+) | GGCTCGTTGGTCTAGTGGTATGATTCTCGCTTTGGGTGCGAGAGGTCC |
| | | CGGGTTCAAATCCCGGACGAGCCC |
| 188 | Pro_TGG_chr11:75946869-75946940 (−) | GGCTCGTTGGTCTAGGGGTATGATTCTCGGTTTGGGTCCGAGAGGTCC |
| | | CGGGTTCAAATCCCGGACGAGCCC |
| 189 | Pro_TGG_chr5:180615854-180615925 (−) | GGCTCGTTGGTCTAGGGGTATGATTCTCGCTTTGGGTGCGAGAGGTCC |
| | | CGGGTTCAAATCCCGGACGAGCCC |
| 190 | SeC_TCA_chr19:45981859-45981945 (−) | GCCCGGATGATCCTCAGTGGTCTGGGGTGCAGGCTTCAAACCTGTAGC |
| | | TGTCTAGCGACAGAGTGGTTCAATTCCACCTTTCGGGCG |
| 191 | SeC_TCA_chr22:44546537-44546620 (+) | GCTCGGATGATCCTCAGTGGTCTGGGGTGCAGGCTTCAAACCTGTAGC |
| | | TGTCTAGTGACAGAGTGGTTCAATTCCACCTTTGTA |
| 192 | Ser_AGA_chr6:27509554-27509635 (−) | GTAGTCGTGGCCGAGTGGTTAAGGCGATGGACTAGAAATCCATTGGGG |
| | | TTTCCCCGCGCAGGTTCGAATCCTGCCGACTACG |
| 193 | Ser_AGA_chr6:26327817-26327898 (+) | GTAGTCGTGGCCGAGTGGTTAAGGCGATGGACTAGAAATCCATTGGGG |
| | | TCTCCCCGCGCAGGTTCGAATCCTGCCGACTACG |
| 194 | Ser_AGA_chr6:27499987-27500068 (+) | GTAGTCGTGGCCGAGTGGTTAAGGCGATGGACTAGAAATCCATTGGGG |
| | | TTTCCCCACGCAGGTTCGAATCCTGCCGACTACG |
| 195 | Ser_AGA_chr6:27521192-27521273 (−) | GTAGTCGTGGCCGAGTGGTTAAGGTGATGGACTAGAAACCCATTGGGG |
| | | TCTCCCCGCGCAGGTTCGAATCCTGCCGACTACG |
| 196 | Ser_CGA_chr17:8042199-8042280 (−) | GCTGTGATGGCCGAGTGGTTAAGGCGTTGGACTCGAAATCCAATGGGG |
| | | TCTCCCCGCGCAGGTTCGAATCCTGCTCACAGCG |
| 197 | Ser_CGA_chr6:27177628-27177709 (+) | GCTGTGATGGCCGAGTGGTTAAGGCGTTGGACTCGAAATCCAATGGGG |
| | | TCTCCCCGCGCAGGTTCAAATCCTGCTCACAGCG |
| 198 | Ser_CGA_chr6:27640229-27640310 (−) | GCTGTGATGGCCGAGTGGTTAAGGTGTTGGACTCGAAATCCAATGGGG |
| | | GTTCCCCGCGCAGGTTCAAATCCTGCTCACAGCG |
| 199 | Ser_CGA_chr12:56584148-56584229 (+) | GTCACGGTGGCCGAGTGGTTAAGGCGTTGGACTCGAAATCCAATGGGG |
| | | TTTCCCCGCACAGGTTCGAATCCTGTTCGTGACG |
| 200 | Ser_GCT_chr6:27065085-27065166 (+) | GACGAGGTGGCCGAGTGGTTAAGGCGATGGACTGCTAATCCATTGTGC |
| | | TCTGCACGCGTGGGTTCGAATCCCACCCTCGTCG |
| 201 | Ser_GCT_chr6:27265775-27265856 (+) | GACGAGGTGGCCGAGTGGTTAAGGCGATGGACTGCTAATCCATTGTGC |
| | | TCTGCACGCGTGGGTTCGAATCCCACCTTCGTCG |
| 202 | Ser_GCT_chr11:66115591-66115672 (+) | GACGAGGTGGCCGAGTGGTTAAGGCGATGGACTGCTAATCCATTGTGC |
| | | TTTGCACGCGTGGGTTCGAATCCCATCCTCGTCG |
| 203 | Ser_GCT_chr6:28565117-28565198 (−) | GACGAGGTGGCCGAGTGGTTAAGGCGATGGACTGCTAATCCATTGTGC |
| | | TCTGCACGCGTGGGTTCGAATCCCATCCTCGTCG |
| 204 | Ser_GCT_chr6:28180815-28180896 (+) | GACGAGGTGGCCGAGTGGTTAAGGCGATGGACTGCTAATCCATTGTGC |
| | | TCTGCACACGTGGGTTCGAATCCCATCCTCGTCG |
| 205 | Ser_GCT_chr6:26305718-26305801 (−) | GGAGAGGCCTGGCCGAGTGGTTAAGGCGATGGACTGCTAATCCATTGT |
| | | GCTCTGCACGCGTGGGTTCGAATCCCATCCTCGTCG |
| 206 | Ser_TGA_chr10:69524261-69524342 (+) | GCAGCGATGGCCGAGTGGTTAAGGCGTTGGACTTGAAATCCAATGGGG |
| | | TCTCCCCGCGCAGGTTCGAACCCTGCTCGCTGCG |
| 207 | Ser_TGA_chr6:27513468-27513549 (+) | GTAGTCGTGGCCGAGTGGTTAAGGCGATGGACTTGAAATCCATTGGGG |
| | | TTTCCCCGCGCAGGTTCGAATCCTGCCGACTACG |
| 208 | Ser_TGA_chr6:26312824-26312905 (−) | GTAGTCGTGGCCGAGTGGTTAAGGCGATGGACTTGAAATCCATTGGGG |
| | | TCTCCCCGCGCAGGTTCGAATCCTGCCGACTACG |
| 209 | Ser_TGA_chr6:27473607-27473688 (−) | GTAGTCGTGGCCGAGTGGTTAAGGCGATGGACTTGAAATCCATTGGGG |
| | | TTTCCCCGCGCAGGTTCGAATCCTGTCGGCTACG |
| 210 | Thr_AGT_chr17:8090478-8090551 (+) | GGCGCCGTGGCTTAGTTGGTTAAAGCGCCTGTCTAGTAAACAGGAGAT |
| | | CCTGGGTTCGAATCCCAGCGGTGCCT |
| 211 | Thr_AGT_chr6:26533145-26533218 (−) | GGCTCCGTGGCTTAGCTGGTTAAAGCGCCTGTCTAGTAAACAGGAGAT |
| | | CCTGGGTTCGAATCCCAGCGGGGCCT |
| 212 | Thr_AGT_chr6:28693795-28693868 (+) | GGCTCCGTAGCTTAGTTGGTTAAAGCGCCTGTCTAGTAAACAGGAGAT |
| | | CCTGGGTTCGACTCCCAGCGGGGCCT |
| 213 | Thr_AGT_chr6:27694473-27694546 (+) | GGCTTCGTGGCTTAGCTGGTTAAAGCGCCTGTCTAGTAAACAGGAGAT |
| | | CCTGGGTTCGAATCCCAGCGAGGCCT |
| 214 | Thr_AGT_chr17:8042770-8042843 (−) | GGCGCCGTGGCTTAGCTGGTTAAAGCGCCTGTCTAGTAAACAGGAGAT |
| | | CCTGGGTTCGAATCCCAGCGGTGCCT |
| 215 | Thr_AGT_chr6:27130050-27130123 (+) | GGCCCTGTGGCTTAGCTGGTCAAAGCGCCTGTCTAGTAAACAGGAGAT |
| | | CCTGGGTTCGAATCCCAGCGGGGCCT |
| 216 | Thr_CGT_chr6:28456770-28456843 (−) | GGCTCTATGGCTTAGTTGGTTAAAGCGCCTGTCTCGTAAACAGGAGAT |
| | | CCTGGGTTCGACTCCCAGTGGGGCCT |
| 217 | Thr_CGT_chr16:14379750-14379821 (+) | GGCGCGGTGGCCAAGTGGTAAGGCGTCGGTCTCGTAAACCGAAGATCA |
| | | CGGGTTCGAACCCCGTCCGTGCCT |
| 218 | Thr_CGT_chr6:28615984-28616057 (−) | GGCTCTGTGGCTTAGTTGGCTAAAGCGCCTGTCTCGTAAACAGGAGAT |
| | | CCTGGGTTCGAATCCCAGCGGGGCCT |
| 219 | Thr_CGT_chr17:29877093-29877164 (+) | GGCGCGGTGGCCAAGTGGTAAGGCGTCGGTCTCGTAAACCGAAGATCG |
| | | CGGGTTCGAACCCCGTCCGTGCCT |
| 220 | Thr_CGT_chr6:27586135-27586208 (+) | GGCCCTGTAGCTCAGCGGTTGGAGCGCTGGTCTCGTAAACCTAGGGGT |
| | | CGTGAGTTCAAATCTCACCAGGGCCT |
| 221 | Thr_TGT_chr6:28442329-28442402 (−) | GGCTCTATGGCTTAGTTGGTTAAAGCGCCTGTCTTGTAAACAGGAGAT |
| | | CCTGGGTTCGAATCCCAGTAGAGCCT |
| 222 | Thr_TGT_chr1:222638347-222638419 (+) | GGCTCCATAGCTCAGTGGTTAGAGCACTGGTCTTGTAAACCAGGGGTC |
| | | GCGAGTTCGATCCTCGCTGGGGCCT |
| 223 | Thr_TGT_chr14:21081949-21082021 (−) | GGCTCCATAGCTCAGGGGTTAGAGCGCTGGTCTTGTAAACCAGGGGTC |
| | | GCGAGTTCAATTCTCGCTGGGGCCT |
| 224 | Thr_TGT_chr14:21099319-21099391 (−) | GGCTCCATAGCTCAGGGGTTAGAGCACTGGTCTTGTAAACCAGGGGTC |
| | | GCGAGTTCAAATCTCGCTGGGGCCT |
| 225 | Thr_TGT_chr14:21149849-21149921 (+) | GGCCCTATAGCTCAGGGGTTAGAGCACTGGTCTTGTAAACCAGGGGTC |
| | | GCGAGTTCAAATCTCGCTGGGGCCT |
| 226 | Thr_TGT_chr5:180618687-180618758 (−) | GGCTCCATAGCTCAGGGGTTAGAGCACTGGTCTTGTAAACCAGGGTCG |
| | | CGAGTTCAAATCTCGCTGGGGCCT |
| 227 | Trp_CCA_chr17:8124187-8124258 (−) | GGCCTCGTGGCGCAACGGTAGCGCGTCTGACTCCAGATCAGAAGGTTG |
| | | CGTGTTCAAATCACGTCGGGGTCA |
| 228 | Trp_CCA_chr17:19411494-19411565 (+) | GACCTCGTGGCGCAATGGTAGCGCGTCTGACTCCAGATCAGAAGGTTG |
| | | CGTGTTCAAGTCACGTCGGGGTCA |
| 229 | Trp_CCA_chr6:26319330-26319401 (−) | GACCTCGTGGCGCAACGGTAGCGCGTCTGACTCCAGATCAGAAGGTTG |
| | | CGTGTTCAAATCACGTCGGGGTCA |
| 230 | Trp_CCA_chr12:98898030-98898101 (+) | GACCTCGTGGCGCAACGGTAGCGCGTCTGACTCCAGATCAGAAGGCTG |
| | | CGTGTTCGAATCACGTCGGGGTCA |
| 231 | Trp_CCA_chr7:99067307-99067378 (+) | GACCTCGTGGCGCAACGGCAGCGCGTCTGACTCCAGATCAGAAGGTTG |
| | | CGTGTTCAAATCACGTCGGGGTCA |
| 232 | Tyr_ATA_chr2:219110549-219110641 (+) | CCTTCAATAGTTCAGCTGGTAGAGCAGAGGACTATAGCTACTTCCTCA |
| | | GTAGGAGACGTCCTTAGGTTGCTGGTTCGATTCCAGCTTGAAGGA |
| 233 | Tyr_GTA_chr6:26569086-26569176 (+) | CCTTCGATAGCTCAGTTGGTAGAGCGGAGGACTGTAGTTGGCTGTGTC |
| | | CTTAGACATCCTTAGGTCGCTGGTTCGAATCCGGCTCGAAGGA |
| 234 | Tyr_GTA_chr2:27273650-27273738 (+) | CCTTCGATAGCTCAGTTGGTAGAGCGGAGGACTGTAGTGGATAGGGCG |
| | | TGGCAATCCTTAGGTCGCTGGTTCGATTCCGGCTCGAAGGA |
| 235 | Tyr_GTA_chr6:26577332-26577420 (+) | CCTTCGATAGCTCAGTTGGTAGAGCGGAGGACTGTAGGCTCATTAAGC |
| | | AAGGTATCCTTAGGTCGCTGGTTCGAATCCGGCTCGGAGGA |
| 236 | Tyr_GTA_chr14:21125623-21125716 (−) | CCTTCGATAGCTCAGCTGGTAGAGCGGAGGACTGTAGATTGTATAGAC |
| | | ATTTGCGGACATCCTTAGGTCGCTGGTTCGATTCCAGCTCGAAGGA |
| 237 | Tyr_GTA_chr8:67025602-67025694 (+) | CCTTCGATAGCTCAGCTGGTAGAGCGGAGGACTGTAGCTACTTCCTCA |
| | | GCAGGAGACATCCTTAGGTCGCTGGTTCGATTCCGGCTCGAAGGA |
| 238 | Tyr_GTA_chr8:67026223-67026311 (+) | CCTTCGATAGCTCAGCTGGTAGAGCGGAGGACTGTAGGCGCGCGCCCG |
| | | TGGCCATCCTTAGGTCGCTGGTTCGATTCCGGCTCGAAGGA |
| 239 | Tyr_GTA_chr14:21121258-21121351 (−) | CCTTCGATAGCTCAGCTGGTAGAGCGGAGGACTGTAGCCTGTAGAAAC |
| | | ATTTGTGGACATCCTTAGGTCGCTGGTTCGATTCCGGCTCGAAGGA |
| 240 | Tyr_GTA_chr14:21131351-21131444 (−) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGA |
| 241 | Tyr_GTA_chr14:21151432-21151520 (+) | CCTTCGATAGCTCAGCTGGTAGAGCGGAGGACTGTAGTACTTAATGTG |
| | | TGGTCATCCTTAGGTCGCTGGTTCGATTCCGGCTCGAAGGA |
| 242 | Tyr_GTA_chr6:26595102-26595190 (+) | CCTTCGATAGCTCAGCTGGTAGAGCGGAGGACTGTAGGGGTTTGAATG |
| | | TGGTCATCCTTAGGTCGCTGGTTCGAATCCGGCTCGGAGGA |
| 243 | Tyr_GTA_chr14:21128117-21128210 (−) | CCTTCGATAGCTCAGCTGGTAGAGCGGAGGACTGTAGACTGCGGAAAC |
| | | GTTTGTGGACATCCTTAGGTCGCTGGTTCAATTCCGGCTCGAAGGA |
| 244 | Tyr_GTA_chr6:26575798-26575887 (+) | CTTTCGATAGCTCAGTTGGTAGAGCGGAGGACTGTAGGTTCATTAAAC |
| | | TAAGGCATCCTTAGGTCGCTGGTTCGAATCCGGCTCGAAGGA |
| 245 | Tyr_GTA_chr8:66609532-66609619 (−) | TCTTCAATAGCTCAGCTGGTAGAGCGGAGGACTGTAGGTGCACGCCCG |
| | | TGGCCATTCTTAGGTGCTGGTTTGATTCCGACTTGGAGAG |
| 246 | Val_AAC_chr3:169490018-169490090 (+) | GTTTCCGTAGTGTAGTGGTTATCACGTTCGCCTAACACGCGAAAGGTC |
| | | CCCGGTTCGAAACCGGGCGGAAACA |
| 247 | Val_AAC_chr5:180615416-180615488 (−) | GTTTCCGTAGTGTAGTGGTCATCACGTTCGCCTAACACGCGAAAGGTC |
| | | CCCGGTTCGAAACCGGGCGGAAACA |
| 248 | Val_AAC_chr6:27618707-27618779 (−) | GTTTCCGTAGTGTAGTGGTTATCACGTTCGCCTAACACGCGAAAGGTC |
| | | CCTGGATCAAAACCAGGCGGAAACA |
| 249 | Val_AAC_chr6:27648885-27648957 (−) | GTTTCCGTAGTGTAGTGGTTATCACGTTCGCCTAACACGCGAAAGGTC |
| | | CGCGGTTCGAAACCGGGCGGAAACA |
| 250 | Val_AAC_chr6:27203288-27203360 (+) | GTTTCCGTAGTGTAGTGGTTATCACGTTTGCCTAACACGCGAAAGGTC |
| | | CCCGGTTCGAAACCGGGCAGAAACA |
| 251 | Val_AAC_chr6:28703206-28703277 (−) | GGGGGTGTAGCTCAGTGGTAGAGCGTATGCTTAACATTCATGAGGCTC |
| | | TGGGTTCGATCCCCAGCACTTCCA |
| 252 | Val_CAC_chr1:161369490-161369562 (−) | GTTTCCGTAGTGTAGTGGTTATCACGTTCGCCTCACACGCGAAAGGTC |
| | | CCCGGTTCGAAACCGGGCGGAAACA |
| 253 | Val_CAC_chr6:27248049-27248121 (−) | GCTTCTGTAGTGTAGTGGTTATCACGTTCGCCTCACACGCGAAAGGTC |
| | | CCCGGTTCGAAACCGGGCAGAAGCA |
| 254 | Val_CAC_chr19:4724647-4724719 (−) | GTTTCCGTAGTGTAGCGGTTATCACATTCGCCTCACACGCGAAAGGTC |
| | | CCCGGTTCGATCCCGGGCGGAAACA |
| 255 | Val_CAC_chr1: 149298555-149298627 (−) | GTTTCCGTAGTGTAGTGGTTATCACGTTCGCCTCACACGCGAAAGGTC |
| | | CCCGGTTCGAAACTGGGCGGAAACA |
| 256 | Val_CAC_chr1:149684088-149684161 (−) | GTTTCCGTAGTGTAGTGGTTATCACGTTCGCCTCACACGCGTAAAGGT |
| | | CCCCGGTTCGAAACCGGGCGGAAACA |
| 257 | Val_CAC_chr6:27173867-27173939 (−) | GTTTCCGTAGTGGAGTGGTTATCACGTTCGCCTCACACGCGAAAGGTC |
| | | CCCGGTTTGAAACCAGGCGGAAACA |
| 258 | Val_TAC_chr11:59318102-59318174 (−) | GGTTCCATAGTGTAGTGGTTATCACGTCTGCTTTACACGCAGAAGGTC |
| | | CTGGGTTCGAGCCCCAGTGGAACCA |
| 259 | Val_TAC_chr11:59318460-59318532 (−) | GGTTCCATAGTGTAGCGGTTATCACGTCTGCTTTACACGCAGAAGGTC |
| | | CTGGGTTCGAGCCCCAGTGGAACCA |
| 260 | Val_TAC_chr10:5895674-5895746 (−) | GGTTCCATAGTGTAGTGGTTATCACATCTGCTTTACACGCAGAAGGTC |
| | | CTGGGTTCAAGCCCCAGTGGAACCA |
| 261 | Val_TAC_chr6:27258405-27258477 (+) | GTTTCCGTGGTGTAGTGGTTATCACATTCGCCTTACACGCGAAAGGTC |
| | | CTCGGGTCGAAACCGAGCGGAAACA |
| 262 | iMet_CAT_chr1:153643726-153643797 (+) | AGCAGAGTGGCGCAGCGGAAGCGTGCTGGGCCCATAACCCAGAGGTCG |
| | | ATGGATCGAAACCATCCTCTGCTA |
| 263 | iMet_CAT_chr6:27745664-27745735 (+) | AGCAGAGTGGCGCAGCGGAAGCGTGCTGGGCCCATAACCCAGAGGTCG |
| | | ATGGATCTAAACCATCCTCTGCTA |
| 264 | Glu_TTC_chr1:16861773-16861845 (−) | TCCCTGGTGGTCTAGTGGCTAGGATTCGGCGCTTTCACCGCCGCGGCC |
| | | CGGGTTCGATTCCCGGTCAGGGAAT |
| 265 | Gly_CCC_chr1:17004765-17004836 (−) | GCGTTGGTGGTTTAGTGGTAGAATTCTCGCCTCCCATGCGGGAGACCC |
| | | GGGTTCAATTCCCGGCCACTGCAC |
| 266 | Gly_CCC_chr1:17053779-17053850 (+) | GGCCTTGGTGGTGCAGTGGTAGAATTCTCGCCTCCCACGTGGGAGACC |
| | | CGGGTTCAATTCCCGGCCAATGCA |
| 267 | Glu_TTC_chr1:17199077-17199149 (+) | GTCCCTGGTGGTCTAGTGGCTAGGATTCGGCGCTTTCACCGCCGCGGC |
| | | CCGGGTTCGATTCCCGGCCAGGGAA |
| 268 | Asn_GTT_chr1:17216171-17216245 (+) | TGTCTCTGTGGCGCAATCGGTTAGCGCGTTCGGCTGTTAACCGAAAGA |
| | | TTGGTGGTTCGAGCCCACCCAGGGACG |
| 269 | Arg_TCT_chr1:94313128-94313213 (+) | TGGCTCCGTGGCGCAATGGATAGCGCATTGGACTTCTAGAGGCTGAAG |
| | | GCATTCAAAGGTTCCGGGTTCGAGTCCCGGCGGAGTCG |
| 270 | Lys_CTT_chr1:145395521-145395594 (−) | GCCCGGCTAGCTCAGTCGGTAGAGCATGAGACTCTTAATCTCAGGGTC |
| | | GTGGGTTCGAGCCCCACGTTGGGCGC |
| 271 | His_GTG_chr1:145396880-145396952 (−) | GCCGTGATCGTATAGTGGTTAGTACTCTGCGTTGTGGCCGCAGCAACC |
| | | TCGGTTCGAATCCGAGTCACGGCAG |
| 272 | Gly_TCC_chr1:145397863-145397935 (−) | GCGTTGGTGGTATAGTGGTGAGCATAGCTGCCTTCCAAGCAGTTGACC |
| | | CGGGTTCGATTCCCGGCCAACGCAG |
| 273 | Glu_CTC_chr1:145399232-145399304 (−) | TCCCTGGTGGTCTAGTGGTTAGGATTCGGCGCTCTCACCGCCGCGGCC |
| | | CGGGTTCGATTCCCGGTCAGGGAAA |
| 274 | Gln_CTG_chr1:145963303-145963375 (+) | AGGTTCCATGGTGTAATGGTGAGCACTCTGGACTCTGAATCCAGCGAT |
| | | CCGAGTTCGAGTCTCGGTGGAACCT |
| 275 | Asn_GTT_chr1:148000804-148000878 (+) | TGTCTCTGTGGCGTAGTCGGTTAGCGCGTTCGGCTGTTAACCGAAAAG |
| | | TTGGTGGTTCGAGCCCACCCAGGAACG |
| 276 | Asn_GTT_chr1:148248114-148248188 (+) | TGTCTCTGTGGCGCAATCGGTTAGCGCGTTCGGCTGTTAACCGAAAGG |
| | | TTGGTGGTTCGAGCCCACCCAGGGACG |
| 277 | Asn_GTT_chr1:148598313-148598387 (−) | GTCTCTGTGGCGCAATCGGTTAGCGCATTCGGCTGTTAACCGAAAGGT |
| | | TGGTGGTTCGAGCCCACCCAGGGACGC |
| 278 | Asn_GTT_chr1:149230569-149230643 (−) | GTCTCTGTGGCGCAATGGGTTAGCGCGTTCGGCTGTTAACCGAAAGGT |
| | | TGGTGGTTCGAGCCCATCCAGGGACGC |
| 279 | Val_CAC_chr1:149294665-149294736 (−) | GCACTGGTGGTTCAGTGGTAGAATTCTCGCCTCACACGCGGGACACCC |
| | | GGGTTCAATTCCCGGTCAAGGCAA |
| 280 | Val_CAC_chr1:149298554-149298627 (−) | GTTTCCGTAGTGTAGTGGTTATCACGTTCGCCTCACACGCGAAAGGTC |
| | | CCCGGTTCGAAACTGGGCGGAAACAG |
| 281 | Gly_CCC_chr1: 149680209-149680280 (−) | GCACTGGTGGTTCAGTGGTAGAATTCTCGCCTCCCACGCGGGAGACCC |
| | | GGGTTTAATTCCCGGTCAAGATAA |
| 282 | Val_CAC_chr1:149684087-149684161 (−) | GTTTCCGTAGTGTAGTGGTTATCACGTTCGCCTCACACGCGTAAAGGT |
| | | CCCCGGTTCGAAACCGGGCGGAAACAT |
| 283 | Met_CAT_chr1:153643725-153643797 (+) | TAGCAGAGTGGCGCAGCGGAAGCGTGCTGGGCCCATAACCCAGAGGTC |
| | | GATGGATCGAAACCATCCTCTGCTA |
| 284 | Val_CAC_chr1:161369489-161369562 (−) | GTTTCCGTAGTGTAGTGGTTATCACGTTCGCCTCACACGCGAAAGGTC |
| | | CCCGGTTCGAAACCGGGCGGAAACAA |
| 285 | Asp_GTC_chr1:161410614-161410686 (−) | TCCTCGTTAGTATAGTGGTGAGTATCCCCGCCTGTCACGCGGGAGACC |
| | | GGGGTTCGATTCCCCGACGGGGAGG |
| 286 | Gly_GCC_chr1:161413093-161413164 (+) | TGCATGGGTGGTTCAGTGGTAGAATTCTCGCCTGCCACGCGGGAGGCC |
| | | CGGGTTCGATTCCCGGCCCATGCA |
| 287 | Glu_CTC_chr1:161417017-161417089 (−) | TCCCTGGTGGTCTAGTGGTTAGGATTCGGCGCTCTCACCGCCGCGGCC |
| | | CGGGTTCGATTCCCGGTCAGGGAAG |
| 288 | Asp_GTC_chr1:161492934-161493006 (+) | ATCCTTGTTACTATAGTGGTGAGTATCTCTGCCTGTCATGCGTGAGAG |
| | | AGGGGGTCGATTCCCCGACGGGGAG |
| 289 | Gly_GCC_chr1:161493636-161493707 (−) | GCATTGGTGGTTCAGTGGTAGAATTCTCGCCTGCCACGCGGGAGGCCC |
| | | GGGTTCGATTCCCGGCCAATGCAC |
| 290 | Leu_CAG_chr1:161500131-161500214 (−) | GTCAGGATGGCCGAGCGGTCTAAGGCGCTGCGTTCAGGTCGCAGTCTC |
| | | CCCTGGAGGCGTGGGTTCGAATCCCACTCCTGACAA |
| 291 | Gly_TCC_chr1:161500902-161500974 (+) | CGCGTTGGTGGTATAGTGGTGAGCATAGCTGCCTTCCAAGCAGTTGAC |
| | | CCGGGTTCGATTCCCGGCCAACGCA |
| 292 | Asn_GTT_chr1:161510030-161510104 (+) | CGTCTCTGTGGCGCAATCGGTTAGCGCGTTCGGCTGTTAACCGAAAGG |
| | | TTGGTGGTTCGATCCCACCCAGGGACG |
| 293 | Glu_TTC_chr1:161582507-161582579 (+) | CGCGTTGGTGGTGTAGTGGTGAGCACAGCTGCCTTTCAAGCAGTTAAC |
| | | GCGGGTTCGATTCCCGGGTAACGAA |
| 294 | Pro_CGG_chr1:167683961-167684033 (+) | CGGCTCGTTGGTCTAGGGGTATGATTCTCGCTTCGGGTGCGAGAGGTC |
| | | CCGGGTTCAAATCCCGGACGAGCCC |
| 295 | Pro_AGG_chr1:167684724-167684796 (−) | GGCTCGTTGGTCTAGGGGTATGATTCTCGCTTAGGGTGCGAGAGGTCC |
| | | CGGGTTCAAATCCCGGACGAGCCCT |
| 296 | Lys_TTT_chr1:204475654-204475727 (+) | CGCCCGGATAGCTCAGTCGGTAGAGCATCAGACTTTTAATCTGAGGGT |
| | | CCAGGGTTCAAGTCCCTGTTCGGGCG |
| 297 | Lys_TTT_chr1:204476157-204476230 (−) | GCCCGGATAGCTCAGTCGGTAGAGCATCAGACTTTTAATCTGAGGGTC |
| | | CAGGGTTCAAGTCCCTGTTCGGGCGT |
| 298 | Leu_CAA_chr1:249168053-249168159 (+) | TGTCAGGATGGCCGAGTGGTCTAAGGCGCCAGACTCAAGGTAAGCACC |
| | | TTGCCTGCGGGCTTTCTGGTCTCCGGATGGAGGCGTGGGTTCGAATCC |
| | | CACTTCTGACA |
| 299 | Glu_CTC_chr1:249168446-249168518 (+) | TTCCCTGGTGGTCTAGTGGTTAGGATTCGGCGCTCTCACCGCCGCGGC |
| | | CCGGGTTCGATTCCCGGTCAGGAAA |
| 300 | Tyr_GTA_chr2:27273649-27273738 (+) | GCCTTCGATAGCTCAGTTGGTAGAGCGGAGGACTGTAGTGGATAGGGC |
| | | GTGGCAATCCTTAGGTCGCTGGTTCGATTCCGGCTCGAAGGA |
| 301 | Ala_AGC_chr2:27274081-27274154 (+) | CGGGGGATTAGCTCAAATGGTAGAGCGCTCGCTTAGCATGCGAGAGGT |
| | | AGCGGGATCGATGCCCGCATCCTCCA |
| 302 | Ile_TAT_chr2:43037675-43037768 (+) | AGCTCCAGTGGCGCAATCGGTTAGCGCGCGGTACTTATACAGCAGTAC |
| | | ATGCAGAGCAATGCCGAGGTTGTGAGTTCGAGCCTCACCTGGAGCA |
| 303 | Gly_CCC_chr2:70476122-70476193 (−) | GCGCCGCTGGTGTAGTGGTATCATGCAAGATTCCCATTCTTGCGACCC |
| | | GGGTTCGATTCCCGGGCGGCGCAT |
| 304 | Glu_TTC_chr2:131094700-131094772 (−) | TCCCATATGGTCTAGCGGTTAGGATTCCTGGTTTTCACCCAGGTGGCC |
| | | CGGGTTCGACTCCCGGTATGGGAAC |
| 305 | Ala_CGC_chr2:157257280-157257352 (+) | GGGGGATGTAGCTCAGTGGTAGAGCGCGCGCTTCGCATGTGTGAGGTC |
| | | CCGGGTTCAATCCCCGGCATCTCCA |
| 306 | Gly_GCC_chr2:157257658-157257729 (−) | GCATTGGTGGTTCAGTGGTAGAATTCTCGCCTGCCACGCGGGAGGCCC |
| | | GGGTTCGATTCCCGGCCAATGCAA |
| 307 | Arg_ACG_chr3:45730490-45730563 (−) | GGGCCAGTGGCGCAATGGATAACGCGTCTGACTACGGATCAGAAGATT |
| | | CTAGGTTCGACTCCTGGCTGGCTCGC |
| 308 | Val_AAC_chr3:169490017-169490090 (+) | GGTTTCCGTAGTGTAGTGGTTATCACGTTCGCCTAACACGCGAAAGGT |
| | | CCCCGGTTCGAAACCGGGCGGAAACA |
| 309 | Val_AAC_chr5:180596609-180596682 (+) | AGTTTCCGTAGTGTAGTGGTTATCACGTTCGCCTAACACGCGAAAGGT |
| | | CCCCGGTTCGAAACCGGGCGGAAACA |
| 310 | Leu_AAG_chr5:180614700-180614782 (+) | AGGTAGCGTGGCCGAGCGGTCTAAGGCGCTGGATTAAGGCTCCAGTCT |
| | | CTTCGGGGGCGTGGGTTCGAATCCCACCGCTGCCA |
| 311 | Val_AAC_chr5:180615415-180615488 (−) | GTTTCCGTAGTGTAGTGGTCATCACGTTCGCCTAACACGCGAAAGGTC |
| | | CCCGGTTCGAAACCGGGCGGAAACAT |
| 312 | Pro_TGG_chr5:180615853-180615925 (−) | GGCTCGTTGGTCTAGGGGTATGATTCTCGCTTTGGGTGCGAGAGGTCC |
| | | CGGGTTCAAATCCCGGACGAGCCCA |
| 313 | Thr_TGT_chr5:180618686-180618758 (−) | GGCTCCATAGCTCAGGGGTTAGAGCACTGGTCTTGTAAACCAGGGTCG |
| | | CGAGTTCAAATCTCGCTGGGGCCTG |
| 314 | Ala_TGC_chr5:180633867-180633939 (+) | TGGGGATGTAGCTCAGTGGTAGAGCGCATGCTTTGCATGTATGAGGCC |
| | | CCGGGTTCGATCCCCGGCATCTCCA |
| 315 | Lys_CTT_chr5:180634754-180634827 (+) | CGCCCGGCTAGCTCAGTCGGTAGAGCATGAGACTCTTAATCTCAGGGT |
| | | CGTGGGTTCGAGCCCCACGTTGGGCG |
| 316 | Val_AAC_chr5:180645269-180645342 (−) | GTTTCCGTAGTGTAGTGGTTATCACGTTCGCCTAACACGCGAAAGGTC |
| | | CCCGGTTCGAAACCGGGCGGAAACAA |
| 317 | Lys_CTT_chr5:180648978-180649051 (−) | GCCCGGCTAGCTCAGTCGGTAGAGCATGAGACTCTTAATCTCAGGGTC |
| | | GTGGGTTCGAGCCCCACGTTGGGCGT |
| 318 | Val_CAC_chr5:180649394-180649467 (−) | GTTTCCGTAGTGTAGTGGTTATCACGTTCGCCTCACACGCGAAAGGTC |
| | | CCCGGTTCGAAACCGGGCGGAAACAC |
| 319 | Met_CAT_chr6:26286753-26286825 (+) | CAGCAGAGTGGCGCAGCGGAAGCGTGCTGGGCCCATAACCCAGAGGTC |
| | | GATGGATCGAAACCATCCTCTGCTA |
| 320 | Ser_GCT_chr6:26305717-26305801 (−) | GGAGAGGCCTGGCCGAGTGGTTAAGGCGATGGACTGCTAATCCATTGT |
| | | GCTCTGCACGCGTGGGTTCGAATCCCATCCTCGTCGC |
| 321 | Gln_TTG_chr6:26311423-26311495 (−) | GGCCCCATGGTGTAATGGTTAGCACTCTGGACTTTGAATCCAGCGATC |
| | | CGAGTTCAAATCTCGGTGGGACCTG |
| 322 | Gln_TTG_chr6:26311974-26312046 (−) | GGCCCCATGGTGTAATGGTTAGCACTCTGGACTTTGAATCCAGCGATC |
| | | CGAGTTCAAATCTCGGTGGGACCTA |
| 323 | Ser_TGA_chr6:26312823-26312905 (−) | GTAGTCGTGGCCGAGTGGTTAAGGCGATGGACTTGAAATCCATTGGGG |
| | | TCTCCCCGCGCAGGTTCGAATCCTGCCGACTACGG |
| 324 | Met_CAT_chr6:26313351-26313423 (−) | AGCAGAGTGGCGCAGCGGAAGCGTGCTGGGCCCATAACCCAGAGGTCG |
| | | ATGGATCGAAACCATCCTCTGCTAT |
| 325 | Arg_TCG_chr6:26323045-26323118 (+) | GGACCACGTGGCCTAATGGATAAGGCGTCTGACTTCGGATCAGAAGAT |
| | | TGAGGGTTCGAATCCCTCCGTGGTTA |
| 326 | Ser_AGA_chr6:26327816-26327898 (+) | TGTAGTCGTGGCCGAGTGGTTAAGGCGATGGACTAGAAATCCATTGGG |
| | | GTCTCCCCGCGCAGGTTCGAATCCTGCCGACTACG |
| 327 | Met_CAT_chr6:26330528-26330600 (−) | AGCAGAGTGGCGCAGCGGAAGCGTGCTGGGCCCATAACCCAGAGGTCG |
| | | ATGGATCGAAACCATCCTCTGCTAG |
| 328 | Leu_CAG_chr6:26521435-26521518 (+) | CGTCAGGATGGCCGAGCGGTCTAAGGCGCTGCGTTCAGGTCGCAGTCT |
| | | CCCCTGGAGGCGTGGGTTCGAATCCCACTCCTGACA |
| 329 | Thr_AGT_chr6:26533144-26533218 (−) | GGCTCCGTGGCTTAGCTGGTTAAAGCGCCTGTCTAGTAAACAGGAGAT |
| | | CCTGGGTTCGAATCCCAGCGGGGCCTG |
| 330 | Arg_ACG_chr6:26537725-26537798 (+) | AGGGCCAGTGGCGCAATGGATAACGCGTCTGACTACGGATCAGAAGAT |
| | | TCCAGGTTCGACTCCTGGCTGGCTCG |
| 331 | Val_CAC_chr6:26538281-26538354 (+) | GGTTTCCGTAGTGTAGTGGTTATCACGTTCGCCTCACACGCGAAAGGT |
| | | CCCCGGTTCGAAACCGGGCGGAAACA |
| 332 | Ala_CGC_chr6:26553730-26553802 (+) | AGGGGATGTAGCTCAGTGGTAGAGCGCATGCTTCGCATGTATGAGGTC |
| | | CCGGGTTCGATCCCCGGCATCTCCA |
| 333 | Ile_AAT_chr6:26554349-26554423 (+) | TGGCCGGTTAGCTCAGTTGGTTAGAGCGTGGTGCTAATAACGCCAAGG |
| | | TCGCGGGTTCGATCCCCGTACGGGCCA |
| 334 | Pro_AGG_chr6:26555497-26555569 (+) | CGGCTCGTTGGTCTAGGGGTATGATTCTCGCTTAGGGTGCGAGAGGTC |
| | | CCGGGTTCAAATCCCGGACGAGCCC |
| 335 | Lys_CTT_chr6:26556773-26556846 (+) | AGCCCGGCTAGCTCAGTCGGTAGAGCATGAGACTCTTAATCTCAGGGT |
| | | CGTGGGTTCGAGCCCCACGTTGGGCG |
| 336 | Tyr_GTA_chr6:26569085-26569176 (+) | TCCTTCGATAGCTCAGTTGGTAGAGCGGAGGACTGTAGTTGGCTGTGT |
| | | CCTTAGACATCCTTAGGTCGCTGGTTCGAATCCGGCTCGAAGGA |
| 337 | Ala_AGC_chr6:26572091-26572164 (−) | GGGGAATTAGCTCAAATGGTAGAGCGCTCGCTTAGCATGCGAGAGGTA |
| | | GCGGGATCGATGCCCGCATTCTCCAG |
| 338 | Met_CAT_chr6:26766443-26766516 (+) | CGCCCTCTTAGCGCAGCGGGCAGCGCGTCAGTCTCATAATCTGAAGGT |
| | | CCTGAGTTCGAGCCTCAGAGAGGGCA |
| 339 | Ile_TAT_chr6:26988124-26988218 (+) | TGCTCCAGTGGCGCAATCGGTTAGCGCGCGGTACTTATATGGCAGTAT |
| | | GTGTGCGAGTGATGCCGAGGTTGTGAGTTCGAGCCTCACCTGGAGCA |
| 340 | His_GTG_chr6:27125905-27125977 (+) | TGCCGTGATCGTATAGTGGTTAGTACTCTGCGTTGTGGCCGCAGCAAC |
| | | CTCGGTTCGAATCCGAGTCACGGCA |
| 341 | Ile_AAT_chr6:27144993-27145067 (−) | GGCCGGTTAGCTCAGTTGGTTAGAGCGTGGTGCTAATAACGCCAAGGT |
| | | CGCGGGTTCGATCCCCGTACGGGCCAC |
| 342 | Val_AAC_chr6:27203287-27203360 (+) | AGTTTCCGTAGTGTAGTGGTTATCACGTTTGCCTAACACGCGAAAGGT |
| | | CCCCGGTTCGAAACCGGGCAGAAACA |
| 343 | Val_CAC_chr6:27248048-27248121 (−) | GCTTCTGTAGTGTAGTGGTTATCACGTTCGCCTCACACGCGAAAGGTC |
| | | CCCGGTTCGAAACCGGGCAGAAGCAA |
| 344 | Asp_GTC_chr6:27447452-27447524 (+) | TTCCTCGTTAGTATAGTGGTGAGTATCCCCGCCTGTCACGCGGGAGAC |
| | | CGGGGTTCGATTCCCCGACGGGGAG |
| 345 | Ser_TGA_chr6:27473606-27473688 (−) | GTAGTCGTGGCCGAGTGGTTAAGGCGATGGACTTGAAATCCATTGGGG |
| | | TTTCCCCGCGCAGGTTCGAATCCTGTCGGCTACGG |
| 346 | Gln_CTG_chr6:27487307-27487379 (+) | AGGTTCCATGGTGTAATGGTTAGCACTCTGGACTCTGAATCCAGCGAT |
| | | CCGAGTTCAAATCTCGGTGGAACCT |
| 347 | Asp_GTC_chr6:27551235-27551307 (−) | TCCTCGTTAGTATAGTGGTGAGTGTCCCCGTCTGTCACGCGGGAGACC |
| | | GGGGTTCGATTCCCCGACGGGGAGA |
| 348 | Val_AAC_chr6:27618706-27618779 (−) | GTTTCCGTAGTGTAGTGGTTATCACGTTCGCCTAACACGCGAAAGGTC |
| | | CCTGGATCAAAACCAGGCGGAAACAA |
| 349 | Ile_AAT_chr6:27655966-27656040 (+) | CGGCCGGTTAGCTCAGTTGGTTAGAGCGTGGTGCTAATAACGCCAAGG |
| | | TCGCGGGTTCGATCCCCGTACTGGCCA |
| 350 | Gln_CTG_chr6:27759134-27759206 (−) | GGCCCCATGGTGTAATGGTCAGCACTCTGGACTCTGAATCCAGCGATC |
| | | CGAGTTCAAATCTCGGTGGGACCCA |
| 351 | Gln_TTG_chr6:27763639-27763711 (−) | GGCCCCATGGTGTAATGGTTAGCACTCTGGACTTTGAATCCAGCGATC |
| | | CGAGTTCAAATCTCGGTGGGACCTT |
| 352 | Ala_AGC_chr6:28574932-28575004 (+) | TGGGGGTGTAGCTCAGTGGTAGAGCGCGTGCTTAGCATGTACGAGGTC |
| | | CCGGGTTCAATCCCCGGCACCTCCA |
| 353 | Ala_AGC_chr6:28626013-28626085 (−) | GGGGATGTAGCTCAGTGGTAGAGCGCATGCTTAGCATGCATGAGGTCC |
| | | CGGGTTCGATCCCCAGCATCTCCAG |
| 354 | Ala_CGC_chr6:28697091-28697163 (+) | AGGGGGTGTAGCTCAGTGGTAGAGCGCGTGCTTCGCATGTACGAGGCC |
| | | CCGGGTTCGACCCCCGGCTCCTCCA |
| 355 | Ala_AGC_chr6:28806220-28806292 (−) | GGGGGTGTAGCTCAGTGGTAGAGCGCGTGCTTAGCATGCACGAGGCCC |
| | | CGGGTTCAATCCCCGGCACCTCCAT |
| 356 | Ala_AGC_chr6:28831461-28831533 (−) | GGGGGTGTAGCTCAGTGGTAGAGCGCGTGCTTAGCATGCACGAGGCCC |
| | | CGGGTTCAATCCCCGGCACCTCCAG |
| 357 | Leu_CAA_chr6:28863999-28864105 (−) | GTCAGGATGGCCGAGTGGTCTAAGGCGCCAGACTCAAGCTAAGCTTCC |
| | | TCCGCGGTGGGGATTCTGGTCTCCAATGGAGGCGTGGGTTCGAATCCC |
| | | ACTTCTGACAC |
| 358 | Leu_CAA_chr6:28908829-28908934 (+) | TGTCAGGATGGCCGAGTGGTCTAAGGCGCCAGACTCAAGCTTGGCTTC |
| | | CTCGTGTTGAGGATTCTGGTCTCCAATGGAGGCGTGGGTTCGAATCCC |
| | | ACTTCTGACA |
| 359 | Gln_CTG_chr6:28909377-28909449 (−) | GGTTCCATGGTGTAATGGTTAGCACTCTGGACTCTGAATCCAGCGATC |
| | | CGAGTTCAAATCTCGGTGGAACCTT |
| 360 | Leu_AAG_chr6:28911398-28911480 (−) | GGTAGCGTGGCCGAGCGGTCTAAGGCGCTGGATTAAGGCTCCAGTCTC |
| | | TTCGGGGGCGTGGGTTCGAATCCCACCGCTGCCAG |
| 36 | Met_CAT_chr6:28912351-28912424 (+) | TGCCTCCTTAGCGCAGTAGGCAGCGCGTCAGTCTCATAATCTGAAGGT |
| | | CCTGAGTTCGAACCTCAGAGGGGGCA |
| 362 | Lys_TTT_chr6:28918805-28918878 (+) | AGCCCGGATAGCTCAGTCGGTAGAGCATCAGACTTTTAATCTGAGGGT |
| | | CCAGGGTTCAAGTCCCTGTTCGGGCG |
| 363 | Met_CAT_chr6:28921041-28921114 (−) | GCCTCCTTAGCGCAGTAGGCAGCGCGTCAGTCTCATAATCTGAAGGTC |
| | | CTGAGTTCGAACCTCAGAGGGGGCAG |
| 364 | Glu_CTC_chr6:28949975-28950047 (+) | TTCCCTGGTGGTCTAGTGGTTAGGATTCGGCGCTCTCACCGCCGCGGC |
| | | CCGGGTTCGATTCCCGGTCAGGGAA |
| 365 | Leu_TAA_chr6:144537683-144537766 (+) | CACCAGGATGGCCGAGTGGTTAAGGCGTTGGACTTAAGATCCAATGGA |
| | | CATATGTCCGCGTGGGTTCGAACCCCACTCCTGGTA |
| 366 | Pro_AGG_chr7:128423503-128423575 (+) | TGGCTCGTTGGTCTAGGGGTATGATTCTCGCTTAGGGTGCGAGAGGTC |
| | | CCGGGTTCAAATCCCGGACGAGCCC |
| 367 | Arg_CCT_chr7:139025445-139025518 (+) | AGCCCCAGTGGCCTAATGGATAAGGCATTGGCCTCCTAAGCCAGGGAT |
| | | TGTGGGTTCGAGTCCCATCTGGGGTG |
| 368 | Cys_GCA_chr7:149388271-149388343 (−) | GGGGATATAGCTCAGGGGTAGAGCATTTGACTGCAGATCAAGAGGTCC |
| | | CCGGTTCAAATCCGGGTGCCCCCCC |
| 369 | Tyr_GTA_chr8:67025601-67025694 (+) | CCCTTCGATAGCTCAGCTGGTAGAGCGGAGGACTGTAGCTACTTCCTC |
| | | AGCAGGAGACATCCTTAGGTCGCTGGTTCGATTCCGGCTCGAAGGA |
| 370 | Tyr_GTA_chr8:67026222-67026311 (+) | CCCTTCGATAGCTCAGCTGGTAGAGCGGAGGACTGTAGGCGCGCGCCC |
| | | GTGGCCATCCTTAGGTCGCTGGTTCGATTCCGGCTCGAAGGA |
| 371 | Ala_AGC_chr8:67026423-67026496 (+) | TGGGGGATTAGCTCAAATGGTAGAGCGCTCGCTTAGCATGCGAGAGGT |
| | | AGCGGGATCGATGCCCGCATCCTCCA |
| 372 | Ser_AGA_chr8:96281884-96281966 (−) | GTAGTCGTGGCCGAGTGGTTAAGGCGATGGACTAGAAATCCATTGGGG |
| | | TCTCCCCGCGCAGGTTCGAATCCTGCCGACTACGG |
| 373 | Met_CAT_chr8:124169469-124169542 (−) | GCCTCGTTAGCGCAGTAGGTAGCGCGTCAGTCTCATAATCTGAAGGTC |
| | | GTGAGTTCGATCCTCACACGGGGCAC |
| 374 | Arg_TCT_chr9:131102354-131102445 (−) | GGCTCTGTGGCGCAATGGATAGCGCATTGGACTTCTAGCTGAGCCTAG |
| | | TGTGGTCATTCAAAGGTTGTGGGTTCGAGTCCCACCAGAGTCGA |
| 375 | Asn_GTT_chr10:22518437-22518511 (−) | GTCTCTGTGGCGCAATCGGTTAGCGCGTTCGGCTGTTAACCGAAAGGT |
| | | TGGTGGTTCGAGCCCACCCAGGGACGC |
| 376 | Ser_TGA_chr10:69524260-69524342 (+) | GGCAGCGATGGCCGAGTGGTTAAGGCGTTGGACTTGAAATCCAATGGG |
| | | GTCTCCCCGCGCAGGTTCGAACCCTGCTCGCTGCG |
| 377 | Val_TAC_chr11:59318101-59318174 (−) | GGTTCCATAGTGTAGTGGTTATCACGTCTGCTTTACACGCAGAAGGTC |
| | | CTGGGTTCGAGCCCCAGTGGAACCAT |
| 378 | Val_TAC_chr11:59318459-59318532 (−) | GGTTCCATAGTGTAGCGGTTATCACGTCTGCTTTACACGCAGAAGGTC |
| | | CTGGGTTCGAGCCCCAGTGGAACCAC |
| 379 | Arg_TCT_chr11:59318766-59318852 (+) | TGGCTCTGTGGCGCAATGGATAGCGCATTGGACTTCTAGATAGTTAGA |
| | | GAAATTCAAAGGTTGTGGGTTCGAGTCCCACCAGAGTCG |
| 380 | Leu_TAA_chr11:59319227-59319310 (+) | TACCAGAATGGCCGAGTGGTTAAGGCGTTGGACTTAAGATCCAATGGA |
| | | TTCATATCCGCGTGGGTTCGAACCCCACTTCTGGTA |
| 381 | Lys_TTT_chr11:59323901-59323974 (+) | GGCCCGGATAGCTCAGTCGGTAGAGCATCAGACTTTTAATCTGAGGGT |
| | | CCGGGGTTCAAGTCCCTGTTCGGGCG |
| 382 | Phe_GAA_chr11:59324969-59325042 (−) | GCCGAAATAGCTCAGTTGGGAGAGCGTTAGACTGAAGATCTAAAGGTC |
| | | CCTGGTTCGATCCCGGGTTTCGGCAG |
| 383 | Lys_TTT_chr11:59327807-59327880 (−) | GCCCGGATAGCTCAGTCGGTAGAGCATCAGACTTTTAATCTGAGGGTC |
| | | CAGGGTTCAAGTCCCTGTTCGGGCGG |
| 384 | Phe_GAA_chr11:59333852-59333925 (−) | GCCGAAATAGCTCAGTTGGGAGAGCGTTAGACTGAAGATCTAAAGGTC |
| | | CCTGGTTCAATCCCGGGTTTCGGCAG |
| 385 | Ser_GCT_chr11:66115590-66115672 (+) | GGACGAGGTGGCCGAGTGGTTAAGGCGATGGACTGCTAATCCATTGTG |
| | | CTTTGCACGCGTGGGTTCGAATCCCATCCTCGTCG |
| 386 | Pro_TGG_chr11:75946868-75946940 (−) | GGCTCGTTGGTCTAGGGGTATGATTCTCGGTTTGGGTCCGAGAGGTCC |
| | | CGGGTTCAAATCCCGGACGAGCCCC |
| 387 | Ser_CGA_chr12:56584147-56584229 (+) | AGTCACGGTGGCCGAGTGGTTAAGGCGTTGGACTCGAAATCCAATGGG |
| | | GTTTCCCCGCACAGGTTCGAATCCTGTTCGTGACG |
| 388 | Asp_GTC_chr12:98897280-98897352 (+) | CTCCTCGTTAGTATAGTGGTTAGTATCCCCGCCTGTCACGCGGGAGAC |
| | | CGGGGTTCAATTCCCCGACGGGGAG |
| 389 | Trp_CCA_chr12:98898029-98898101 (+) | GGACCTCGTGGCGCAACGGTAGCGCGTCTGACTCCAGATCAGAAGGCT |
| | | GCGTGTTCGAATCACGTCGGGGTCA |
| 390 | Ala_TGC_chr12:125406300-125406372 (−) | GGGGATGTAGCTCAGTGGTAGAGCGCATGCTTTGCATGTATGAGGCCC |
| | | CGGGTTCGATCCCCGGCATCTCCAT |
| 391 | Phe_GAA_chr12:125412388-125412461 (−) | GCCGAAATAGCTCAGTTGGGAGAGCGTTAGACTGAAGATCTAAAGGTC |
| | | CCTGGTTCGATCCCGGGTTTCGGCAC |
| 392 | Ala_TGC_chr12:125424511-125424583 (+) | AGGGGATGTAGCTCAGTGGTAGAGCGCATGCTTTGCACGTATGAGGCC |
| | | CCGGGTTCAATCCCCGGCATCTCCA |
| 393 | Asn_GTT_chr13:31248100-31248174 (−) | GTCTCTGTGGCGCAATCGGTTAGCGCGTTCGGCTGTTAACCGAAAGGT |
| | | TGGTGGTTCGAGCCCACCCAGGGACGG |
| 394 | Glu_TTC_chr13:45492061-45492133 (−) | TCCCACATGGTCTAGCGGTTAGGATTCCTGGTTTTCACCCAGGCGGCC |
| | | CGGGTTCGACTCCCGGTGTGGGAAC |
| 395 | Thr_TGT_chr14:21081948-21082021 (−) | GGCTCCATAGCTCAGGGGTTAGAGCGCTGGTCTTGTAAACCAGGGGTC |
| | | GCGAGTTCAATTCTCGCTGGGGCCTG |
| 396 | Leu_TAG_chr14:21093528-21093610 (+) | TGGTAGTGTGGCCGAGCGGTCTAAGGCGCTGGATTTAGGCTCCAGTCT |
| | | CTTCGGGGGCGTGGGTTCGAATCCCACCACTGCCA |
| 397 | Thr_TGT_chr14:21099318-21099391 (−) | GGCTCCATAGCTCAGGGGTTAGAGCACTGGTCTTGTAAACCAGGGGTC |
| | | GCGAGTTCAAATCTCGCTGGGGCCTC |
| 398 | Pro_TGG_chr14:21101164-21101236 (+) | TGGCTCGTTGGTCTAGTGGTATGATTCTCGCTTTGGGTGCGAGAGGTC |
| | | CCGGGTTCAAATCCCGGACGAGCCC |
| 399 | Tyr_GTA_chr14:21131350-21131444 (−) | CCTTCGATAGCTCAGCTGGTAGAGCGGAGGACTGTAGATTGTACAGAC |
| | | ATTTGCGGACATCCTTAGGTCGCTGGTTCGATTCCGGCTCGAAGGAA |
| 400 | Thr_TGT_chr14:21149848-21149921 (+) | AGGCCCTATAGCTCAGGGGTTAGAGCACTGGTCTTGTAAACCAGGGGT |
| | | CGCGAGTTCAAATCTCGCTGGGGCCT |
| 401 | Tyr_GTA_chr14:21151431-21151520 (+) | TCCTTCGATAGCTCAGCTGGTAGAGCGGAGGACTGTAGTACTTAATGT |
| | | GTGGTCATCCTTAGGTCGCTGGTTCGATTCCGGCTCGAAGGA |
| 402 | Pro_TGG_chr14:21152174-21152246 (+) | TGGCTCGTTGGTCTAGGGGTATGATTCTCGCTTTGGGTGCGAGAGGTC |
| | | CCGGGTTCAAATCCCGGACGAGCCC |
| 403 | Lys_CTT_chr14:58706612-58706685 (−) | GCCCGGCTAGCTCAGTCGGTAGAGCATGGGACTCTTAATCCCAGGGTC |
| | | GTGGGTTCGAGCCCCACGTTGGGCGC |
| 404 | Ile_AAT_chr14:102783428-102783502 (+) | CGGCCGGTTAGCTCAGTTGGTTAGAGCGTGGTGCTAATAACGCCAAGG |
| | | TCGCGGGTTCGATCCCCGTACGGGCCA |
| 405 | Glu_TTC_chr15:26327380-26327452 (−) | TCCCACATGGTCTAGCGGTTAGGATTCCTGGTTTTCACCCAGGCGGCC |
| | | CGGGTTCGACTCCCGGTGTGGGAAT |
| 406 | Ser_GCT_chr15:40886022-40886104 (−) | GACGAGGTGGCCGAGTGGTTAAGGCGATGGACTGCTAATCCATTGTGC |
| | | TCTGCACGCGTGGGTTCGAATCCCATCCTCGTCGA |
| 407 | His_GTG_chr15:45490803-45490875 (−) | GCCGTGATCGTATAGTGGTTAGTACTCTGCGTTGTGGCCGCAGCAACC |
| | | TCGGTTCGAATCCGAGTCACGGCAT |
| 408 | His_GTG_chr15:45493348-45493420 (+) | CGCCGTGATCGTATAGTGGTTAGTACTCTGCGTTGTGGCCGCAGCAAC |
| | | CTCGGTTCGAATCCGAGTCACGGCA |
| 409 | Gln_CTG_chr15:66161399-66161471 (−) | GGTTCCATGGTGTAATGGTTAGCACTCTGGACTCTGAATCCAGCGATC |
| | | CGAGTTCAAATCTCGGTGGAACCTG |
| 410 | Lys_CTT_chr15:79152903-79152976 (+) | TGCCCGGCTAGCTCAGTCGGTAGAGCATGGGACTCTTAATCCCAGGGT |
| | | CGTGGGTTCGAGCCCCACGTTGGGCG |
| 411 | Arg_TCG_chr15:89878303-89878376 (+) | GGGCCGCGTGGCCTAATGGATAAGGCGTCTGACTTCGGATCAGAAGAT |
| | | TGCAGGTTCGAGTCCTGCCGCGGTCG |
| 412 | Gly_CCC_chr16:686735-686806 (−) | GCGCCGCTGGTGTAGTGGTATCATGCAAGATTCCCATTCTTGCGACCC |
| | | GGGTTCGATTCCCGGGCGGCGCAC |
| 413 | Arg_CCG_chr16:3200674-3200747 (+) | GGGCCGCGTGGCCTAATGGATAAGGCGTCTGATTCCGGATCAGAAGAT |
| | | TGAGGGTTCGAGTCCCTTCGTGGTCG |
| 414 | Arg_CCT_chr16:3202900-3202973 (+) | CGCCCCGGTGGCCTAATGGATAAGGCATTGGCCTCCTAAGCCAGGGAT |
| | | TGTGGGTTCGAGTCCCACCCGGGGTA |
| 415 | Lys_CTT_chr16:3207405-3207478 (−) | GCCCGGCTAGCTCAGTCGGTAGAGCATGAGACCCTTAATCTCAGGGTC |
| | | GTGGGTTCGAGCCCCACGTTGGGCGT |
| 416 | Thr_CGT_chr16:14379749-14379821 (+) | AGGCGCGGTGGCCAAGTGGTAAGGCGTCGGTCTCGTAAACCGAAGATC |
| | | ACGGGTTCGAACCCCGTCCGTGCCT |
| 417 | Leu_TAG_chr16:22207031-22207113 (−) | GGTAGCGTGGCCGAGTGGTCTAAGGCGCTGGATTTAGGCTCCAGTCAT |
| | | TTCGATGGCGTGGGTTCGAATCCCACCGCTGCCAC |
| 418 | Leu_AAG_chr16:22308460-22308542 (+) | GGGTAGCGTGGCCGAGCGGTCTAAGGCGCTGGATTAAGGCTCCAGTCT |
| | | CTTCGGGGGCGTGGGTTCGAATCCCACCGCTGCCA |
| 419 | Leu_CAG_chr16:57333862-57333945 (+) | AGTCAGGATGGCCGAGCGGTCTAAGGCGCTGCGTTCAGGTCGCAGTCT |
| | | CCCCTGGAGGCGTGGGTTCGAATCCCACTTCTGACA |
| 420 | Leu_CAG_chr16:57334391-57334474 (−) | GTCAGGATGGCCGAGCGGTCTAAGGCGCTGCGTTCAGGTCGCAGTCTC |
| | | CCCTGGAGGCGTGGGTTCGAATCCCACTTCTGACAG |
| 421 | Met_CAT_chr16:87417627-87417700 (−) | GCCTCGTTAGCGCAGTAGGCAGCGCGTCAGTCTCATAATCTGAAGGTC |
| | | GTGAGTTCGAGCCTCACACGGGGCAG |
| 422 | Leu_TAG_chr17:8023631-8023713 (−) | GGTAGCGTGGCCGAGCGGTCTAAGGCGCTGGATTTAGGCTCCAGTCTC |
| | | TTCGGAGGCGTGGGTTCGAATCCCACCGCTGCCAG |
| 423 | Arg_TCT_chr17:8024242-8024330 (+) | TGGCTCTGTGGCGCAATGGATAGCGCATTGGACTTCTAGTGACGAATA |
| | | GAGCAATTCAAAGGTTGTGGGTTCGAATCCCACCAGAGTCG |
| 424 | Gly_GCC_chr17:8029063-8029134 (+) | CGCATTGGTGGTTCAGTGGTAGAATTCTCGCCTGCCACGCGGGAGGCC |
| | | CGGGTTCGATTCCCGGCCAATGCA |
| 425 | Ser_CGA_chr17:8042198-8042280 (−) | GCTGTGATGGCCGAGTGGTTAAGGCGTTGGACTCGAAATCCAATGGGG |
| | | TCTCCCCGCGCAGGTTCGAATCCTGCTCACAGCGT |
| 426 | Thr_AGT_chr17:8042769-8042843 (−) | GGCGCCGTGGCTTAGCTGGTTAAAGCGCCTGTCTAGTAAACAGGAGAT |
| | | CCTGGGTTCGAATCCCAGCGGTGCCTG |
| 427 | Trp_CCA_chr17:8089675-8089747 (+) | CGACCTCGTGGCGCAACGGTAGCGCGTCTGACTCCAGATCAGAAGGTT |
| | | GCGTGTTCAAATCACGTCGGGGTCA |
| 428 | Ser_GCT_chr17:8090183-8090265 (+) | AGACGAGGTGGCCGAGTGGTTAAGGCGATGGACTGCTAATCCATTGTG |
| | | CTCTGCACGCGTGGGTTCGAATCCCATCCTCGTCG |
| 429 | Thr_AGT_chr17:8090477-8090551 (+) | CGGCGCCGTGGCTTAGTTGGTTAAAGCGCCTGTCTAGTAAACAGGAGA |
| | | TCCTGGGTTCGAATCCCAGCGGTGCCT |
| 430 | Trp_CCA_chr17:8124186-8124258 (−) | GGCCTCGTGGCGCAACGGTAGCGCGTCTGACTCCAGATCAGAAGGTTG |
| | | CGTGTTCAAATCACGTCGGGGTCAA |
| 431 | Gly_TCC_chr17:8124865-8124937 (+) | AGCGTTGGTGGTATAGTGGTAAGCATAGCTGCCTTCCAAGCAGTTGAC |
| | | CCGGGTTCGATTCCCGGCCAACGCA |
| 432 | Asp_GTC_chr17:8125555-8125627 (−) | TCCTCGTTAGTATAGTGGTGAGTATCCCCGCCTGTCACGCGGGAGACC |
| | | GGGGTTCGATTCCCCGACGGGGAGA |
| 433 | Pro_CGG_chr17:8126150-8126222 (−) | GGCTCGTTGGTCTAGGGGTATGATTCTCGCTTCGGGTGCGAGAGGTCC |
| | | CGGGTTCAAATCCCGGACGAGCCCT |
| 434 | Thr_AGT_chr17:8129552-8129626 (−) | GGCGCCGTGGCTTAGTTGGTTAAAGCGCCTGTCTAGTAAACAGGAGAT |
| | | CCTGGGTTCGAATCCCAGCGGTGCCTT |
| 435 | Ser_AGA_chr17:8129927-8130009 (−) | GTAGTCGTGGCCGAGTGGTTAAGGCGATGGACTAGAAATCCATTGGGG |
| | | TCTCCCCGCGCAGGTTCGAATCCTGCCGACTACGT |
| 436 | Trp_CCA_chr17:19411493-19411565 (+) | TGACCTCGTGGCGCAATGGTAGCGCGTCTGACTCCAGATCAGAAGGTT |
| | | GCGTGTTCAAGTCACGTCGGGGTCA |
| 437 | Thr_CGT_chr17:29877092-29877164 (+) | AGGCGCGGTGGCCAAGTGGTAAGGCGTCGGTCTCGTAAACCGAAGATC |
| | | GCGGGTTCGAACCCCGTCCGTGCCT |
| 438 | Cys_GCA_chr17:37023897-37023969 (+) | AGGGGGTATAGCTCAGTGGTAGAGCATTTGACTGCAGATCAAGAGGTC |
| | | CCCGGTTCAAATCCGGGTGCCCCCT |
| 439 | Cys_GCA_chr17:37025544-37025616 (−) | GGGGGTATAGCTCAGTGGTAGAGCATTTGACTGCAGATCAAGAGGTCC |
| | | CTGGTTCAAATCCGGGTGCCCCCTC |
| 440 | Cys_GCA_chr17:37309986-37310058 (−) | GGGGGTATAGCTCAGTGGTAGAGCATTTGACTGCAGATCAAGAGGTCC |
| | | CCGGTTCAAATCCGGGTGCCCCCTC |
| 441 | Gln_TTG_chr17:47269889-47269961 (+) | AGGTCCCATGGTGTAATGGTTAGCACTCTGGACTTTGAATCCAGCGAT |
| | | CCGAGTTCAAATCTCGGTGGGACCT |
| 442 | Arg_CCG_chr17:66016012-66016085 (−) | GACCCAGTGGCCTAATGGATAAGGCATCAGCCTCCGGAGCTGGGGATT |
| | | GTGGGTTCGAGTCCCATCTGGGTCGC |
| 443 | Arg_CCT_chr17:73030000-73030073 (+) | AGCCCCAGTGGCCTAATGGATAAGGCACTGGCCTCCTAAGCCAGGGAT |
| | | TGTGGGTTCGAGTCCCACCTGGGGTA |
| 444 | Arg_CCT_chr17:73030525-73030598 (−) | GCCCCAGTGGCCTAATGGATAAGGCACTGGCCTCCTAAGCCAGGGATT |
| | | GTGGGTTCGAGTCCCACCTGGGGTGT |
| 445 | Arg_TCG_chr17:73031207-73031280 (+) | AGACCGCGTGGCCTAATGGATAAGGCGTCTGACTTCGGATCAGAAGAT |
| | | TGAGGGTTCGAGTCCCTTCGTGGTCG |
| 446 | Asn_GTT_chr19:1383561-1383635 (+) | CGTCTCTGTGGCGCAATCGGTTAGCGCGTTCGGCTGTTAACCGAAAGG |
| | | TTGGTGGTTCGAGCCCACCCAGGGACG |
| 447 | Gly_TCC_chr19:4724081-4724153 (+) | GGCGTTGGTGGTATAGTGGTTAGCATAGCTGCCTTCCAAGCAGTTGAC |
| | | CCGGGTTCGATTCCCGGCCAACGCA |
| 448 | Val_CAC_chr19:4724646-4724719 (−) | GTTTCCGTAGTGTAGCGGTTATCACATTCGCCTCACACGCGAAAGGTC |
| | | CCCGGTTCGATCCCGGGCGGAAACAG |
| 449 | Thr_AGT_chr19:33667962-33668036 (+) | TGGCGCCGTGGCTTAGTTGGTTAAAGCGCCTGTCTAGTAAACAGGAGA |
| | | TCCTGGGTTCGAATCCCAGCGGTGCCT |
| 450 | Ile_TAT_chr19:39902807-39902900 (−) | GCTCCAGTGGCGCAATCGGTTAGCGCGCGGTACTTATATGACAGTGCG |
| | | AGCGGAGCAATGCCGAGGTTGTGAGTTCGATCCTCACCTGGAGCAC |
| 451 | Gly_GCC_chr21:18827106-18827177 (−) | GCATGGGTGGTTCAGTGGTAGAATTCTCGCCTGCCACGCGGGAGGCCC |
| | | GGGTTCGATTCCCGGCCCATGCAG |

In an embodiment, a TREM, e.g., an exogenous TREM, comprises 1, 2, 3, or 4 of the following properties:
(a) differs by at least one nucleotide or one post transcriptional modification from the closest sequence tRNA in a reference cell, e.g., a cell into which the exogenous nucleic acid is introduced;
(b) has been introduced into a cell other than the cell in which it was transcribed;
(c) is present in a cell other than one in which it naturally occurs; or
(d) has an expression profile, e.g., level or distribution, that is non-wildtype, e.g., it is expressed at a higher level than wildtype.

In an embodiment, the expression profile can be mediated by a change introduced into a nucleic acid that modulates expression, or by addition of an agent that modulates expression of the RNA molecule.

In an embodiment, a TREM, e.g., an exogenous TREM comprises (a), (b), (c) and (d).

In an embodiment, a TREM, e.g., an exogenous TREM comprises (a), (b) and (c).

In an embodiment, a TREM, e.g., an exogenous TREM comprises (a), (b) and (d).

In an embodiment, a TREM, e.g., an exogenous TREM comprises (a), (c) and (d).

In an embodiment, a TREM, e.g., an exogenous TREM comprises (b), (c) and (d).

In an embodiment, a TREM, e.g., an exogenous TREM comprises (a) and (d).

In an embodiment, a TREM, e.g., an exogenous TREM comprises (c) and (d).

### TREM Fragments

In an embodiment, a TREM comprises a fragment (sometimes referred to herein as a TREM fragment), e.g., a fragment of a RNA encoded by a deoxyribonucleic acid sequence disclosed in Table 1. E.g., the TREM includes less than the full sequence of a tRNA, e.g., less than the full sequence of a tRNA with the same anticodon, from the same species as the subject being treated, or both. In an embodiment, the production of a TREM fragment, e.g., from a full length TREM or a longer fragment, can be catalyzed by an enzyme, e.g., an enzyme having nuclease activity (e.g., endonuclease activity or ribonuclease activity), e.g., Dicer, Angiogenin, RNaseP, RNaseZ, Rnyl, or PrrC.

In an embodiment, a TREM fragment can be produced in vivo, ex vivo or in vitro. In an embodiment, a TREM fragment is produced in vivo, in the host cell. In an embodiment, a TREM fragment is produced ex vivo. In an embodiment, a TREM fragment is produced in vitro, e.g., as described in Example 12. In an embodiment, the TREM fragment is produced by fragmenting an expressed TREM after production of the TREM by the cell, e.g., a TREM produced by the host cell is fragmented after release or purification from the host cell, e.g., the TREM is fragmented ex vivo or in vitro.

Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM fragment comprises at least 5%, 10%, 15%, 20%, 25%, 30%,
35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in

Table 1, e.g., any one of SEQ ID NOs: 1-451 disclosed in Table 1. In an embodiment, a TREM fragment comprises at least 5 ribonucleotides (nt), 10 nt, 15 nt, 20 nt, 25 nt, 30 nt, 35 nt, 40 nt,
45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ ID NOs: 1-451 disclosed in Table 1.

In an embodiment, a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt.

In an embodiment, a TREM fragment comprises a TREM structure, domain, or activity, e.g., as described herein above. In an embodiment, a TREM fragment comprises adaptor function, e.g., as described herein. In an embodiment, a TREM fragment comprises cognate adaptor function, e.g., as described herein. In an embodiment, a TREM fragment comprises non-cognate adaptor function, e.g., as described herein. In an embodiment, a TREM fragment comprises regulatory function, e.g., as described herein.

In an embodiment, a TREM fragment comprises translation inhibition function, e.g., displacement of an initiation factor, e.g., eIF4 G.

In an embodiment, a TREM fragment comprises epigenetic function, e.g., epigenetic inheritance of a disorder, e.g., a metabolic disorder. In some embodiments, an epigenetic inheritance function can have a generational impact, e.g., as compared to somatic epigenetic regulation.

In an embodiment, a TREM fragment comprises retroviral regulation function, e.g., regulation of retroviral reverse transcription, e.g., HERV regulation.

In an embodiment, a TREM fragment comprises gene silencing function, e.g., by binding to AGO and/or PIWI.

In an embodiment, a TREM fragment comprises neuroprotectant function, e.g., by the sequestration of a translation initiation factor, e.g., in stress granules, to promote, e.g., motor neuron survival under cellular stress.

In an embodiment, a TREM fragment comprises anti-cancer function, e.g., by preventing cancer progression through the binding and/or sequestration of, e.g., metastatic transcript-stabilizing proteins.

In an embodiment, a TREM fragment comprises cell survival function, e.g., increased cell survival, by binding to, e.g., cytochrome c and/or cyt c ribonucleoprotein complex.

In an embodiment, a TREM fragment comprises ribosome biogenesis function, e.g., a TREM fragment can regulate ribosome biogenesis by, e.g., regulation of, e.g., binding to, an mRNA coding for ribosomal proteins.

### TREM Modifications

A TREM described herein can comprise a moiety, often referred to herein as a modification, e.g., a moiety described in any one of Tables 2-4. While the term modification as used herein should not generally be construed to be the product of any particular process, in embodiments, the formation of a modification can be mediated by an enzyme in Table 2. In embodiments, the modification is formed post-transcriptionally. In embodiments, the modification is formed co-transcriptionally. In an embodiment, the modification occurs in vivo, e.g., in the host cell.

In an embodiment, the modification is a modification listed in any of the rows of Table 2. In an embodiment, the modification is a modification listed in any of the rows of Table 2, and the formation of the modification is mediated by an enzyme in Table 2. In an embodiment the modification is selected from a row in Table 2 and the formation of the modification is mediated by an enzyme from the same row in Table 2. In an embodiment, the modification is a modification listed in any of the rows of Table 3. In an embodiment, the modification is a modification listed in any of the rows of Table 4.

In an embodiment the host cell is an insect cell or cell line and the modification is a modification listed in any of the rows of Table 2. In an embodiment the host cell is an insect cell or cell line and the modification is a modification listed in any of the rows of Table 2 and the formation of the modification is mediated by an enzyme in Table 2. In an embodiment the host cell is an insect cell or cell line and the modification is selected from a row in Table 2 and the formation of the modification is mediated by an enzyme from the same row in Table 2.

In an embodiment the host cell is a fungal cell or cell line and the modification is a modification listed in any of the rows of Table 3.

In an embodiment the host cell is a plant, plant cell or cell line and the modification is a modification listed in any of the rows of Table 4.

TABLE 2
List of insect tRNA modifications and associated enzymes.

| | Short | | |
|---|---|---|---|
| | Name | Modification | Enzyme list |
| 1 | m5U | 5-methyluridine | Trm2 |
| 2 | m2, 2G | N2,N2-dimethylguanosine | Trm1 |
| 3 | D | dihydrouridine | Dus1, Dus2, |
| | | | Dus3, Dus4 |
| 4 | m5C | 5-methylcytidine | Trm4 |
| 5 | Ar(p) | 2′-O-ribosyladenosine (phosphate) | Rit1 |
| 6 | m1G | 1-methylguanosine | Trm10, Trm5 |
| 7 | m1I | 1-methylinosine | Tad1, Trm5 |
| 8 | mcm5U | 5-methoxycarbonylmethyluridine | Trm9 |
| 9 | Gm | 2′-O-methylguanosine | Trm3, Trm7 |
| 10 | yW | wybutosine | Trm5, TYW1, |
| | | | TYW2, TYW3, |
| | | | TYW4 |
| 11 | Gr(p) | 2′-O-ribosylguanosine (phosphate) | |
| 12 | m2G | N2-methylguanosine | Trm11 |
| 13 | ncm5U | 5-carbamoylmethyluridine | |
| 14 | cmnm5s2U | 5-carboxymethylaminomethyl-2- | Mto1, Mtu1, |
| | | thiouridine | Ncs6 |
| 15 | Um | 2′-O-methyluridine | Trm44 |
| 16 | m3C | 3-methylcytidine | Trm140 |
| 17 | i6A | N6-isopentenyladenosine | Mod5 |
| 18 | xG | unknown modified guanosine | |
| 19 | mcm5s2U | 5-methoxycarbonylmethyl-2- | Ncs2, Trm9 |
| | | thiouridine | |
| 20 | xA | unknown modified adenosine | |
| 21 | xU | unknown modified uridine | |
| 22 | ac4C | N4-acetylcytidine | Rra1, Tan1 |
| 23 | m1A | 1-methyladenosine | Trm61 |
| 24 | Cm | 2′-O-methylcytidine | Trm13, Trm7 |
| 25 | cmnm5U | 5- | |
| | | carboxymethylaminomethyluridine | |
| 26 | I | inosine | Tad2, Tad3 |
| 27 | m7G | 7-methylguanosine | Trm8 |
| 28 | Y | pseudouridine | Cbf5, Pus1, Pus2, |
| | | | Pus3, Pus4, Pus6, |
| | | | Pus7, Pus8, Pus9 |
| 29 | Am | 2′-O-methyladenosine | Trm13 |
| 30 | t6A | N6-threonylcarbamoyladenosine | Bud32, Cgi121, |
| | | | Pcc1, Qri7, Sua5 |

TABLE 3
List of fungal tRNA modifications

| | Short Name | Modification |
|---|---|---|
| 1 | io6A | N6-(cis-hydroxyisopentenyl)adenosine |
| 2 | m5U | 5-methyluridine |
| 3 | m2, 2G | N2,N2-dimethylguanosine |
| 4 | D | dihydrouridine |
| 5 | m6t6A | N6-methyl-N6-threonylcarbamoyladenosine |
| 6 | m5C | 5-methylcytidine |
| 7 | m1G | 1-methylguanosine |
| 8 | Gm | 2′-O-methylguanosine |
| 9 | m2G | N2-methylguanosine |
| 10 | m2A | 2-methyladenosine |
| 11 | ncm5U | 5-carbamoylmethyluridine |
| 12 | Um | 2′-O-methyluridine |
| 13 | ms216A | 2-methylthio-N6-isopentenyladenosine |
| 14 | m3C | 3-methylcytidine |
| 15 | i6A | N6-isopentenyladenosine |
| 16 | QtRNA | queuosine |
| 17 | xG | unknown modified guanosine |
| 18 | xA | unknown modified adenosine |
| 19 | acp3U | 3-(3-amino-3-carboxypropyl)uridine |
| 20 | xU | unknown modified uridine |
| 21 | ac4C | N4-acetylcytidine |
| 22 | m1A | 1-methyladenosine |
| 23 | Cm | 2′-O-methylcytidine |
| 24 | cmnm5U | 5-carboxymethylaminomethyluridine |
| 25 | m6A | N6-methyladenosine |
| 26 | Ym | 2′-O-methylpseudouridine |
| 27 | mnm5s2U | 5-methylaminomethyl-2-thiouridine |
| 28 | I | inosine |
| 29 | m7G | 7-methylguanosine |
| 30 | Y | pseudouridine |
| 31 | o2yW | peroxywybutosine |
| 32 | t6A | N6-threonylcarbamoyladenosine |

TABLE 4
List of plant tRNA modifications

| | Short Name | Modification |
|---|---|---|
| 1 | m5U | 5-methyluridine |
| 2 | m2, 2G | N2,N2-dimethylguanosine |
| 3 | D | dihydrouridine |
| 4 | m6t6A | N6-methyl-N6-threonylcarbamoyladenosine |
| 5 | m5C | 5-methylcytidine |
| 6 | m1G | 1-methylguanosine |
| 7 | cmnm5Um | 5-carboxymethylaminomethyl-2′-O-methyluridine |
| 8 | m1I | 1-methylinosine |
| 9 | mcm5U | 5-methoxycarbonylmethyluridine |
| 10 | Gm | 2′-O-methylguanosine |
| 11 | m2G | N2-methylguanosine |
| 12 | Um | 2′-O-methyluridine |
| 13 | m3C | 3-methylcytidine |
| 14 | i6A | N6-isopentenyladenosine |
| 15 | QtRNA | queuosine |
| 16 | xG | unknown modified guanosine |
| 17 | acp3U | 3-(3-amino-3-carboxypropyl)uridine |
| 18 | xU | unknown modified uridine |
| 19 | ac4C | N4-acetylcytidine |
| 20 | m1A | 1-methyladenosine |
| 21 | Cm | 2′-O-methylcytidine |

### TREM Fusion

In an embodiment, a TREM disclosed herein comprises an additional moiety, e.g., a fusion moiety. In an embodiment, the fusion moiety can be used for purification, to alter folding of the TREM, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM.

In an embodiment, the fusion moiety can be encoded by the same or different nucleic acid molecule that encodes the TREM.

### TREM Consensus Sequence

In an embodiment, a TREM disclosed herein comprises a consensus sequence provided herein.

In an embodiment, a TREM disclosed herein comprises a consensus sequence of Formula I zzz, wherein zzz indicates any of the twenty amino acids and Formula I corresponds to all species.

In an embodiment, a TREM disclosed herein comprises a consensus sequence of Formula II zzz, wherein zzz indicates any of the twenty amino acids and Formula II corresponds to mammals.

In an embodiment, a TREM disclosed herein comprises a consensus sequence of Formula III zzz, wherein zzz indicates any of the twenty amino acids and Formula III corresponds to humans.

In an embodiment, zzz indicates any of the twenty amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In an embodiment, a TREM disclosed herein comprises a property selected from the following:
a) under physiological conditions residue R₀ forms a linker region, e.g., a Linker 1 region;
b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;
c) under physiological conditions residues R₈-R₉ forms a linker region, e.g., a Linker 2 region;
d) under physiological conditions residues-R₁₀-R₁₁-R₁₂-R₁-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;
e) under physiological conditions residue-R₂₉ forms a linker region, e.g., a Linker 3 Region;
f) under physiological conditions residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;
g) under physiological conditions residue-[R₄₇]ₓ comprises a variable region, e.g., as described herein;
h) under physiological conditions residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or
i) under physiological conditions residue R₇₂ forms a linker region, e.g., a Linker 4 region.

### Alanine TREM Consensus Sequence

In an embodiment, a TREM disclosed herein comprises the sequence of Formula I_{ALA}(SEQ ID NO: 562),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂ wherein R is a ribonucleotide residue and the consensus for Ala is:
R₀=absent;
R₁₄, R₅₇=are independently A or absent;
R₂₆=A, C, G or absent;
R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₅, R₅₉, R₆₃, R₆₄, R₆₆, R₆₇=are independently N or absent;
R₁₁, R₃₅, R₆₅=are independently A, C, U or absent;
R₁, R₉, R₂₀, R₃₈, R₄₀, R₅₁, R₅₂, R₅₆=are independently A, G or absent;
R₇, R₂₂, R₂₅, R₂₇, R₂₉, R₄₆, R₅₃, R₇₂=are independently A, G, U or absent;
R₂₄, R₆₉=are independently A, U or absent;
R₇₀, R₇₁=are independently C or absent;
R₃, R₄=are independently C, G or absent;
R₁₂, R₃₃, R₃₆, R₆₂, R₆₈=are independently C, G, U or absent;
R₁₃, R₁₇, R₂₅, R₃₉, R₅₅, R₆₀, R₆₁=are independently C, U or absent;
R₁₀, R₁₉, R₂₃=are independently G or absent;
R₂=G, U or absent;
R₈, R₁₈, R₅₄ are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ALA}(SEQ ID NO: 563), R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃1, R₅₀, R₅₉=are independently N or absent;
R₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇1=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₈, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA}(SEQ ID NO: 564),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₁-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆4 are independently A, C, G or absent;
R₁₆, R₃₁, R₅₀=are independently N or absent;
R₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆3 are independently A, G, U or absent;
R₆, R₃₅=are independently A, U or absent;
R₅₅, R₆₀, R₆₁, R₇₀, R₇1=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂ are independently G or absent;
R₃₃, R₃₆=are independently G, U or absent;
R₈, R₂₁, R₅₄, R₆₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

Arginine TREM Consensus sequence

In an embodiment, a TREM disclosed herein comprises the sequence of Formula I_{ARG} (SEQ ID NO: 565),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₅₇=A or absent;
R₉, R₂₇=are independently A,C,G or absent;
R₁, R₂, R₃, R₄, R₅, R₆, R₇, R₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅,
R₄₆, R₄₈, R₄₉, R₅₀, R₅₁, R₅₈, R₆₂, R₆₃, R₆₄, R₆₅, R₆₆, R₆₇, R₆₈, R₆₉, R₇₀, R₇₁=are independently N or absent;
R₁₃, R₁₇, R₄₁=are independently A,C,U or absent;
R₁₉, R₂₀, R₂₄, R₄₀, R₅₆=are independently A,G or absent;
R₁₄, R₁₅, R₇₂=are independently A,G,U or absent;
R₁₈=A,U or absent;
R₃₈=C or absent;
R₃₅, R₄₃, R₆₁=are independently C,G,U or absent;
R₂₈, R₅₅, R₅₉, R₆₀=are independently C,U or absent;
R₀, R₁₀, R₅₂=are independently G or absent;
R₈, R₃₉=are independently G,U or absent;
R₃₆, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ARG} (SEQ ID NO: 566),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=absent;
R₂₄, R₅₇=are independently A or absent;
R₄₁=A,C or absent;
R₃, R₇, R₃₄, R₅₀=are independently A,C,G or absent;
R₂, R₅, R₆, R₁₂, R₂₆, R₃₂, R₃₇, R₄₄, R₅₈, R₆₆, R₆₇, R₆₈, R₇₀=are independently N or absent;
R₄₉, R₇₁=are independently A,C,U or absent;
R₁, R₁₅, R₁₉, R₂₅, R₂₇, R₄₀, R₄₅, R₄₆, R₅₆, R₇₂=are independently A,G or absent;
R₁₄, R₂₉, R₆₃=are independently A,G,U or absent;
R₁₆, R₂₁=are independently A,U or absent;
R₃₈, R₆₁=are independently C or absent;
R₃₃, R₄₈=are independently C,G or absent;
R₄, R₉, R₁₁, R₄₃, R₆₂, R₆₄, R₆₉=are independently C,G,U or absent;
R₁₃, R₂₂, R₂₈, R₃₀, R₃₁, R₃₅, R₅₈, R₆₀, R₆₅=are independently C,U or absent;
R₀, R₁₀, R₂₀, R₂₃, R₅₁, R₅₂=are independently G or absent;
R₈, R₃₉, R₄₂=are independently G,U or absent;
R₁₇, R₃₆, R₅₃, R₅₄, R₅₉=are independently U or absent;
[R₄₇]x=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ARG} (SEQ ID NO: 567), R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Arg is:
R₁₈=is absent;
R₁₅, R₂₁, R₂₄, R₄₁, R₅₇=are independently A or absent;
R₃₄, R₄₄=are independently A,C or absent;
R₃, R₈, R₅₈=are independently A,C,G or absent;
R₂, R₆, R₆₆, R₇₀=are independently N or absent;
R₃₇, R₄₉=are independently A,C,U or absent;
R₁, R₂₅, R₂₉, R₄₀, R₄₅, R₄₆, R₅₀=are independently A,G or absent;
R₁₄, R₆₃, R₆₅=are independently A,G,U or absent;
R₁₆=A,U or absent;
R₃₈, R₆₁=are independently C or absent;
R₇, R₁₁, R₁₂, R₂₆, R₄₈=are independently C,G or absent;
R₆₄, R₆₇, R₆₉=are independently C,G,U or absent;
R₄, R₁₃, R₂₂, R₂₈, R₃0, R₃₁, R₃₅, R₄₃, R₅₈, R₆₀, R₆₂, R₆₅, R₇₁=are independently C,U or absent;
R₀, R₁₀, R₁₉, R₂₀, R₂₃, R₂₇, R₃₃, R₅₁, R₅₂, R₅₆, R₇₂=are independently G or absent;
R₈, R₉, R₃₂, R₃₉, R₄₂=are independently G,U or absent;
R₁₇, R₃₆, R₅₃, R₅₄, R₅₉=are independently U or absent;
[R₄₇]x=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, 10 x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I _{ASN} (SEQ ID NO: 568),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₅=are absent;
R₄₁=A or absent;
R₁₄, R₄₈, R₅₆=are independently A,C,G or absent;
R₂, R₄, R₅, R₆, R₁₂, R₁₇, R₂₆, R₂₉, R₃₀, R₃₁, R₄₄, R₄₅, R₄₆, R₄₉, R₀, R₅, R₆₂, R₆₃, R₆₅, R₆₆, R₆₇, R₆₅, R₇₀, R₇₁=
are independently N or absent;
R₁₁, R₁₃, R₂₂, R₄₂, R₅₅, R₅₉=are independently A,C,U or absent;
R₉, R₁₅, R₂₄, R₂₇, R₃₄, R₃₇, R₅₁, R₇₂=are independently A,G or absent;
R₁, R₇, R₂₅, R₆₉=are independently A,G,U or absent;
R₄₀, R₅₇=are independently A,U or absent;
R₆₀=C or absent;
R₃₃=C,G or absent;
R₂₁, R₃₂, R₄₃, R₆₄=are independently C,G,U or absent;
R₃, R₁₆, R₂₈, R₃₅, R₃₆, R₆₁=are independently C,U or absent;
R₁₀, R₁₉, R₂₀, R₅₂=are independently G or absent;
R₅₄=G,U or absent;
R₅, R₂₃, R₃₈, R₃₉, R₅₃=are independently U or absent;
[R₄₇]x=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{ASN} (SEQ ID NO: 569),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₁-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent
R₂₄, R₄₁, R₄₆, R₆₂=are independently A or absent;
R₅₉=A,C or absent;
R₁₄, R₅₆, R₆₆=are independently A,C,G or absent;
R₁₇, R₂₉=are independently N or absent;
R₁₁, R₂₆, R₄₂, R₅₅=are independently A,C,U or absent;
R₁, R₉, R₁₂, R₁₅, R₂₅, R₃₄, R₃₇, R₄₈, R₅₁, R₆₇, R₆₈, R₆₉, R₇₀, R₇₂=are independently A,G or absent;
R₄₄, R₄₅, R₅₈=are independently A,G,U or absent;
R₄₀, R₅₇=are independently A,U or absent;
R₈, R₂₈, R₆₀=are independently C or absent;
R₃₃, R₆₅=are independently C,G or absent;
R₂₁, R₄₃, R₇₁=are independently C,G,U or absent;
R₃, R₆, R₁₃, R₂₂, R₃₂, R₃₅, R₃₆, R₆₁, R₆₃, R₆₄=are independently C,U or absent;
R₇, R₁₀, R₁₉, R₂₀, R₂₇, R₄₉, R₅₂=are independently G or absent;
R₅₄=G,U or absent;
R₂, R₄, R₈, R₁₆, R₂₃, R₃₀, R₃₁, R₃₈, R₃₉, R₅₀, R₅₃=are independently U or absent;
[R₄₇]x=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{ASN} (SEQ ID NO: 570),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asn is:
R₀, R₁₈=are absent
R₂₄, R₄₀, R₄₁, R₄₆, R₆₂=are independently A or absent;
R₅₉=A,C or absent;
R₁₄, R₅₆, R₆₆=are independently A,C,G or absent;
R₁₁, R₂₆, R₄₂, R₅₅=are independently A,C,U or absent;
R₁, R₉, R₁₂, R₁₅, R₃₄, R₃₇, R₄₈, R₅₁, R₆₇, R₆₈, R₆₉, R₇₀=are independently A,G or absent;
R₄₄, R₄₅, R₅₈=are independently A,G,U or absent;
R₅₇=A,U or absent;
R₅, R₂₈, R₆₀=are independently C or absent;
R₃₃, R₆₅=are independently C,G or absent;
R₁₇, R₂₁, R₂₉=are independently C,G,U or absent;
R₃, R₆, R₁₃, R₂₂, R₃₂, R₃₅, R₃₆, R₄₃, R₆₁, R₆₃, R₆₄, R₇₁=are independently C,U or absent;
R₇, R₁₀, R₁₉, R₂₀, R₂₅, R₂₇, R₄₉, R₅₂, R₇₂=are independently G or absent;
R₅₄=G,U or absent;
R₂, R₄, R₈, R₁₆, R₂₃, R₃₀, R₃₁, R₃₈, R₃₉, R₅₀, R₅₃=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I_{ASP} (SEQ ID NO: 571),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀=absent
R₂₄, R₇₁=are independently A,C or absent;
R₃₃, R₄₆=are independently A,C,G or absent;
R₂, R₃, R₄, R₅, R₆, R₁₂, R₁₆, R₂₂, R₂₆, R₂₉, R₃₁, R₃₂, R₄₄, R₄₈, R₄₉, R₅₈, R₆₃, R₆₄, R₆₆, R₆₇, R₆₈, R₆₉=are
independently N or absent;
R₁₃, R₂₁, R₃₄, R₄₁, R₅₇, R₆₅=are independently A,C,U or absent;
R₉, R₁₀, R₁₄, R₁₅, R₂₀, R₂₇, R₃₇, R₄₀, R₅₁, R₅₆, R₇₂=are independently A,G or absent;
R₇, R₂₅, R₄₂=are independently A,G,U or absent;
R₃₉=C or absent;
R₅₀, R₆₂=are independently C,G or absent;
R₃₀, R₄₃, R₄₅, R₅₅, R₇₀=are independently C,G,U or absent;
R₈, R₁₁, R₁₇, R₁₅, R₂₈, R₃₅, R₅₃, R₅₉, R₆₀, R₆₁=are independently C,U or absent;
R₁₉, R₅₂=are independently G or absent;
R₁=G,U or absent;
R₂₃, R₃₆, R₃₈, R₅₄=are independently U or absent;
[R₄₇]x=N or absent;
wherein, e.g., x=1-271 (e.g., x=1-250, x=1-225, x=1-200, x=1-175, x=1-150, x=1-125, x=1-100,x=1-75,x=1-50,x=1-40,x=1-30,x=1-29,x=1-28,x=1-27,x=1-26,x=1-25,x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ASP} (SEQ ID NO: 572),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are independently absent;
R₉, R₄₀=are independently A or absent;
R₂₄, R₇₁=are independently A,C or absent;
R₆₇, R₆₈=are independently A,C,G or absent;
R₂, R₆, R₆₆=are independently N or absent;
R₅₇, R₆₃=are independently A,C,U or absent;
R₁₀, R₁₄, R₂₇, R₃₃, R₃₇, R₄₄, R₄₆, R₅₁, R₅₆, R₆₄, R₇₂=are independently A,G or absent;
R₇, R₁₂, R₂₆, R₆₅=are independently A,U or absent;
R₃₉, R₆₁, R₆₂=are independently C or absent;
R₃, R₃₁, R₄₅, R₇₀=are independently C,G or absent;
R₄, R₅, R₂₉, R₄₃, R₅₅=are independently C,G,U or absent;
R₅, R₁₁, R₁₃, R₃0, R₃₂, R₃₄, R₃₅, R₄₁, R₄₈, R₅₃, R₅₉, R₆₀=are independently C,U or absent;
R₁₅, R₁₉, R₂₀, R₂₅, R₄₂, R₅₀, R₅₂=are independently G or absent;
R₁, R₂₂, R₄₉, R₅₈, R₆₉=are independently G,U or absent;
R₁₆, R₂₁, R₂₈, R₃₆, R₃₈, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ASP} (SEQ ID NO: 573),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Asp is:
R₀, R₁₇, R₁₈, R₂₃=are absent
R₉, R₁₂, R₄₀, R₆₅, R₇₁=are independently A or absent;
R₂, R₂₄, R₅₇=are independently A,C or absent;
R₆, R₁₄, R₂₇, R₄₆, R₅₁, R₅₆, R₆₄, R₆₇, R₆₈=are independently A,G or absent;
R₃, R₃₁, R₃₅, R₃₉, R₆₁, R₆₂=are independently C or absent;
R₆₆=C,G or absent;
R₅, R₅, R₂₉, R₃₀, R₃₂, R₃₄, R₄₁, R₄₃, R₄₈, R₅₅, R₅₉, R₆₀, R₆₃=are independently C,U or absent;
R₁₀, R₁₅, R₁₉, R₂₀, R₂₅, R₃₃, R₃₇, R₄₂, R₄₄, R₄₅, R₄₉, R₅₀, R₅₂, R₆₉, R₇₀, R₇₂=are independently G or
absent;
R₂₂, R₅₈=are independently G,U or absent;
R₁, R₄, R₇, R₁₁, R₁₃, R₁₆, R₂₁, R₂₆, R₂₈, R₃₆, R₃₈, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I _{CYS} (SEQ ID NO: 574),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀=absent
R₁₄, R₃₉, R₅₇=are independently A or absent;
R₄₁=A,C or absent;
R₁₀, R₁₅, R₂₇, R₃₃, R₆₂=are independently A,C,G or absent;
R₃, R₄, R₅, R₆, R₁₂, R₁₃, R₁₆, R₂₄, R₂₆, R₂₉, R₃₀, R₃₁, R₃₂, R₃₄, R₄₂, R₄₄, R₄₅, R₄₆, R₄₅, R₄₉, R₅₅, R₆₃, R₆₄, R₆₆,
R₆₇, R₆₈, R₆₉, R₇₀=are independently N or absent;
R₆₅=A,C,U or absent;
R₉, R₂₅, R₃₇, R₄₀, R₅₂, R₅₆=are independently A,G or absent;
R₇, R₂₀, R₅₁=are independently A,G,U or absent;
R₁₈, R₃₈, R₅₅=are independently C or absent;
R₂=C, G or absent;
R₂₁, R₂₅, R₄₃, R₅₀=are independently C,G,U or absent;
R₁, R₂₂, R₂₃, R₃₅, R₃₆, R₅₉, R₆₀, R₆₁, R₇₁, R₇₂=are independently C,U or absent;
R₁, R₁₉=are independently G or absent;
R₁₇=G,U or absent;
R₅, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{CYS} (SEQ ID NO: 575),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀, R₁₈, R₂₃=are absent;
R₁₄, R₂₄, R₂₆, R₂₉, R₃₉, R₄₁, R₄₅, R₅₇=are independently A or absent;
R₄₄=A,C or absent;
R₂₇, R₆₂=are independently A,C,G or absent;
R₁₆=A,C,G,U or absent;
R₃₀, R₇₀=are independently A,C,U or absent;
R₅, R₇, R₉, R₂₅, R₃₄, R₃₇, R₄₀, R₄₆, R₅₂, R₅₆, R₅₈, R₆₆=are independently A,G or absent;
R₂₀, R₅₁=are independently A,G,U or absent;
R₃₅, R₃₈, R₄₃, R₅₅, R₆₉=are independently C or absent;
R₂, R₄, R₁₅=are independently C,G or absent;
R₁₃=C,G,U or absent;
R₆, R₁₁, R₂₈, R₃₆, R₄₈, R₄₉, R₅₀, R₆₀, R₆₁, R₆₇, R₆₈, R₇₁, R₇₂=are independently C,U or absent;
R₁, R₃, R₁₀, R₁₉, R₃₃, R₆₃=are independently G or absent;
R₈, R₁₇, R₂₁, R₆₄=are independently G,U or absent;
R₁₂, R₂₂, R₃₁, R₃₂, R₄₂, R₅₃, R₅₄, R₆₅=are independently U or absent;
R₅₉=U, or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{CYS} (SEQ ID NO: 576),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₅-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Cys is:
R₀, R₁₈, R₂₃=are absent
R₁₄, R₂₄, R₂₆, R₂₉, R₃₄, R₃₉, R₄₁, R₄₅, R₅₇, R₅₈=are independently A or absent;
R₄₄, R₇₀=are independently A,C or absent;
R₆₂=A,C,G or absent;
R₁₆=N or absent;
R₅, R₇, R₉, R₂₀, R₄₀, R₄₆, R₅₁, R₅₂, R₅₆, R₆₆=are independently A,G or absent;
R₂₈, R₃₅, R₃₈, R₄₃, R₅₅, R₆₇, R₆₉=are independently C or absent;
R₄, R₁₅=are independently C,G or absent;
R₆, R₁, R₁₃, R₃0, R₄₈, R₄₉, R₅₀, R₆₀, R₆₁, R₆₈, R₇₁, R₇₂=are independently C,U or absent;
R₁, R₂, R₃, R₁₀, R₁₉, R₂₅, R₂₇, R₃₃, R₃₇, R₆₃=are independently G or absent;
R₅, R₂₁, R₆₄=are independently G,U or absent;
R₁₂, R₁₇, R₂₂, R₃₁, R₃₂, R₃₆, R₄₂, R₅₃, R₅₄, R₅₉, R₆₅=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I _{GLN}(SEQ ID NO: 577),
R₀-R₁-R₂-R₃-R₄-R₅-R₆-R₇-R₈-R₉-R₁₀-R₁₁-R₁₂-R₁₃-R₄-R₁₅-R₁₆-R₁₇-R₁-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₅-R₆₇-R₆₈-R₆₉-R₇₀-R₇₁-R₇₂

wherein R is a ribonucleotide residue and the consensus for Gln is:
R₀, R₁₅=are absent;
R₁₄, R₂₄, R₅₇=are independently A or absent;
R₉, R₂₆, R₂₇, R₃₃, R₅₆=are independently A,C,G or absent;
R₂, R₄, R₅, R₆, R₁₂, R₁₃, R₁₆, R₂₁, R₂₂, R₂, R₂₉, R₃₀, R₃₁, R₃₂, R₃₄, R₄₁, R₄₂, R₄₄, R₄, R₄₆, R₄₅, R₄₉, R₅₀, R₈, R
R₆₂, R₆₃, R₆₆, R₆₇, R₆₈, R₆₉, R₇₀-are independently N or absent;
R₁₇, R₂₃, R₄₃, R₆₅, R₇₁=are independently A,C,U or absent;
R₁₅, R₄₀, R₅₁, R₅₂=are independently A,G or absent;
R₁, R₇, R₇₂=are independently A,G,U or absent;
R₃, R₁₁, R₃₇, R₆₀, R₆₄=are independently C,G,U or absent;
R₂₈, R₃₅, R₃₅, R₅₉, R₆₁=are independently C,U or absent;
R₁₀, R₁₉, R₂₀=are independently G or absent;
R₃₉=G,U or absent;
R₅, R₃₆, R₃₅, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLN}(SEQ ID NO: 578),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈, R₂₃=are absent
R₁₄, R₂₄, R₅₇=are independently A or absent;
R₁₇, R₇₁=are independently A,C or absent;
R₂₅, R₂₆, R₃₃, R₄₄, R₄₆, R₅₆, R₆₉=are independently A,C,G or absent;
R₄, R₅, R₁₂, R₂₂, R₂₉, R₃₀, R₄₈, R₄₉, R₆₃, R₆₇, R₆₅=are independently N or absent;
R₃₁, R₄₃, R₆₂, R₆₅, R₇₀=are independently A,C,U or absent;
R₁₅, R₂₇, R₃₄, R₄₀, R₄₁, R₅₁, R₅₂=are independently A,G or absent;
R₂, R₇, R₂₁, R₄₅, R₅₀, R₅₈, R₆₆, R₇₂=are independently A,G,U or absent;
R₃, R₁₃, R₃₂, R₃₇, R₄₂, R₆₀, R₆₄=are independently C,G,U or absent;
R₆, R₁₁, R₂₈, R₃₅, R₅₅, R₅₉, R₆₁=are independently C,U or absent;
R₉, R₁₀, R₁₉, R₂₀=are independently G or absent;
R₁, R₁₆, R₃₉=are independently G,U or absent;
R₅, R₃₆, R₃₈, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLN}(SEQ ID NO: 579),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gln is:
R₀, R₁₈, R₂₃=are absent
R₁₄, R₂₄, R₄₁, R₅₇=are independently A or absent;
R₁₇, R₇₁=are independently A,C or absent;
R₈, R₂₅, R₂₆, R₄₆, R₅₆, R₆₉=are independently A,C,G or absent;
R₄, R₂₂, R₂₉, R₃₀, R₄₈, R₄₉, R₆₃, R₆₈=are independently N or absent;
R₄₃, R₆₂, R₆₅, R₇₀=are independently A,C,U or absent;
R₁₅, R₂₇, R₃₃, R₃₄, R₄₀, R₅₁, R₅₂=are independently A,G or absent;
R₂, R₇, R₁₂, R₄₅, R₅₀, R₅₈, R₆₆=are independently A,G,U or absent;
R₃₁=A,U or absent;
R₃₂, R₄₄, R₆₀=are independently C,G or absent;
R₃, R₁₃, R₃₇, R₄₂, R₆₄, R₆₇=are independently C,G,U or absent;
R₆, R₁₁, R₂₈, R₃₅, R₅₅, R₅₉, R₆₁=are independently C,U or absent;
R₉, R₁₀, R₁₉, R₂₀=are independently G or absent;
R₁, R₂₁, R₃₉, R₇₂=are independently G,U or absent;
R₈, R₁₆, R₃₆, R₃₈, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I _{GLU} (SEQ ID NO: 580),
R₀-R₁-R₂-R₃-R₄-R₅-R₆-R₇-R₈-R₉-R₁₀-R₁₁-R₁₂-R₁₃-R₁₄-R₁₅-R₁₆-R₁-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃-R₃₆-R₃-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀=absent;
R₃₄, R₄₃, R₆₈, R₆₉=are independently A,C,G or absent;
R₁, R₂, R₅, R₆, R₉, R₁₂, R₁₆, R₂₀, R₂₁, R₂₆, R₂₇, R₂₉, R₃₀, R₃₁, R₃₂, R₃₃, R₄₁, R₄₄, R₄, R₄₆, R₄₅, R₅₀, R₅₁, R₅₅, R₆
3, R₆₄, R₆₅, R₆₆, R₇₀, R₇₁=are independently N or absent;
R₁₃, R₁₇, R₂₃, R₆₁=are independently A,C,U or absent;
R₁₀, R₁₄, R₂₄, R₄₀, R₅₂, R₅₆=are independently A,G or absent;
R₇, R₁₅, R₂₅, R₆₇, R₇₂=are independently A,G,U or absent;
R₁₁, R₅₇=are independently A,U or absent;
R₃₉=C,G or absent;
R₃, R₄, R₂₂, R₄₂, R₄₉, R₅₅, R₆₂=are independently C,G,U or absent;
R₁₅, R₂₈, R₃₅, R₃₇, R₅₃, R₅₉, R₆₀=are independently C,U or absent;
R₁₉=G or absent;
R₅, R₃₆, R₃₈, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLU} (SEQ ID NO: 581),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀, R₁₈, R₂₃=are absent
R₁₇, R₄₀=are independently A or absent;
R₂₆, R₂₇, R₃₄, R₄₃, R₆₈, R₆₉, R₇₁=are independently A,C,G or absent;
R₁, R₂, R₅, R₁₂, R₂₁, R₃₁, R₃₃, R₄₁, R₄₅, R₄₈, R₅₁, R₅₈, R₆₆, R₇₀=are independently N or absent;
R₄₄, R₆₁=are independently A,C,U or absent;
R₉, R₁₄, R₂₄, R₂₅, R₅₂, R₅₆, R₆₃=are independently A,G or absent;
R₇, R₁₅, R₄₆, R₅₀, R₆₇, R₇₂=are independently A,G,U or absent;
R₂₉, R₅₇=are independently A,U or absent;
R₆₀=C or absent;
R₃₉=C,G or absent;
R₃, R₆, R₂₀, R₃₀, R₃₂, R₄₂, R₅₅, R₆₂, R₆₅=are independently C,G,U or absent;
R₄, R₈, R₁₆, R₂₈, R₃₅, R₃₇, R₄₉, R₅₃, R₅₉=are independently C,U or absent;
R₁₀, R₁₉=are independently G or absent;
R₂₂, R₆₄=are independently G,U or absent;
R₁₁, R₁₃, R₃₆, R₃₈, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLU} (SEQ ID NO: 582),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Glu is:
R₀, R₁₇, R₁₈, R₂₃=are absent
R₁₄, R₂₇, R₄₀, R₇₁=are independently A or absent;
R₄₄=A,C or absent;
R₄₃=A,C,G or absent;
R₁, R₃₁, R₃₃, R₄₅, R₅₁, R₆₆=are independently N or absent;
R₂₁, R₄₁=are independently A,C,U or absent;
R₇, R₂₄, R₂₅, R₅₀, R₅₂, R₅₆, R₆₃, R₆₈, R₇₀=are independently A,G or absent;
R₈, R₄₆=are independently A,G,U or absent;
R₂₉, R₅₇, R₆₇, R₇₂=are independently A,U or absent;
R₂, R₃₉, R₆₀=are independently C or absent;
R₃, R₁₂, R₂₀, R₂₆, R₃₄, R₆₉=are independently C,G or absent;
R₆, R₃₀, R₄₂, R₄₈, R₆₅=are independently C,G,U o rabsent;
R₄, R₁₆, R₂₈, R₃₅, R₃₇, R₄₉, R₅₃, R₅₅, R₅₈, R₆₁, R₆₂=are independently C,U or absent;
R₉, R₁₀, R₁₉, R₆₄=are independently G or absent;
R₁₅, R₂₂, R₃₂=are independently G,U or absent;
R₅, R₁₁, R₁₃, R₃₆, R₃₅, R₅₄, R₅₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I _{GLY}(SEQ ID NO: 583),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀=absent;
R₂₄=A or absent;
R₃, R₉, R₄₀, R₅₀, R₅₁=are independently A,C,G or absent;
R₄, R₅, R₆, R₇, R₁₂, R₁₆, R₂₁, R₂₂, R₂₆, R₂₉, R₃₀, R₃₁, R₃₂, R₃₃, R₃₄, R₄₁, R₄₂, R₄₃, R₄₄, R₄, R₄₆, R₄₅, R₄₉, R₈, R
R₆₃, R₆₄, R₆₅, R₆₆, R₆₇, R₆₈=are independently N or absent;
R₅₉=A,C,U or absent;
R₁, R₁₀, R₁₄, R₁₅, R₂₇, R₅₆=are independently A,G or absent;
R₂₀, R₂₅=are independently A,G,U or absent;
R₅₇, R₇₂=are independently A,U or absent;
R₃₈, R₃₉, R₆₀=are independently C or absent;
R₅₂=C,G or absent;
R₂, R₁₉, R₃₇, R₅₄, R₅₅, R₆₁, R₆₂, R₆₉, R₇₀=are independently C,G,U or absent;
R₁₁, R₁₃, R₁₇, R₂₈, R₃₅, R₃₆, R₇₁=are independently C,U or absent;
R₈, R₁₅, R₂₃, R₅₃=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{GLY}(SEQ ID NO: 584),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀, R₁₈, R₂₃=are absent
R₂₄, R₂₇, R₄₀, R₇₂=are independently A or absent;
R₂₆=A,C or absent;
R₃, R₇, R₆₅=are independently A,C,G or absent;
R₅, R₃₀, R₄₁, R₄₂, R₄₄, R₄₉, R₆₇=are independently A,C,G,U or absent;
R₃₁, R₃₂, R₃₄=are independently A,C,U or absent;
R₉, R₁₀, R₁₄, R₁₅, R₃₃, R₅₀, R₅₆=are independently A,G or absent;
R₁₂, R₁₆, R₂₂, R₂₅, R₂₉, R₄₆=are independently A,G,U or absent;
R₅₇=A,U or absent;
R₁₇, R₃₈, R₃₉, R₆₀, R₆₁, R₇₁=are independently C or absent;
R₆, R₅₂, R₆₄, R₆₆=are independently C,G or absent;
R₂, R₄, R₃₇, R₄₈, R₅₅, R₆₅=are independently C,G,U or absent;
R₁₃, R₃₅, R₄₃, R₆₂, R₆₉=are independently C,U or absent;
R₁, R₁₉, R₂₀, R₅₁, R₇₀=are independently G or absent;
R₂₁, R₄₅, R₆₃=are independently G,U or absent;
R₅, R₁₁, R₂₈, R₃₆, R₅₃, R₅₄, R₅₈, R₅₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{GLY}(SEQ ID NO: 585),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₁-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Gly is:
R₀, R₁₈, R₂₃=are absent
R₂₄, R₂₇, R₄₀, R₇₂=are independently A or absent;
R₂₆=A,C or absent;
R₃, R₇, R₄₉, R₆₈=are independently A,C,G or absent;
R₅, R₃₀, R₄₁, R₄₄, R₆₇=are independently N or absent;
R₃₁, R₃₂, R₃₄=are independently A,C,U or absent;
R₉, R₁₀, R₁₄, R₁₅, R₃₃, R₅₀, R₅₆=are independently A,G or absent;
R₁₂, R₂₅, R₂₉, R₄₂, R₄₆=are independently A,G,U or absent;
R₁₆, R₅₇=are independently A,U or absent;
R₁₇, R₃₅, R₃₉, R₆₀, R₆₁, R₇₁=are independently C or absent;
R₆, R₅₂, R₆₄, R₆₆=are independently C,G or absent;
R₃₇, R₄₈, R₆₅=are independently C,G,U or absent;
R₂, R₄, R₁₃, R₃₅, R₄₃, R₅₅, R₆₂, R₆₉=are independently C,U or absent;
R₁, R₁₉, R₂₀, R₅₁, R₇₀=are independently G or absent;
R₂₁, R₂₂, R₄₅, R₆₃=are independently G,U or absent;
R₅, R₁₁, R₂₈, R₃₆, R₅₃, R₅₄, R₅₈, R₅₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I _{HIS} (SEQ ID NO: 586),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂

wherein R is a ribonucleotide residue and the consensus for His is:
R₂₃=absent;
R₁₄, R₂₄, R₅₇=are independently A or absent;
R₇₂=A,C or absent;
R₉, R₂₇, R₄₃, R₄₈, R₆₉=are independently A,C,G or absent;
R₃, R₄, R₅, R₆, R₁₂, R₂₅, R₂₆, R₂₉, R₃₀, R₃₁, R₃₄, R₄₂, R₄₅, R₄₆, R₄₉, R₀, R₅, R₆₂, R₆₃, R₆₆, R₆₇, R₆₅=are
independently N or absent;
R₁₃, R₂₁, R₄₁, R₄₄, R₆₅=are independently A,C,U or absent;
R₄₀, R₅₁, R₅₆, R₇₀=are independently A,G or absent;
R₇, R₃₂=are independently A,G,U or absent;
R₅₅, R₆₀=are independently C or absent;
R₁₁, R₁₆, R₃₃, R₆₄=are independently C,G,U or absent;
R₂, R₁₇, R₂₂, R₂₈, R₃₅, R₅₃, R₅₉, R₆₁, R₇₁=are independently C,U or absent;
R₁, R₁₀, R₁₅, R₁₉, R₂, R₃₇, R₃₉, R₅₂=are independently G or absent;
R₀=G,U or absent;
R₈, R₁₅, R₃₆, R₃₈, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{HIS} (SEQ ID NO: 587),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀, R₁₇, R₁₈, R₂₃=are absent;
R₇, R₁₂, R₁₄, R₂₄, R₂₇, R₄₅, R₅₇, R₅₈, R₆₃, R₆₇, R₇₂=are independently A or absent;
R₃=A,C,U or absent;
R₄, R₄₃, R₅₆, R₇₀=are independently A,G or absent;
R₄₉=A,U or absent;
R₂, R₂₈, R₃₀, R₄₁, R₄₂, R₄₄, R₄₈, R₅₈, R₆₀, R₆₆, R₇₁=are independently C or absent;
R₂₅=C,G or absent;
R₉=C,G,U or absent;
R₅, R₁₃, R₂₆, R₃₃, R₃₅, R₅₀, R₅₃, R₆₁, R₆₈=are independently C,U or absent;
R₁, R₆, R₁₀, R₁₅, R₁₉, R₂₀, R₃₂, R₃₄, R₃₇, R₃₉, R₄₀, R₄₆, R₅₁, R₅₂, R₆₂, R₆₄, R₆₉=are independently G or
absent;
R₁₆=G,U or absent;
R₅, R₁₁, R₂₁, R₂₂, R₂₉, R₃₁, R₃₆, R₃₈, R₅₄, R₅₉, R₆₅=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, 25 x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{HIS} (SEQ ID NO: 588),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for His is:
R₀, R₁₇, R₁₈, R₂₃=are absent
R₇, R₁₂, R₁₄, R₂₄, R₂₇, R₄₅, R₅₇, R₅₈, R₆₃, R₆₇, R₇₂=are independently A or absent;
R₃=A,C or absent;
R₄, R₄₃, R₅₆, R₇₀=are independently A,G or absent;
R₄₉=A,U or absent;
R₂, R₂₈, R₃₀, R₄₁, R₄₂, R₄₄, R₄₈, R₅₈, R₆₀, R₆₆, R₇₁=are independently C or absent;
R₅, R₉, R₂₆, R₃₃, R₃₅, R₅₀, R₆₁, R₆₈=are independently C,U or absent;
R₁, R₆, R₁₀, R₁₅, R₁₉, R₂₀, R₂₅, R₃₂, R₃₄, R₃₇, R₃₉, R₄₀, R₄₆, R₅₁, R₅₂, R₆₂, R₆₄, R₆₉=are independently G
or absent;
R₅, R₁, R₁₃, R₁₆, R₂₁, R₂₂, R₂₉, R₃₁, R₃₆, R₃₈, R₅₃, R₅₄, R₅₉, R₆₅=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

Isoleucine TREM Consensus sequence

In an embodiment, a TREM disclosed herein comprises the sequence of Formula I _{ILE} (SEQ ID NO: 589),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₂₃=absent;
R₃₈, R₄₁, R₅₇, R₇₂=are independently A or absent;
R₁, R₂₆=are independently A,C,G or absent;
R₀, R₃, R₄, R₆, R₁₆, R₃₁, R₃₂, R₃₄, R₃₇, R₄₂, R₄₃, R₄₄, R₄, R₄₆, R₄₅, R₄₉, R₅₀, R₅, R₅₉, R₆₂, R₆₃, R₆₄, R₆₆, R₆₇, R
R₆₈, R₆₉=are independently N or absent;
R₂₂, R₆₁, R₆₅=are independently A,C,U or absent;
R₉, R₁₄, R₁₅, R₂₄, R₂₇, R₄₀=are independently A,G or absent;
R₇, R₂₅, R₂₉, R₅₁, R₅₆=are independently A,G,U or absent;
R₁₈, R₅₄=are independently A,U or absent;
R₆₀=C or absent;
R₂, R₅₂, R₇₀=are independently C,G or absent;
R₅, R₁₂, R₂₁, R₃₀, R₃₃, R₇₁=are independently C,G,U or absent;
R₁₁, R₁₃, R₁₇, R₂₈, R₃₅, R₅₃, R₅₅=are independently C,U or absent;
R₁₀, R₁₉, R₂₀=are independently G or absent;
R₅, R₃₆, R₃₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ILE (SEQ ID NO: 590),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀, R₁₈, R₂₃=are absent
R₂₄, R₃₈, R₄₀, R₄₁, R₅₇, R₇₂=are independently A or absent;
R₂₆, R₆₅=are independently A,C or absent;
R₅₈, R₅₉, R₆₇=are independently N or absent;
R₂₂=A,C,U or absent;
R₆, R₉, R₁₄, R₁₅, R₂₉, R₃₄, R₄₃, R₄₆, R₄₈, R₅₀, R₅₁, R₆₃, R₆₉=are independently A,G or absent;
R₃₇, R₅₆=are independently A,G,U or absent;
R₅₄=A,U or absent;
R₂₈, R₃₅, R₆₀, R₆₂, R₇₁=are independently C or absent;
R₂, R₅₂, R₇₀=are independently C,G or absent;
R₅=C,G,U or absent;
R₃, R₄, R₁₁, R₁₃, R₁₇, R₂₁, R₃₀, R₄₂, R₄₄, R₄₅, R₄₉, R₅₃, R₅₅, R₆₁, R₆₄, R₆₆=are independently C,U or absent;
R₁, R₁₀, R₁₉, R₂₀, R₂₅, R₂₇, R₃₁, R₆₅=are independently G or absent;
R₇, R₁₂, R₃₂=are independently G,U or absent;
R₈, R₁₆, R₃₃, R₃₆, R₃₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ILE (SEQ ID NO: 591),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ile is:
R₀, R₁₈, R₂₃=are absent
R₁₄, R₂₄, R₃₅, R₄₀, R₄₁, R₅₇, R₇₂=are independently A or absent;
R₂₆, R₆₅=are independently A,C or absent;
R₂₂, R₅₉=are independently A,C,U or absent;
R₆, R₉, R₁₅, R₃₄, R₄₃, R₄₆, R₅₁, R₅₆, R₆₃, R₆₉=are independently A,G or absent;
R₃₇=A,G,U or absent;
R₁₃, R₂₈, R₃₅, R₄₄, R₅₈, R₆₀, R₆₂, R₇₁=are independently C or absent;
R₂, R₈, R₇₀=are independently C,G or absent;
R₅₈, R₆₇=are independently C,G,U or absent;
R₃, R₄, R₁₁, R₁₇, R₂₁, R₃₀, R₄₂, R₄₅, R₄₉, R₅₃, R₆₁, R₆₄, R₆₆=are independently C,U or absent;
R₁, R₁₀, R₁₉, R₂₀, R₂₅, R₂₇, R₂₉, R₃₁, R₃₂, R₄₈, R₅₀, R₅₂, R₆₅=are independently G or absent;
R₇, R₁₂=are independently G,U or absent;
R₈, R₁₆, R₃₃, R₃₆, R₃₉, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I _{MET} (SEQ ID NO: 592),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₂₃=are absent;
R₁₄, R₃₅, R₄₀, R₅₇=are independently A or absent;
R₆₀=A,C or absent;
R₃₃, R₄₈, R₇₀=are independently A,C,G or absent;
R₁, R₃, R₄, R₅, R₆, R₁, R₁₂, R₁₆, R₁₇, R₂₁, R₂₂, R₂₆, R₂₇, R₂₉, R₃₀, R₃₁, R₃₂, R₄₂, R₄₄, R₄₅, R₄₆, R₄₉, R₅₀, R₅₈, R₆₂, R₆₃, R₆₆, R₆₇, R₆₅, R₆₉, R₇₁=are independently N or absent;
R₁₈, R₃₅, R₄₁, R₅₉, R₆₅=are independently A,C,U or absent;
R₉, R₁₅, R₅₁=are independently A,G or absent;
R₇, R₂₄, R₂₅, R₃₄, R₅₃, R₅₆=are independently A,G,U or absent;
R₇₂=A,U or absent;
R₃₇=C or absent;
R₁₀, R₅₅=are independently C,G or absent;
R₂, R₁₃, R₂₈, R₄₃, R₆₄=are independently C,G,U or absent;
R₃₆, R₆₁=are independently C,U or absent;
R₁₉, R₂₀, R₅₂=are independently G or absent;
R₈, R₃₉, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{MET}(SEQ ID NO: 593),
R₀-R₁-R₂-R₃-R₄-R₅-R₆-R₇-R₈-R₉-R₁₀-R₁₁-R₁₂-R₁₃-R₁₄-R₁₅-R₁₆-R₁-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₁₈, R₂₂, R₂₃=are absent
R₁₄, R₂₄, R₃₅, R₄₀, R₄₁, R₅₇, R₇₂=are independently A or absent;
R₅₉, R₆₀, R₆₂, R₆₅=are independently A,C or absent;
R₆, R₄₅, R₆₇=are independently A,C,G or absent;
R₄=N or absent;
R₂₁, R₄₂=are independently A,C,U or absent;
R₁, R₉, R₂₇, R₂₉, R₃₂, R₄₆, R₅₁=are independently A,G or absent;
R₁₇, R₄₉, R₅₃, R₅₆, R₅₈=are independently A,G,U or absent;
R₆3=A,U or absent;
R₃, R₁₃, R₃₇=are independently C or absent;
R₄₈, R₅₅, R₆₄, R₇₀=are independently C,G or absent;
R₂, R₅, R₆₆, R₆₈=are independently C,G,U or absent;
R₁, R₁₆, R₂₆, R₂₈, R₃₀, R₃₁, R₃₅, R₃₆, R₄₃, R₄₄, R₆₁, R₇₁=are independently C,U or absent;
R₁₀, R₁₂, R₁₅, R₁₉, R₂₀, R₂₅, R₃₃, R₅₂, R₆₉=are independently G or absent;
R₇, R₃₄, R₅₀=are independently G,U or absent;
R₈, R₃₉, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{MET}(SEQ ID NO: 594),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₂, R₆₅=are independently A,C or absent;
R₆, R₆₇=are independently A,C,G or absent;
R₄, R₂₁=are independently A,C,U or absent;
R₁, R₉, R₂₇, R₂₉, R₃₂, R₄₅, R₄₆, R₅₁=are independently A,G or absent;
R₁₇, R₅₆, R₅₈=are independently A,G,U or absent;
R₄₉, R₅₃, R₆₃=are independently A,U or absent;
R₃, R₁₃, R₂₆, R₃₇, R₄₃, R₆₀=are independently C or absent;
R₂, R₄₈, R₅₅, R₆₄, R₇₀=are independently C,G or absent;
R₅, R₆₆=are independently C,G,U or absent;
R₁₁, R₁₆, R₂₈, R₃₀, R₃₁, R₃₅, R₃₆, R₄₂, R₄₄, R₆₁, R₇₁=are independently C,U or absent;
R₁₀, R₁₂, R₁₅, R₁₉, R₂₀, R₂₅, R₃₃, R₅₂, R₆₉=are independently G or absent;
R₇, R₃₄, R₅₀, R₆₈=are independently G,U or absent;
R₈, R₃₉, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I _{LEU} (SEQ ID NO: 595),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃-R₃₆-R₃-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent;
R₃₈, R₅₇=are independently A or absent;
R₆₀=A,C or absent;
R₁, R₁₃, R₂₇, R₄₈, R₅₁, R₅₆=are independently A,C,G or absent;
R₂, R₃, R₄, R₅, R₆, R₇, R₉, R₁₀, R₁, R₂, R₁₆, R₃, R₂₆, R₂₈, R₂₉, R₃₀, R₃₁, R₃₂, R₃₃, R₃₄, R₃₇, R₄₁, R₄₂, R₄₃, R₄₄,
R₄₅, R₄₆, R₄₉, R₅₀, R₅₈, R₆₂, R₆₃, R₆₅, R₆₆, R₆₇, R₆₈, R₆₉, R₇₀=are independently N or absent;
R₁₇, R₁₅, R₂₁, R₂₂, R₂₅, R₃₅, R₅₅=are independently A,C,U or absent;
R₁₄, R₁₅, R₃₉, R₇₂=are independently A,G or absent;
R₂₄, R₄₀=are independently A,G,U or absent;
R₅₂, R₆₁, R₆₄, R₇₁=are independently C,G,U or absent;
R₃₆, R₅₃, R₅₉=are independently C,U or absent;
R₁₉=G or absent;
R₂₀=G,U or absent;
R₈, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{LEU} (SEQ ID NO: 596),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent
R₃₅, R₅₇, R₇₂=are independently A or absent;
R₆₀=A,C or absent;
R₄, R₅, R₄₈, R₅₀, R₅₆, R₆₉=are independently A,C,G or absent;
R₆, R₃₃, R₄₁, R₄₃, R₄₆, R₄₉, R₅₈, R₆₃, R₆₆, R₇₀=are independently N or absent;
R₁₁, R₁₂, R₁₇, R₂, R₂₂, R₂₅, R₃₁, R₃₇, R₄₄, R₅₅=are independently A,C,U or absent;
R₁, R₉, R₁₄, R₁₅, R₂₄, R₂₇, R₃₄, R₃₉=are independently A,G or absent;
R₇, R₂₉, R₃₂, R₄₀, R₄₅=are independently A,G,U or absent;
R₂₅=A,U or absent;
R₁₃=C,G or absent;
R₂, R₃, R₁₆, R₂₆, R₃0, R₅₂, R₆₂, R₆₄, R₆₅, R₆₇, R₆₈=are independently C,G,U or absent;
R₁₈, R₃₅, R₄₂, R₅₃, R₅₉, R₆₁, R₇₁=are independently C,U or absent;
R₁₉, R₅₁=are independently G or absent;
R₁₀, R₂₀=are independently G,U or absent;
R₅, R₂₃, R₃₆, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{LEU} (SEQ ID NO: 597),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Leu is:
R₀=absent
R₃₅, R₅₇, R₇₂=are independently A or absent;
R₆₀=A,C or absent;
R₄, R₈, R₄₈, R₈, R₅₆, R₅₈, R₆₉=are independently A,C,G or absent;
R₆, R₃₃, R₄₃, R₄₆, R₄₉, R₆₃, R₆₆, R₇₀=are independently N or absent;
R₁₁, R₁₂, R₁₇, R₂₁, R₂₂, R₂₅, R₃₁, R₃₇, R₄₁, R₄₄, R₅₅=are independently A,C,U or absent;
R₁, R₉, R₁₄, R₁₅, R₂₄, R₂₇, R₃₄, R₃₉=are independently A,G or absent;
R₇, R₂₉, R₃₂, R₄₀, R₄₅=are independently A,G,U or absent;
R₂₅=A,U or absent;
R₁₃=C,G or absent;
R₂, R₃, R₁₆, R₃₀, R₅₂, R₆₂, R₆₄, R₆₇, R₆₈=are independently C,G,U or absent;
R₁₅, R₃₅, R₄₂, R₅₃, R₅₉, R₆₁, R₆₅, R₇₁=are independently C,U or absent;
R₁₉, R₅₁=are independently G or absent;
R₁₀, R₂₀, R₂₆=are independently G,U or absent;
R₈, R₂₃, R₃₆, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I _{LYS} (SEQ ID NO: 598),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀=absent
R₁₄=A or absent;
R₄₀, R₄₁=are independently A,C or absent;
R₃₄, R₄₃, R₅₁=are independently A,C,G or absent;
R₁, R₂, R₃, R₄, R₅, R₆, R₇, R 1, R₁₂, R₁₆, R₂₁, R₂₆, R₃, R₃₁, R₃₂, R₄₄, R₄₅, R₄₆, R₄₈, R₄₉, R₅₀, R₅₈, R₆₂, R₆₃, R₆₅,
R₆₆, R₆₇, R₆₈, R₆₉, R₇₀=are independently N or absent;
R₁₃, R₁₇, R₅₉, R₇₁=are independently A,C,U or absent;
R₉, R₁₅, R₁₉, R₂₀, R₂₅, R₂₇, R₅₂, R₅₆=are independently A,G or absent;
R₂₄, R₂₉, R₇₂=are independently A,G,U or absent;
R₁₅, R₅₇=are independently A,U or absent;
R₁₀, R₃₃=are independently C,G or absent;
R₄₂, R₆₁, R₆₄=are independently C,G,U or absent;
R₂₈, R₃₅, R₃₆, R₃₇, R₅₃, R₅₈, R₆₀=are independently C,U or absent;
R₈, R₂₂, R₂₃, R₃₈, R₃₉, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{LYS} (SEQ ID NO: 599),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀, R₁₈, R₂₃=are absent
R₁₄=A or absent;
R₄₀, R₄₁, R₄₃=are independently A,C or absent;
R₃, R₇=are independently A,C,G or absent;
R₁, R₆, R₁₁, R₃₁, R₄₅, R₄₈, R₄₉, R₆₃, R₆₅, R₆₆, R₆₈=are independently N or absent;
R₂, R₁₂, R₁₃, R₁₇, R₄₄, R₆₇, R₇₁=are independently A,C,U or absent;
R₉, R₁₅, R₁₉, R₂₀, R₂₅, R₂₇, R₃₄, R₅₀, R₅₂, R₅₆, R₇₀, R₇₂=are independently A,G or absent;
R₅, R₂₄, R₂₆, R₂₉, R₃₂, R₄₆, R₆₉=are independently A,G,U or absent;
R₅₇=A,U or absent;
R₁₀, R₆₁=are independently C,G or absent;
R₄, R₁₆, R₂₁, R₃₀, R₅₈, R₆₄=are independently C,G,U or absent;
R₂₈, R₃₅, R₃₆, R₃₇, R₄₂, R₅₃, R₅₅, R₅₉, R₆₀, R₆₂=are independently C,U or absent;
R₃₃, R₅₁=are independently G or absent;
R₈=G,U or absent;
R₂₂, R₃₈, R₃₉, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{LYS} (SEQ ID NO: 600),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Lys is:
R₀, R₁₈, R₂₃=absent
R₉, R₁₄, R₃₄, R₄₁=are independently A or absent;
R₄₀=A,C or absent;
R₁, R₃, R₇, R₃1=are independently A,C,G or absent;
R₄₈, R₆₅, R₆₈=are independently N or absent;
R₂, R₁₃, R₁₇, R₄₄, R₆₃, R₆₆=are independently A,C,U or absent;
R₈, R₁₅, R₁₉, R₂₀, R₂₅, R₂₇, R₂₉, R₅₀, R₅₂, R₅₆, R₇₀, R₇₂=are independently A,G or absent;
R₆, R₂₄, R₃₂, R₄₉=are independently A,G,U or absent;
R₁₂, R₂₆, R₄₆, R₅₇=are independently A,U or absent;
R₁₁, R₂₈, R₃₅, R₄₃=are independently C or absent;
R₁₀, R₄₅, R₆₁=are independently C,G or absent;
R₄, R₂₁, R₆₄=are independently C,G,U or absent;
R₃₇, R₅₃, R₅₅, R₅₉, R₆₀, R₆₂, R₆₇, R₇₁=are independently C,U or absent;
R₃₃, R₅₁=are independently G or absent;
R₈, R₃₀, R₅₅, R₆₉=are independently G,U or absent;
R₁₆, R₂₂, R₃₆, R₃₈, R₃₉, R₄₂, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I _{PHE} (SEQ ID NO: 601),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂

wherein R is a ribonucleotide residue and the consensus for Phe is:
R₀, R₂₃=are absent
R₉, R₁₄, R₃₈, R₃₉, R₅₇, R₇₂=are independently A or absent;
R₇₁=A,C or absent;
R₄₁, R₇₀=are independently A,C,G or absent;
R₄, R₅, R₆, R₃₀, R₃₁, R₃₂, R₃₄, R₄₂, R₄₄, R₄₅, R₄₆, R₄₈, R₄₉, R₅₈, R₆₂, R₆₃, R₆₆, R₆₇, R₆₈, R₆₉=are
independently N or absent;
R₁₆, R₆₁, R₆₅=are independently A,C,U or absent;
R₁₅, R₂₆, R₂₇, R₂₉, R₄₀, R₅₆=are independently A,G or absent;
R₇, R₅₁=are independently A,G,U or absent;
R₂₂, R₂₄=are independently A,U or absent;
R₅₅, R₆₀=are independently C or absent;
R₂, R₃, R₂₁, R₃₃, R₄₃, R₅₀, R₆₄=are independently C,G,U or absent;
R₁₁, R₁₂, R₁₃, R₁₇, R₂₈, R₃₅, R₃₆, R₅₉=are independently C,U or absent;
R₁₀, R₁₉, R₂₀, R₂₅, R₃₇, R₅₂=are independently G or absent;
R₁=G,U or absent;
R₈, R₁₈, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II PHE (SEQ ID NO: 602),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₁₈, R₂₃=absent
R₁₄, R₂₄, R₃₈, R₃₉, R₅₇, R₇₂=are independently A or absent;
R₄₆, R₇₁=are independently A,C or absent;
R₄, R₇₀=are independently A,C,G or absent;
R₄₅=A,C,U or absent;
R₆, R₇, R₁₅, R₂₆, R₂₇, R₃₂, R₃₄, R₄₀, R₄₁, R₅₆, R₆₉=are independently A,G or absent;
R₂₉=A,G,U or absent;
R₅, R₉, R₆₇=are independently A,U or absent;
R₃₅, R₄₉, R₅₈, R₆₀=are independently C or absent;
R₂₁, R₄₃, R₆₂=are independently C,G or absent;
R₂, R₃₃, R₆₅=are independently C,G,U or absent;
R₃, R₁₁, R₁₂, R₁₃, R₂₈, R₃0, R₃₆, R₄₂, R₄₄, R₄₈, R₅₈, R₅₉, R₆₁, R₆₆=are independently C,U or absent;
R₁₀, R₁₉, R₂₀, R₂₅, R₃₇, R₅₁, R₅₂, R₆₃, R₆₄=are independently G or absent;
R₁, R₃₁, R₅₀=are independently G,U or absent;
R₈, R₁₆, R₁₇, R₂₂, R₅₃, R₅₄, R₆₅=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PHE (SEQ ID NO: 603),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Phe is:
R₀, R₁₈, R₂₂, R₂₃=absent
R₅, R₇, R₁₄, R₂₄, R₂₆, R₃₂, R₃₄, R₃₈, R₃₉, R₄₁, R₅₇, R₇₂=are independently A or absent;
R₄₆=A,C or absent;
R₇₀=A,C,G or absent;
R₄, R₆, R₁₈, R₅₆, R₆₉=are independently A,G or absent;
R₉, R₄₅=are independently A,U or absent;
R₂, R₁₁, R₁₃, R₃₅, R₄₃, R₄₉, R₅₈, R₆₀, R₆₈, R₇₁=are independently C or absent;
R₃₃=C,G or absent;
R₃, R₂₈, R₃₆, R₄₈, R₅₈, R₅₉, R₆₁=are independently C,U or absent;
R₁, R₁₀, R₁₉, R₂₀, R₂₁, R₂₅, R₂₇, R₂₉, R₃₇, R₄₀, R₅₁, R₅₂, R₆₂, R₆₃, R₆₄=are independently G or absent;
R₅, R₁₂, R₁₆, R₁₇, R₃₀, R₃₁, R₄₂, R₄₄, R₅₀, R₅₃, R₅₄, R₆₅, R₆₆, R₆₇=are independently U or absent;
[R₄₇]x=N or absent;

wherein, e.g., x=1-271 (e.g., x=1-250, x=1-225, x=1-200, x=1-175, x=1-150, x=1-125, 25 x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2,x=3,x=4,x=5,x=6, x=7,x=8,x=9,x=10,x=11,x=12,x=13,x=14,x=15,x=16,x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I _{PRO} (SEQ ID NO: 604),
R₀-R₁-R₂-R₃-R₄-R₅-R₆-R₇-R₈-R₉-R₁₀-R₁₁-R₁₂-R₁₃-R₁₄-R₁₅-R₁₆-R₁-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀=absent
R₁₄, R₅₇=are independently A or absent;
R₇₀, R₇₂=are independently A,C or absent;
R₉, R₂₆, R₂₇=are independently A,C,G or absent;
R₄, R₅, R₆, R₁₆, R₂₁, R₂₉, R₃₀, R₃₁, R₃₂, R₃₃, R₃₄, R₃₇, R₄₁, R₄₂, R₄₃, R₄₄, R₄₅, R₄₆, R₄₅, R₄₉, R₅₀, R₅₅, R₆₁, R₆₂,
R₆₃, R₆₄, R₆₆, R₆₇, R₆₈=are independently N or absent;
R₃₅, R₆₅=are independently A,C,U or absent;
R₂₄, R₄₀, R₅₆=are independently A,G or absent;
R₇, R₂₅, R₅₁=are independently A,G,U or absent;
R₅₅, R₆₀=are independently C or absent;
R₁, R₃, R₇₁=are independently C,G or absent;
R₁₁, R₁₂, R₂₀, R₆₉=are independently C,G,U or absent;
R₁₃, R₁₇, R₁₅, R₂₂, R₂₃, R₂₈, R₅₉=are independently C,U or absent;
R₁₀, R₁₅, R₁₉, R₃₈, R₃₉, R₅₂=are independently G or absent;
R₂=are independently G,U or absent;
R₅, R₃₆, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{PRO} (SEQ ID NO: 605),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀, R₁₇, R₁₈, R₂₂, R₂₃=absent;
R₁₄, R₄₅, R₅₆, R₅₇, R₅₈, R₆₅, R₆₈=are independently A or absent;
R₆₁=A,C,G or absent;
R₄₃=N or absent;
R₃₇=A, C,U or absent;
R₂₄, R₂₇, R₃₃, R₄₀, R₄₄, R₆₃=are independently A,G or absent;
R₃, R₁₂, R₃₀, R₃₂, R₄₈, R₅₈, R₆₀, R₇₀, R₇₁, R₇₂=are independently C or absent;
R₈, R₃₄, R₄₂, R₆₆=are independently C,G or absent;
R₂₀=C,G,U or absent;
R₃₅, R₄₁, R₄₉, R₆₂=are independently C,U or absent;
R₁, R₂, R₆, R₉, R₁₀, R₁₅, R₁₉, R₂₆, R₃₈, R₃₉, R₄₆, R₅₀, R₅₁, R₅₂, R₆₄, R₆₇, R₆₉=are independently G or absent;
R₁₁, R₁₆=are independently G,U or absent;
R₄, R₇, R₅, R₁₃, R₂₁, R₂₅, R₂, R₂₉, R₃₁, R₃₆, R₅₃, R₅₄, R₅₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{PRO} (SEQ ID NO: 606),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Pro is:
R₀, R₁₇, R₁₈, R₂₂, R₂₃=absent
R₁₄, R₄₅, R₅₆, R₅₇, R₅₈, R₆₅, R₆₈=are independently A or absent;
R₃₇=A,C,U or absent;
R₂₄, R₂₇, R₄₀=are independently A,G or absent;
R₃, R₅, R₁₂, R₃₀, R₃₂, R₄₈, R₄₉, R₅₅, R₆₀, R₆₁, R₆₂, R₆₆, R₇₀, R₇₁, R₇₂=are independently C or absent;
R₃₄, R₄₂=are independently C,G or absent;
R₄₃=C,G,U or absent;
R₄₁=C,U or absent;
R₁, R₂, R₆, R₉, R₁₀, R₁₅, R₁₉, R₂₀, R₂₆, R₃₃, R₃₅, R₃₉, R₄₄, R₄₆, R₅₀, R₅₁, R₅₂, R₆₃, R₆₄, R₆₇, R₆₉=are independently G or absent;
R₁₆=G,U or absent;
R₄, R₇, R₈, R₁₁, R₁₃, R₂₁, R₂₅, R₂₈, R₂₉, R₃₁, R₃₅, R₃₆, R₅₃, R₅₄, R₅₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Serine TREM Consensus Sequence

In an embodiment, a TREM disclosed herein comprises the sequence of Formula I _{SER} (SEQ ID NO: 607),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₅-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀=absent;
R₁₄, R₂₄, R₅₇=are independently A or absent;
R₄₁=A,C or absent;
R₂, R₃, R₄, R₅, R₆, R₇, R₉, R₁₀, R₁₁, R₁₂, R₁₃, R₁₆, R₂₁, R₂₅, R₂₆, R₂₇, R₂₈, R₃₀, R₃₁, R₃₂, R₃₃, R₃₄, R₃₇, R₄₂, R₄₃,
R₄₄, R₄₅, R₄₆, R₄₈, R₄₉, R₅₀, R₆₂, R₆₃, R₆₄, R₆₅, R₆₆, R₆₇, R₆₈, R₆₉, R₇0=are independently N or absent;
R₁₈=A,C,U or absent;
R₁₅, R₄₀, R₅₁, R₅₆=are independently A,G or absent;
R₁, R₂₉, R₅₈, R₇₂=are independently A,G,U or absent;
R₃₉=A,U or absent;
R₆₀=C or absent;
R₃₈=C,G or absent;
R₁₇, R₂₂, R₂₃, R₇₁=are independently C,G,U or absent;
R₈, R₃₅, R₃₆, R₅₅, R₅₉, R₆₁=are independently C,U or absent;
R₁₉, R₂₀=are independently G or absent;
R₅₂=G,U or absent;
R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{SER}(SEQ ID NO: 608),
R₀-R₁-R₂-R₃-R₄-R₅-R₆-R₇-R₈-R₉-R₁₀-R₁₁-R₁₂-R₁₃-R₁₄-R₁₅-R₁₆-R₁-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀, R₂₃=absent
R₁₄, R₂₄, R₄₁, R₅₇=are independently A or absent;
R₄₄=A,C or absent;
R₂₅, R₄₅, R₄₈=are independently A,C,G or absent;
R₂, R₃, R₄, R₅, R₃₇, R₅₀, R₆₂, R₆₆, R₆₇, R₆₉, R₇₀=are independently N or absent;
R₁₂, R₂₈, R₆₅=are independently A,C,U or absent;
R₉, R₁₅, R₂₉, R₃₄, R₄₀, R₅₆, R₆₃=are independently A,G or absent;
R₇, R₂₆, R₃₀, R₃₃, R₄₆, R₅₈, R₇₂=are independently A,G,U or absent;
R₃₉=A,U or absent;
R₁₁, R₃₅, R₆₀, R₆₁=are independently C or absent;
R₁₃, R₃₅=are independently C,G or absent;
R₆, R₁₇, R₃₁, R₄₃, R₆₄, R₆₈=are independently C,G,U or absent;
R₃₆, R₄₂, R₄₉, R₅₅, R₅₉, R₇₁=are independently C,U or absent;
R₁₀, R₁₉, R₂₀, R₂₇, R₅₁=are independently G or absent;
R₁, R₁₆, R₃₂, R₅₂=are independently G,U or absent;
R₈, R₁₅, R₂₁, R₂₂, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{SER}(SEQ ID NO: 609),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Ser is:
R₀, R₂₃=absent
R₁₄, R₂₄, R₄₁, R₅₇, R₅₈=are independently A or absent;
R₄₄=A,C or absent;
R₂₅, R₄₈=are independently A,C,G or absent;
R₂, R₃, R₅, R₃₇, R₆₆, R₆₇, R₆₉, R₇₀=are independently N or absent;
R₁₂, R₂₈, R₆₂=are independently A,C,U or absent;
R₇, R₉, R₁₅, R₂₉, R₃₃, R₃₄, R₄₀, R₄₅, R₅₆, R₆₃=are independently A,G or absent;
R₄, R₂₆, R₄₆, R₅₀=are independently A,G,U or absent;
R₃₀, R₃₉=are independently A,U or absent;
R₁₁, R₁₇, R₃₅, R₆₀, R₆₁=are independently C or absent;
R₁₃, R₃₅=are independently C,G or absent;
R₆, R₆₄=are independently C,G,U or absent;
R₃₁, R₄₂, R₄₃, R₄₉, R₅₅, R₅₉, R₆₈, R₆₅, R₇₁=are independently C,U or absent;
R₁₀, R₁₉, R₂₀, R₂₇, R₅₁, R₅₂=are independently G or absent;
R₁, R₁₆, R₃₂, R₇₂=are independently G,U or absent;
R₈, R₁₈, R₂₁, R₂₂, R₃₆, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I _{THR} (SEQ ID NO: 610),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₂₃=absent
R₁₄, R₄₁, R₅₇=are independently A or absent;
R₅₆, R₇₀=are independently A,C,G or absent;
R₄, R₅, R₆, R₇, R₁₂, R₁₆, R₂₆, R₃₀, R₃₁, R₃₂, R₃₄, R₃₇, R₄₂, R₄₄, R₄₅, R₄₆, R₄₅, R₄₉, R₅₀, R₅₅, R₆₂, R₆₃, R₆₄, R₆₅, R
R₆₆, R₆₇, R₆₈, R₇₂=are independently N or absent;
R₁₃, R₁₇, R₂₁, R₃₅, R₆₁=are independently A,C,U or absent;
R₁, R₉, R₂₄, R₂₇, R₂₉, R₆₉=are independently A,G or absent;
R₁₅, R₂₅, R₅₁=are independently A,G,U or absent;
R₄₀, R₅₃=are independently A,U or absent;
R₃₃, R₄₃=are independently C,G or absent;
R₂, R₃, R₅₉=are independently C,G,U or absent;
R₁₁, R₁₅, R₂₂, R₂₈, R₃₆, R₅₄, R₅₈, R₆₀, R₇₁=are independently C,U or absent;
R₁₀, R₂₀, R₃₅, R₅₂=are independently G or absent;
R₁₉=G,U or absent;
R₈, R₃₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{THR}(SEQ ID NO: 611),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₁₈, R₂₃=absent
R₁₄, R₄₁, R₅₇=are independently A or absent;
R₉, R₄₂, R₄₄, R₄₈, R₅₆, R₇₀=are independently A,C,G or absent;
R₄, R₆, R₁₂, R₂₆, R₄₉, R₅₈, R₆₃, R₆₄, R₆₆, R₆₈=are independently N or absent;
R₁₃, R₂₁, R₃₁, R₃₇, R₆₂=are independently A,C,U or absent;
R₁, R₁₅, R₂₄, R₂₇, R₂₉, R₄₆, R₅₁, R₆₉=are independently A,G or absent;
R₇, R₂₅, R₄₅, R₅₀, R₆₇=are independently A,G,U or absent;
R₄₀, R₅₃=are independently A,U or absent;
R₃₅=C or absent;
R₃₃, R₄₃=are independently C,G or absent;
R₂, R₃, R₅, R₁₆, R₃₂, R₃₄, R₅₉, R₆₅, R₇₂=are independently C,G,U or absent;
R₁₁, R₁₇, R₂₂, R₂₈, R₃₀, R₃₆, R₅₅, R₆₀, R₆₁, R₇₁=are independently C,U or absent;
R₁₀, R₁₉, R₂₀, R₃₅, R₅₂=are independently G or absent;
R₈, R₃₉, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100,x=1-75,x=1-50,x=1-40,x=1-30,x=1-29,x=1-28,x=1-27,x=1-26,x=1-25,x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{THR}(SEQ ID NO: 612),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Thr is:
R₀, R₁₈, R₂₃=absent
R₁₄, R₄₀, R₄₁, R₅₇=are independently A or absent;
R₄₄=A,C or absent;
R₉, R₄₂, R₄₈, R₅₆=are independently A,C,G or absent;
R₄, R₆, R₁₂, R₂₆, R₅₈, R₆₄, R₆₆, R₆₈=are independently N or absent;
R₁₃, R₂₁, R₃₁, R₃₇, R₄₉, R₆₂=are independently A,C,U or absent;
R₁, R₁₅, R₂₄, R₂₇, R₂₉, R₄₆, R₅₁, R₆₉=are independently A,G or absent;
R₇, R₂₅, R₄₅, R₅₀, R₆₃, R₆₇=are independently A,G,U or absent;
R₅₃=A,U or absent;
R₃₅=C or absent;
R₂, R₃₃, R₄₃, R₇₀=are independently C,G or absent;
R₈, R₁₆, R₃₄, R₅₉, R₆₅=are independently C,G,U or absent;
R₃, R₁₁, R₂₂, R₂₈, R₃₀, R₃₆, R₅₅, R₆₀, R₆₁, R₇₁=are independently C,U or absent;
R₁₀, R₁₉, R₂₀, R₃₅, R₅₂=are independently G or absent;
R₃₂=G,U or absent;
R₈, R₁₇, R₃₉, R₅₄, R₇₂=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I _{TRP} (SEQ ID NO: 613),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀=absent;
R₂₄, R₃₉, R₄₁, R₅₇=are independently A or absent;
R₂, R₃, R₂₆, R₂₇, R₄₀, R₄₈=are independently A,C,G or absent;
R₄, R₅, R₆, R₂₉, R₃₀, R₃₁, R₃₂, R₃₄, R₄₂, R₄₄, R₄₅, R₄₆, R₄₉, R₅₁, R₅₅, R₆₃, R₆₆, R₆₇, R₆₅=are independently
N or absent;
R₁₃, R₁₄, R₁₆, R₁₈, R₂₁, R₆₁, R₆₅, R₇₁=are independently A,C,U or absent;
R₁, R₉, R₁₀, R₁₅, R₃₃, R₅₀, R₅₆=are independently A,G or absent;
R₇, R₂₅, R₇₂=are independently A,G,U or absent;
R₃₇, R₃₈, R₅₈, R₆₀=are independently C or absent;
R₁₂, R₃₅, R₄₃, R₆₄, R₆₉, R₇₀=are independently C,G,U or absent;
R₁, R₁₇, R₂₂, R₂₅, R₅₉, R₆₂=are independently C,U or absent;
R₁₉, R₂₀, R₅₂=are independently G or absent;
R₈, R₂₃, R₃₆, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{TRP}(SEQ ID NO: 614),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A,C or absent;
R₆, R₄₄=are independently A,C,G or absent;
R₂₁=A,C,U or absent;
R₂, R₇, R₁₈, R₂₅, R₃₃, R₃₄, R₄₅, R₅₆, R₆₃=are independently A,G or absent;
R₅₈=A,G,U or absent;
R₄₆=A,U or absent;
R₃₇, R₃₈, R₅₈, R₆₀, R₆₂=are independently C or absent;
R₁₂, R₂₆, R₂, R₃₅, R₄₀, R₄₈, R₆₇=are independently C,G or absent;
R₃₂, R₄₃, R₆₅=are independently C,G,U or absent;
R₁₁, R₁₆, R₂₈, R₃₁, R₄₉, R₅₉, R₆₅, R₇₀=are independently C,U or absent;
R₁, R₉, R₁₀, R₁₉, R₂₀, R₅₀, R₅₂, R₆₉=are independently G or absent;
R₅, R₅, R₂₉, R₃₀, R₄₂, R₅₁, R₆₄, R₆₆=are independently G,U or absent;
R₁₇, R₃₆, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{TRP}(SEQ ID NO: 615),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A,C or absent;
R₆, R₄₄=are independently A,C,G or absent;
R₂₁=A,C,U or absent;
R₂, R₇, R₁₈, R₂₅, R₃₃, R₃₄, R₄₅, R₅₆, R₆₃=are independently A,G or absent;
R₅₈=A,G,U or absent;
R₄₆=A,U or absent;
R₃₇, R₃₈, R₅₈, R₆₀, R₆₂=are independently C or absent;
R₁₂, R₂₆, R₂₇, R₃₅, R₄₀, R₄₈, R₆₇=are independently C,G or absent;
R₃₂, R₄₃, R₆₅=are independently C,G,U or absent;
R₁₁, R₁₆, R₂₈, R₃₁, R₄₉, R₅₉, R₆₅, R₇₀=are independently C,U or absent;
R₁, R₉, R₁₀, R₁₉, R₂₀, R₅₀, R₅₂, R₆₉=are independently G or absent;
R₅, R₅, R₂₉, R₃₀, R₄₂, R₅₁, R₆₄, R₆₆=are independently G,U or absent;
R₁₇, R₃₆, R₅₃, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I _{TYR} (SEQ ID NO: 616),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀=absent
R₁₄, R₃₉, R₅₇=are independently A or absent;
R₄₁, R₄₈, R₅₁, R₇₁=are independently A,C,G or absent;
R₃, R₄, R₅, R₆, R₉, R₁₀, R₁₂, R₁₃, R₁₆, R₂₅, R₂₆, R₃₀, R₃₁, R₃₂, R₄₂, R₄₄, R₄₅, R₄₆, R₄₉, R₅₀, R₅₅, R₆₂, R₆₃, R₆₆,
R₆₇, R₆₈, R₆₉, R₇₀=are independently N or absent;
R₂₂, R₆₅=are independently A,C,U or absent;
R₁₅, R₂₄, R₂₇, R₃₃, R₃₇, R₄₀, R₅₆=are independently A,G or absent;
R₇, R₂₉, R₃₄, R₇₂=are independently A,G,U or absent;
R₂₃, R₅₃=are independently A,U or absent;
R₃₅, R₆₀=are independently C or absent;
R₂₀=C,G or absent;
R₁, R₂, R₂₅, R₆₁, R₆₄=are independently C,G,U or absent;
R₁₁, R₁₇, R₂₁, R₄₃, R₅₅=are independently C,U or absent;
R₁₉, R₅₂=are independently G or absent;
R₈, R₁₅, R₃₆, R₃₅, R₅₄, R₅₉=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{TYR}(SEQ ID NO: 617),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇=are independently A or absent;
R₄₄, R₆₉=are independently A,C or absent;
R₇₁=A,C,G or absent;
R₆₈=N or absent;
R₅₈=A,C,U or absent;
R₃₃, R₃₇, R₄₁, R₅₆, R₆₂, R₆₃=are independently A,G or absent;
R₆, R₂₉, R₇₂=are independently A,G,U or absent;
R₃₁, R₄₅, R₅₃=are independently A,U or absent;
R₁₃, R₃₅, R₄₉, R₆₀=are independently C or absent;
R₂₀, R₄₈, R₆₄, R₆₇, R₇₀=are independently C,G or absent;
R₁, R₂, R₅, R₁₆, R₆₆=are independently C,G,U or absent;
R₁₁, R₂₁, R₂₅, R₄₃, R₅₅, R₆₁=are independently C,U or absent;
R₁₀, R₁₅, R₁₉, R₂₅, R₂₇, R₄₀, R₅₁, R₅₂=are independently G or absent;
R₃, R₄, R₃₀, R₃₂, R₄₂, R₄₆=are independently G,U or absent;
R₅, R₁₂, R₁₇, R₂₂, R₃₆, R₃₈, R₅₀, R₅₄, R₅₉, R₆₅=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{TYR}(SEQ ID NO: 618),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇, R₇₂=are independently A or absent;
R₄₄, R₆₉=are independently A,C or absent;
R₇₁=A,C,G or absent;
R₃₇, R₄₁, R₅₆, R₆₂, R₆₃=are independently A,G or absent;
R₆, R₂₉, R₆₅=are independently A,G,U or absent;
R₃₁, R₄₅, R₅₅=are independently A,U or absent;
R₁₃, R₂₈, R₃₅, R₄₉, R₆₀, R₆₁=are independently C or absent;
R₈, R₄₈, R₆₄, R₆₇, R₇₀=are independently C,G or absent;
R₁, R₂=are independently C,G,U or absent;
R₁₁, R₁₆, R₂₁, R₄₃, R₅₅, R₆₆=are independently C,U or absent;
R₁₀, R₁₅, R₁₉, R₂₀, R₂₅, R₂₇, R₃₃, R₄₀, R₅₁, R₅₂=are independently G or absent;
R₃, R₄, R₃₀, R₃₂, R₄₂, R₄₆=are independently G,U or absent;
R₅, R₁₂, R₁₇, R₂₂, R₃₆, R₃₈, R₅₀, R₅₃, R₅₄, R₅₉, R₆₅=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, 30 x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Valine TREM Consensus Sequence

In an embodiment, a TREM disclosed herein comprises the sequence of Formula I _{VAL} (SEQ ID NO: 619),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₅-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₂₃=absent;
R₂₄, R₃₈, R₅₇=are independently A or absent;
R₉, R₇₂=are independently A,C,G or absent;
R₂, R₄, R₅, R₆, R₇, R₁₂, R₁₅, R₁₆, R₂₁, R₂₅, R₂₆, R₂₉, R₃₁, R₃₂, R₃₃, R₃₄, R₃₇, R₄₁, R₄₂, R₄₃, R₄₄, R₄₅, R₄₆, R₄₈, R₄
₉, R₅₀, R₅₈, R₆₁, R₆₂, R₆₃, R₆₄, R₆₅, R₆₆, R₆₇, R₆₈, R₆₉, R₇₀=are independently N or absent;
R₁₇, R₃₅, R₅₉=are independently A,C,U or absent;
R₁₀, R₁₄, R₂₇, R₄₀, R₅₂, R₅₆=are independently A,G or absent;
R₁, R₃, R₅₁, R₅₃=are independently A,G,U or absent;
R₃₉=C or absent;
R₁₃, R₃₀, R₅₅=are independently C,G,U or absent;
R₁₁, R₂₂, R₂₈, R₆₀, R₇₁=are independently C,U or absent;
R₁₉=G or absent;
R₂₀=G,U or absent;
R₈, R₁₈, R₃₆, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{VAL} (SEQ ID NO: 620),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₅-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₁₈, R₂₃=absent;
R₂₄, R₃₈, R₅₇=are independently A or absent;
R₆₄, R₇₀, R₇₂=are independently A,C,G or absent;
R₁₅, R₁₆, R₂₆, R₂₉, R₃₁, R₃₂, R₄₃, R₄₄, R₄₅, R₄₉, R₅₀, R₅₈, R₆₂, R₆₅=are independently N or absent;
R₆, R₁₇, R₃₄, R₃₇, R₄₁, R₅₉=are independently A,C,U or absent;
R₉, R₁₀, R₁₄, R₂₇, R₄₀, R₄₆, R₅₁, R₅₂, R₅₆=are independently A,G or absent;
R₇, R₁₂, R₂₅, R₃₃, R₅₃, R₆₃, R₆₆, R₆₈=are independently A,G,U or absent;
R₆₉=A,U or absent;
R₃₉=C or absent;
R₅, R₆₇=are independently C,G or absent;
R₂, R₄, R₁₃, R₄₈, R₅₅, R₆₁=are independently C,G,U or absent;
R₁₁, R₂₂, R₂₈, R₃₀, R₃₅, R₆₀, R₇₁=are independently C,U or absent;
R₁₉=G or absent;
R₁, R₃, R₂₀, R₄₂=are independently G,U or absent;
R₈, R₂₁, R₃₆, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{VAL} (SEQ ID NO: 621),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂

wherein R is a ribonucleotide residue and the consensus for Val is:
R₀, R₁₈, R₂₃=absent
R₂₄, R₃₈, R₄₀, R₅₇, R₇₂=are independently A or absent;
R₂₉, R₆₄, R₇₀=are independently A,C,G or absent;
R₄₉, R₅₀, R₆₂=are independently N or absent;
R₁₆, R₂₆, R₃₁, R₃₂, R₃₇, R₄₁, R₄₃, R₅₉, R₆₅=are independently A,C,U or absent;
R₉, R₁₄, R₂₇, R₄₆, R₅₂, R₅₆, R₆₆=are independently A,G or absent;
R₇, R₁₂, R₂₅, R₃₃, R₄₄, R₄₅, R₅₃, R₅₈, R₆₃, R₆₈=are independently A,G,U or absent;
R₆₉=A,U or absent;
R₃₉=C or absent;
R₈, R₆₇=are independently C,G or absent;
R₂, R₄, R₁₃, R₁₈, R₄₈, R₅₅=are independently C,G,U or absent;
R₆, R₁₁, R₂₂, R₂₈, R₃₀, R₃₄, R₃₅, R₆₀, R₆₁, R₇₁=are independently C,U or absent;
R₁₀, R₁₉, R₅₁=are independently G or absent;
R₁, R₃, R₂₀, R₄₂=are independently G,U or absent;
R₈, R₁₇, R₂₁, R₃₆, R₅₄=are independently U or absent;
[R₄₇]x=N or absent;

wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1,2, 3,4, 5, 6,7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) sequence encoded by a deoxyribonucleic acid (DNA) sequence disclosed in Table 5, e.g., any one of SEQ ID NOs: 452-561 disclosed in Table 5.

TABLE 5
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

### Method of Making TREMs

Methods for designing and constructing expression vectors and modifying a host cell, e.g., a fungal cell or cell line, a plant cell or cell line, or an insect cell or cell line, for production of a target (e.g., a TREM or an enzyme disclosed herein) use techniques known in the art. For example, a cell is genetically modified to express an exogenous TREM using cultured insect cells, plant cells, or fungal cells under the control of appropriate promoters, e.g., promoters that are active in insect cells, plant cells, or fungal cells, or promoters that are native to insect cells, plant cells, or fungal cells. Generally, recombinant methods may be used. See, in general, Pharmaceutical Biotechnology: Fundamentals and Applications, Springer (2013); Green and Sambrook (Eds.), Molecular Cloning: A Laboratory Manual (Fourth Edition), Cold Spring Harbor Laboratory Press (2012). For example, expression vectors may comprise non-transcribed elements such as an origin of replication, a suitable promoter and enhancer, and other 5′ or 3′ flanking non-transcribed sequences.

A method of making a TREM or TREM composition disclosed herein comprises use of a host cell, e.g., a fungal cell or cell line, a plant cell or cell line, or an insect cell or cell line, e.g., a modified host cell, expressing a TREM.

The host cell or modified host cell is cultured under conditions that allow for expression of the TREM. In an embodiment, the culture conditions can be modulated to increase expression of the TREM. The method of making a TREM further comprises purifying the expressed TREM from the host cell culture to produce a TREM composition. In an embodiment the TREM is a TREM fragment, e.g., a fragment of a tRNA encoded by a deoxyribonucleic acid sequence disclosed in Table 1. E.g., the TREM includes less than the full sequence of a tRNA, e.g., less than the full sequence of a tRNA with the same anticodon, from the same species as the subject being treated, or both.

In an embodiment, a method of making a TREM described herein comprises contacting (e.g., transducing, electroporating or transfecting) a host cell (e.g., as described herein, e.g., a modified host cell) with an exogenous nucleic acid described herein, e.g., a DNA or RNA, encoding a TREM under conditions sufficient to express the TREM. In an embodiment, the exogenous nucleic acid comprises an RNA (or DNA encoding an RNA) that comprises a ribonucleic acid (RNA) sequence of an RNA encoded by a DNA sequence disclosed in Table 1.

In an embodiment, the exogenous nucleic acid comprises an RNA sequence (or DNA encoding an RNA sequence) that is at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 1. In an embodiment, the exogenous nucleic acid comprises an RNA sequence (or DNA encoding an RNA sequence) that comprises at least 30 consecutive nucleotides of a ribonucleic acid (RNA) sequence encoded by a deoxyribonucleic acid (DNA) sequence disclosed in Table 1. In an embodiment, the exogenous nucleic acid comprises an RNA sequence (or DNA encoding an RNA sequence) that comprises at least 30 consecutive nucleotides of an RNA sequence at least 60%, 65%, 70%, 75%, 80%, 82%, 85%, 87%, 88%, 90%, 92%, 95%, 96%, 97%, 98%, 99% or 100% identical to an RNA sequence encoded by a DNA sequence provided in Table 1.

In an embodiment, the host cell is transduced with a virus (e.g., a lentivirus, adenovirus or retrovirus) expressing a TREM.

The expressed TREM can be purified from the host cell or host cell culture to produce a TREM composition, e.g., as described herein. Purification of the TREM can be performed by affinity purification, e.g., as described in the MACS Isolation of specific tRNA molecules protocol, or other methods known in the art.

In an embodiment, a method of making a TREM, e.g., a TREM composition, comprises contacting a TREM with a reagent, e.g., a capture reagent comprising a nucleic acid sequence complimentary with a TREM. A single capture reagent or a plurality of capture reagents can be used to make a TREM, e.g., a TREM composition. When a single capture reagent is used, the capture reagent can have at least 50%, 55%, 60%, 65%, 70%, 75%, 80%, 85%, 90%, or 95% complimentary sequence with the TREM. When a plurality of capture reagents is used, a composition of TREMs having a plurality of different TREMs can be made. In an embodiment, the capture reagent can be conjugated to an agent, e.g., biotin.

In an embodiment, the method comprises denaturing the TREM, e.g., prior to hybridization with the capture reagent. In an embodiment, the method comprises, renaturing the TREM, after hybridization and/or release from the capture reagent.

In an embodiment, a method of making a TREM, e.g., a TREM composition, comprises contacting a TREM with a reagent, e.g., a separation reagent, e.g., a chromatography reagent. In an embodiment, a chromatography reagent includes a column chromatography reagent, a planar chromatography reagent, a displacement chromatography reagent, a gas chromatography reagent, a liquid chromatography reagent, an affinity chromatography reagent, an ion-exchange chromatography reagent, or a size-exclusion chromatography reagent.

In an embodiment, a TREM made by any of the methods described herein can be: (i) charged with an amino acid, e.g., a cognate amino acid; (ii) charged with a non-cognate amino acid (e.g., a mischarged TREM (mTREM); or (iii) not charged with an amino acid, e.g., an uncharged TREM (uTREM).

In an embodiment, a TREM made by any of the methods described herein is an uncharged TREM (uTREM). In an embodiment, a method of making a uTREM comprises culturing the host cell in media that has a limited amount of one or more nutrients, e.g., the media is nutrient starved.

In an embodiment, a charged TREM, e.g., a TREM charged with a cognate AA or a non-cognate AA, can be uncharged, e.g., by dissociating the AA, e.g., by incubating the TREM at a high temperature.

### Exogenous Nucleic Acid Encoding a TREM or a TREM Fragment

In an embodiment, an exogenous nucleic acid, e.g., a DNA or RNA, encoding a TREM comprises a nucleic acid sequence comprising a nucleic acid sequence of one or a plurality of RNA sequences encoded by a DNA sequence disclosed in Table 1, e.g., any one of SEQ ID NOs: 1-451 as disclosed in Table 1. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, or 99% identical to an RNA sequence encoded by a DNA sequence disclosed in Table 1, e.g., any one of SEQ ID NOs: 1-451 as disclosed in Table 1. In one embodiment, the exogenous nucleic acid, e.g., a DNA or RNA, encoding a TREM comprises a nucleic acid sequence less than 100% identical to an RNA sequence encoded by a DNA sequence disclosed in Table 1, e.g., any one of SEQ ID NOs: 1-451 as disclosed in Table 1.

In an embodiment, an exogenous nucleic acid, e.g., a DNA or RNA, encoding a TREM comprises the nucleic acid sequence of an RNA sequence encoded by a DNA sequence disclosed in Table 1, e.g., any one of SEQ ID NOs: 1-451 as disclosed in Table 1. In an embodiment, an exogenous nucleic acid, e.g., a DNA or RNA, encoding a TREM comprises a nucleic acid sequence at least 60%, 65%, 70%, 75%, 80%, 85%, 90%, 95%, 96%, 97%, 98%, 99%, or 100% identical to a plurality of RNA sequences encoded by a DNA sequence disclosed in Table 1, e.g., any one of SEQ ID NOs: 1-451 as disclosed in Table 1. In an embodiment, an exogenous nucleic acid encoding a TREM comprises an RNA sequence encoded by a DNA sequence at least 60%, 65%, 70%, 75%, 80%, 85%, 90%, 95%, 96%, 97%, 98%, or 99% identical to a DNA sequence disclosed in Table 1, e.g., any one of SEQ ID NOs: 1-451 as disclosed in Table 1. In an embodiment, the exogenous nucleic acid encoding a TREM comprises an RNA sequence encoded by a DNA sequence less than 100% identical to a DNA sequence disclosed in Table 1, e.g., any one of SEQ ID NOs: 1-451 as disclosed in Table 1.

In an embodiment, an exogenous nucleic acid, e.g., a DNA or RNA, encoding a TREM comprises an RNA sequence of one or a plurality of TREM fragments, e.g., a fragment of an RNA encoded by a DNA sequence disclosed in Table 1, e.g., as described herein, e.g., a fragment of any one of SEQ ID NOs: 1-451 as disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence of an RNA encoded by a DNA sequence provided in Table 1, e.g., any one of SEQ ID NOs: 1-451 as disclosed in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence at least 60%, 65%, 70%, 75%, 80%, 85%, 90%, 95%, 96%, 97%, 98%, or 99% identical to an RNA encoded by a DNA sequence provided in Table 1. In an embodiment, a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of a nucleic acid sequence encoded by a DNA sequence at least 60%, 65%, 70%, 75%, 80%, 85%, 90%, 95%, 96%, 97%, 98%, or 99% identical to a DNA sequence provided in Table 1, e.g., any one of SEQ ID NOs: 1-451 as disclosed in Table 1.

In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence disclosed in Table 1 e.g., any one of SEQ ID NOs: 1-451 as disclosed in Table 1. In an embodiment, a TREM fragment comprises at least 5, 6, 7, 8, 9, 10, 11, 12, 13, 14, 15, 16, 17, 18, 19,20,21,22,2324,25,26,27,28,29 or30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1 e.g., any one of SEQ ID NOs: 1-451 as disclosed in Table 1. In an embodiment, a TREM fragment comprises at least 5, 6, 7, 8, 9, 10, 11, 12, 13, 14, 15, 16, 17, 18, 19, 20, 21, 22, 23 24, 25, 26, 27, 28, 29 or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1 e.g., any one of SEQ ID NOs: 1-451 as disclosed in Table 1.

In an embodiment, the exogenous nucleic acid comprises a DNA, which upon transcription, expresses a TREM.

In an embodiment, the exogenous nucleic acid comprises an RNA, which upon reverse transcription, results in a DNA which can be transcribed to provide the TREM.

In an embodiment, the exogenous nucleic acid encoding a TREM comprises: (i) a control region sequence; (ii) a sequence encoding a modified TREM; (iii) a sequence encoding more than one TREM; or (iv) a sequence other than a tRNA^{Met} sequence.

In an embodiment, the exogenous nucleic acid encoding a TREM comprises a promoter sequence. In an embodiment, the exogenous nucleic acid comprises an RNA Polymerase III (Pol III) recognition sequence, e.g., a Pol III binding sequence. In an embodiment, the promoter sequence comprises a U6 promoter sequence or fragment thereof. In an embodiment, the nucleic acid sequence comprises a promoter sequence that comprises a mutation, e.g., a promoter-up mutation, e.g., a mutation that increases transcription initiation, e.g., a mutation that increases TFIIIB binding. In an embodiment, the nucleic acid sequence comprises a promoter sequence which increases Pol III binding and results in increased tRNA production, e.g., TREM production.

Also disclosed herein is a plasmid comprising an exogenous nucleic acid encoding a TREM. In an embodiment, the plasmid comprises a promoter sequence, e.g., as described herein.

### TREM Composition

In an embodiment, a TREM composition, e.g., a TREM pharmaceutical composition, comprises a pharmaceutically acceptable excipient. Exemplary excipients include those provided in the FDA Inactive Ingredient Database (https://www.accessdata.fda.gov/scripts/cder/iig/index.Cfm).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM.

In an embodiment, a TREM composition, e.g., a TREM pharmaceutical composition, is at least 10, 20, 30, 40, 50, 60, 70, 80, 90, 95 or 99% dry weight TREMs.

In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition produced by any of the methods of making disclosed herein can be charged with an amino acid using an in vitro charging reaction as disclosed in Example 11, or as known in the art.

In an embodiment, a TREM composition comprise one or more species of TREMs. In an embodiment, a TREM composition comprises a single species of TREMs. In an embodiment, a TREM composition comprises a first TREM species and a second TREM species. In an embodiment, the TREM composition comprises X TREM species, wherein X=2, 3, 4, 5, 6, 7, 8, 9, or 10.

In an embodiment, the TREM has at least 70, 75, 80, 85, 90, or 95, or has 100%, identity with a sequence encoded by a nucleic acid in Table 1.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, e.g., a separation reagent described herein.

In some embodiments, a TREM composition can be formulated with water for injection.

In some embodiments, a TREM composition formulated with water for injection is suitable for pharmaceutical use, e.g., comprises a pharmaceutical TREM composition.

### TREM Purification

A TREM composition, e.g., a TREM pharmaceutical composition, may be purified from host cells by nucleotide purification techniques. In one embodiment, a TREM composition is purified by affinity purification, e.g., as described in the MACS Isolation of specific tRNA molecules protocol, or by a method described in Example 1-3 or 7. In one embodiment, a TREM composition is purified by liquid chromatography, e.g., reverse-phase ion-pair chromatography (IP-RP), ion-exchange chromatography (IE), affinity chromatography (AC), size-exclusion chromatography (SEC), and combinations thereof. See, e.g., Baronti et al. Analytical and Bioanalytical Chemistry (2018) 410:3239-3252.

In an embodiment, a TREM composition can be purified with a purification method comprising one, two or all of the following steps, e.g., in the order recited: (i) separating nucleic acids from protein to provide and RNA preparation; (ii) separating RNA with of less than 200 nt from larger RNA species; and/or (iii) separating a TREM from other RNA species by affinity-based separation, e.g., sequence affinity.

In an embodiment, steps (i)-(iii) are performed in the order recited.

In an embodiment, the purification method comprises step (i). In an embodiment, step (i) comprises extracting nucleic acids from protein in a sample, e.g., as described in Example 1. In an embodiment, the extraction method comprises a phenol chloroform extraction,

In an embodiment, the purification method comprises step (ii). In an embodiment, step (ii) is performed on a sample, after step (i). In an embodiment, step (ii) comprises separating RNA of less than a threshold size, e.g., less than 500 nt, 400 nt, 300 nt, 250 nt, or 200 nt in size from larger RNAs, e.g., using a miRNeasy kit as described in Example 1. In an embodiment, step (ii) comprises performing a salt precipitation, e.g., LiCl precipitation, to enrich for small RNAs (e.g., remove large RNAs), as described in Example 1. In an embodiment, separation of the RNA of less than a threshold size from larger RNAs, e.g., using a miRNeasy kit, is performed prior to the salt precipitation, e.g., LiCl precipitation. In an embodiment, step (ii) further comprises performing a desalting or buffer exchange step, e.g., with a G25 column.

In an embodiment, the purification method comprises step (iii). In an embodiment, step (iii) comprises performing an affinity-based separation to enrich for a TREM. In an embodiment, step (iii) is performed on a sample after step (i) and/or step (ii). In an embodiment, the affinity based separation comprises a sequence based separation, e.g., using a probe (e.g., oligo) comprising a sequence that binds to a TREM, e.g., as described in Example 1. In an embodiment, the probe (e.g., oligo) comprises one or more tags, e.g., a biotin tag and/or a fluorescent tag.

In an embodiment, the TREM purification method comprising steps (i), (ii) and (iii) results in a purified TREM composition. In an embodiment, a TREM composition purified according to a method described herein results in lesser RNA contaminants, e.g., as compared to a Trizol RNA extraction purification method.

### TREM Quality Control and Production Assessment

A TREM or a TREM composition, e.g., a pharmaceutical TREM composition, produced by any of the methods disclosed herein can be assessed for a characteristic associated with the TREM or the TREM preparation, such as purity, host cell protein or DNA content, endotoxin level, sterility, TREM concentration, TREM structure, functional activity of the TREM; or differential modification of the TREM, e.g., presence, location and/or level of a modification characteristic of a fungal (e.g., yeast), insect, or plant cell or cell line. Any of the above-mentioned characteristics can be evaluated by providing a value for the characteristic, e.g., by evaluating or testing the TREM, the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of HCP in the composition; (iii) decrease the amount of DNA in the composition; (iv) decrease the amount of fragments in the composition; (v) decrease the amount of endotoxins in the composition; (vi) increase the in vitro translation activity of the composition; (vii) increase the TREM concentration of the composition; or (viii) inactivate or remove any viral contaminants present in the composition, e.g., by reducing the pH of the composition or by filtration.

In an embodiment, the TREM (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has a host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml,
50 ng/ml, 60 ng/ml, 70 ng/ml, 80 ng/ml, 90 ng/ml, 100 ng/ml, 200 ng/ml, 300 ng/ml, 400 ng/ml, or
500 ng/ml.

In an embodiment, the TREM (e.g., TREM composition or an intermediate in the production of the TREM composition) has a host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, 100 ng, 200 ng, 300 ng, 400 ng, or 500 ng per milligram (mg) of the TREM composition.

In an embodiment, the TREM (e.g., TREM composition or an intermediate in the production of the TREM composition) has a DNA content, e.g., host cell DNA content, of less than 1 ng/ml, 5 ng/ml, 10 ng/ml, 15 ng/ml, 20 ng/ml, 25 ng/ml, 30 ng/ml, 35 ng/ml, 40 ng/ml,
50 ng/ml, 60 ng/ml, 70 ng/ml, 80 ng/ml, 90 ng/ml, 100 ng/ml, 200 ng/ml, 300 ng/ml, 400 ng/ml, or
500 ng/ml.

In an embodiment, the TREM (e.g., TREM composition or an intermediate in the production of the TREM composition) has less than 0.1%, 0,5%, 1%, 2%, 3%, 4%, 5%, 6%, 7%, 8%, 9%, 10%, 15%, 20%, 25% TREM fragments relative to full length TREMs.

In an embodiment, the TREM (e.g., TREM composition or an intermediate in the production of the TREM composition) has low levels or absence of endotoxins, e.g., a negative result as measured by the Limulus amebocyte lysate (LAL) test; In an embodiment, the TREM (e.g., TREM composition or an intermediate in the production of the TREM composition) has in-vitro translation activity, e.g., as measured by an assay described in Example 10.

In an embodiment, the TREM (e.g., TREM composition or an intermediate in the production of the TREM composition) has a TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP<71>, and/or the composition or preparation meets the standard of USP<85>.

In an embodiment, the TREM (e.g., TREM composition or an intermediate in the production of the TREM composition) comprises a differential modification, e.g., has a modification characteristic of a fungal (e.g., yeast), insect, or plant cell or cell line.

In an embodiment, the TREM (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### TREM Administration

An TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

### Vectors and Carriers.

In some embodiments the TREM, or TREM composition made according to a method described herein, e.g., by expression in a fungal, plant or insect host cell, followed by purification from the host cell, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition made according to a method described herein, e.g., by expression in a fungal, plant or insect host cell, followed by purification from the host cell, may comprise, may be formulated with, or may be delivered in, a carrier.

### Viral Vectors

The carrier may be a viral vector (e.g., a viral vector comprising a sequence encoding a TREM). The viral vector may be administered to a cell or to a subject (e.g., a human subject or animal model) to deliver a TREM, a TREM composition or a pharmaceutical TREM composition.

A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996).

Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM composition or a pharmaceutical TREM composition made according to a method described herein, e.g., by expression in a fungal, plant or insect host cell, followed by purification from the host cell, can be administered to a cell, e.g., a mammalian cell or human cell, in a vesicle or other membrane-based carrier.

In embodiments, a TREM or TREM composition, or pharmaceutical TREM composition made according to a method described herein, e.g., by expression in a fungal, plant or insect host cell, followed by purification from the host cell, is administered to a subject, e.g., a human, in or via a cell, vesicle or other membrane-based carrier. In one embodiment, the TREM or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the TREM or TREM compositions or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exemplary lipid nanoparticles are disclosed in International Application PCT/US204/053907, the entire content of which are hereby incorporated by reference. For example, an LNP described in paragraphs [403-406] or [410-413] of PCT/US2014/053907 can be used as a carrier for the TREM, TREM core fragment, TREM fragment, or TREM composition or pharmaceutical TREM composition described ehrein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the TREM, 30 TREM core fragment, TREM fragment, or TREM composition or pharmaceutical TREM compositions described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

IN some embodiment, s conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

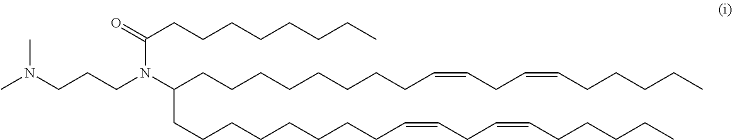 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a TREM composition described herein to the liver and/or hepatocyte cells.

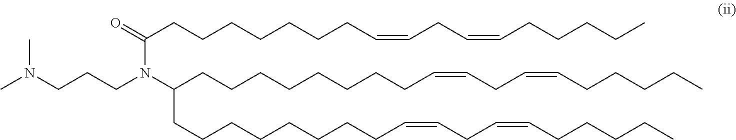 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a TREM composition described herein to the liver and/or hepatocyte cells.

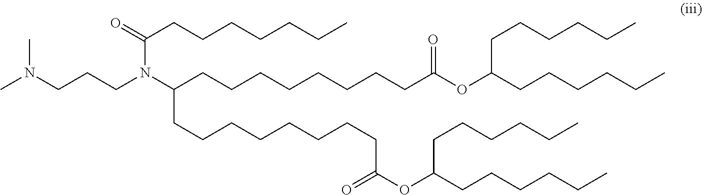 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a TREM composition described herein to the liver and/or hepatocyte cells.

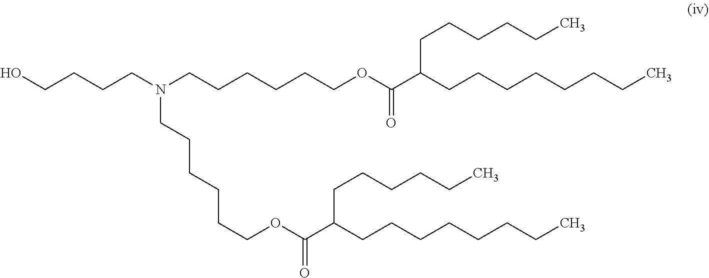 [SMILES: CCCCCCCCC(CCCCCC)C(=O)OCCCCCCN(CCCCO)CCCCCCOC(=O)C(CCCCCC)CCCCCCCC]

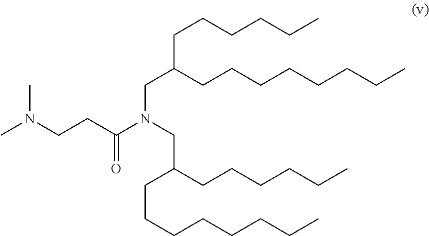 [SMILES: CCCCCCCCC(CCCCCC)CN(CC(CCCCCC)CCCCCCCC)C(=O)CCN(C)C]

In some embodiments an LNP comprising Formula (v) is used to deliver a TREM composition described herein to the liver and/or hepatocyte cells.

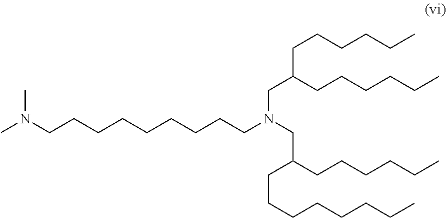 [SMILES: CCCCCCCCC(CCCCCC)CN(CCCCCCCCCN(C)C)CC(CCCCCC)CCCCCC]

In some embodiments an LNP comprising Formula (vi) is used to deliver a TREM composition described herein to the liver and/or hepatocyte cells.

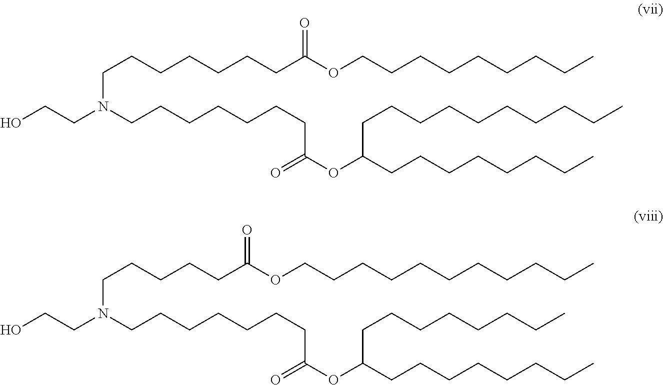 [SMILES: CCCCCCCCCCC(CCCCCCCC)OC(=O)CCCCCCCN(CCO)CCCCCCCC(=O)OCCCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a TREM composition described herein to the liver and/or hepatocyte cells.

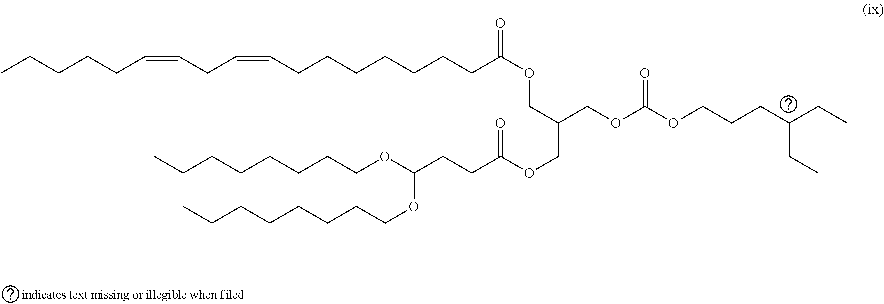 [SMILES: CCCCC/C=C\C/C=C\CCCCCCCC(=O)OCC(COC(=O)CCC(OCCCCCCCC)OCCCCCCCC)COC(=O)OCCCC(CC)CC]

In some embodiments an LNP comprising Formula (ix) is used to deliver a TREM composition described herein to the liver and/or hepatocyte cells.

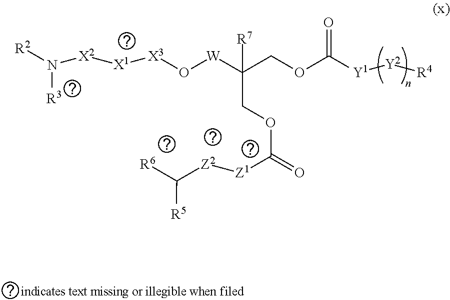

wherein X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

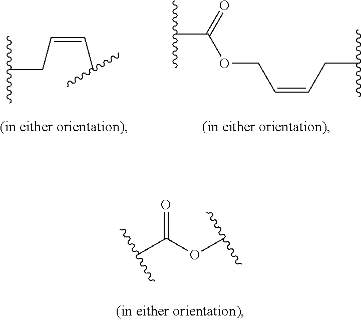 [SMILES: */C=C\C* ; *C/C=C\COC(*)=O ; *OC(*)=O]

n is 0 to 3, R⁴ is Ci-15 alkyl, Z¹ is Ci-6 alkylene or a direct bond, Z² is

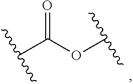 [SMILES: *OC(*)=O]

(in either orientation) or absent, provided that Z¹ is a direct bond, Z² is absent; R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is 0, X² is linear C3 alkylene, X³ is C(=0), Y¹ is linear Ce alkylene, (Y²)n-R⁴ is:

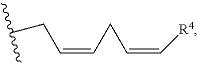

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a TREM composition described herein to the liver and/or hepatocyte cells.

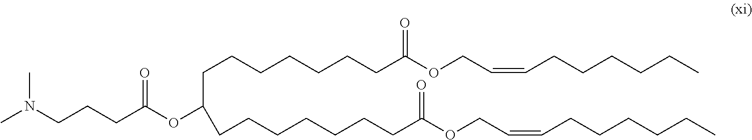 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a TREM composition described herein to the liver and/or hepatocyte cells.

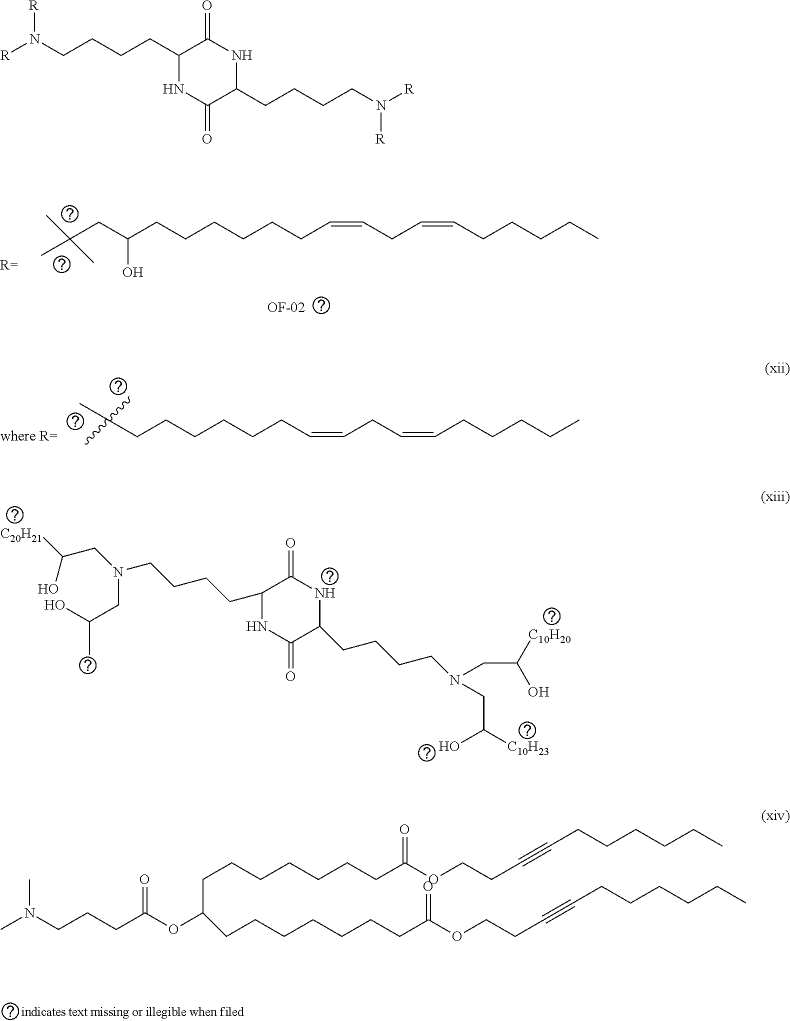

In some embodiments an LNP comprises a compound of Formula (xiii) and a compound of Formula (xiv).

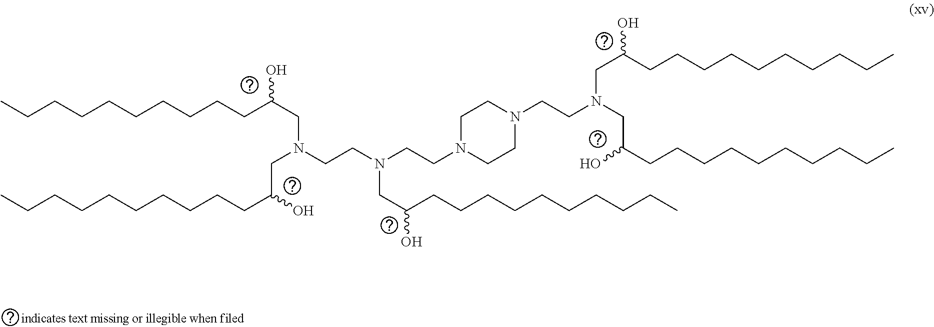 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a TREM composition described herein to the liver and/or hepatocyte cells.

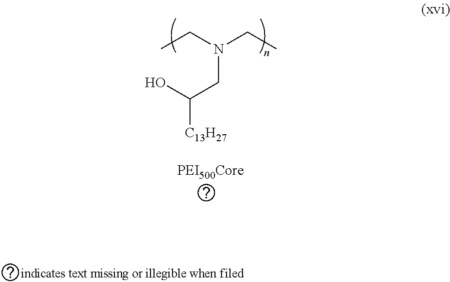

In some embodiments an LNP comprising a formulation of Formula (xvi) is used to deliver a TREM composition described herein to the lung endothelial cells.

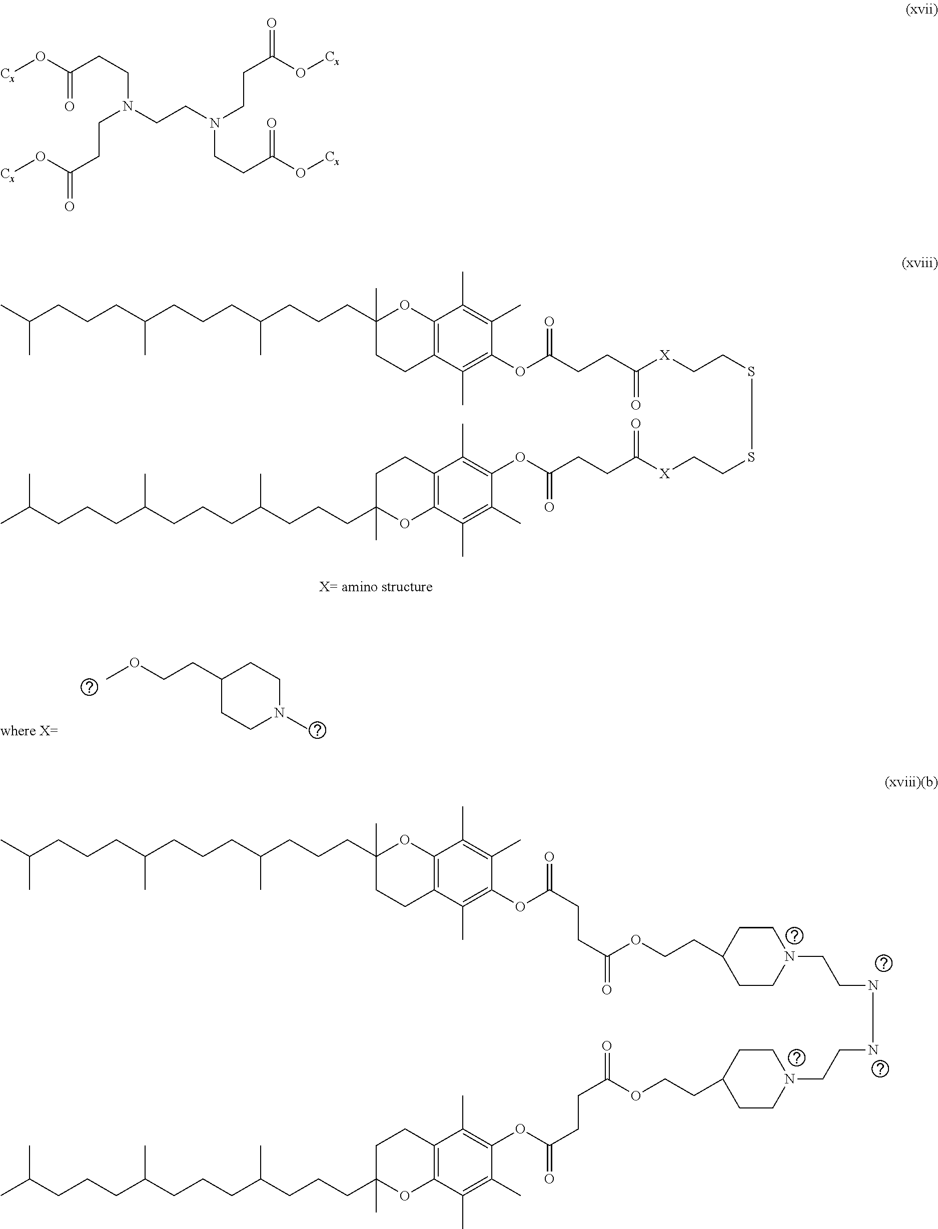

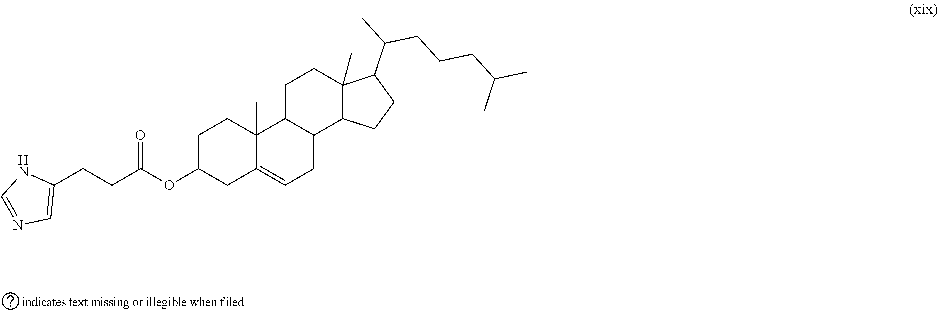

In some embodiments, a lipid compound used to form lipid nanoparticles for the delivery of compositions described herein, e.g., a TREM described herein is made by one of the following reactions:

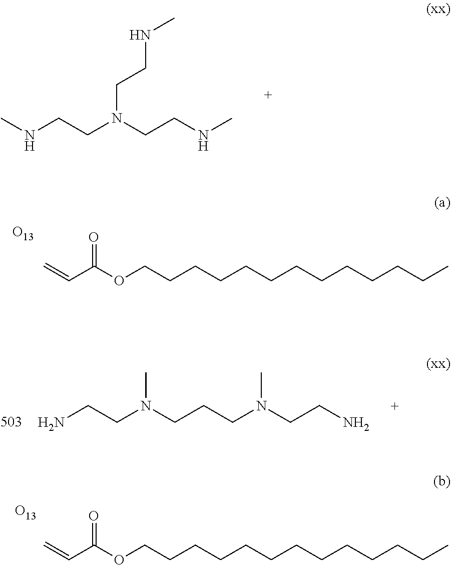

In some embodiments, a composition described herein (e.g., TREM composition) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety).

In some embodiments, the ionizable lipid is 9Z, 12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety).

In some embodiments, the ionizable lipid is Di((Z)-non-2-en-1-yl) 9-((4-dimethylamino)-butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10-R, 13-R, 17-R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a TREM described herein, encapsulated within or associated with the lipid nanoparticle. In some embodiments, the TREM is co-formulated with the cationic lipid. The TREM may be adsorbed to the surface of an LNP, e.g., an LNP comprising a cationic lipid. In some embodiments, the TREM may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a TREM.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z, 9Z, 28Z, 3 1Z)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z, 16Z)-A,A-dimethyl-3-nonyldocosa-13, 16-dien-1-amine (Compound
32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-0-dimethyl 25 PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

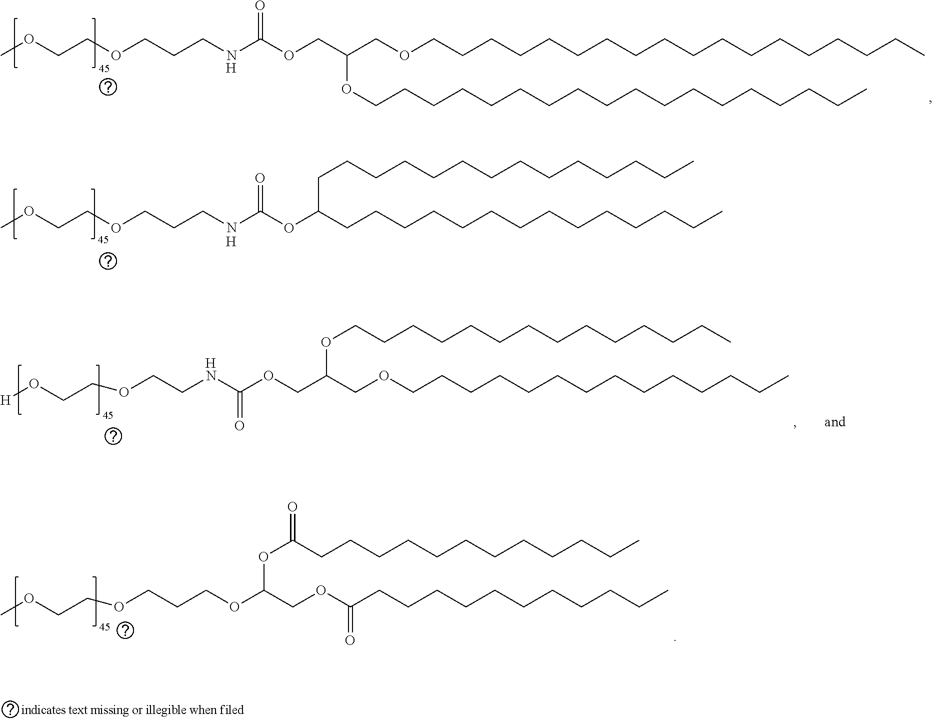

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5: 1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5: 1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z, 12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10 s of nm and 100 s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a TREM describes the amount of TREM that is encapsulated or otherwise associated with a LNP after preparation, relative to the initial amount provided. The encapsulation efficiency is desirably high (e.g., close to 100%). The encapsulation efficiency may be measured, for example, by comparing the amount of TREM in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free TREM in a solution. For the lipid nanoparticles described herein, the encapsulation efficiency of a TREM may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exosomes can also be used as drug delivery vehicles for the TREM or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM or TREM composition, or pharmaceutical TREM composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the TREM or TREM composition, or pharmaceutical TREM composition described herein.

Virosomes and virus-like particles (VLPs) can also be used as carriers to deliver a TREM, TREM core fragment, TREM fragment, or TREM composition, or pharmaceutical TREM composition described herein to targeted cells.

Plant nanovesicles, e.g., as described in WO2011097480A1, WO2013070324A1, or WO2017004526A1 can also be used as carriers to deliver the TREM, TREM core fragment, TREM fragment, or TREM composition, or pharmaceutical TREM composition described herein.

Delivery without a carrier A TREM, a TREM core fragment or a TREM fragment, a TREM composition or a pharmaceutical TREM composition described herein can be administered to a cell without a carrier, e.g., via naked delivery of the TREM, a TREM core fragment or a TREM fragment, a TREM composition or a pharmaceutical TREM composition.

In some embodiments, naked delivery as used herein refers to delivery without a carrier.

In some embodiments, delivery without a carrier, e.g., naked delivery, comprises delivery with a moiety, e.g., a targeting peptide.

In some embodiments, a TREM, a TREM core fragment or a TREM fragment, or TREM composition, or pharmaceutical TREM composition described herein is delivered to a cell without a carrier, e.g., via naked delivery. In some embodiments, the delivery without a carrier, e.g., naked delivery, comprises delivery with a moiety, e.g., a targeting peptide.

### Use of TREMs

A TREM composition (e.g., a pharmaceutical TREM composition described herein) can modulate a function in a cell, tissue or subject. In embodiments, a TREM composition (e.g., a pharmaceutical TREM composition) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein stability; protein transduction; protein compartmentalization. A parameter may be modulated, e.g., by at least 5% (e.g., at least 10%, 15%, 20%, 25%, 30%, 40%. 50%. 60%. 70%, 80%, 90%, 100%, 150%, 200% or more) compared to a reference tissue, cell or subject (e.g., a healthy, wild-type or control cell, tissue or subject).

All references and publications cited herein are hereby incorporated by reference.

### Enumerated Embodiments

1. A method of making a purified tRNA effector molecule (TREM) composition, e.g., a TREM pharmaceutical composition, comprising:
   providing a host cell, e.g., a fungal cell or cell line, an insect cell or cell line, or a plant, plant cell or cell line, comprising an exogenous nucleic acid, e.g., a DNA or RNA, encoding the TREM;
   maintaining the host cell under conditions sufficient to express the TREM;
   purifying the TREM from the host cell, e.g., according to a method described herein; and
   formulating the purified TREM as a pharmaceutical composition, e.g., by combining the TREM with a pharmaceutical excipient,
   thereby making the TREM composition.
2. The method of embodiment 1, wherein the nucleic acid comprises an RNA, which upon reverse transcription, results in a DNA which can be transcribed into the TREM.
3. The method of embodiment 1 or 2, wherein the nucleic acid comprises an RNA sequence at least 90% identical to an RNA sequence encoded by a DNA sequence listed in Table 1, or a fragment or functional fragment thereof.
4. The method of embodiment 1 or 2, wherein the nucleic acid comprises an RNA sequence comprising a consensus sequence provided herein.
5. The method of any one of the preceding embodiments, wherein the host cell comprises a fungal cell or cell line.
6. The method of embodiment 5, wherein the fungal cell or cell line is chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.
7. The method of embodiment 5 or 6, wherein the fungal cell or cell line is a Saccharomyces cell or cell line.
8. The method of embodiment 5 or 6, wherein the the fungal cell or cell line is a Saccharomyces cerevisiae fungal cell or cell line.
9. The method of embodiment 5 or 6, wherein the the fungal cell or cell line is a Schizosaccharomyces pombe fungal cell or cell line.
10. The method of embodiment 5 or 6, wherein the fungal cell or cell line is a Candida cylindracea fungal cell or cell line.
11. The method of embodiment 5 or 6, wherein the fungal cell or cell line is a Candida albicans fungal cell or cell line.
12. The method of embodiment 5 or 6, wherein the fungal cell or cell line is a Neurospora crassa fungal cell or cell line.
13. The method of embodiment 5 or 6, wherein the fungal cell or cell line is a Pichia jadinii fungal cell or cell line.
14. The method of any one of embodiments 1-5, wherein the host cell comprises an insect cell or cell line.
15. The method of embodiment 14, wherein the insect cell or cell line is chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.
16. The method of embodiment 14 or 15, wherein the insect cell is a Spodoptera frugiperda cell, optionally wherein the Spodoptera frugiperda cell is an Sf9 cell.
17. The method of embodiment 14 or 15, wherein the insect cell is a Trichoplusia ni cell, optionally wherein the insect cell is a H5 cell (High Five™, Invitrogen, Sorrento, CA).
18. The method of any one of embodiments 1-5, wherein the host cell comprises a plant, a plant cell or cell line.
19. The method of embodiment 18, wherein the host plant, plant cell or cell line is a monocotyledonous plant, cell or cell line.
20. The method of embodiment 18, wherein the host plant, plant cell or cell line is a dicotyledonous plant, cell or cell line.
21. The method of any one of claims 18-20, wherein the plant, plant cell or cell line is a plant, cell or cell line chosen from wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.
22. The method of embodiment 21, wherein the plant, cell or cell line is an Arabidopsis plant, cell or cell line.
23. The method of claim 22, wherein the Arabidopsis plant, cell or cell line is an A. thaliana plant, cell or cell line.
24. The method of any one of the preceding embodiments, wherein the purification step comprises one, two or all of the following steps, e.g., in the order recited:
   (i) separating nucleic acids from cellular debris to provide an RNA preparation;
   (ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
   (iii) separating a TREM from other RNA species in the small RNA preparation by affinity-based separation, e.g., sequence affinity-based separation.
25. A composition comprising a purified tRNA effector molecule (TREM) (e.g., a purified TREM composition made according to a method described herein), comprising:
   (i) an RNA sequence at least 90% identical to an RNA sequence encoded by a DNA sequence listed in Table 1, or a fragment or functional fragment thereof; or
   (ii) an RNA sequence comprising a consensus sequence provided herein.
26. A GMP-grade, recombinant TREM composition (e.g., a TREM composition made in compliance with cGMP, and/or in accordance with similar requirements) comprising:
   (i) an RNA sequence at least 90% identical to an RNA sequence encoded by a DNA sequence listed in Table 1, or a fragment or functional fragment thereof; or
   (ii) an RNA sequence comprising a consensus sequence provided herein.
27. The TREM composition of embodiment 25 or 26, wherein the composition comprises one or more, e.g., a plurality, of TREMs.
28. The TREM composition of any one of embodiments 25 to 27, wherein the composition comprises at least 1, 2, 3, 4, 5, 6, 7, 8, 9 or 10 species of TREMs.
29. The TREM composition of any one of embodiments 25-28, wherein the TREM composition (or an intermediate in the production of a TREM composition) comprises one or more of the following characteristics:
   (i) purity of at least 30%, 40%, 50%, 60%, 70%, 80%, 85%, 90%, 91%, 92%, 93%, 94%, 95%, 96%, 97%, 98%, or 99%;
   (ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
   (iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (v) less than 0.1%, 0.5%, 1%, 2%, 3%, 4%, 5%, 6%, 7%, 8%, 9% or 10% TREM fragments relative to full length TREMs;
   (vi) low levels or absence of endotoxins, e.g., a negative result as measured by the Limulus amebocyte lysate (LAL) test;
   (vii) in-vitro translation activity, e.g., as measured by an assay described in Example 10;
   (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
   (ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>;
   (x) viral contamination, e.g., the composition or preparation has an absence of, or an undetectable level of viral contamination; or
   (xi) differential modification, e.g., comprising a modification characteristic of a fungal cell or cell line; an insect cell or cell line; or a plant, plant cell or cell line.
30. A method of modulating a tRNA pool in a cell, e.g., a mammalian cell, comprising:
   providing a purified TREM composition, e.g., made according to a method described herein, and contacting the cell with the TREM composition,
   thereby modulating the tRNA pool in the cell.
31. The method of embodiment 30, wherein the TREM composition is made by:
   providing a host cell, e.g., a fungal cell or cell line, an insect cell or cell line, or a plant, plant cell or cell line, comprising an exogenous nucleic acid, e.g., a DNA or RNA, encoding the TREM;
   maintaining the cell under conditions sufficient to express the TREM; and/or
   purifying the TREM from the host cell, e.g., according to a method described herein.
32. The method of embodiment 30 or 31, wherein the host cell is a fungal cell or cell line, e.g., as described herein.
33. The method of embodiment 30 or 31, wherein the host cell is an insect cell or cell line, e.g., as described herein.
34. The method of embodiment 30 or 31, wherein the host cell is a plant, plant cell or cell line, e.g., as described herein.
35. The method of any one of claims 31-34, wherein the purification step comprises one, two or all of the following steps, e.g., in the order recited:
   (i) separating nucleic acids from cellular debris to provide an RNA preparation;
   (ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
   (iii) separating a TREM from other RNA species in the small RNA preparation by affinity-based separation, e.g., sequence affinity-based separation.
36. The method of any one of claims 30-35, wherein the TREM comprises:
   (i) an RNA sequence at least 80% identical to an RNA sequence encoded by a DNA sequence listed in Table 1, or a fragment or functional fragment thereof; or
   (ii) an RNA sequence comprising a consensus sequence provided herein.
37. A method of making a tRNA effector molecule (TREM) composition, comprising:
   (a) providing a host cell, e.g., a fungal cell or cell line, an insect cell or cell line, or a plant, plant cell or cell line, comprising exogenous nucleic acid, e.g., a DNA or RNA, encoding a TREM under conditions sufficient to express the TREM, and
   (b) purifying the expressed TREM from the host cell to produce a TREM composition, thereby making the TREM composition.
38. A method of making a pharmaceutical TREM composition comprising: combining
   a) a TREM, e.g., a purified TREM composition, e.g., a TREM composition made by a method described herein; and
   b) a pharmaceutically acceptable component, e.g., an excipient,
   thereby making a pharmaceutical TREM composition.
39. A method of making a purified tRNA effector molecule (TREM) pharmaceutical composition, comprising:
   purifying the TREM from a host cell, e.g., a fungal cell or cell line, an insect cell or cell line, or a plant, plant cell or cell line;
   formulating the purified TREM as a pharmaceutical composition, e.g., by combining the TREM with a pharmaceutical excipient,
   thereby making the TREM pharmaceutical composition.
40. A method of making a TREM composition, comprising:
   contacting a TREM containing a reaction mixture with a reagent, e.g., a capture reagent or a separation reagent, comprising a nucleic acid sequence complimentary with a TREM;
   thereby making a TREM composition.
41. A method of making a pharmaceutical composition, comprising:
   a) providing a purified TREM composition, e.g., a purified TREM composition made by culturing a host cell, e.g., a fungal cell or cell line, an insect cell or cell line, or a plant, plant cell or cell line, comprising DNA or RNA encoding a TREM under conditions sufficient to express the TREM, and purifying the expressed TREM from the host cell culture to produce a purified TREM composition,
   b) providing a value, e.g., by evaluating or testing, for one or more of the following characteristics of the purified TREM composition:
      (i) purity of at least 30%, 40%, 50%, 60%, 70%, 80%, 85%, 90%, 91%, 92%, 93%, 94%, 95%, 96%, 97%, 98%, or 99%;
      (ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
      (iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
      (iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
      (v) less than 0.1%, 0.5%, 1%, 2%, 3%, 4%, 5%, 6%, 7%, 8%, 9% or 10% TREM fragments relative to full length TREMs;
      (vi) low levels or absence of endotoxins, e.g., a negative result as measured by the Limulus amebocyte lysate (LAL) test;
      (vii) in-vitro translation activity, e.g., as measured by an assay described in Example 10; (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
      (ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>;
      (x) viral contamination, e.g., the composition or preparation has an absence of, or an undetectable level of viral contamination, or
      (xi) differential modification, e.g., comprising a modification characteristic of a fungal cell or cell line; an insect cell or cell line; or a plant, plant cell or cell line,
   c) optionally, formulating the purified TREM composition as a pharmaceutical drug product (e.g., combining the TREM composition with a pharmaceutical excipient) if it meets a reference criteria for the one or more characteristics, thereby making a pharmaceutical composition.
42. A pharmaceutical tRNA effector molecule (TREM) composition, comprising
   (i) an RNA sequence at least 80% identical to an RNA sequence encoded by a DNA sequence listed in Table 1, or a fragment or functional fragment thereof; or
   (ii) an RNA sequence comprising a consensus sequence provided herein.
43. A recombinant TREM composition of at least 0.5 g, 1 g, 2 g, 3 g, 4 g, 5 g, 6 g, 7 g, 8 g, 9 g, 10 g, 15 g, 20 g, 30 g, 40 g, 50 g, 100 g, 200 g, 300 g, 400 g or 500 g.
44. A recombinant TREM composition of between 0.5 g to 500 g, between 0.5 g to 400 g, between 0.5 g to 300 g, between 0.5 g to 200 g, between 0.5 g to 100 g, between 0.5 g to 50 g, between 0.5 g to 40 g, between 0.5 g to 30 g, between 0.5 g to 20 g, between 0.5 g to 10 g, between 0.5 g to 9 g, between 0.5 g to 8 g, between 0.5 g to 7 g, between 0.5 g to 6 g, between 0.5 g to 5 g, between 0.5 g to 4 g, between 0.5 g to 3 g, between 0.5 g to 2 g, between 0.5 g to 1 g, between 1 g to
500 g, between 2 g to 500 g, between 5 g to 500 g, between 10 g to 500 g, between 20 g to 500 g, between 30 g to 500 g, between 40 g to 500 g, between 50 g to 500 g, between 100 g to 500 g, between 200 g to 500 g, between 300 g to 500 g, or between 400 g to 500 g.
45. A TREM composition comprising a consensus sequence of Formula I zzz,
   R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein:
   R is a ribonucleotide residue;
      (i) zzz indicates any of the twenty amino acids;
      (ii) Formula I corresponds to all species; and
      (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3,x=4,x=5,x=6,x=7,x=8,x=9,x=10,x=11,x=12,x=13,x=14,x=15,x=16,x=17,x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
46. A TREM composition comprising a consensus sequence of Formula II zzz,
   R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein:
      R is a ribonucleotide residue;
         (i) zzz indicates any of the twenty amino acids;
         (ii) Formula II corresponds to mammals; and
         (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
47. A TREM composition comprising a consensus sequence of Formula III zzz,
   R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
   wherein:
      R is a ribonucleotide residue;
         (i) zzz indicates any of the twenty amino acids;
         (ii) Formula III corresponds to humans; and
         (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).
48. A method of contacting a cell, tissue, or subject, e.g., a mammalian cell, tissue or subject, with a TREM, comprising
   contacting the cell, tissue or subject with a purified TREM composition, thereby contacting a cell, tissue, or subject with the TREM.
49. A method of delivering a TREM to a cell, tissue, or subject, e.g., a mammalian cell, tissue or subject, comprising:
   providing a cell, tissue, or subject, and contacting the cell, tissue, or subject, with a TREM composition, e.g., a purified TREM composition, e.g., a pharmaceutical TREM composition.
50. A method of treating a subject, e.g., modulating the metabolism, e.g., the translational capacity of a cell, in a subject, e.g., a mammal, comprising:
   providing, e.g., administering to the subject, an exogenous nucleic acid, e.g., a DNA or RNA, which encodes a TREM,
   thereby treating the subject.
51. A cell, e.g., a mammalian cell, comprising a TREM made according to a method of making a TREM disclosed herein.
52. A reaction mixture comprising a TREM and a reagent, e.g., a capture reagent, or a separation reagent.
53. A bioreactor comprising a plurality of host cells described herein comprising exogenous DNA or RNA.
54. A method of evaluating a composition of TREM, e.g., a GMP-grade TREM (i.e., a TREM made in compliance with cGMP, and/or in accordance with similar requirements), comprising acquiring a value for one or more of the following characteristics of the purified TREM composition:
   (i) purity of at least 30%, 40%, 50%, 60%, 70%, 80%, 85%, 90%, 91%, 92%, 93%, 94%, 95%, 96%, 97%, 98%, or 99%;
   (ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
   (iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
   (v) less than 0.1%, 0.5%, 1%, 2%, 3%, 4%, 5%, 6%, 7%, 8%, 9% or 10% TREM fragments relative to full length TREMs;
   (vi) low levels or absence of endotoxins, e.g., a negative result as measured by the Limulus amebocyte lysate (LAL) test;
   (vii) in-vitro translation activity, e.g., as measured by an assay described in Example 10;
   (viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
   (ix) sterility, e.g., the composition or preparation supports the growth of fewer than 100 viable microorganisms as tested under aseptic conditions, the composition or preparation meets the standard of USP<71>, and/or the composition or preparation meets the standard of USP<85> as described by cGMP guidelines for sterile drug products produced by aseptic processing;
   (x) viral contamination, e.g., the composition or preparation has an absence of, or an undetectable level ofviral contamination or
   (xi) differential modification, e.g., comprising a modification characteristic of a fungal cell or cell line; an insect cell or cell line; or a plant, plant cell or cell line,

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

Table of Contents for Examples
Manufacture and preparation of TREMs

| | |
|---|---|
| Example 1 | Manufacture of a TREM in a fungal production host cell |
| Example 2 | Manufacture of a TREM in a monocotyledonous plant |
| | production host cell |
| Example 3 | Manufacture of a TREM in a dicotyledonous plant |
| | production host cell |
| Example 4 | Manufacture of a TREM in an insect production host cell |
| Example 5 | Manufacture of a mischarged TREM |
| Example 6 | Manufacture of a TREM fragment (in vitro) |
| Example 7 | Manufacture of a TREM fragment in a fungal cell |
| | expression system |
| Example 8 | Manufacture of a TREM fragment in a plant cell |
| | expression system |

Delivery of TREMs

| | |
|---|---|
| Example 9 | Delivery of TREMs to mammalian cells |

Assays to analyze TREM activity

| | |
|---|---|
| Example 10 | TREM translational activity assay in mammalian cells |
| Example 11 | Assay for modulation of cell state |
| Example 12 | Assay for the activity of an uncharged TREM to |
| | modulate autophagy |
| Example 13 | Assay for activity of a mischarged TREM (mTREM) |

### Example 11 Assay for Modulation of Cell State

Example 12 Assay for the activity of an uncharged TREM to modulate autophagy Example 13 Assay for activity of a mischarged TREM (mTREM) Example 1: Manufacture of a TREM in a fungal production host cell This example demonstrates the manufacturing of TREMs produced in fungal host cells.

### Plasmid Generation

To generate a plasmid comprising a sequence encoding a TREM, in this example, iMet CAT TREM, a DNA fragment containing the TREM sequence AGCAGAGTGGCGCAGCGGAAGCGTGCTGGGCCCATAACCCAGAGGTCGATGGATCG AAACCATCCTCTGCTA is PCR-amplified from human genomic DNA using the following primer pairs: 5′-TGAGTTGGCAACCTGTGGTA and 5′-TTGGGTGTCCATGAAAATCA and cloned into the corresponding sites of the high copy vector pRS425 as done in Christianson et al.

### Gene (1992) 110: 119-122.

### Transformation

Yeast transformation procedures are done as described in Burke, D., Dawson, D., and Steams, T. 2000. Methods in yeast genetics. CSHL Press. Briefly, 0.lug of plasmid described above is used to transform 100 uL of competent yeast cells. 600 pL of freshly prepared PEG-TE-LiAc solution is added and tube is vortexed, and incubated at 30° C. for 30 minutes with shaking. Cells are spun for 3 seconds, resuspended in sterile water and plated using appropriate synthetic complete drop-out medium. Yeast transformants are selected using blue-white screening.

### Purification

The selected cells expressing the plasmid encoding the TREM are lysed and separation from the lysate of RNAs smaller than 200 nucleotides is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe that is complementary to a unique region of the target TREM being purified. In this example, a probe with the sequence 5′ biotin-TAGCAGAGGATGGTTTCGATCCATCA is used to purify the iMET CAT TREM. The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner. The beads are then washed until the absorbance of the wash solution at 260 nm is close to zero. The TREM retained on the beads are eluted three times using RNase-free water, and then admixed with a pharmaceutically acceptable excipient to make a test TREM product.

### Use

One microgram of the test TREM preparation and a control agent are contacted by transfection, electroporation or liposomal delivery, with a cultured cell such as a HeLa cell or a HEK293T cell, a tissue or a subject, for a time sufficient for the TREM preparation to modulate a translation level or activity of the cell, relative to the control agent.

### Example 2: Manufacture of a TREM in a Monocotyledonous Plant Production Host Cell

This example demonstrates the manufacturing of TREMs produced in monocotyledonous plant host cell.

### Plant Materials

Maize Black Mexican sweet (BMS) suspension cultures (ATCC 54022) are used to generate stable transformants that produce TREM.

### Plasmid Construction and Agrobacterium-Mediated Transformation

To generate a plasmid comprising a sequence encoding a TREM, in this example, iMet CAT TREM, a DNA fragment containing the TREM sequence AGCAGAGTGGCGCAGCGGAAGCGTGCTGGGCCCATAACCCAGAGGTCGATGGATCG AAACCATCCTCTGCTA) is PCR-amplified from human genomic DNA using the following primer pairs: 5′-TGAGTTGGCAACCTGTGGTA and 5′-TTGGGTGTCCATGAAAATCA and cloned into a binary vector which is driven by a constitutive wheat RNA polymerase III promoter. The resulting construct is introduced into Agrobacterium tumefaciens strain LBA4404 using freeze-thaw method as described in Holsters et al., (1978) Mol Gen Genet.; 163(2):181-7.

### Agrobacterium-Mediated Transformation of Maize BMS

The Agrobacterium containing the TREM construct is co-cultured with maize Black Mexican sweet suspension culture. To recover putative transgenic cells, the explants were transferred to MSI medium supplemented with timentin to eliminate A. tumefaciens.

### Purification

The injected leaves or dipped seedlings are ground and total RNA is isolated from them using TRIZOL as described in Box et al (2011) Plant Methods 7,7. Separation of RNA smaller in size than 200 nucleotides from total RNA is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe that is complementary to a unique region of the target TREM being purified. In this example, a probe with the sequence 5′ biotin-TAGCAGAGGATGGTTTCGATCCATCA is used to purify the iMet CAT TREM. The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner. The beads are then washed until the absorbance of the wash solution at 260 nm is close to zero. The TREM retained on the beads are eluted three times using RNase-free water, and then admixed with a pharmaceutically acceptable excipient to make a test TREM product.

### Use

One microgram of the test TREM preparation and a control agent are contacted by transfection, electroporation or liposomal delivery, with a cultured cell such as a HeLa cell or a HEK293T cell, a tissue or a subject, for a time sufficient for the TREM preparation to modulate a translation level or activity of the cell, relative to the control agent.

### Example 3: Manufacture of a TREM in a Dicotyledonous Plant Production Host

This example demonstrates the manufacturing of TREMs produced in dicotyledonous plant host.

### Plant Materials

Tobacco (Nicotiana tabacum L.), cultivar “Wisconsin 38,” is used to generate transgenic plants that produce a TREM. The preparation of sterilized seedlings and the procedure for transformation is the same as described previously in Musa T. A., et al. (2009). Plant Biotechnol. Rep. 3 157-165.

### Plasmid Construction and Agrobacterium-Mediated Transformation

To generate a plasmid comprising a sequence encoding a TREM, in this example, iMet CAT TREM, a DNA fragment containing the TREM sequence AGCAGAGTGGCGCAGCGGAAGCGTGCTGGGCCCATAACCCAGAGGTCGATGGATCG AAACCATCCTCTGCTA) is PCR-amplified from human genomic DNA using the following primer pairs: 5′-TGAGTTGGCAACCTGTGGTA and 5′-TTGGGTGTCCATGAAAATCA and cloned into a binary vector which is driven by a constitutive plant RNA polymerase III promoter, such as the Arabidopsis U6-1 promoter and a “TTTTTT” transcription terminator. The resulting construct is introduced into Agrobacterium tumefaciens strain LBA4404 using freeze-thaw method as described in Holsters et al., (1978) Mol Gen Genet.; 163(2):181-7.

Agrobacterium-mediated Transformation of Nicotiana tabacum The Agrobacterium containing the TREM construct is transformed into 2-10 cm plant seedlings through floral dip or leaf disc transformation. Briefly, seedlings are dipped for a minute into agrobacterium medium as described in Zhang et al (2006) Nat. Protoc. 1, 641-646 or diffusion of the agrobacterium medium through the leaf is performed by placing the tip of the syringe against the underside of the leaf and injecting its content as described in Sparkes et al (2006) Nat. Protoc. 1, 2019-2025.

### Purification

The injected leaves or dipped seedlings are ground and total RNA is isolated from them using TRIZOL as described in Box et al (2011) Plant Methods 7,7. Separation of RNA smaller in size than 200 nucleotides from total RNA is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe that is complementary to a unique region of the target TREM being purified. In this example, a probe with the sequence 5′ biotin-TAGCAGAGGATGGTTTCGATCCATCA is used to purify the iMet CAT TREM. The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner. The beads are then washed until the absorbance of the wash solution at 260 nm is close to zero. The TREM retained on the beads are eluted three times using RNase-free water, and then admixed with a pharmaceutically acceptable excipient to make a test TREM product.

### Use

One microgram of the test TREM preparation and a control agent are contacted by transfection, electroporation or liposomal delivery, with a cultured cell such as a HeLa cell or a HEK293T cell, a tissue or a subject, for a time sufficient for the TREM preparation to modulate a translation level or activity of the cell, relative to the control agent.

### Example 4: Manufacture of a TREM in an Insect Production Host Cell

This example demonstrates the manufacturing of TREMs produced in insect host cells.

### Viral Vector Generation

To generate a plasmid comprising a sequence encoding a TREM, in this example, iMet CAT TREM, a DNA fragment containing the TREM sequence AGCAGAGTGGCGCAGCGGAAGCGTGCTGGGCCCATAACCCAGAGGTCGATGGATCG AAACCATCCTCTGCTA) is PCR-amplified from human genomic DNA using the following primer pairs: 5′-TGAGTTGGCAACCTGTGGTA and 5′-TTGGGTGTCCATGAAAATCA. is cloned into a viral vector, such as pAcBac.

### Recombinant Baculovirus Generation

Recombinant baculovirus carrying the TREM is generated from shuttle vectors according to the instruction manual for Bac-to-Bac baculovirus expression systems (Invitrogen). In brief, the shuttle vector is transformed into E. coli DH10 Bac (Invitrogen) carrying the AcNPV genome as a bacmid. Recombinant DNA is isolated using blue-white screening and is transfected into Sf9 cells cultured in Sf-900 III SFM serum-free media using Cellfectin reagent according to manufacturer's instructions. Primary baculovirus stocks are harvested from the supernatant 96 h post-transfection and amplified by re-infecting Sf9 cells at low MOI (<1). Titers of virus stocks are measured using BacPAK baculovirus rapid titer kit (Clontech).

### Infection

Infect cells, in this example suspension Sf9 cells in mid-logarithmic phase of growth, with a desired MOI of the baculovirus and collect cells by centrifugation at the optimal expression time point, for example between 48-96 hours post-infection.

### Purification

The collected cell pellets are lysed and separation from the lysate of RNAs smaller than 200 nucleotides is performed using a small RNA isolation kit per manufacturer's instructions, to generate a small RNA (sRNA) fraction.

To prepare the affinity purification reagents, streptavidin-conjugated RNase-free magnetic beads are incubated at room temperature for 30 min with 200 mM of biotinylated oligonucleotides corresponding to a DNA probe that is complementary to a unique region of the target TREM being purified. In this example, a probe with the sequence 5′ biotin-TAGCAGAGGATGGTTTCGATCCATCA is used to purify the TREM comprising iMet CAT TREM. The beads are washed and heated for 10 min at 75° C.

The sRNA fraction is heated for 10 min at 75° C. and then mixed with the affinity purification reagent described above. The admixture is incubated at room temperature for 3 hours to allow binding of the TREMs to the bead-bound DNA probe in a sequence specific manner. The beads are then washed until the absorbance of the wash solution at 260 nm is close to zero. The TREM retained on the beads are eluted three times using RNase-free water, and then admixed with a pharmaceutically acceptable excipient to make a test TREM product.

### Use

One microgram of the test TREM preparation and a control agent are contacted by transfection, electroporation or liposomal delivery, with a cultured cell such as a HeLa cell or a HEK293T cell, a tissue or a subject, for a time sufficient for the TREM preparation to modulate a translation level or activity of the cell, relative to the control agent.

### Example 5: Production of Mischarged TREMs

This example demonstrates the production of a TREM charged with an amino acid that does not correspond to its anticodon.

A TREM is produced as described in any of Examples 1-4. The TREM product is charged with a heterologous amino acid using an in vitro charging reaction known in the art (see, e.g., Walker & Fredrick (2008) Methods (San Diego, Calif.) 44(2):81-6). Briefly, the purified TREM, for example an Arg AGA TREM, is placed in a buffer with the heterologous amino acid of interest (for example glutamic acid), and the corresponding aminoacyl-tRNA synthetase (for example a Serine-tRNA synthetase mutated to enhance tRNA mischarging), to induce TREM charging. To isolate the aminoacyl-TREM, the in vitro charging reaction is passed through a size exclusion column to isolate the charged tRNA fraction and the concentration based on the A260 absorbance is determined as is the extent of aminoacylation using acid gel electrophoresis.

### Example 6: Production of TREM Fragments (In Vitro)

This example demonstrates the production of TREM fragments in-vitro, from a TREM manufactured in fungal, insect or plant host cells.

A TREM is made as described in any Example above. An enzymatic cleavage assay with enzymes known to generate tRNA fragments, such as RNase A or angiogenin, is used to produce fragments for administration to a cell, tissue or subject.

Briefly, a TREM manufactured as describe above is incubated in one of: 0.1M Hepes/NaOH, pH 7.4 with 10 nM final concentration of RNase A for 10 min at 30° C., or 0.1M MES, 0.1M NaCl, pH 6.0, with an effective amount of angiogenin, and BSA for 6 hours at
37° C.

To isolate a target TREM fragment after enzymatic treatment, a sequence affinity purification procedure is performed, as described above.

### Example 7: Production of TREM Fragments in a Fungal Cell Expression System

This example demonstrates the production of TREM fragments in a fungal cell expression system.

A cell line stably overexpressing a TREM is generated as described in Example 1. The yeast cells are stressed by method of heat shock to produce TREM fragments as described by Bgkowska-Zywicka, K., et al. (2016). FEBS open bio, 6(12), 1186-1200. TREM fragments are isolated from cells as described by Bgkowska-Zywicka, K., et al. (2016). FEBS open bio, 6(12), 1186-1200. Briefly, size selection of RNAs smaller than 200 nucleotides is performed using phenol extraction followed by LiCl extraction and ethanol recovery. Streptavidin-conjugated RNase-free magnetic beads are incubated at room temperature for 3 hrs with 200 mM of biotinylated oligonucleotides corresponding to a DNA probe that is complementary to a unique region of the tRNA half being purified. The beads are washed and the TREM retained on the beads are eluted through heating for 10 min at 75° C. in RNase-free water.

### Example 8: Production of TREM Fragments in a Plant Cell Expression System

This example demonstrates the production of TREM fragments in a plant cell expression system.

A cell line stably overexpressing a TREM is generated as described in Example 3. The plants are stressed by method of phosphate starvation to produce TREM fragments as described by Megel C. et al. (2019) Nucleic Acids Research 47, 2: 941-952. TREM fragments are isolated from cells. The small RNA fraction (smaller than 200 nucleotides) is isolated from leaves as described by Mardchal-Drouard L., et al (1995) Methods Enzymol.; 260:310-327. Streptavidin-conjugated RNase-free magnetic beads are incubated at room temperature for 3 hrs with 200 mM of biotinylated oligonucleotides corresponding to a DNA probe that is complementary to a unique region of the tRNA half being purified. The beads are washed and the TREM retained on the beads are eluted through heating for 10 min at 75° C. in RNase-free water.

### Example 9: Delivery of TREMs to Mammalian Cells

This example demonstrates the delivery of TREMs manufactured in fungal, insect or plant host cells to mammalian cells.

100 nM of the TREM isolated from fungal, insect or plant cells is electroporated or transfected in human cultured cell such as a HeLa cell or a HEK293T cells using lipofectamine 2000 reagents according to the manufacturer's instructions. After 6-24 hours, the media is replaced with fresh media.

### Example 10: TREM Translational Activity Assays

This example demonstrates assays for the ability of a TREM to be incorporated into a nascent polypeptide chain.

### Translation of the FLAG-AA-his Peptide Seqsuence

A test TREM is assayed in an in-vitro translation reaction with an mRNA encoding the peptide FLAG-XXX-His6x, where XXX are 3 consecutive codons corresponding to the test TREM anticodon.

A tRNA-depleted rabbit reticulocyte lysate (Jackson et al. 2001-RNA 7:765-773) is incubated 1 hour at 30° C. with 10-25 ug/mL of the test TREM in addition to 10-25 ug/mL of the tRNAs required for the FLAG and His tag translation. In this example, the TREM used is tRNA-Ile-GAT, therefore the peptide used is FLAG-LLL-His6x and the tRNAs added are tRNA-Ile-GAT, in addition to the following, which are added for translate the peptide FLAG and HIS tags: tRNA-Asp-GAC, tRNA-Tyr-TAC, tRNA-Lys-AAA, tRNA-Lys-AAAG, tRNA-Asp-GAT, tRNA-His-CAT. To determine if the test TREM is functionally able to be incorporated into a 15 nascent peptide, an ELISA capture assay is performed. Briefly, an immobilized anti-His6× antibody is used to capture the FLAG-LLL-His6x peptide from the reaction mixture. The reaction mixture is then washed off and the peptide is detected with an enzyme-conjugated anti-FLAG antibody, which reacts to a substrate in the ELISA detection step. If the TREM produced is functional, the FLAG-LLL-His6 peptide is produced and detection occurs by the ELISA capture assay.

If the TREM produced is not functional, the FLAG-LLL-His6 peptide is not produced and no detection occurs by the ELISA capture assay.

### Translational Suppression Assay

This assay demonstrates that a test TREM has translational adaptor molecule function by rescuing a suppression mutation and allowing the full protein to be translated. The test TREM, in this example Arg-AGA TREM, is produced such that it contains the sequence of the tRNA-Ile-GAT body but with the anticodon sequence corresponding to TCA instead of AGA. HeLa cells are co-transfected with 50 ng of TREM and with 200 ng of a DNA plasmid encoding a mutant GFP containing a UGA stop codon at the S29 position as described in Geslain et al. 2010. J Mol Biol. 396:821-831. HeLa cells transfected with the GFP plasmid alone serve as a negative control. After 24 hours, cells are collected and analyzed for fluorescence recovery by flow cytometry. The fluorescence is read out with an emission peak at 509 nm (excitation at 395 nm). It is expected that if the test TREM is functional, it will be sufficient to rescue the stop mutation in the GFP molecule and produce the full-length fluorescent protein, which is detected by flow cytometry. If the test TREM is not functional, the stop mutation is not rescued, and no fluorescence is emitted from the GFP molecule and detected by flow cytometry.

### In Vitro Translational Assay

This assay demonstrates that a test TREM has translational adaptor molecule function by successfully being incorporated into a nascent polypeptide chain in an in vitro translation reaction. First, a HEK293T-derived human lysate that is depleted of the endogenous tRNA using an antisense oligonucleotide targeting the sequence between the anticodon and variable loop is generated (see, e.g., Cui et al. 2018. Nucleic Acids Res. 46(12):6387-6400). 10-25 ug/mL of the test TREM is added in addition to 2 ug/uL of a GFP-encoding mRNA to the depleted lysate. A non-depleted lysate with the GFP mRNA and with or without test TREM added are used as a positive control. A depleted lysate with the GFP mRNA but without the test TREM added is used as a negative control. The progress of GFP mRNA translation is monitored by fluorescence increase on a microplate reader at 37° C. for 3-5 h using)λₑₓ485/λₑₘ528. It is expected for the experimental sample to produce similar levels of fluorescence over time as the positive control and to produce higher levels of fluorescence over time compared to the negative control. If so, these results would indicate that the test TREM is sufficient to complement the depleted lysate and is thus functional.

### Example 11: Assay for Modulation of Cell State

This example describes an assay for detecting activity of a TREM in modulating cell status, e.g., cell death.

TREM fragments are produced as described in Example 7 or 8. 1 uM of TREM fragments are transfected into HEK293T cells with Lipofectamine 3000 and incubated for 1-6 hours in hour-long intervals followed by cell lysis. Cell lysates are analyzed by Western blotting and blots are probed with antibodies against total and cleaved caspase 3 and 9 as readouts of apoptosis. To measure cellular viability, cells are washed and fixed with 4% paraformaldehyde in PBS for 15 minutes at room temperature. Fixed and washed cells are then treated with 0.1% Triton X-100 for 10 minutes at room temperature and washed with PBS three times. Finally, cells are treated with TUNEL assay reaction mixture at 37° C. for 1 hour in the dark. Samples are analyzed by flow cytometry.

### Example 12: Assay for the activity of an uncharged TREM to modulate autophagy

This example describes an assay to test an uncharged TREM for ability to modulate, e.g., induce, autophagy, e.g., the ability to activate GCN2-dependent stress response (starvation) pathway signaling, inhibit mTOR or activate autophagy.

A test uncharged TREM (uTREM) preparation made according to any of Examples 1-8 is delivered to HEK293T or HeLa cells through transfection or liposomal delivery. Once the uTREM is delivered, a time course is performed ranging from 30 minutes to 6 hours with hour-long interval time points. Cells are then trypsinized, washed and lysed. The same procedure is executed with a charged control TREM as well as random RNA oligos as controls. Cell lysates are analyzed by Western blotting and blots are probed with antibodies against known readouts of GCN2 pathway activation, mTOR pathway inhibition or autophagy induction, including but not limited to phospho-eIF2a, ATF4, phospho-ULK1, phospho-4EBP1, phospho-eIF2a, phospho-Akt and phospho-p70S6K. A total protein loading control, such as GAPDH, actin or tubulin, as well as the non-modified (i.e. non-phosphorylated) signaling protein, i.e. using eIF2a as a control for phospho-eIF2a, are probed as loading controls. Delivery of the uTREM, compared to controls, is expected to show activation of GCN2 starvation signaling pathway, autophagy pathway and/or inhibition of the mTOR pathway as determined by Western blot analysis.

### Example 13: Assay for Activity of a Mischarged TREM (mTREM)

This example describes an assay to test the functionality of a mTREM produced in a host cell system using plasmid transfection followed by in vitro mischarging.

In this example, an mTREM can translate a mutant mRNA into a wild type (WT) protein by incorporation of the WT amino acid in the protein despite an mRNA containing a mutated codon. GFP mRNA molecules with either a T203I or E222 G mutation, which prevent GFP excitation at the 470 nm and 390 nm wavelengths, respectively, are used for this example. GFP mutants which prevent GFP fluorescence could also be used as reporter proteins in this assay. Briefly, an in-vitro translation assay is used, using a human cell lysate containing the GFP E222 G mutated mRNA (GAG->GGG mutation) and an excess of the mTREM, in this case tRNA-Glu-CCC. As a negative control, no mischarged TREM is added to the reaction. If the mTREM is functional, it is expected that the GFP protein produced fluoresces when illuminated with a 390 nm excitation wavelength using a fluorimeter. If the mTREM is not functional, the GFP protein produced fluoresces only when excited with a 470 nm wavelength, as is observed in the negative control.

What is claimed is:

1. A method of making a purified tRNA effector molecule (TREM) composition, e.g., a TREM pharmaceutical composition, comprising:
providing a host cell selected from a fungal cell or cell line, an insect cell or cell line, or a plant, plant cell or cell line, comprising an exogenous nucleic acid, e.g., a DNA or RNA, encoding the TREM;
maintaining the host cell under conditions sufficient to express the TREM;
purifying the TREM from the host cell, e.g., according to a method described herein; and
formulating the purified TREM as a pharmaceutical composition, e.g., by combining the TREM with a pharmaceutical excipient,
thereby making the TREM composition.

2. The method of claim 1, wherein the nucleic acid comprises an RNA, which upon reverse transcription, results in a DNA which can be transcribed into the TREM.

3. The method of claim 1 or 2, wherein the nucleic acid comprises an RNA sequence at least 90% identical to an RNA sequence encoded by a DNA sequence listed in Table 1, or a fragment or functional fragment thereof.

4. The method of claim 1 or 2, wherein the nucleic acid comprises an RNA sequence comprising a consensus sequence provided herein.

5. The method of any one of the preceding claims, wherein the host cell comprises a fungal cell or cell line.

6. The method of claim 5, wherein the fungal cell or cell line is chosen from the following genera: Saccharomyces, Yarrowia, Pichia, Schwanniomyces, Kluyveromyces, Arxula, Trichosporon, Candida, Ustilago, Torulopsis, Zygosaccharomyces, Trigonopsis, Cryptococcus, Rhodotorula, Phaffia, Sporobolomyces, Neurospora, Pichia or Pachysolen.

7. The method of claim 5 or 6, wherein the fungal cell or cell line is a Saccharomyces cell or cell line.

8. The method of claim 5 or 6, wherein the the fungal cell or cell line is a Saccharomyces cerevisiae fungal cell or cell line.

9. The method of claim 5 or 6, wherein the the fungal cell or cell line is a Schizosaccharomyces pombe fungal cell or cell line.

10. The method of claim 5 or 6, wherein the fungal cell or cell line is a Candida cylindracea fungal cell or cell line.

11. The method of claim 5 or 6, wherein the fungal cell or cell line is a Candida albicans fungal cell or cell line.

12. The method of claim 5 or 6, wherein the fungal cell or cell line is a Neurospora crassa fungal cell or cell line.

13. The method of claim 5 or 6, wherein the fungal cell or cell line is a Pichia jadinii fungal cell or cell line.

14. The method of any one of claims 1-5, wherein the host cell comprises an insect cell or cell line.

15. The method of claim 14, wherein the insect cell or cell line is chosen from Autographa californica, Bombyx mori, Spodoptera frugiperda, Choristoneura fumiferana, Heliothis zea, Orgyia pseudotsugata, Lymantira dispar, Plutelia xylostella, Malacostoma disstria, Trichoplusia ni, Pieris rapae, Mamestra configurata, Hyalophora cecropia, Aedes albopictus, or Drosophila melanogaster.

16. The method of claim 14 or 15, wherein the insect cell is a Spodoptera frugiperda cell, optionally wherein the Spodoptera frugiperda cell is an Sf9 cell.

17. The method of claim 14 or 15, wherein the insect cell is a Trichoplusia ni cell, optionally wherein the insect cell is a H5 cell (High Five™, Invitrogen, Sorrento, CA).

18. The method of any one of claims 1-5, wherein the host cell comprises a plant, a plant cell or cell line.

19. The method of claim 18, wherein the host plant, plant cell or cell line is a monocotyledonous plant, cell or cell line.

20. The method of claim 18, wherein the host plant, plant cell or cell line is a dicotyledonous plant, cell or cell line.

21. The method of any one of claims 18-20, wherein the plant, plant cell or cell line is a plant, cell or cell line chosen from wheat (e.g., Triticum aestivum), rice, maize (e.g., Zea mays), barley (e.g., Hordeum vulgare), tobacco (e.g., Nicotiana rustica or Nicotiana tabacum), lupins (e.g., Lupinus albus), bean (e.g., Phaseolus vulgaris), pea (e.g., Pisum sativum), potato (e.g., Solanum tuberosum), spinach (e.g., Spinacia oleracea), or Arabidopsis.

22. The method of claim 21, wherein the plant, cell or cell line is an Arabidopsis plant, cell or cell line.

23. The method of claim 22, wherein the Arabidopsis plant, cell or cell line is an A. thaliana plant, cell or cell line.

24. The method of any one of the preceding claims, wherein the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity-based separation.

25. A composition comprising a purified tRNA effector molecule (TREM) (e.g., a purified TREM composition made according to a method described herein), comprising:
(i) an RNA sequence at least 90% identical to an RNA sequence encoded by a DNA sequence listed in Table 1, or a fragment or functional fragment thereof; or
(ii) an RNA sequence comprising a consensus sequence provided herein.

26. A GMP-grade, recombinant TREM composition (e.g., a TREM composition made in compliance with cGMP, and/or in accordance with similar requirements) comprising:
(i) an RNA sequence at least 90% identical to an RNA sequence encoded by a DNA sequence listed in Table 1, or a fragment or functional fragment thereof; or
(ii) an RNA sequence comprising a consensus sequence provided herein.

27. The TREM composition of claim 25 or 26, wherein the composition comprises one or more, e.g., a plurality, of TREMs.

28. The TREM composition of any one of claims 25 to 27, wherein the composition comprises at least 1, 2, 3, 4, 5, 6, 7, 8, 9 or 10 species of TREMs.

29. The TREM composition of any one of claims 25-28, wherein the TREM composition (or an intermediate in the production of a TREM composition) comprises one or more of the following characteristics:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng, per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 10;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>;
(x) viral contamination, e.g., the composition or preparation has an absence of, or an undetectable level of viral contamination; or
(xi) differential modification, e.g., comprising a modification characteristic of a fungal cell or cell line; an insect cell or cell line; or a plant, plant cell or cell line.

30. A method of modulating a tRNA pool in a cell, e.g., a mammalian cell, comprising:
providing a purified TREM composition, e.g., made according to a method described herein, and contacting the cell with the TREM composition,
thereby modulating the tRNA pool in the cell.

31. The method of claim 30, wherein the TREM composition is made by:
providing a host cell, e.g., a fungal cell or cell line, an insect cell or cell line, or a plant, plant cell or cell line, comprising an exogenous nucleic acid, e.g., a DNA or RNA, encoding the TREM;
maintaining the cell under conditions sufficient to express the TREM; and/or
purifying the TREM from the host cell, e.g., according to a method described herein.

32. The method of claim 30 or 31, wherein the host cell is a fungal cell or cell line, e.g., as described herein.

33. The method of claim 30 or 31, wherein the host cell is an insect cell or cell line, e.g., as described herein.

34. The method of claim 30 or 31, wherein the host cell is a plant, plant cell or cell line, e.g., as described herein.

35. The method of any one of claims 31-34, wherein the purification step comprises one, two or all of the following steps, e.g., in the order recited:
(i) separating nucleic acids from cellular debris to provide an RNA preparation;
(ii) separating RNA of less than a threshold number of nucleotides, e.g., less than 500 nt, less than 400 nt, less than 300 nt, less than 250 nt, less than 200 nt, less than 150 nt, from larger RNA species in the RNA preparation to produce a small RNA preparation; and/or
(iii) separating a TREM from other RNA species in the small RNA preparation by affinity-based separation, e.g., sequence affinity-based separation.

36. The method of any one of claims 30-35, wherein the TREM comprises:
(i) an RNA sequence at least 80% identical to an RNA sequence encoded by a DNA sequence listed in Table 1, or a fragment or functional fragment thereof; or
(ii) an RNA sequence comprising a consensus sequence provided herein.

37. A method of making a tRNA effector molecule (TREM) composition, comprising:
(a) providing a host cell, e.g., a fungal cell or cell line, an insect cell or cell line, or a plant, plant cell or cell line, comprising exogenous nucleic acid, e.g., a DNA or RNA, encoding a TREM under conditions sufficient to express the TREM, and
(b) purifying the expressed TREM from the host cell to produce a TREM composition, thereby making the TREM composition.

38. A method of making a pharmaceutical TREM composition comprising:
combining
a) a TREM, e.g., a purified TREM composition, e.g., a TREM composition made by a method described herein; and
b) a pharmaceutically acceptable component, e.g., an excipient,
thereby making a pharmaceutical TREM composition.

39. A method of making a purified tRNA effector molecule (TREM) pharmaceutical composition, comprising:
purifying the TREM from a host cell, e.g., a fungal cell or cell line, an insect cell or cell line, or a plant, plant cell or cell line;
formulating the purified TREM as a pharmaceutical composition, e.g., by combining the TREM with a pharmaceutical excipient,
thereby making the TREM pharmaceutical composition.

40. A method of making a TREM composition, comprising:
contacting a TREM containing a reaction mixture with a reagent, e.g., a capture reagent or a separation reagent, comprising a nucleic acid sequence complimentary with a TREM;
thereby making a TREM composition.

41. A method of making a pharmaceutical composition, comprising:
a) providing a purified TREM composition, e.g., a purified TREM composition made by culturing a host cell, e.g., a fungal cell or cell line, an insect cell or cell line, or a plant, plant cell or cell line, comprising DNA or RNA encoding a TREM under conditions sufficient to express the TREM, and purifying the expressed TREM from the host cell culture to produce a purified TREM composition,
b) providing a value, e.g., by evaluating or testing, for one or more of the following characteristics of the purified TREM composition:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 10;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., as per cGMP guidelines for sterile drug products, e.g., the composition or preparation supports the growth of fewer than 100 viable microorganisms as tested under aseptic conditions, the composition or preparation meets the standard of USP<71>, and/or the composition or preparation meets the standard of USP<85>;
(x) viral contamination, e.g., the composition or preparation has an absence of, or an undetectable level of viral contamination, or
(xi) differential modification, e.g., comprising a modification characteristic of a fungal cell or cell line; an insect cell or cell line; or a plant, plant cell or cell line,
c) optionally, formulating the purified TREM composition as a pharmaceutical drug product (e.g., combining the TREM composition with a pharmaceutical excipient) if it meets a reference criteria for the one or more characteristics,
thereby making a pharmaceutical composition.

42. A pharmaceutical tRNA effector molecule (TREM) composition, comprising (i) an RNA sequence at least 80% identical to an RNA sequence encoded by a DNA sequence listed in Table 1, or a fragment or functional fragment thereof; or
(ii) an RNA sequence comprising a consensus sequence provided herein.

43. A recombinant TREM composition of at least 0.5 g, 1 g, 2 g, 3 g, 4 g, 5 g, 6 g, 7 g, 8 g, 9 g, 10 g, 15 g, 20 g, 30 g, 40 g, 50 g, 100 g, 200 g, 300 g, 400 g or 500 g.

44. A recombinant TREM composition of between 0.5 g to 500 g, between 0.5 g to 400 g, between 0.5 g to 300 g, between 0.5 g to 200 g, between 0.5 g to 100 g, between 0.5 g to 50 g, between 0.5 g to 40 g, between 0.5 g to 30 g, between 0.5 g to 20 g, between 0.5 g to 10 g, between 0.5 g to 9 g, between 0.5 g to 8 g, between 0.5 g to 7 g, between 0.5 g to 6 g, between 0.5 g to 5 g, between 0.5 g to 4 g, between 0.5 g to 3 g, between 0.5 g to 2 g, between 0.5 g to 1 g, between 1 g to
500 g, between 2 g to 500 g, between 5 g to 500 g, between 10 g to 500 g, between 20 g to 500 g, between 30 g to 500 g, between 40 g to 500 g, between 50 g to 500 g, between 100 g to 500 g, between 200 g to 500 g, between 300 g to 500 g, or between 400 g to 500 g.

45. A TREM composition comprising a consensus sequence of Formula I zzz,
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein:
R is a ribonucleotide residue;
(i) zzz indicates any of the twenty amino acids;
(ii) Formula I corresponds to all species; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).

46. A TREM composition comprising a consensus sequence of Formula II zzz,
R₀-R₁-R₂-R₃-R₄-R₅-R₆-R₇-R₈-R₉-R₁₀-R₁₁-R₁₂-R₁₃-R₁₄-R₁₅-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein:
R is a ribonucleotide residue;
(i) zzz indicates any of the twenty amino acids;
(ii) Formula II corresponds to mammals; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).

47. A TREM composition comprising a consensus sequence of Formula III zzz,
R₀-R₁-R₂-R₃-R₄-R₅-R₆-R₇-R₈-R₉-R₁₀-R₁₁-R₁₂-R₁₃-R₁₄-R₁₅-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein:
R is a ribonucleotide residue;
(i) zzz indicates any of the twenty amino acids;
(ii) Formula III corresponds to humans; and
(iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).

48. A method of contacting a cell, tissue, or subject, e.g., a mammalian cell, tissue or subject, with a TREM, comprising contacting the cell, tissue or subject with a purified TREM composition,
thereby contacting a cell, tissue, or subject with the TREM.

49. A method of delivering a TREM to a cell, tissue, or subject, e.g., a mammalian cell, tissue or subject, comprising:
providing a cell, tissue, or subject, and contacting the cell, tissue, or subject, with a TREM composition, e.g., a purified TREM composition, e.g., a pharmaceutical TREM composition.

50. A method of treating a subject, e.g., modulating the metabolism, e.g., the translational capacity of a cell, in a subject, e.g., a mammal, comprising:
providing, e.g., administering to the subject, an exogenous nucleic acid, e.g., a DNA or RNA, which encodes a TREM,
thereby treating the subject.

51. A cell, e.g., a mammalian cell, comprising a TREM made according to a method of making a TREM disclosed herein.

52. A reaction mixture comprising a TREM and a reagent, e.g., a capture reagent, or a separation reagent.

53. A bioreactor comprising a plurality of host cells described herein comprising exogenous DNA or RNA.

54. A method of evaluating a composition of TREM, e.g., a GMP-grade TREM (i.e., a TREM made in compliance with cGMP, and/or in accordance with similar requirements), comprising acquiring a value for one or more of the following characteristics of the purified TREM composition:
(i) purity of at least 30%, 40%, 50%, 60%, 70%, 80%, 85%, 90%, 91%, 92%, 93%, 94%, 95%, 96%, 97%, 98%, or 99%;
(ii) host cell protein (HCP) contamination of less than 0.1 ng/ml, 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(iii) host cell protein (HCP) contamination of less than 0.1 ng, 1 ng, 5 ng, 10 ng, 15 ng, 20 ng, 25 ng, 30 ng, 35 ng, 40 ng, 50 ng, 60 ng, 70 ng, 80 ng, 90 ng, or 100 ng per milligram (mg) of the TREM composition;
(iv) DNA, e.g., host cell DNA, of less than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, or 100 ng/ml;
(v) less than 0.1%, 0.5%, 1%, 2%, 3%, 4%, 5%, 6%, 7%, 8%, 9% or 10% TREM fragments relative to full length TREMs;
(vi) low levels or absence of endotoxins, e.g., a negative result as measured by the Limulus amebocyte lysate (LAL) test;
(vii) in-vitro translation activity, e.g., as measured by an assay described in Example 10;
(viii) TREM concentration of at least 0.1 ng/mL, 0.5 ng/mL, 1 ng/mL, 5 ng/mL, 10 ng/mL, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL;
(ix) sterility, e.g., the composition or preparation supports the growth of fewer than 100 viable microorganisms as tested under aseptic conditions, the composition or preparation meets the standard of USP<71>, and/or the composition or preparation meets the standard of USP<85> as described by cGMP guidelines for sterile drug products produced by aseptic processing;
(x) viral contamination, e.g., the composition or preparation has an absence of, or an undetectable level of viral contamination or
(xi) differential modification, e.g., comprising a modification characteristic of a fungal cell or cell line; an insect cell or cell line; or a plant, plant cell or cell line,


SEQUENCE LISTING (625 sequences)

SEQ ID NO: 1 - DNA, 72 bp, Homo sapiens
  source 1..72: mol_type = genomic DNA
gggggtatagctcagtggtagagcgcgtgcttagcatgcacgaggtcctgggttcgatccccagtacctcca

SEQ ID NO: 2 - DNA, 73 bp, Homo sapiens
  source 1..73: mol_type = genomic DNA
ggggaattagctcaagtggtagagcgcttgcttagcacgcaagaggtagtgggatcgatgcccacattctcca

SEQ ID NO: 3 - DNA, 73 bp, Homo sapiens
  source 1..73: mol_type = genomic DNA
ggggaattagctcaaatggtagagcgctcgcttagcatgcgagaggtagcgggatcgatgcccgcattctcca

SEQ ID NO: 4 - DNA, 73 bp, Homo sapiens
  source 1..73: mol_type = genomic DNA
ggggaattagctcaagtggtagagcgcttgcttagcatgcaagaggtagtgggatcgatgcccacattctcca

SEQ ID NO: 5 - DNA, 73 bp, Homo sapiens
  source 1..73: mol_type = genomic DNA
ggggaattagctcaagcggtagagcgcttgcttagcatgcaagaggtagtgggatcgatgcccacattctcca

SEQ ID NO: 6 - DNA, 73 bp, Homo sapiens
  source 1..73: mol_type = genomic DNA
ggggaattagctcaagtggtagagcgcttgcttagcatgcaagaggtagtgggatcaatgcccacattctcca

SEQ ID NO: 7 - DNA, 73 bp, Homo sapiens
  source 1..73: mol_type = genomic DNA
ggggaattagctcaagtggtagagcgctcgcttagcatgcgagaggtagtgggatcgatgcccgcattctcca

SEQ ID NO: 8 - DNA, 73 bp, Homo sapiens
  source 1..73: mol_type = genomic DNA
ggggaattagcccaagtggtagagcgcttgcttagcatgcaagaggtagtgggatcgatgcccacattctcca

SEQ ID NO: 9 - DNA, 72 bp, Homo sapiens
  source 1..72: mol_type = genomic DNA
gggggtgtagctcagtggtagagcgcgtgcttagcatgcacgaggccccgggttcaatccccggcacctcca

SEQ ID NO: 10 - DNA, 72 bp, Homo sapiens
  source 1..72: mol_type = genomic DNA
gggggtgtagctcagtggtagagcgcgtgcttagcatgtacgaggtcccgggttcaatccccggcacctcca

SEQ ID NO: 11 - DNA, 72 bp, Homo sapiens
  source 1..72: mol_type = genomic DNA
ggggatgtagctcagtggtagagcgcatgcttagcatgcatgaggtcccgggttcgatccccagcatctcca

SEQ ID NO: 12 - DNA, 72 bp, Homo sapiens
  source 1..72: mol_type = genomic DNA
gggggtgtagctcagtggtagagcgcgtgcttagcatgcacgaggccctgggttcaatccccagcacctcca

SEQ ID NO: 13 - DNA, 72 bp, Homo sapiens
  source 1..72: mol_type = genomic DNA
gggggtatagctcagcggtagagcgcgtgcttagcatgcacgaggtcctgggttcaatccccaatacctcca

SEQ ID NO: 14 - DNA, 72 bp, Homo sapiens
  source 1..72: mol_type = genomic DNA
gggggtgtagctcagtggtagagcgcgtgcttagcatgcacgaggccccgggttcaatccctggcacctcca

SEQ ID NO: 15 - DNA, 73 bp, Homo sapiens
  source 1..73: mol_type = genomic DNA
gggggattagctcaaatggtagagcgctcgcttagcatgcgagaggtagcgggatcgatgcccgcatcctcca

SEQ ID NO: 16 - DNA, 73 bp, Homo sapiens
  source 1..73: mol_type = genomic DNA
ggggaattagctcaggcggtagagcgctcgcttagcatgcgagaggtagcgggatcgacgcccgcattctcca

SEQ ID NO: 17 - DNA, 72 bp, Homo sapiens
  source 1..72: mol_type = genomic DNA
ggggatgtagctcagtggtagagcgcatgcttcgcatgtatgaggtcccgggttcgatccccggcatctcca

SEQ ID NO: 18 - DNA, 72 bp, Homo sapiens
  source 1..72: mol_type = genomic DNA
ggggatgtagctcagtggtagagcgcatgcttcgcatgtatgaggccccgggttcgatccccggcatctcca

SEQ ID NO: 19 - DNA, 72 bp, Homo sapiens
  source 1..72: mol_type = genomic DNA
ggggatgtagctcagtggtagagcgcgcgcttcgcatgtgtgaggtcccgggttcaatccccggcatctcca

SEQ ID NO: 20 - DNA, 72 bp, Homo sapiens
  source 1..72: mol_type = genomic DNA
gggggtgtagctcagtggtagagcgcgtgcttcgcatgtacgaggccccgggttcgacccccggctcctcca

SEQ ID NO: 21 - DNA, 72 bp, Homo sapiens
  source 1..72: mol_type = genomic DNA
gggggtgtagctcagtggtagagcgcatgctttgcatgtatgaggtcccgggttcgatccccggcacctcca

SEQ ID NO: 22 - DNA, 72 bp, Homo sapiens
  source 1..72: mol_type = genomic DNA
ggggatgtagctcagtggtagagcgcatgctttgcatgtatgaggtcccgggttcgatccccggcatctcca

SEQ ID NO: 23 - DNA, 72 bp, Homo sapiens
  source 1..72: mol_type = genomic DNA
ggggatgtagctcagtggtagagcgcatgctttgcatgtatgaggccccgggttcgatccccggcatctcca

SEQ ID NO: 24 - DNA, 72 bp, Homo sapiens
  source 1..72: mol_type = genomic DNA
ggggatgtagctcagtggtagagcgcatgctttgcacgtatgaggccccgggttcaatccccggcatctcca

SEQ ID NO: 25 - DNA, 72 bp, Homo sapiens
  source 1..72: mol_type = genomic DNA
gggggtgtagctcagtggtagagcgcatgctttgcatgtatgaggcctcgggttcgatccccgacacctcca

SEQ ID NO: 26 - DNA, 72 bp, Homo sapiens
  source 1..72: mol_type = genomic DNA
gggggtgtagctcagtggtagagcacatgctttgcatgtgtgaggccccgggttcgatccccggcacctcca

SEQ ID NO: 27 - DNA, 71 bp, Homo sapiens
  source 1..71: mol_type = genomic DNA
gggggtgtagctcagtggtagagcgcatgctttgcatgtatgaggcctcggttcgatccccgacacctcca

SEQ ID NO: 28 - DNA, 73 bp, Homo sapiens
  source 1..73: mol_type = genomic DNA
gggccagtggcgcaatggataacgcgtctgactacggatcagaagattccaggttcgactcctggctggctcg

SEQ ID NO: 29 - DNA, 73 bp, Homo sapiens
  source 1..73: mol_type = genomic DNA
gggccagtggcgcaatggataacgcgtctgactacggatcagaagattctaggttcgactcctggctggctcg

SEQ ID NO: 30 - DNA, 73 bp, Homo sapiens
  source 1..73: mol_type = genomic DNA
ggccgcgtggcctaatggataaggcgtctgattccggatcagaagattgagggttcgagtcccttcgtggtcg

SEQ ID NO: 31 - DNA, 73 bp, Homo sapiens
  source 1..73: mol_type = genomic DNA
gacccagtggcctaatggataaggcatcagcctccggagctggggattgtgggttcgagtcccatctgggtcg

SEQ ID NO: 32 - DNA, 73 bp, Homo sapiens
  source 1..73: mol_type = genomic DNA
gccccagtggcctaatggataaggcactggcctcctaagccagggattgtgggttcgagtcccacctggggta

SEQ ID NO: 33 - DNA, 73 bp, Homo sapiens
  source 1..73: mol_type = genomic DNA
gccccagtggcctaatggataaggcactggcctcctaagccagggattgtgggttcgagtcccacctggggtg

SEQ ID NO: 34 - DNA, 73 bp, Homo sapiens
  source 1..73: mol_type = genomic DNA
gccccggtggcctaatggataaggcattggcctcctaagccagggattgtgggttcgagtcccacccggggta

SEQ ID NO: 35 - DNA, 73 bp, Homo sapiens
  source 1..73: mol_type = genomic DNA
gccccagtggcctaatggataaggcattggcctcctaagccagggattgtgggttcgagtcccatctggggtg

SEQ ID NO: 36 - DNA, 73 bp, Homo sapiens
  source 1..73: mol_type = genomic DNA
gccccagtggcctgatggataaggtactggcctcctaagccagggattgtgggttcgagttccacctggggta

SEQ ID NO: 37 - DNA, 73 bp, Homo sapiens
  source 1..73: mol_type = genomic DNA
ggccgcgtggcctaatggataaggcgtctgacttcggatcagaagattgcaggttcgagtcctgccgcggtcg

SEQ ID NO: 38 - DNA, 73 bp, Homo sapiens
  source 1..73: mol_type = genomic DNA
gaccacgtggcctaatggataaggcgtctgacttcggatcagaagattgagggttcgaatccctccgtggtta

SEQ ID NO: 39 - DNA, 73 bp, Homo sapiens
  source 1..73: mol_type = genomic DNA
gaccgcgtggcctaatggataaggcgtctgacttcggatcagaagattgagggttcgagtcccttcgtggtcg

SEQ ID NO: 40 - DNA, 73 bp, Homo sapiens
  source 1..73: mol_type = genomic DNA
gaccacgtggcctaatggataaggcgtctgacttcggatcagaagattgagggttcgaatcccttcgtggtta

SEQ ID NO: 41 - DNA, 73 bp, Homo sapiens
  source 1..73: mol_type = genomic DNA
gaccacgtggcctaatggataaggcgtctgacttcggatcagaagattgagggttcgaatcccttcgtggttg

SEQ ID NO: 42 - DNA, 73 bp, Homo sapiens
  source 1..73: mol_type = genomic DNA
ggccgtgtggcctaatggataaggcgtctgacttcggatcaaaagattgcaggtttgagttctgccacggtcg

SEQ ID NO: 43 - DNA, 85 bp, Homo sapiens
  source 1..85: mol_type = genomic DNA
ggctccgtggcgcaatggatagcgcattggacttctagaggctgaaggcattcaaaggttccgggttcgagtcccggcggagtcg

SEQ ID NO: 44 - DNA, 88 bp, Homo sapiens
  source 1..88: mol_type = genomic DNA
ggctctgtggcgcaatggatagcgcattggacttctagtgacgaatagagcaattcaaaggttgtgggttcgaatcccaccagagtcg

SEQ ID NO: 45 - DNA, 91 bp, Homo sapiens
  source 1..91: mol_type = genomic DNA
ggctctgtggcgcaatggatagcgcattggacttctagctgagcctagtgtggtcattcaaaggttgtgggttcgagtcccaccagagtcg

SEQ ID NO: 46 - DNA, 86 bp, Homo sapiens
  source 1..86: mol_type = genomic DNA
ggctctgtggcgcaatggatagcgcattggacttctagatagttagagaaattcaaaggttgtgggttcgagtcccaccagagtcg

SEQ ID NO: 47 - DNA, 74 bp, Homo sapiens
  source 1..74: mol_type = genomic DNA
gtctctgtggcgcaatggacgagcgcgctggacttctaatccagaggttccgggttcgagtcccggcagagatg

SEQ ID NO: 48 - DNA, 87 bp, Homo sapiens
  source 1..87: mol_type = genomic DNA
ggctctgtggcgcaatggatagcgcattggacttctagcctaaatcaagagattcaaaggttgcgggttcgagtccctccagagtcg

SEQ ID NO: 49 - DNA, 74 bp, Homo sapiens
  source 1..74: mol_type = genomic DNA
gtctctgtggcgcaatcggttagcgcgttcggctgttaaccgaaaggttggtggttcgatcccacccagggacg

SEQ ID NO: 50 - DNA, 74 bp, Homo sapiens
  source 1..74: mol_type = genomic DNA
gtctctgtggcgcaatcggctagcgcgtttggctgttaactaaaaggttggcggttcgaacccacccagaggcg

SEQ ID NO: 51 - DNA, 74 bp, Homo sapiens
  source 1..74: mol_type = genomic DNA
gtctctgtggtgcaatcggttagcgcgttccgctgttaaccgaaagcttggtggttcgagcccacccagggatg

SEQ ID NO: 52 - DNA, 74 bp, Homo sapiens
  source 1..74: mol_type = genomic DNA
gtctctgtggcgcaatcggctagcgcgtttggctgttaactaaaaagttggtggttcgaacacacccagaggcg

SEQ ID NO: 53 - DNA, 74 bp, Homo sapiens
  source 1..74: mol_type = genomic DNA
gtctctgtggcgcaatcggttagcgcgttcggctgttaaccgaaaggttggtggttcgagcccacccagggacg

SEQ ID NO: 54 - DNA, 74 bp, Homo sapiens
  source 1..74: mol_type = genomic DNA
gtctctgtggcgcaatcggttagcgcattcggctgttaaccgaaaggttggtggttcgagcccacccagggacg

SEQ ID NO: 55 - DNA, 74 bp, Homo sapiens
  source 1..74: mol_type = genomic DNA
gtctctgtggcgcaatcggttagcgcgttcggctgttaaccgaaagattggtggttcgagcccacccagggacg

SEQ ID NO: 56 - DNA, 74 bp, Homo sapiens
  source 1..74: mol_type = genomic DNA
gtctctgtggcgcaatcggttagcgcgttcggctgttaactgaaaggttggtggttcgagcccacccagggacg

SEQ ID NO: 57 - DNA, 74 bp, Homo sapiens
  source 1..74: mol_type = genomic DNA
gtctctgtggcgcaatgggttagcgcgttcggctgttaaccgaaaggttggtggttcgagcccatccagggacg

SEQ ID NO: 58 - DNA, 74 bp, Homo sapiens
  source 1..74: mol_type = genomic DNA
gtctctgtggcgtagtcggttagcgcgttcggctgttaaccgaaaagttggtggttcgagcccacccaggaacg

SEQ ID NO: 59 - DNA, 74 bp, Homo sapiens
  source 1..74: mol_type = genomic DNA
gtctctgtggcgcaatcggctagcgcgtttggctgttaactaaaaggttggtggttcgaacccacccagaggcg

SEQ ID NO: 60 - DNA, 74 bp, Homo sapiens
  source 1..74: mol_type = genomic DNA
gtctctgtggcgcaatcggttagcgcgttcggctgttaactgaaaggttagtggttcgagcccacccggggacg

SEQ ID NO: 61 - DNA, 72 bp, Homo sapiens
  source 1..72: mol_type = genomic DNA
tcctcgttagtatagtggttagtatccccgcctgtcacgcgggagaccggggttcaattccccgacggggag

SEQ ID NO: 62 - DNA, 72 bp, Homo sapiens
  source 1..72: mol_type = genomic DNA
tcctcgttagtatagtggtgagtatccccgcctgtcacgcgggagaccggggttcgattccccgacggggag

SEQ ID NO: 63 - DNA, 72 bp, Homo sapiens
  source 1..72: mol_type = genomic DNA
tcctcgttagtatagtggtgagtgtccccgtctgtcacgcgggagaccggggttcgattccccgacggggag

SEQ ID NO: 64 - DNA, 72 bp, Homo sapiens
  source 1..72: mol_type = genomic DNA
gggggcatagctcagtggtagagcatttgactgcagatcaagaggtccctggttcaaatccaggtgccccct

SEQ ID NO: 65 - DNA, 72 bp, Homo sapiens
  source 1..72: mol_type = genomic DNA
gggggtatagctcaggggtagagcatttgactgcagatcaagaggtccctggttcaaatccaggtgcccccc

SEQ ID NO: 66 - DNA, 72 bp, Homo sapiens
  source 1..72: mol_type = genomic DNA
gggggtatagcttagcggtagagcatttgactgcagatcaagaggtccccggttcaaatccgggtgccccct

SEQ ID NO: 67 - DNA, 72 bp, Homo sapiens
  source 1..72: mol_type = genomic DNA
gggggtatagcttaggggtagagcatttgactgcagatcaaaaggtccctggttcaaatccaggtgcccctt

SEQ ID NO: 68 - DNA, 72 bp, Homo sapiens
  source 1..72: mol_type = genomic DNA
gggggtatagctcaggggtagagcatttgactgcagatcaagaggtccccagttcaaatctgggtgccccct

SEQ ID NO: 69 - DNA, 72 bp, Homo sapiens
  source 1..72: mol_type = genomic DNA
gggggtatagctcaggggtagagcatttgactgcagatcaagaagtccccggttcaaatccgggtgccccct

SEQ ID NO: 70 - DNA, 72 bp, Homo sapiens
  source 1..72: mol_type = genomic DNA
gggggtatagctcaggggtagagcatttgactgcagatcaagaggtctctggttcaaatccaggtgccccct

SEQ ID NO: 71 - DNA, 72 bp, Homo sapiens
  source 1..72: mol_type = genomic DNA
gggggtatagctcaggggtagagcacttgactgcagatcaagaagtccttggttcaaatccaggtgccccct

SEQ ID NO: 72 - DNA, 72 bp, Homo sapiens
  source 1..72: mol_type = genomic DNA
ggggatatagctcaggggtagagcatttgactgcagatcaagaggtccccggttcaaatccgggtgcccccc

SEQ ID NO: 73 - DNA, 72 bp, Homo sapiens
  source 1..72: mol_type = genomic DNA
gggggtatagttcaggggtagagcatttgactgcagatcaagaggtccctggttcaaatccaggtgccccct

SEQ ID NO: 74 - DNA, 72 bp, Homo sapiens
  source 1..72: mol_type = genomic DNA
gggggtatagctcaggggtagagcatttgactgcaaatcaagaggtccctgattcaaatccaggtgccccct

SEQ ID NO: 75 - DNA, 72 bp, Homo sapiens
  source 1..72: mol_type = genomic DNA
gggggtatagctcagtggtagagcatttgactgcagatcaagaggtccccggttcaaatccgggtgccccct

SEQ ID NO: 76 - DNA, 72 bp, Homo sapiens
  source 1..72: mol_type = genomic DNA
gggcgtatagctcaggggtagagcatttgactgcagatcaagaggtccccagttcaaatctgggtgccccct

SEQ ID NO: 77 - DNA, 72 bp, Homo sapiens
  source 1..72: mol_type = genomic DNA
gggggtatagctcacaggtagagcatttgactgcagatcaagaggtccccggttcaaatctgggtgccccct

SEQ ID NO: 78 - DNA, 70 bp, Homo sapiens
  source 1..70: mol_type = genomic DNA
gggcgtatagctcaggggtagagcatttgactgcagatcaagaggtccccagttcaaatctgggtgccca

SEQ ID NO: 79 - DNA, 72 bp, Homo sapiens
  source 1..72: mol_type = genomic DNA
gggggtatagctcacaggtagagcatttgactgcagatcaagaggtccccggttcaaatccggttactccct

SEQ ID NO: 80 - DNA, 72 bp, Homo sapiens
  source 1..72: mol_type = genomic DNA
gggggtatagctcaggggtagagcacttgactgcagatcaagaggtccctggttcaaatccaggtgccccct

SEQ ID NO: 81 - DNA, 72 bp, Homo sapiens
  source 1..72: mol_type = genomic DNA
gggggtatagctcagtggtagagcatttgactgcagatcaagaggtccctggttcaaatccgggtgccccct

SEQ ID NO: 82 - DNA, 73 bp, Homo sapiens
  source 1..73: mol_type = genomic DNA
gggggtatagctcagtgggtagagcatttgactgcagatcaagaggtccccggttcaaatccgggtgccccct

SEQ ID NO: 83 - DNA, 72 bp, Homo sapiens
  source 1..72: mol_type = genomic DNA
gggggtgtagctcagtggtagagcatttgactgcagatcaagaggtccctggttcaaatccaggtgccccct

SEQ ID NO: 84 - DNA, 73 bp, Homo sapiens
  source 1..73: mol_type = genomic DNA
gggggtatagctcaggtggtagagcatttgactgcagatcaagaggtccccggttcaaatccgggtgccccct

SEQ ID NO: 85 - DNA, 72 bp, Homo sapiens
  source 1..72: mol_type = genomic DNA
gggggtatagctcaggggtagagcatttgactgcagatcaagaggtccccggttcaaatccgggtgccccct

SEQ ID NO: 86 - DNA, 72 bp, Homo sapiens
  source 1..72: mol_type = genomic DNA
gggggtatagctcaggggtagagcatttgactgcagatcaagaggtccctggttcaaatccaggtgccccct

SEQ ID NO: 87 - DNA, 72 bp, Homo sapiens
  source 1..72: mol_type = genomic DNA
ggttccatggtgtaatggttagcactctggactctgaatccagcgatccgagttcaaatctcggtggaacct

SEQ ID NO: 88 - DNA, 72 bp, Homo sapiens
  source 1..72: mol_type = genomic DNA
ggttccatggtgtaatggttagcactctggactctgaatccagcgatccgagttcaagtctcggtggaacct

SEQ ID NO: 89 - DNA, 72 bp, Homo sapiens
  source 1..72: mol_type = genomic DNA
ggttccatggtgtaatggtgagcactctggactctgaatccagcgatccgagttcgagtctcggtggaacct

SEQ ID NO: 90 - DNA, 72 bp, Homo sapiens
  source 1..72: mol_type = genomic DNA
ggttccatggtgtaatggtaagcactctggactctgaatccagcgatccgagttcgagtctcggtggaacct

SEQ ID NO: 91 - DNA, 72 bp, Homo sapiens
  source 1..72: mol_type = genomic DNA
ggttccatggtgtaatggttagcactctggactctgaatccggtaatccgagttcaaatctcggtggaacct

SEQ ID NO: 92 - DNA, 72 bp, Homo sapiens
  source 1..72: mol_type = genomic DNA
ggccccatggtgtaatggtcagcactctggactctgaatccagcgatccgagttcaaatctcggtgggaccc

SEQ ID NO: 93 - DNA, 72 bp, Homo sapiens
  source 1..72: mol_type = genomic DNA
ggttccatggtgtaatggtaagcactctggactctgaatccagccatctgagttcgagtctctgtggaacct

SEQ ID NO: 94 - DNA, 72 bp, Homo sapiens
  source 1..72: mol_type = genomic DNA
ggtcccatggtgtaatggttagcactctggactttgaatccagcgatccgagttcaaatctcggtgggacct

SEQ ID NO: 95 - DNA, 72 bp, Homo sapiens
  source 1..72: mol_type = genomic DNA
ggtcccatggtgtaatggttagcactctggactttgaatccagcaatccgagttcgaatctcggtgggacct

SEQ ID NO: 96 - DNA, 72 bp, Homo sapiens
  source 1..72: mol_type = genomic DNA
ggccccatggtgtaatggttagcactctggactttgaatccagcgatccgagttcaaatctcggtgggacct

SEQ ID NO: 97 - DNA, 72 bp, Homo sapiens
  source 1..72: mol_type = genomic DNA
ggtcccatggtgtaatggttagcactctgggctttgaatccagcaatccgagttcgaatcttggtgggacct

SEQ ID NO: 98 - DNA, 72 bp, Homo sapiens
  source 1..72: mol_type = genomic DNA
tccctggtggtctagtggttaggattcggcgctctcaccgccgcggcccgggttcgattcccggtcagggaa

SEQ ID NO: 99 - DNA, 72 bp, Homo sapiens
  source 1..72: mol_type = genomic DNA
tccctggtggtctagtggttaggattcggcgctctcaccgccgcggcccgggttcgattcccggtcaggaaa

SEQ ID NO: 100 - DNA, 72 bp, Homo sapiens
  source 1..72: mol_type = genomic DNA
tcccatatggtctagcggttaggattcctggttttcacccaggtggcccgggttcgactcccggtatgggaa

SEQ ID NO: 101 - DNA, 72 bp, Homo sapiens
  source 1..72: mol_type = genomic DNA
tcccacatggtctagcggttaggattcctggttttcacccaggcggcccgggttcgactcccggtgtgggaa

SEQ ID NO: 102 - DNA, 72 bp, Homo sapiens
  source 1..72: mol_type = genomic DNA
tccctggtggtctagtggctaggattcggcgctttcaccgccgcggcccgggttcgattcccggccagggaa

SEQ ID NO: 103 - DNA, 72 bp, Homo sapiens
  source 1..72: mol_type = genomic DNA
tccctggtggtctagtggctaggattcggcgctttcaccgccgcggcccgggttcgattcccggtcagggaa

SEQ ID NO: 104 - DNA, 71 bp, Homo sapiens
  source 1..71: mol_type = genomic DNA
gcattggtggttcagtggtagaattctcgcctcccacgcgggagacccgggttcaattcccggccaatgca

SEQ ID NO: 105 - DNA, 71 bp, Homo sapiens
  source 1..71: mol_type = genomic DNA
gcgccgctggtgtagtggtatcatgcaagattcccattcttgcgacccgggttcgattcccgggcggcgca

SEQ ID NO: 106 - DNA, 71 bp, Homo sapiens
  source 1..71: mol_type = genomic DNA
gcattggtggttcaatggtagaattctcgcctcccacgcaggagacccaggttcgattcctggccaatgca

SEQ ID NO: 107 - DNA, 71 bp, Homo sapiens
  source 1..71: mol_type = genomic DNA
gcatgggtggttcagtggtagaattctcgcctgccacgcgggaggcccgggttcgattcccggcccatgca

SEQ ID NO: 108 - DNA, 71 bp, Homo sapiens
  source 1..71: mol_type = genomic DNA
gcattggtggttcagtggtagaattctcgcctgccacgcgggaggcccgggttcgattcccggccaatgca

SEQ ID NO: 109 - DNA, 71 bp, Homo sapiens
  source 1..71: mol_type = genomic DNA
gcattggtggttcagtggtagaattctcgcctgccacgcgggaggcccgggtttgattcccggccagtgca

SEQ ID NO: 110 - DNA, 71 bp, Homo sapiens
  source 1..71: mol_type = genomic DNA
gcataggtggttcagtggtagaattcttgcctgccacgcaggaggcccaggtttgattcctggcccatgca

SEQ ID NO: 111 - DNA, 71 bp, Homo sapiens
  source 1..71: mol_type = genomic DNA
gcattggtggttcagtggtagaattctcgcctgccatgcgggcggccgggcttcgattcctggccaatgca

SEQ ID NO: 112 - DNA, 72 bp, Homo sapiens
  source 1..72: mol_type = genomic DNA
gcgttggtggtatagtggttagcatagctgccttccaagcagttgacccgggttcgattcccggccaacgca

SEQ ID NO: 113 - DNA, 72 bp, Homo sapiens
  source 1..72: mol_type = genomic DNA
gcgttggtggtatagtggtgagcatagctgccttccaagcagttgacccgggttcgattcccggccaacgca

SEQ ID NO: 114 - DNA, 72 bp, Homo sapiens
  source 1..72: mol_type = genomic DNA
gcgttggtggtatagtggtaagcatagctgccttccaagcagttgacccgggttcgattcccggccaacgca

SEQ ID NO: 115 - DNA, 72 bp, Homo sapiens
  source 1..72: mol_type = genomic DNA
gcgttggtggtatagtggtgagcatagttgccttccaagcagttgacccgggctcgattcccgcccaacgca

SEQ ID NO: 116 - DNA, 72 bp, Homo sapiens
  source 1..72: mol_type = genomic DNA
gccgtgatcgtatagtggttagtactctgcgttgtggccgcagcaacctcggttcgaatccgagtcacggca

SEQ ID NO: 117 - DNA, 72 bp, Homo sapiens
  source 1..72: mol_type = genomic DNA
gccatgatcgtatagtggttagtactctgcgctgtggccgcagcaacctcggttcgaatccgagtcacggca

SEQ ID NO: 118 - DNA, 74 bp, Homo sapiens
  source 1..74: mol_type = genomic DNA
ggccggttagctcagttggttagagcgtggcgctaataacgccaaggtcgcgggttcgatccccgtacgggcca

SEQ ID NO: 119 - DNA, 74 bp, Homo sapiens
  source 1..74: mol_type = genomic DNA
ggccggttagctcagttggttagagcgtggtgctaataacgccaaggtcgcgggttcgatccccgtactggcca

SEQ ID NO: 120 - DNA, 74 bp, Homo sapiens
  source 1..74: mol_type = genomic DNA
ggctggttagctcagttggttagagcgtggtgctaataacgccaaggtcgcgggttcgatccccgtactggcca

SEQ ID NO: 121 - DNA, 74 bp, Homo sapiens
  source 1..74: mol_type = genomic DNA
ggccggttagctcagttggttagagcgtggtgctaataacgccaaggtcgcgggttcgaaccccgtacgggcca

SEQ ID NO: 122 - DNA, 74 bp, Homo sapiens
  source 1..74: mol_type = genomic DNA
ggccggttagctcagttggttagagcgtggtgctaataacgccaaggtcgcgggttcgatccccgtacgggcca

SEQ ID NO: 123 - DNA, 74 bp, Homo sapiens
  source 1..74: mol_type = genomic DNA
ggccggttagctcagttggttagagcgtggtgctaataacgctaaggtcgcgggttcgatccccgtactggcca

SEQ ID NO: 124 - DNA, 74 bp, Homo sapiens
  source 1..74: mol_type = genomic DNA
ggccggttagctcagttggtcagagcgtggtgctaataacgccaaggtcgcgggttcgatccccgtacgggcca

SEQ ID NO: 125 - DNA, 74 bp, Homo sapiens
  source 1..74: mol_type = genomic DNA
ggccggttagctcagtcggctagagcgtggtgctaataacgccaaggtcgcgggttcgatccccgtacgggcca

SEQ ID NO: 126 - DNA, 74 bp, Homo sapiens
  source 1..74: mol_type = genomic DNA
ggctggttagttcagttggttagagcgtggtgctaataacgccaaggtcgtgggttcgatccccatatcggcca

SEQ ID NO: 127 - DNA, 74 bp, Homo sapiens
  source 1..74: mol_type = genomic DNA
ggccggttagctcagttggtaagagcgtggtgctgataacaccaaggtcgcgggctcgactcccgcaccggcca

SEQ ID NO: 128 - DNA, 93 bp, Homo sapiens
  source 1..93: mol_type = genomic DNA
gctccagtggcgcaatcggttagcgcgcggtacttatatgacagtgcgagcggagcaatgccgaggttgtgagttcgatcctcacctggagca

SEQ ID NO: 129 - DNA, 93 bp, Homo sapiens
  source 1..93: mol_type = genomic DNA
gctccagtggcgcaatcggttagcgcgcggtacttatacagcagtacatgcagagcaatgccgaggttgtgagttcgagcctcacctggagca

SEQ ID NO: 130 - DNA, 94 bp, Homo sapiens
  source 1..94: mol_type = genomic DNA
gctccagtggcgcaatcggttagcgcgcggtacttatatggcagtatgtgtgcgagtgatgccgaggttgtgagttcgagcctcacctggagca

SEQ ID NO: 131 - DNA, 94 bp, Homo sapiens
  source 1..94: mol_type = genomic DNA
gctccagtggcgcaatcggttagcgcgcggtacttatacaacagtatatgtgcgggtgatgccgaggttgtgagttcgagcctcacctggagca

SEQ ID NO: 132 - DNA, 94 bp, Homo sapiens
  source 1..94: mol_type = genomic DNA
gctccagtggcgcaatcggttagcgcgcggtacttataagacagtgcacctgtgagcaatgccgaggttgtgagttcaagcctcacctggagca

SEQ ID NO: 133 - DNA, 82 bp, Homo sapiens
  source 1..82: mol_type = genomic DNA
ggtagcgtggccgagcggtctaaggcgctggattaaggctccagtctcttcggaggcgtgggttcgaatcccaccgctgcca

SEQ ID NO: 134 - DNA, 82 bp, Homo sapiens
  source 1..82: mol_type = genomic DNA
ggtagcgtggccgagcggtctaaggcgctggattaaggctccagtctcttcgggggcgtgggttcgaatcccaccgctgcca

SEQ ID NO: 135 - DNA, 82 bp, Homo sapiens
  source 1..82: mol_type = genomic DNA
ggtagcgtggccgagcggtctaaggcgctggattaaggctccagtctcttcgggggcgtgggttcaaatcccaccgctgcca

SEQ ID NO: 136 - DNA, 82 bp, Homo sapiens
  source 1..82: mol_type = genomic DNA
ggtagcgtggccgagtggtctaagacgctggattaaggctccagtctcttcgggggcgtgggtttgaatcccaccgctgcca

SEQ ID NO: 137 - DNA, 106 bp, Homo sapiens
  source 1..106: mol_type = genomic DNA
gtcaggatggccgagtggtctaaggcgccagactcaagctaagcttcctccgcggtggggattctggtctccaatggaggcgtgggttcgaatcccacttctgaca

SEQ ID NO: 138 - DNA, 105 bp, Homo sapiens
  source 1..105: mol_type = genomic DNA
gtcaggatggccgagtggtctaaggcgccagactcaagcttggcttcctcgtgttgaggattctggtctccaatggaggcgtgggttcgaatcccacttctgaca

SEQ ID NO: 139 - DNA, 108 bp, Homo sapiens
  source 1..108: mol_type = genomic DNA
gtcaggatggccgagtggtctaaggcgccagactcaagcttactgcttcctgtgttcgggtcttctggtctccgtatggaggcgtgggttcgaatcccacttctgaca

SEQ ID NO: 140 - DNA, 107 bp, Homo sapiens
  source 1..107: mol_type = genomic DNA
gtcaggatggccgagtggtctaaggcgccagactcaagttgctacttcccaggtttggggcttctggtctccgcatggaggcgtgggttcgaatcccacttctgaca

SEQ ID NO: 141 - DNA, 106 bp, Homo sapiens
  source 1..106: mol_type = genomic DNA
gtcaggatggccgagtggtctaaggcgccagactcaaggtaagcaccttgcctgcgggctttctggtctccggatggaggcgtgggttcgaatcccacttctgaca

SEQ ID NO: 142 - DNA, 74 bp, Homo sapiens
  source 1..74: mol_type = genomic DNA
gcctccttagtgcagtaggtagcgcatcagtctcaaaatctgaatggtcctgagttcaagcctcagagggggca

SEQ ID NO: 143 - DNA, 84 bp, Homo sapiens
  source 1..84: mol_type = genomic DNA
gtcaggatggccgagcagtcttaaggcgctgcgttcaaatcgcaccctccgctggaggcgtgggttcgaatcccacttttgaca

SEQ ID NO: 144 - DNA, 83 bp, Homo sapiens
  source 1..83: mol_type = genomic DNA
gtcaggatggccgagcggtctaaggcgctgcgttcaggtcgcagtctcccctggaggcgtgggttcgaatcccactcctgaca

SEQ ID NO: 145 - DNA, 83 bp, Homo sapiens
  source 1..83: mol_type = genomic DNA
gtcaggatggccgagcggtctaaggcgctgcgttcaggtcgcagtctcccctggaggcgtgggttcgaatcccacttctgaca

SEQ ID NO: 146 - DNA, 83 bp, Homo sapiens
  source 1..83: mol_type = genomic DNA
accaggatggccgagtggttaaggcgttggacttaagatccaatggacatatgtccgcgtgggttcgaaccccactcctggta

SEQ ID NO: 147 - DNA, 83 bp, Homo sapiens
  source 1..83: mol_type = genomic DNA
accgggatggccgagtggttaaggcgttggacttaagatccaatgggctggtgcccgcgtgggttcgaaccccactctcggta

SEQ ID NO: 148 - DNA, 83 bp, Homo sapiens
  source 1..83: mol_type = genomic DNA
accagaatggccgagtggttaaggcgttggacttaagatccaatggattcatatccgcgtgggttcgaaccccacttctggta

SEQ ID NO: 149 - DNA, 83 bp, Homo sapiens
  source 1..83: mol_type = genomic DNA
accgggatggctgagtggttaaggcgttggacttaagatccaatggacaggtgtccgcgtgggttcgagccccactcccggta

SEQ ID NO: 150 - DNA, 82 bp, Homo sapiens
  source 1..82: mol_type = genomic DNA
ggtagcgtggccgagcggtctaaggcgctggatttaggctccagtctcttcggaggcgtgggttcgaatcccaccgctgcca

SEQ ID NO: 151 - DNA, 82 bp, Homo sapiens
  source 1..82: mol_type = genomic DNA
ggtagtgtggccgagcggtctaaggcgctggatttaggctccagtctcttcgggggcgtgggttcgaatcccaccactgcca

SEQ ID NO: 152 - DNA, 82 bp, Homo sapiens
  source 1..82: mol_type = genomic DNA
ggtagcgtggccgagtggtctaaggcgctggatttaggctccagtcatttcgatggcgtgggttcgaatcccaccgctgcca

SEQ ID NO: 153 - DNA, 73 bp, Homo sapiens
  source 1..73: mol_type = genomic DNA
gcccggctagctcagtcggtagagcatgggactcttaatcccagggtcgtgggttcgagccccacgttgggcg

SEQ ID NO: 154 - DNA, 73 bp, Homo sapiens
  source 1..73: mol_type = genomic DNA
gcccagctagctcagtcggtagagcataagactcttaatctcagggttgtggattcgtgccccatgctgggtg

SEQ ID NO: 155 - DNA, 74 bp, Homo sapiens
  source 1..74: mol_type = genomic DNA
gcagctagctcagtcggtagagcatgagactcttaatctcagggtcatgggttcgtgccccatgttgggtgcca

SEQ ID NO: 156 - DNA, 73 bp, Homo sapiens
  source 1..73: mol_type = genomic DNA
gcccggctagctcagtcggtagagcatgagactcttaatctcagggtcgtgggttcgagccccacgttgggcg

SEQ ID NO: 157 - DNA, 73 bp, Homo sapiens
  source 1..73: mol_type = genomic DNA
gcccggctagctcagtcggtagagcatgagacccttaatctcagggtcgtgggttcgagccccacgttgggcg

SEQ ID NO: 158 - DNA, 73 bp, Homo sapiens
  source 1..73: mol_type = genomic DNA
gcccggctagctcagtcggtagagcatgggactcttaatctcagggtcgtgggttcgagccccacgttgggcg

SEQ ID NO: 159 - DNA, 73 bp, Homo sapiens
  source 1..73: mol_type = genomic DNA
gcccggctagctcagtcgatagagcatgagactcttaatctcagggtcgtgggttcgagccgcacgttgggcg

SEQ ID NO: 160 - DNA, 73 bp, Homo sapiens
  source 1..73: mol_type = genomic DNA
gcccagctagctcagtcggtagagcatgagactcttaatctcagggtcatgggtttgagccccacgtttggtg

SEQ ID NO: 161 - DNA, 73 bp, Homo sapiens
  source 1..73: mol_type = genomic DNA
gcctggctagctcagtcggcaaagcatgagactcttaatctcagggtcgtgggctcgagctccatgttgggcg

SEQ ID NO: 162 - DNA, 73 bp, Homo sapiens
  source 1..73: mol_type = genomic DNA
gcccgactacctcagtcggtggagcatgggactcttcatcccagggttgtgggttcgagccccacattgggca

SEQ ID NO: 163 - DNA, 73 bp, Homo sapiens
  source 1..73: mol_type = genomic DNA
gcctggatagctcagttggtagagcatcagacttttaatctgagggtccagggttcaagtccctgttcaggca

SEQ ID NO: 164 - DNA, 73 bp, Homo sapiens
  source 1..73: mol_type = genomic DNA
acccagatagctcagtcagtagagcatcagacttttaatctgagggtccaaggttcatgtccctttttgggtg

SEQ ID NO: 165 - DNA, 73 bp, Homo sapiens
  source 1..73: mol_type = genomic DNA
gcctggatagctcagttggtagagcatcagacttttaatctgagggtccagggttcaagtccctgttcaggcg

SEQ ID NO: 166 - DNA, 73 bp, Homo sapiens
  source 1..73: mol_type = genomic DNA
gcccggatagctcagtcggtagagcatcagacttttaatctgagggtccagggttcaagtccctgttcgggcg

SEQ ID NO: 167 - DNA, 73 bp, Homo sapiens
  source 1..73: mol_type = genomic DNA
gcctggatagctcagtcggtagagcatcagacttttaatctgagggtccagggttcaagtccctgttcaggcg

SEQ ID NO: 168 - DNA, 73 bp, Homo sapiens
  source 1..73: mol_type = genomic DNA
gcccggatagctcagtcggtagagcatcagacttttaatctgagggtccggggttcaagtccctgttcgggcg

SEQ ID NO: 169 - DNA, 73 bp, Homo sapiens
  source 1..73: mol_type = genomic DNA
gcctgggtagctcagtcggtagagcatcagacttttaatctgagggtccagggttcaagtccctgtccaggcg

SEQ ID NO: 170 - DNA, 73 bp, Homo sapiens
  source 1..73: mol_type = genomic DNA
gcctggatagctcagttggtagaacatcagacttttaatctgacggtgcagggttcaagtccctgttcaggcg

SEQ ID NO: 171 - DNA, 73 bp, Homo sapiens
  source 1..73: mol_type = genomic DNA
gcctcgttagcgcagtaggtagcgcgtcagtctcataatctgaaggtcgtgagttcgatcctcacacggggca

SEQ ID NO: 172 - DNA, 73 bp, Homo sapiens
  source 1..73: mol_type = genomic DNA
gccctcttagcgcagtgggcagcgcgtcagtctcataatctgaaggtcctgagttcgagcctcagagagggca

SEQ ID NO: 173 - DNA, 73 bp, Homo sapiens
  source 1..73: mol_type = genomic DNA
gcctccttagcgcagtaggcagcgcgtcagtctcataatctgaaggtcctgagttcgaacctcagagggggca

SEQ ID NO: 174 - DNA, 73 bp, Homo sapiens
  source 1..73: mol_type = genomic DNA
gccctcttagcgcagcgggcagcgcgtcagtctcataatctgaaggtcctgagttcgagcctcagagagggca

SEQ ID NO: 175 - DNA, 73 bp, Homo sapiens
  source 1..73: mol_type = genomic DNA
gccctcttagcgcagctggcagcgcgtcagtctcataatctgaaggtcctgagttcaagcctcagagagggca

SEQ ID NO: 176 - DNA, 73 bp, Homo sapiens
  source 1..73: mol_type = genomic DNA
gcctcgttagcgcagtaggcagcgcgtcagtctcataatctgaaggtcgtgagttcgagcctcacacggggca

SEQ ID NO: 177 - DNA, 73 bp, Homo sapiens
  source 1..73: mol_type = genomic DNA
gccctcttagtgcagctggcagcgcgtcagtttcataatctgaaagtcctgagttcaagcctcagagagggca

SEQ ID NO: 178 - DNA, 73 bp, Homo sapiens
  source 1..73: mol_type = genomic DNA
gccgaaatagctcagttgggagagcgttagactgaagatctaaaggtccctggttcgatcccgggtttcggca

SEQ ID NO: 179 - DNA, 73 bp, Homo sapiens
  source 1..73: mol_type = genomic DNA
gccgaaatagctcagttgggagagcgttagactgaagatctaaaggtccctggttcaatcccgggtttcggca

SEQ ID NO: 180 - DNA, 73 bp, Homo sapiens
  source 1..73: mol_type = genomic DNA
gccgagatagctcagttgggagagcgttagactgaagatctaaaggtccctggttcaatcccgggtttcggca

SEQ ID NO: 181 - DNA, 74 bp, Homo sapiens
  source 1..74: mol_type = genomic DNA
gccgaaatagctcagttgggagagcgttagaccgaagatcttaaaggtccctggttcaatcccgggtttcggca

SEQ ID NO: 182 - DNA, 74 bp, Homo sapiens
  source 1..74: mol_type = genomic DNA
gctgaaatagctcagttgggagagcgttagactgaagatcttaaagttccctggttcaaccctgggtttcagcc

SEQ ID NO: 183 - DNA, 72 bp, Homo sapiens
  source 1..72: mol_type = genomic DNA
ggctcgttggtctaggggtatgattctcgcttaggatgcgagaggtcccgggttcaaatcccggacgagccc

SEQ ID NO: 184 - DNA, 72 bp, Homo sapiens
  source 1..72: mol_type = genomic DNA
ggctcgttggtctaggggtatgattctcgcttagggtgcgagaggtcccgggttcaaatcccggacgagccc

SEQ ID NO: 185 - DNA, 72 bp, Homo sapiens
  source 1..72: mol_type = genomic DNA
ggctcgttggtctaggggtatgattctcgcttcgggtgcgagaggtcccgggttcaaatcccggacgagccc

SEQ ID NO: 186 - DNA, 72 bp, Homo sapiens
  source 1..72: mol_type = genomic DNA
ggctcgttggtctaggggtatgattctcgcttcgggtgtgagaggtcccgggttcaaatcccggacgagccc

SEQ ID NO: 187 - DNA, 72 bp, Homo sapiens
  source 1..72: mol_type = genomic DNA
ggctcgttggtctagtggtatgattctcgctttgggtgcgagaggtcccgggttcaaatcccggacgagccc

SEQ ID NO: 188 - DNA, 72 bp, Homo sapiens
  source 1..72: mol_type = genomic DNA
ggctcgttggtctaggggtatgattctcggtttgggtccgagaggtcccgggttcaaatcccggacgagccc

SEQ ID NO: 189 - DNA, 72 bp, Homo sapiens
  source 1..72: mol_type = genomic DNA
ggctcgttggtctaggggtatgattctcgctttgggtgcgagaggtcccgggttcaaatcccggacgagccc

SEQ ID NO: 190 - DNA, 87 bp, Homo sapiens
  source 1..87: mol_type = genomic DNA
gcccggatgatcctcagtggtctggggtgcaggcttcaaacctgtagctgtctagcgacagagtggttcaattccacctttcgggcg

SEQ ID NO: 191 - DNA, 84 bp, Homo sapiens
  source 1..84: mol_type = genomic DNA
gctcggatgatcctcagtggtctggggtgcaggcttcaaacctgtagctgtctagtgacagagtggttcaattccacctttgta

SEQ ID NO: 192 - DNA, 82 bp, Homo sapiens
  source 1..82: mol_type = genomic DNA
gtagtcgtggccgagtggttaaggcgatggactagaaatccattggggtttccccgcgcaggttcgaatcctgccgactacg

SEQ ID NO: 193 - DNA, 82 bp, Homo sapiens
  source 1..82: mol_type = genomic DNA
gtagtcgtggccgagtggttaaggcgatggactagaaatccattggggtctccccgcgcaggttcgaatcctgccgactacg

SEQ ID NO: 194 - DNA, 82 bp, Homo sapiens
  source 1..82: mol_type = genomic DNA
gtagtcgtggccgagtggttaaggcgatggactagaaatccattggggtttccccacgcaggttcgaatcctgccgactacg

SEQ ID NO: 195 - DNA, 82 bp, Homo sapiens
  source 1..82: mol_type = genomic DNA
gtagtcgtggccgagtggttaaggtgatggactagaaacccattggggtctccccgcgcaggttcgaatcctgccgactacg

SEQ ID NO: 196 - DNA, 82 bp, Homo sapiens
  source 1..82: mol_type = genomic DNA
gctgtgatggccgagtggttaaggcgttggactcgaaatccaatggggtctccccgcgcaggttcgaatcctgctcacagcg

SEQ ID NO: 197 - DNA, 82 bp, Homo sapiens
  source 1..82: mol_type = genomic DNA
gctgtgatggccgagtggttaaggcgttggactcgaaatccaatggggtctccccgcgcaggttcaaatcctgctcacagcg

SEQ ID NO: 198 - DNA, 82 bp, Homo sapiens
  source 1..82: mol_type = genomic DNA
gctgtgatggccgagtggttaaggtgttggactcgaaatccaatgggggttccccgcgcaggttcaaatcctgctcacagcg

SEQ ID NO: 199 - DNA, 82 bp, Homo sapiens
  source 1..82: mol_type = genomic DNA
gtcacggtggccgagtggttaaggcgttggactcgaaatccaatggggtttccccgcacaggttcgaatcctgttcgtgacg

SEQ ID NO: 200 - DNA, 82 bp, Homo sapiens
  source 1..82: mol_type = genomic DNA
gacgaggtggccgagtggttaaggcgatggactgctaatccattgtgctctgcacgcgtgggttcgaatcccaccctcgtcg

SEQ ID NO: 201 - DNA, 82 bp, Homo sapiens
  source 1..82: mol_type = genomic DNA
gacgaggtggccgagtggttaaggcgatggactgctaatccattgtgctctgcacgcgtgggttcgaatcccaccttcgtcg

SEQ ID NO: 202 - DNA, 82 bp, Homo sapiens
  source 1..82: mol_type = genomic DNA
gacgaggtggccgagtggttaaggcgatggactgctaatccattgtgctttgcacgcgtgggttcgaatcccatcctcgtcg

SEQ ID NO: 203 - DNA, 82 bp, Homo sapiens
  source 1..82: mol_type = genomic DNA
gacgaggtggccgagtggttaaggcgatggactgctaatccattgtgctctgcacgcgtgggttcgaatcccatcctcgtcg

SEQ ID NO: 204 - DNA, 82 bp, Homo sapiens
  source 1..82: mol_type = genomic DNA
gacgaggtggccgagtggttaaggcgatggactgctaatccattgtgctctgcacacgtgggttcgaatcccatcctcgtcg

SEQ ID NO: 205 - DNA, 84 bp, Homo sapiens
  source 1..84: mol_type = genomic DNA
ggagaggcctggccgagtggttaaggcgatggactgctaatccattgtgctctgcacgcgtgggttcgaatcccatcctcgtcg

SEQ ID NO: 206 - DNA, 82 bp, Homo sapiens
  source 1..82: mol_type = genomic DNA
gcagcgatggccgagtggttaaggcgttggacttgaaatccaatggggtctccccgcgcaggttcgaaccctgctcgctgcg

SEQ ID NO: 207 - DNA, 82 bp, Homo sapiens
  source 1..82: mol_type = genomic DNA
gtagtcgtggccgagtggttaaggcgatggacttgaaatccattggggtttccccgcgcaggttcgaatcctgccgactacg

SEQ ID NO: 208 - DNA, 82 bp, Homo sapiens
  source 1..82: mol_type = genomic DNA
gtagtcgtggccgagtggttaaggcgatggacttgaaatccattggggtctccccgcgcaggttcgaatcctgccgactacg

SEQ ID NO: 209 - DNA, 82 bp, Homo sapiens
  source 1..82: mol_type = genomic DNA
gtagtcgtggccgagtggttaaggcgatggacttgaaatccattggggtttccccgcgcaggttcgaatcctgtcggctacg

SEQ ID NO: 210 - DNA, 74 bp, Homo sapiens
  source 1..74: mol_type = genomic DNA
ggcgccgtggcttagttggttaaagcgcctgtctagtaaacaggagatcctgggttcgaatcccagcggtgcct

SEQ ID NO: 211 - DNA, 74 bp, Homo sapiens
  source 1..74: mol_type = genomic DNA
ggctccgtggcttagctggttaaagcgcctgtctagtaaacaggagatcctgggttcgaatcccagcggggcct

SEQ ID NO: 212 - DNA, 74 bp, Homo sapiens
  source 1..74: mol_type = genomic DNA
ggctccgtagcttagttggttaaagcgcctgtctagtaaacaggagatcctgggttcgactcccagcggggcct

SEQ ID NO: 213 - DNA, 74 bp, Homo sapiens
  source 1..74: mol_type = genomic DNA
ggcttcgtggcttagctggttaaagcgcctgtctagtaaacaggagatcctgggttcgaatcccagcgaggcct

SEQ ID NO: 214 - DNA, 74 bp, Homo sapiens
  source 1..74: mol_type = genomic DNA
ggcgccgtggcttagctggttaaagcgcctgtctagtaaacaggagatcctgggttcgaatcccagcggtgcct

SEQ ID NO: 215 - DNA, 74 bp, Homo sapiens
  source 1..74: mol_type = genomic DNA
ggccctgtggcttagctggtcaaagcgcctgtctagtaaacaggagatcctgggttcgaatcccagcggggcct

SEQ ID NO: 216 - DNA, 74 bp, Homo sapiens
  source 1..74: mol_type = genomic DNA
ggctctatggcttagttggttaaagcgcctgtctcgtaaacaggagatcctgggttcgactcccagtggggcct

SEQ ID NO: 217 - DNA, 72 bp, Homo sapiens
  source 1..72: mol_type = genomic DNA
ggcgcggtggccaagtggtaaggcgtcggtctcgtaaaccgaagatcacgggttcgaaccccgtccgtgcct

SEQ ID NO: 218 - DNA, 74 bp, Homo sapiens
  source 1..74: mol_type = genomic DNA
ggctctgtggcttagttggctaaagcgcctgtctcgtaaacaggagatcctgggttcgaatcccagcggggcct

SEQ ID NO: 219 - DNA, 72 bp, Homo sapiens
  source 1..72: mol_type = genomic DNA
ggcgcggtggccaagtggtaaggcgtcggtctcgtaaaccgaagatcgcgggttcgaaccccgtccgtgcct

SEQ ID NO: 220 - DNA, 74 bp, Homo sapiens
  source 1..74: mol_type = genomic DNA
ggccctgtagctcagcggttggagcgctggtctcgtaaacctaggggtcgtgagttcaaatctcaccagggcct

SEQ ID NO: 221 - DNA, 74 bp, Homo sapiens
  source 1..74: mol_type = genomic DNA
ggctctatggcttagttggttaaagcgcctgtcttgtaaacaggagatcctgggttcgaatcccagtagagcct

SEQ ID NO: 222 - DNA, 73 bp, Homo sapiens
  source 1..73: mol_type = genomic DNA
ggctccatagctcagtggttagagcactggtcttgtaaaccaggggtcgcgagttcgatcctcgctggggcct

SEQ ID NO: 223 - DNA, 73 bp, Homo sapiens
  source 1..73: mol_type = genomic DNA
ggctccatagctcaggggttagagcgctggtcttgtaaaccaggggtcgcgagttcaattctcgctggggcct

SEQ ID NO: 224 - DNA, 73 bp, Homo sapiens
  source 1..73: mol_type = genomic DNA
ggctccatagctcaggggttagagcactggtcttgtaaaccaggggtcgcgagttcaaatctcgctggggcct

SEQ ID NO: 225 - DNA, 73 bp, Homo sapiens
  source 1..73: mol_type = genomic DNA
ggccctatagctcaggggttagagcactggtcttgtaaaccaggggtcgcgagttcaaatctcgctggggcct

SEQ ID NO: 226 - DNA, 72 bp, Homo sapiens
  source 1..72: mol_type = genomic DNA
ggctccatagctcaggggttagagcactggtcttgtaaaccagggtcgcgagttcaaatctcgctggggcct

SEQ ID NO: 227 - DNA, 72 bp, Homo sapiens
  source 1..72: mol_type = genomic DNA
ggcctcgtggcgcaacggtagcgcgtctgactccagatcagaaggttgcgtgttcaaatcacgtcggggtca

SEQ ID NO: 228 - DNA, 72 bp, Homo sapiens
  source 1..72: mol_type = genomic DNA
gacctcgtggcgcaatggtagcgcgtctgactccagatcagaaggttgcgtgttcaagtcacgtcggggtca

SEQ ID NO: 229 - DNA, 72 bp, Homo sapiens
  source 1..72: mol_type = genomic DNA
gacctcgtggcgcaacggtagcgcgtctgactccagatcagaaggttgcgtgttcaaatcacgtcggggtca

SEQ ID NO: 230 - DNA, 72 bp, Homo sapiens
  source 1..72: mol_type = genomic DNA
gacctcgtggcgcaacggtagcgcgtctgactccagatcagaaggctgcgtgttcgaatcacgtcggggtca

SEQ ID NO: 231 - DNA, 72 bp, Homo sapiens
  source 1..72: mol_type = genomic DNA
gacctcgtggcgcaacggcagcgcgtctgactccagatcagaaggttgcgtgttcaaatcacgtcggggtca

SEQ ID NO: 232 - DNA, 93 bp, Homo sapiens
  source 1..93: mol_type = genomic DNA
ccttcaatagttcagctggtagagcagaggactatagctacttcctcagtaggagacgtccttaggttgctggttcgattccagcttgaagga

SEQ ID NO: 233 - DNA, 91 bp, Homo sapiens
  source 1..91: mol_type = genomic DNA
ccttcgatagctcagttggtagagcggaggactgtagttggctgtgtccttagacatccttaggtcgctggttcgaatccggctcgaagga

SEQ ID NO: 234 - DNA, 89 bp, Homo sapiens
  source 1..89: mol_type = genomic DNA
ccttcgatagctcagttggtagagcggaggactgtagtggatagggcgtggcaatccttaggtcgctggttcgattccggctcgaagga

SEQ ID NO: 235 - DNA, 89 bp, Homo sapiens
  source 1..89: mol_type = genomic DNA
ccttcgatagctcagttggtagagcggaggactgtaggctcattaagcaaggtatccttaggtcgctggttcgaatccggctcggagga

SEQ ID NO: 236 - DNA, 94 bp, Homo sapiens
  source 1..94: mol_type = genomic DNA
ccttcgatagctcagctggtagagcggaggactgtagattgtatagacatttgcggacatccttaggtcgctggttcgattccagctcgaagga

SEQ ID NO: 237 - DNA, 93 bp, Homo sapiens
  source 1..93: mol_type = genomic DNA
ccttcgatagctcagctggtagagcggaggactgtagctacttcctcagcaggagacatccttaggtcgctggttcgattccggctcgaagga

SEQ ID NO: 238 - DNA, 89 bp, Homo sapiens
  source 1..89: mol_type = genomic DNA
ccttcgatagctcagctggtagagcggaggactgtaggcgcgcgcccgtggccatccttaggtcgctggttcgattccggctcgaagga

SEQ ID NO: 239 - DNA, 94 bp, Homo sapiens
  source 1..94: mol_type = genomic DNA
ccttcgatagctcagctggtagagcggaggactgtagcctgtagaaacatttgtggacatccttaggtcgctggttcgattccggctcgaagga

SEQ ID NO: 240 - DNA, 94 bp, Homo sapiens
  source 1..94: mol_type = genomic DNA
ccttcgatagctcagctggtagagcggaggactgtagattgtacagacatttgcggacatccttaggtcgctggttcgattccggctcgaagga

SEQ ID NO: 241 - DNA, 89 bp, Homo sapiens
  source 1..89: mol_type = genomic DNA
ccttcgatagctcagctggtagagcggaggactgtagtacttaatgtgtggtcatccttaggtcgctggttcgattccggctcgaagga

SEQ ID NO: 242 - DNA, 89 bp, Homo sapiens
  source 1..89: mol_type = genomic DNA
ccttcgatagctcagctggtagagcggaggactgtaggggtttgaatgtggtcatccttaggtcgctggttcgaatccggctcggagga

SEQ ID NO: 243 - DNA, 94 bp, Homo sapiens
  source 1..94: mol_type = genomic DNA
ccttcgatagctcagctggtagagcggaggactgtagactgcggaaacgtttgtggacatccttaggtcgctggttcaattccggctcgaagga

SEQ ID NO: 244 - DNA, 90 bp, Homo sapiens
  source 1..90: mol_type = genomic DNA
ctttcgatagctcagttggtagagcggaggactgtaggttcattaaactaaggcatccttaggtcgctggttcgaatccggctcgaagga

SEQ ID NO: 245 - DNA, 88 bp, Homo sapiens
  source 1..88: mol_type = genomic DNA
tcttcaatagctcagctggtagagcggaggactgtaggtgcacgcccgtggccattcttaggtgctggtttgattccgacttggagag

SEQ ID NO: 246 - DNA, 73 bp, Homo sapiens
  source 1..73: mol_type = genomic DNA
gtttccgtagtgtagtggttatcacgttcgcctaacacgcgaaaggtccccggttcgaaaccgggcggaaaca

SEQ ID NO: 247 - DNA, 73 bp, Homo sapiens
  source 1..73: mol_type = genomic DNA
gtttccgtagtgtagtggtcatcacgttcgcctaacacgcgaaaggtccccggttcgaaaccgggcggaaaca

SEQ ID NO: 248 - DNA, 73 bp, Homo sapiens
  source 1..73: mol_type = genomic DNA
gtttccgtagtgtagtggttatcacgttcgcctaacacgcgaaaggtccctggatcaaaaccaggcggaaaca

SEQ ID NO: 249 - DNA, 73 bp, Homo sapiens
  source 1..73: mol_type = genomic DNA
gtttccgtagtgtagtggttatcacgttcgcctaacacgcgaaaggtccgcggttcgaaaccgggcggaaaca

SEQ ID NO: 250 - DNA, 73 bp, Homo sapiens
  source 1..73: mol_type = genomic DNA
gtttccgtagtgtagtggttatcacgtttgcctaacacgcgaaaggtccccggttcgaaaccgggcagaaaca

SEQ ID NO: 251 - DNA, 72 bp, Homo sapiens
  source 1..72: mol_type = genomic DNA
gggggtgtagctcagtggtagagcgtatgcttaacattcatgaggctctgggttcgatccccagcacttcca

SEQ ID NO: 252 - DNA, 73 bp, Homo sapiens
  source 1..73: mol_type = genomic DNA
gtttccgtagtgtagtggttatcacgttcgcctcacacgcgaaaggtccccggttcgaaaccgggcggaaaca

SEQ ID NO: 253 - DNA, 73 bp, Homo sapiens
  source 1..73: mol_type = genomic DNA
gcttctgtagtgtagtggttatcacgttcgcctcacacgcgaaaggtccccggttcgaaaccgggcagaagca

SEQ ID NO: 254 - DNA, 73 bp, Homo sapiens
  source 1..73: mol_type = genomic DNA
gtttccgtagtgtagcggttatcacattcgcctcacacgcgaaaggtccccggttcgatcccgggcggaaaca

SEQ ID NO: 255 - DNA, 73 bp, Homo sapiens
  source 1..73: mol_type = genomic DNA
gtttccgtagtgtagtggttatcacgttcgcctcacacgcgaaaggtccccggttcgaaactgggcggaaaca

SEQ ID NO: 256 - DNA, 74 bp, Homo sapiens
  source 1..74: mol_type = genomic DNA
gtttccgtagtgtagtggttatcacgttcgcctcacacgcgtaaaggtccccggttcgaaaccgggcggaaaca

SEQ ID NO: 257 - DNA, 73 bp, Homo sapiens
  source 1..73: mol_type = genomic DNA
gtttccgtagtggagtggttatcacgttcgcctcacacgcgaaaggtccccggtttgaaaccaggcggaaaca

SEQ ID NO: 258 - DNA, 73 bp, Homo sapiens
  source 1..73: mol_type = genomic DNA
ggttccatagtgtagtggttatcacgtctgctttacacgcagaaggtcctgggttcgagccccagtggaacca

SEQ ID NO: 259 - DNA, 73 bp, Homo sapiens
  source 1..73: mol_type = genomic DNA
ggttccatagtgtagcggttatcacgtctgctttacacgcagaaggtcctgggttcgagccccagtggaacca

SEQ ID NO: 260 - DNA, 73 bp, Homo sapiens
  source 1..73: mol_type = genomic DNA
ggttccatagtgtagtggttatcacatctgctttacacgcagaaggtcctgggttcaagccccagtggaacca

SEQ ID NO: 261 - DNA, 73 bp, Homo sapiens
  source 1..73: mol_type = genomic DNA
gtttccgtggtgtagtggttatcacattcgccttacacgcgaaaggtcctcgggtcgaaaccgagcggaaaca

SEQ ID NO: 262 - DNA, 72 bp, Homo sapiens
  source 1..72: mol_type = genomic DNA
agcagagtggcgcagcggaagcgtgctgggcccataacccagaggtcgatggatcgaaaccatcctctgcta

SEQ ID NO: 263 - DNA, 72 bp, Homo sapiens
  source 1..72: mol_type = genomic DNA
agcagagtggcgcagcggaagcgtgctgggcccataacccagaggtcgatggatctaaaccatcctctgcta

SEQ ID NO: 264 - DNA, 73 bp, Homo sapiens
  source 1..73: mol_type = genomic DNA
tccctggtggtctagtggctaggattcggcgctttcaccgccgcggcccgggttcgattcccggtcagggaat

SEQ ID NO: 265 - DNA, 72 bp, Homo sapiens
  source 1..72: mol_type = genomic DNA
gcgttggtggtttagtggtagaattctcgcctcccatgcgggagacccgggttcaattcccggccactgcac

SEQ ID NO: 266 - DNA, 72 bp, Homo sapiens
  source 1..72: mol_type = genomic DNA
ggccttggtggtgcagtggtagaattctcgcctcccacgtgggagacccgggttcaattcccggccaatgca

SEQ ID NO: 267 - DNA, 73 bp, Homo sapiens
  source 1..73: mol_type = genomic DNA
gtccctggtggtctagtggctaggattcggcgctttcaccgccgcggcccgggttcgattcccggccagggaa

SEQ ID NO: 268 - DNA, 75 bp, Homo sapiens
  source 1..75: mol_type = genomic DNA
tgtctctgtggcgcaatcggttagcgcgttcggctgttaaccgaaagattggtggttcgagcccacccagggacg

SEQ ID NO: 269 - DNA, 86 bp, Homo sapiens
  source 1..86: mol_type = genomic DNA
tggctccgtggcgcaatggatagcgcattggacttctagaggctgaaggcattcaaaggttccgggttcgagtcccggcggagtcg

SEQ ID NO: 270 - DNA, 74 bp, Homo sapiens
  source 1..74: mol_type = genomic DNA
gcccggctagctcagtcggtagagcatgagactcttaatctcagggtcgtgggttcgagccccacgttgggcgc

SEQ ID NO: 271 - DNA, 73 bp, Homo sapiens
  source 1..73: mol_type = genomic DNA
gccgtgatcgtatagtggttagtactctgcgttgtggccgcagcaacctcggttcgaatccgagtcacggcag

SEQ ID NO: 272 - DNA, 73 bp, Homo sapiens
  source 1..73: mol_type = genomic DNA
gcgttggtggtatagtggtgagcatagctgccttccaagcagttgacccgggttcgattcccggccaacgcag

SEQ ID NO: 273 - DNA, 73 bp, Homo sapiens
  source 1..73: mol_type = genomic DNA
tccctggtggtctagtggttaggattcggcgctctcaccgccgcggcccgggttcgattcccggtcagggaaa

SEQ ID NO: 274 - DNA, 73 bp, Homo sapiens
  source 1..73: mol_type = genomic DNA
aggttccatggtgtaatggtgagcactctggactctgaatccagcgatccgagttcgagtctcggtggaacct

SEQ ID NO: 275 - DNA, 75 bp, Homo sapiens
  source 1..75: mol_type = genomic DNA
tgtctctgtggcgtagtcggttagcgcgttcggctgttaaccgaaaagttggtggttcgagcccacccaggaacg

SEQ ID NO: 276 - DNA, 75 bp, Homo sapiens
  source 1..75: mol_type = genomic DNA
tgtctctgtggcgcaatcggttagcgcgttcggctgttaaccgaaaggttggtggttcgagcccacccagggacg

SEQ ID NO: 277 - DNA, 75 bp, Homo sapiens
  source 1..75: mol_type = genomic DNA
gtctctgtggcgcaatcggttagcgcattcggctgttaaccgaaaggttggtggttcgagcccacccagggacgc

SEQ ID NO: 278 - DNA, 75 bp, Homo sapiens
  source 1..75: mol_type = genomic DNA
gtctctgtggcgcaatgggttagcgcgttcggctgttaaccgaaaggttggtggttcgagcccatccagggacgc

SEQ ID NO: 279 - DNA, 72 bp, Homo sapiens
  source 1..72: mol_type = genomic DNA
gcactggtggttcagtggtagaattctcgcctcacacgcgggacacccgggttcaattcccggtcaaggcaa

SEQ ID NO: 280 - DNA, 74 bp, Homo sapiens
  source 1..74: mol_type = genomic DNA
gtttccgtagtgtagtggttatcacgttcgcctcacacgcgaaaggtccccggttcgaaactgggcggaaacag

SEQ ID NO: 281 - DNA, 72 bp, Homo sapiens
  source 1..72: mol_type = genomic DNA
gcactggtggttcagtggtagaattctcgcctcccacgcgggagacccgggtttaattcccggtcaagataa

SEQ ID NO: 282 - DNA, 75 bp, Homo sapiens
  source 1..75: mol_type = genomic DNA
gtttccgtagtgtagtggttatcacgttcgcctcacacgcgtaaaggtccccggttcgaaaccgggcggaaacat

SEQ ID NO: 283 - DNA, 73 bp, Homo sapiens
  source 1..73: mol_type = genomic DNA
tagcagagtggcgcagcggaagcgtgctgggcccataacccagaggtcgatggatcgaaaccatcctctgcta

SEQ ID NO: 284 - DNA, 74 bp, Homo sapiens
  source 1..74: mol_type = genomic DNA
gtttccgtagtgtagtggttatcacgttcgcctcacacgcgaaaggtccccggttcgaaaccgggcggaaacaa

SEQ ID NO: 285 - DNA, 73 bp, Homo sapiens
  source 1..73: mol_type = genomic DNA
tcctcgttagtatagtggtgagtatccccgcctgtcacgcgggagaccggggttcgattccccgacggggagg

SEQ ID NO: 286 - DNA, 72 bp, Homo sapiens
  source 1..72: mol_type = genomic DNA
tgcatgggtggttcagtggtagaattctcgcctgccacgcgggaggcccgggttcgattcccggcccatgca

SEQ ID NO: 287 - DNA, 73 bp, Homo sapiens
  source 1..73: mol_type = genomic DNA
tccctggtggtctagtggttaggattcggcgctctcaccgccgcggcccgggttcgattcccggtcagggaag

SEQ ID NO: 288 - DNA, 73 bp, Homo sapiens
  source 1..73: mol_type = genomic DNA
atccttgttactatagtggtgagtatctctgcctgtcatgcgtgagagagggggtcgattccccgacggggag

SEQ ID NO: 289 - DNA, 72 bp, Homo sapiens
  source 1..72: mol_type = genomic DNA
gcattggtggttcagtggtagaattctcgcctgccacgcgggaggcccgggttcgattcccggccaatgcac

SEQ ID NO: 290 - DNA, 84 bp, Homo sapiens
  source 1..84: mol_type = genomic DNA
gtcaggatggccgagcggtctaaggcgctgcgttcaggtcgcagtctcccctggaggcgtgggttcgaatcccactcctgacaa

SEQ ID NO: 291 - DNA, 73 bp, Homo sapiens
  source 1..73: mol_type = genomic DNA
cgcgttggtggtatagtggtgagcatagctgccttccaagcagttgacccgggttcgattcccggccaacgca

SEQ ID NO: 292 - DNA, 75 bp, Homo sapiens
  source 1..75: mol_type = genomic DNA
cgtctctgtggcgcaatcggttagcgcgttcggctgttaaccgaaaggttggtggttcgatcccacccagggacg

SEQ ID NO: 293 - DNA, 73 bp, Homo sapiens
  source 1..73: mol_type = genomic DNA
cgcgttggtggtgtagtggtgagcacagctgcctttcaagcagttaacgcgggttcgattcccgggtaacgaa

SEQ ID NO: 294 - DNA, 73 bp, Homo sapiens
  source 1..73: mol_type = genomic DNA
cggctcgttggtctaggggtatgattctcgcttcgggtgcgagaggtcccgggttcaaatcccggacgagccc

SEQ ID NO: 295 - DNA, 73 bp, Homo sapiens
  source 1..73: mol_type = genomic DNA
ggctcgttggtctaggggtatgattctcgcttagggtgcgagaggtcccgggttcaaatcccggacgagccct

SEQ ID NO: 296 - DNA, 74 bp, Homo sapiens
  source 1..74: mol_type = genomic DNA
cgcccggatagctcagtcggtagagcatcagacttttaatctgagggtccagggttcaagtccctgttcgggcg

SEQ ID NO: 297 - DNA, 74 bp, Homo sapiens
  source 1..74: mol_type = genomic DNA
gcccggatagctcagtcggtagagcatcagacttttaatctgagggtccagggttcaagtccctgttcgggcgt

SEQ ID NO: 298 - DNA, 107 bp, Homo sapiens
  source 1..107: mol_type = genomic DNA
tgtcaggatggccgagtggtctaaggcgccagactcaaggtaagcaccttgcctgcgggctttctggtctccggatggaggcgtgggttcgaatcccacttctgaca

SEQ ID NO: 299 - DNA, 73 bp, Homo sapiens
  source 1..73: mol_type = genomic DNA
ttccctggtggtctagtggttaggattcggcgctctcaccgccgcggcccgggttcgattcccggtcaggaaa

SEQ ID NO: 300 - DNA, 90 bp, Homo sapiens
  source 1..90: mol_type = genomic DNA
gccttcgatagctcagttggtagagcggaggactgtagtggatagggcgtggcaatccttaggtcgctggttcgattccggctcgaagga

SEQ ID NO: 301 - DNA, 74 bp, Homo sapiens
  source 1..74: mol_type = genomic DNA
cgggggattagctcaaatggtagagcgctcgcttagcatgcgagaggtagcgggatcgatgcccgcatcctcca

SEQ ID NO: 302 - DNA, 94 bp, Homo sapiens
  source 1..94: mol_type = genomic DNA
agctccagtggcgcaatcggttagcgcgcggtacttatacagcagtacatgcagagcaatgccgaggttgtgagttcgagcctcacctggagca

SEQ ID NO: 303 - DNA, 72 bp, Homo sapiens
  source 1..72: mol_type = genomic DNA
gcgccgctggtgtagtggtatcatgcaagattcccattcttgcgacccgggttcgattcccgggcggcgcat

SEQ ID NO: 304 - DNA, 73 bp, Homo sapiens
  source 1..73: mol_type = genomic DNA
tcccatatggtctagcggttaggattcctggttttcacccaggtggcccgggttcgactcccggtatgggaac

SEQ ID NO: 305 - DNA, 73 bp, Homo sapiens
  source 1..73: mol_type = genomic DNA
gggggatgtagctcagtggtagagcgcgcgcttcgcatgtgtgaggtcccgggttcaatccccggcatctcca

SEQ ID NO: 306 - DNA, 72 bp, Homo sapiens
  source 1..72: mol_type = genomic DNA
gcattggtggttcagtggtagaattctcgcctgccacgcgggaggcccgggttcgattcccggccaatgcaa

SEQ ID NO: 307 - DNA, 74 bp, Homo sapiens
  source 1..74: mol_type = genomic DNA
gggccagtggcgcaatggataacgcgtctgactacggatcagaagattctaggttcgactcctggctggctcgc

SEQ ID NO: 308 - DNA, 74 bp, Homo sapiens
  source 1..74: mol_type = genomic DNA
ggtttccgtagtgtagtggttatcacgttcgcctaacacgcgaaaggtccccggttcgaaaccgggcggaaaca

SEQ ID NO: 309 - DNA, 74 bp, Homo sapiens
  source 1..74: mol_type = genomic DNA
agtttccgtagtgtagtggttatcacgttcgcctaacacgcgaaaggtccccggttcgaaaccgggcggaaaca

SEQ ID NO: 310 - DNA, 83 bp, Homo sapiens
  source 1..83: mol_type = genomic DNA
aggtagcgtggccgagcggtctaaggcgctggattaaggctccagtctcttcgggggcgtgggttcgaatcccaccgctgcca

SEQ ID NO: 311 - DNA, 74 bp, Homo sapiens
  source 1..74: mol_type = genomic DNA
gtttccgtagtgtagtggtcatcacgttcgcctaacacgcgaaaggtccccggttcgaaaccgggcggaaacat

SEQ ID NO: 312 - DNA, 73 bp, Homo sapiens
  source 1..73: mol_type = genomic DNA
ggctcgttggtctaggggtatgattctcgctttgggtgcgagaggtcccgggttcaaatcccggacgagccca

SEQ ID NO: 313 - DNA, 73 bp, Homo sapiens
  source 1..73: mol_type = genomic DNA
ggctccatagctcaggggttagagcactggtcttgtaaaccagggtcgcgagttcaaatctcgctggggcctg

SEQ ID NO: 314 - DNA, 73 bp, Homo sapiens
  source 1..73: mol_type = genomic DNA
tggggatgtagctcagtggtagagcgcatgctttgcatgtatgaggccccgggttcgatccccggcatctcca

SEQ ID NO: 315 - DNA, 74 bp, Homo sapiens
  source 1..74: mol_type = genomic DNA
cgcccggctagctcagtcggtagagcatgagactcttaatctcagggtcgtgggttcgagccccacgttgggcg

SEQ ID NO: 316 - DNA, 74 bp, Homo sapiens
  source 1..74: mol_type = genomic DNA
gtttccgtagtgtagtggttatcacgttcgcctaacacgcgaaaggtccccggttcgaaaccgggcggaaacaa

SEQ ID NO: 317 - DNA, 74 bp, Homo sapiens
  source 1..74: mol_type = genomic DNA
gcccggctagctcagtcggtagagcatgagactcttaatctcagggtcgtgggttcgagccccacgttgggcgt

SEQ ID NO: 318 - DNA, 74 bp, Homo sapiens
  source 1..74: mol_type = genomic DNA
gtttccgtagtgtagtggttatcacgttcgcctcacacgcgaaaggtccccggttcgaaaccgggcggaaacac

SEQ ID NO: 319 - DNA, 73 bp, Homo sapiens
  source 1..73: mol_type = genomic DNA
cagcagagtggcgcagcggaagcgtgctgggcccataacccagaggtcgatggatcgaaaccatcctctgcta

SEQ ID NO: 320 - DNA, 85 bp, Homo sapiens
  source 1..85: mol_type = genomic DNA
ggagaggcctggccgagtggttaaggcgatggactgctaatccattgtgctctgcacgcgtgggttcgaatcccatcctcgtcgc

SEQ ID NO: 321 - DNA, 73 bp, Homo sapiens
  source 1..73: mol_type = genomic DNA
ggccccatggtgtaatggttagcactctggactttgaatccagcgatccgagttcaaatctcggtgggacctg

SEQ ID NO: 322 - DNA, 73 bp, Homo sapiens
  source 1..73: mol_type = genomic DNA
ggccccatggtgtaatggttagcactctggactttgaatccagcgatccgagttcaaatctcggtgggaccta

SEQ ID NO: 323 - DNA, 83 bp, Homo sapiens
  source 1..83: mol_type = genomic DNA
gtagtcgtggccgagtggttaaggcgatggacttgaaatccattggggtctccccgcgcaggttcgaatcctgccgactacgg

SEQ ID NO: 324 - DNA, 73 bp, Homo sapiens
  source 1..73: mol_type = genomic DNA
agcagagtggcgcagcggaagcgtgctgggcccataacccagaggtcgatggatcgaaaccatcctctgctat

SEQ ID NO: 325 - DNA, 74 bp, Homo sapiens
  source 1..74: mol_type = genomic DNA
ggaccacgtggcctaatggataaggcgtctgacttcggatcagaagattgagggttcgaatccctccgtggtta

SEQ ID NO: 326 - DNA, 83 bp, Homo sapiens
  source 1..83: mol_type = genomic DNA
tgtagtcgtggccgagtggttaaggcgatggactagaaatccattggggtctccccgcgcaggttcgaatcctgccgactacg

SEQ ID NO: 327 - DNA, 73 bp, Homo sapiens
  source 1..73: mol_type = genomic DNA
agcagagtggcgcagcggaagcgtgctgggcccataacccagaggtcgatggatcgaaaccatcctctgctag

SEQ ID NO: 328 - DNA, 84 bp, Homo sapiens
  source 1..84: mol_type = genomic DNA
cgtcaggatggccgagcggtctaaggcgctgcgttcaggtcgcagtctcccctggaggcgtgggttcgaatcccactcctgaca

SEQ ID NO: 329 - DNA, 75 bp, Homo sapiens
  source 1..75: mol_type = genomic DNA
ggctccgtggcttagctggttaaagcgcctgtctagtaaacaggagatcctgggttcgaatcccagcggggcctg

SEQ ID NO: 330 - DNA, 74 bp, Homo sapiens
  source 1..74: mol_type = genomic DNA
agggccagtggcgcaatggataacgcgtctgactacggatcagaagattccaggttcgactcctggctggctcg

SEQ ID NO: 331 - DNA, 74 bp, Homo sapiens
  source 1..74: mol_type = genomic DNA
ggtttccgtagtgtagtggttatcacgttcgcctcacacgcgaaaggtccccggttcgaaaccgggcggaaaca

SEQ ID NO: 332 - DNA, 73 bp, Homo sapiens
  source 1..73: mol_type = genomic DNA
aggggatgtagctcagtggtagagcgcatgcttcgcatgtatgaggtcccgggttcgatccccggcatctcca

SEQ ID NO: 333 - DNA, 75 bp, Homo sapiens
  source 1..75: mol_type = genomic DNA
tggccggttagctcagttggttagagcgtggtgctaataacgccaaggtcgcgggttcgatccccgtacgggcca

SEQ ID NO: 334 - DNA, 73 bp, Homo sapiens
  source 1..73: mol_type = genomic DNA
cggctcgttggtctaggggtatgattctcgcttagggtgcgagaggtcccgggttcaaatcccggacgagccc

SEQ ID NO: 335 - DNA, 74 bp, Homo sapiens
  source 1..74: mol_type = genomic DNA
agcccggctagctcagtcggtagagcatgagactcttaatctcagggtcgtgggttcgagccccacgttgggcg

SEQ ID NO: 336 - DNA, 92 bp, Homo sapiens
  source 1..92: mol_type = genomic DNA
tccttcgatagctcagttggtagagcggaggactgtagttggctgtgtccttagacatccttaggtcgctggttcgaatccggctcgaagga

SEQ ID NO: 337 - DNA, 74 bp, Homo sapiens
  source 1..74: mol_type = genomic DNA
ggggaattagctcaaatggtagagcgctcgcttagcatgcgagaggtagcgggatcgatgcccgcattctccag

SEQ ID NO: 338 - DNA, 74 bp, Homo sapiens
  source 1..74: mol_type = genomic DNA
cgccctcttagcgcagcgggcagcgcgtcagtctcataatctgaaggtcctgagttcgagcctcagagagggca

SEQ ID NO: 339 - DNA, 95 bp, Homo sapiens
  source 1..95: mol_type = genomic DNA
tgctccagtggcgcaatcggttagcgcgcggtacttatatggcagtatgtgtgcgagtgatgccgaggttgtgagttcgagcctcacctggagca

SEQ ID NO: 340 - DNA, 73 bp, Homo sapiens
  source 1..73: mol_type = genomic DNA
tgccgtgatcgtatagtggttagtactctgcgttgtggccgcagcaacctcggttcgaatccgagtcacggca

SEQ ID NO: 341 - DNA, 75 bp, Homo sapiens
  source 1..75: mol_type = genomic DNA
ggccggttagctcagttggttagagcgtggtgctaataacgccaaggtcgcgggttcgatccccgtacgggccac

SEQ ID NO: 342 - DNA, 74 bp, Homo sapiens
  source 1..74: mol_type = genomic DNA
agtttccgtagtgtagtggttatcacgtttgcctaacacgcgaaaggtccccggttcgaaaccgggcagaaaca

SEQ ID NO: 343 - DNA, 74 bp, Homo sapiens
  source 1..74: mol_type = genomic DNA
gcttctgtagtgtagtggttatcacgttcgcctcacacgcgaaaggtccccggttcgaaaccgggcagaagcaa

SEQ ID NO: 344 - DNA, 73 bp, Homo sapiens
  source 1..73: mol_type = genomic DNA
ttcctcgttagtatagtggtgagtatccccgcctgtcacgcgggagaccggggttcgattccccgacggggag

SEQ ID NO: 345 - DNA, 83 bp, Homo sapiens
  source 1..83: mol_type = genomic DNA
gtagtcgtggccgagtggttaaggcgatggacttgaaatccattggggtttccccgcgcaggttcgaatcctgtcggctacgg

SEQ ID NO: 346 - DNA, 73 bp, Homo sapiens
  source 1..73: mol_type = genomic DNA
aggttccatggtgtaatggttagcactctggactctgaatccagcgatccgagttcaaatctcggtggaacct

SEQ ID NO: 347 - DNA, 73 bp, Homo sapiens
  source 1..73: mol_type = genomic DNA
tcctcgttagtatagtggtgagtgtccccgtctgtcacgcgggagaccggggttcgattccccgacggggaga

SEQ ID NO: 348 - DNA, 74 bp, Homo sapiens
  source 1..74: mol_type = genomic DNA
gtttccgtagtgtagtggttatcacgttcgcctaacacgcgaaaggtccctggatcaaaaccaggcggaaacaa

SEQ ID NO: 349 - DNA, 75 bp, Homo sapiens
  source 1..75: mol_type = genomic DNA
cggccggttagctcagttggttagagcgtggtgctaataacgccaaggtcgcgggttcgatccccgtactggcca

SEQ ID NO: 350 - DNA, 73 bp, Homo sapiens
  source 1..73: mol_type = genomic DNA
ggccccatggtgtaatggtcagcactctggactctgaatccagcgatccgagttcaaatctcggtgggaccca

SEQ ID NO: 351 - DNA, 73 bp, Homo sapiens
  source 1..73: mol_type = genomic DNA
ggccccatggtgtaatggttagcactctggactttgaatccagcgatccgagttcaaatctcggtgggacctt

SEQ ID NO: 352 - DNA, 73 bp, Homo sapiens
  source 1..73: mol_type = genomic DNA
tgggggtgtagctcagtggtagagcgcgtgcttagcatgtacgaggtcccgggttcaatccccggcacctcca

SEQ ID NO: 353 - DNA, 73 bp, Homo sapiens
  source 1..73: mol_type = genomic DNA
ggggatgtagctcagtggtagagcgcatgcttagcatgcatgaggtcccgggttcgatccccagcatctccag

SEQ ID NO: 354 - DNA, 73 bp, Homo sapiens
  source 1..73: mol_type = genomic DNA
agggggtgtagctcagtggtagagcgcgtgcttcgcatgtacgaggccccgggttcgacccccggctcctcca

SEQ ID NO: 355 - DNA, 73 bp, Homo sapiens
  source 1..73: mol_type = genomic DNA
gggggtgtagctcagtggtagagcgcgtgcttagcatgcacgaggccccgggttcaatccccggcacctccat

SEQ ID NO: 356 - DNA, 73 bp, Homo sapiens
  source 1..73: mol_type = genomic DNA
gggggtgtagctcagtggtagagcgcgtgcttagcatgcacgaggccccgggttcaatccccggcacctccag

SEQ ID NO: 357 - DNA, 107 bp, Homo sapiens
  source 1..107: mol_type = genomic DNA
gtcaggatggccgagtggtctaaggcgccagactcaagctaagcttcctccgcggtggggattctggtctccaatggaggcgtgggttcgaatcccacttctgacac

SEQ ID NO: 358 - DNA, 106 bp, Homo sapiens
  source 1..106: mol_type = genomic DNA
tgtcaggatggccgagtggtctaaggcgccagactcaagcttggcttcctcgtgttgaggattctggtctccaatggaggcgtgggttcgaatcccacttctgaca

SEQ ID NO: 359 - DNA, 73 bp, Homo sapiens
  source 1..73: mol_type = genomic DNA
ggttccatggtgtaatggttagcactctggactctgaatccagcgatccgagttcaaatctcggtggaacctt

SEQ ID NO: 360 - DNA, 83 bp, Homo sapiens
  source 1..83: mol_type = genomic DNA
ggtagcgtggccgagcggtctaaggcgctggattaaggctccagtctcttcgggggcgtgggttcgaatcccaccgctgccag

SEQ ID NO: 361 - DNA, 74 bp, Homo sapiens
  source 1..74: mol_type = genomic DNA
tgcctccttagcgcagtaggcagcgcgtcagtctcataatctgaaggtcctgagttcgaacctcagagggggca

SEQ ID NO: 362 - DNA, 74 bp, Homo sapiens
  source 1..74: mol_type = genomic DNA
agcccggatagctcagtcggtagagcatcagacttttaatctgagggtccagggttcaagtccctgttcgggcg

SEQ ID NO: 363 - DNA, 74 bp, Homo sapiens
  source 1..74: mol_type = genomic DNA
gcctccttagcgcagtaggcagcgcgtcagtctcataatctgaaggtcctgagttcgaacctcagagggggcag

SEQ ID NO: 364 - DNA, 73 bp, Homo sapiens
  source 1..73: mol_type = genomic DNA
ttccctggtggtctagtggttaggattcggcgctctcaccgccgcggcccgggttcgattcccggtcagggaa

SEQ ID NO: 365 - DNA, 84 bp, Homo sapiens
  source 1..84: mol_type = genomic DNA
caccaggatggccgagtggttaaggcgttggacttaagatccaatggacatatgtccgcgtgggttcgaaccccactcctggta

SEQ ID NO: 366 - DNA, 73 bp, Homo sapiens
  source 1..73: mol_type = genomic DNA
tggctcgttggtctaggggtatgattctcgcttagggtgcgagaggtcccgggttcaaatcccggacgagccc

SEQ ID NO: 367 - DNA, 74 bp, Homo sapiens
  source 1..74: mol_type = genomic DNA
agccccagtggcctaatggataaggcattggcctcctaagccagggattgtgggttcgagtcccatctggggtg

SEQ ID NO: 368 - DNA, 73 bp, Homo sapiens
  source 1..73: mol_type = genomic DNA
ggggatatagctcaggggtagagcatttgactgcagatcaagaggtccccggttcaaatccgggtgccccccc

SEQ ID NO: 369 - DNA, 94 bp, Homo sapiens
  source 1..94: mol_type = genomic DNA
cccttcgatagctcagctggtagagcggaggactgtagctacttcctcagcaggagacatccttaggtcgctggttcgattccggctcgaagga

SEQ ID NO: 370 - DNA, 90 bp, Homo sapiens
  source 1..90: mol_type = genomic DNA
cccttcgatagctcagctggtagagcggaggactgtaggcgcgcgcccgtggccatccttaggtcgctggttcgattccggctcgaagga

SEQ ID NO: 371 - DNA, 74 bp, Homo sapiens
  source 1..74: mol_type = genomic DNA
tgggggattagctcaaatggtagagcgctcgcttagcatgcgagaggtagcgggatcgatgcccgcatcctcca

SEQ ID NO: 372 - DNA, 83 bp, Homo sapiens
  source 1..83: mol_type = genomic DNA
gtagtcgtggccgagtggttaaggcgatggactagaaatccattggggtctccccgcgcaggttcgaatcctgccgactacgg

SEQ ID NO: 373 - DNA, 74 bp, Homo sapiens
  source 1..74: mol_type = genomic DNA
gcctcgttagcgcagtaggtagcgcgtcagtctcataatctgaaggtcgtgagttcgatcctcacacggggcac

SEQ ID NO: 374 - DNA, 92 bp, Homo sapiens
  source 1..92: mol_type = genomic DNA
ggctctgtggcgcaatggatagcgcattggacttctagctgagcctagtgtggtcattcaaaggttgtgggttcgagtcccaccagagtcga

SEQ ID NO: 375 - DNA, 75 bp, Homo sapiens
  source 1..75: mol_type = genomic DNA
gtctctgtggcgcaatcggttagcgcgttcggctgttaaccgaaaggttggtggttcgagcccacccagggacgc

SEQ ID NO: 376 - DNA, 83 bp, Homo sapiens
  source 1..83: mol_type = genomic DNA
ggcagcgatggccgagtggttaaggcgttggacttgaaatccaatggggtctccccgcgcaggttcgaaccctgctcgctgcg

SEQ ID NO: 377 - DNA, 74 bp, Homo sapiens
  source 1..74: mol_type = genomic DNA
ggttccatagtgtagtggttatcacgtctgctttacacgcagaaggtcctgggttcgagccccagtggaaccat

SEQ ID NO: 378 - DNA, 74 bp, Homo sapiens
  source 1..74: mol_type = genomic DNA
ggttccatagtgtagcggttatcacgtctgctttacacgcagaaggtcctgggttcgagccccagtggaaccac

SEQ ID NO: 379 - DNA, 87 bp, Homo sapiens
  source 1..87: mol_type = genomic DNA
tggctctgtggcgcaatggatagcgcattggacttctagatagttagagaaattcaaaggttgtgggttcgagtcccaccagagtcg

SEQ ID NO: 380 - DNA, 84 bp, Homo sapiens
  source 1..84: mol_type = genomic DNA
taccagaatggccgagtggttaaggcgttggacttaagatccaatggattcatatccgcgtgggttcgaaccccacttctggta

SEQ ID NO: 381 - DNA, 74 bp, Homo sapiens
  source 1..74: mol_type = genomic DNA
ggcccggatagctcagtcggtagagcatcagacttttaatctgagggtccggggttcaagtccctgttcgggcg

SEQ ID NO: 382 - DNA, 74 bp, Homo sapiens
  source 1..74: mol_type = genomic DNA
gccgaaatagctcagttgggagagcgttagactgaagatctaaaggtccctggttcgatcccgggtttcggcag

SEQ ID NO: 383 - DNA, 74 bp, Homo sapiens
  source 1..74: mol_type = genomic DNA
gcccggatagctcagtcggtagagcatcagacttttaatctgagggtccagggttcaagtccctgttcgggcgg

SEQ ID NO: 384 - DNA, 74 bp, Homo sapiens
  source 1..74: mol_type = genomic DNA
gccgaaatagctcagttgggagagcgttagactgaagatctaaaggtccctggttcaatcccgggtttcggcag

SEQ ID NO: 385 - DNA, 83 bp, Homo sapiens
  source 1..83: mol_type = genomic DNA
ggacgaggtggccgagtggttaaggcgatggactgctaatccattgtgctttgcacgcgtgggttcgaatcccatcctcgtcg

SEQ ID NO: 386 - DNA, 73 bp, Homo sapiens
  source 1..73: mol_type = genomic DNA
ggctcgttggtctaggggtatgattctcggtttgggtccgagaggtcccgggttcaaatcccggacgagcccc

SEQ ID NO: 387 - DNA, 83 bp, Homo sapiens
  source 1..83: mol_type = genomic DNA
agtcacggtggccgagtggttaaggcgttggactcgaaatccaatggggtttccccgcacaggttcgaatcctgttcgtgacg

SEQ ID NO: 388 - DNA, 73 bp, Homo sapiens
  source 1..73: mol_type = genomic DNA
ctcctcgttagtatagtggttagtatccccgcctgtcacgcgggagaccggggttcaattccccgacggggag

SEQ ID NO: 389 - DNA, 73 bp, Homo sapiens
  source 1..73: mol_type = genomic DNA
ggacctcgtggcgcaacggtagcgcgtctgactccagatcagaaggctgcgtgttcgaatcacgtcggggtca

SEQ ID NO: 390 - DNA, 73 bp, Homo sapiens
  source 1..73: mol_type = genomic DNA
ggggatgtagctcagtggtagagcgcatgctttgcatgtatgaggccccgggttcgatccccggcatctccat

SEQ ID NO: 391 - DNA, 74 bp, Homo sapiens
  source 1..74: mol_type = genomic DNA
gccgaaatagctcagttgggagagcgttagactgaagatctaaaggtccctggttcgatcccgggtttcggcac

SEQ ID NO: 392 - DNA, 73 bp, Homo sapiens
  source 1..73: mol_type = genomic DNA
aggggatgtagctcagtggtagagcgcatgctttgcacgtatgaggccccgggttcaatccccggcatctcca

SEQ ID NO: 393 - DNA, 75 bp, Homo sapiens
  source 1..75: mol_type = genomic DNA
gtctctgtggcgcaatcggttagcgcgttcggctgttaaccgaaaggttggtggttcgagcccacccagggacgg

SEQ ID NO: 394 - DNA, 73 bp, Homo sapiens
  source 1..73: mol_type = genomic DNA
tcccacatggtctagcggttaggattcctggttttcacccaggcggcccgggttcgactcccggtgtgggaac

SEQ ID NO: 395 - DNA, 74 bp, Homo sapiens
  source 1..74: mol_type = genomic DNA
ggctccatagctcaggggttagagcgctggtcttgtaaaccaggggtcgcgagttcaattctcgctggggcctg

SEQ ID NO: 396 - DNA, 83 bp, Homo sapiens
  source 1..83: mol_type = genomic DNA
tggtagtgtggccgagcggtctaaggcgctggatttaggctccagtctcttcgggggcgtgggttcgaatcccaccactgcca

SEQ ID NO: 397 - DNA, 74 bp, Homo sapiens
  source 1..74: mol_type = genomic DNA
ggctccatagctcaggggttagagcactggtcttgtaaaccaggggtcgcgagttcaaatctcgctggggcctc

SEQ ID NO: 398 - DNA, 73 bp, Homo sapiens
  source 1..73: mol_type = genomic DNA
tggctcgttggtctagtggtatgattctcgctttgggtgcgagaggtcccgggttcaaatcccggacgagccc

SEQ ID NO: 399 - DNA, 95 bp, Homo sapiens
  source 1..95: mol_type = genomic DNA
ccttcgatagctcagctggtagagcggaggactgtagattgtacagacatttgcggacatccttaggtcgctggttcgattccggctcgaaggaa

SEQ ID NO: 400 - DNA, 74 bp, Homo sapiens
  source 1..74: mol_type = genomic DNA
aggccctatagctcaggggttagagcactggtcttgtaaaccaggggtcgcgagttcaaatctcgctggggcct

SEQ ID NO: 401 - DNA, 90 bp, Homo sapiens
  source 1..90: mol_type = genomic DNA
tccttcgatagctcagctggtagagcggaggactgtagtacttaatgtgtggtcatccttaggtcgctggttcgattccggctcgaagga

SEQ ID NO: 402 - DNA, 73 bp, Homo sapiens
  source 1..73: mol_type = genomic DNA
tggctcgttggtctaggggtatgattctcgctttgggtgcgagaggtcccgggttcaaatcccggacgagccc

SEQ ID NO: 403 - DNA, 74 bp, Homo sapiens
  source 1..74: mol_type = genomic DNA
gcccggctagctcagtcggtagagcatgggactcttaatcccagggtcgtgggttcgagccccacgttgggcgc

SEQ ID NO: 404 - DNA, 75 bp, Homo sapiens
  source 1..75: mol_type = genomic DNA
cggccggttagctcagttggttagagcgtggtgctaataacgccaaggtcgcgggttcgatccccgtacgggcca

SEQ ID NO: 405 - DNA, 73 bp, Homo sapiens
  source 1..73: mol_type = genomic DNA
tcccacatggtctagcggttaggattcctggttttcacccaggcggcccgggttcgactcccggtgtgggaat

SEQ ID NO: 406 - DNA, 83 bp, Homo sapiens
  source 1..83: mol_type = genomic DNA
gacgaggtggccgagtggttaaggcgatggactgctaatccattgtgctctgcacgcgtgggttcgaatcccatcctcgtcga

SEQ ID NO: 407 - DNA, 73 bp, Homo sapiens
  source 1..73: mol_type = genomic DNA
gccgtgatcgtatagtggttagtactctgcgttgtggccgcagcaacctcggttcgaatccgagtcacggcat

SEQ ID NO: 408 - DNA, 73 bp, Homo sapiens
  source 1..73: mol_type = genomic DNA
cgccgtgatcgtatagtggttagtactctgcgttgtggccgcagcaacctcggttcgaatccgagtcacggca

SEQ ID NO: 409 - DNA, 73 bp, Homo sapiens
  source 1..73: mol_type = genomic DNA
ggttccatggtgtaatggttagcactctggactctgaatccagcgatccgagttcaaatctcggtggaacctg

SEQ ID NO: 410 - DNA, 74 bp, Homo sapiens
  source 1..74: mol_type = genomic DNA
tgcccggctagctcagtcggtagagcatgggactcttaatcccagggtcgtgggttcgagccccacgttgggcg

SEQ ID NO: 411 - DNA, 74 bp, Homo sapiens
  source 1..74: mol_type = genomic DNA
gggccgcgtggcctaatggataaggcgtctgacttcggatcagaagattgcaggttcgagtcctgccgcggtcg

SEQ ID NO: 412 - DNA, 72 bp, Homo sapiens
  source 1..72: mol_type = genomic DNA
gcgccgctggtgtagtggtatcatgcaagattcccattcttgcgacccgggttcgattcccgggcggcgcac

SEQ ID NO: 413 - DNA, 74 bp, Homo sapiens
  source 1..74: mol_type = genomic DNA
gggccgcgtggcctaatggataaggcgtctgattccggatcagaagattgagggttcgagtcccttcgtggtcg

SEQ ID NO: 414 - DNA, 74 bp, Homo sapiens
  source 1..74: mol_type = genomic DNA
cgccccggtggcctaatggataaggcattggcctcctaagccagggattgtgggttcgagtcccacccggggta

SEQ ID NO: 415 - DNA, 74 bp, Homo sapiens
  source 1..74: mol_type = genomic DNA
gcccggctagctcagtcggtagagcatgagacccttaatctcagggtcgtgggttcgagccccacgttgggcgt

SEQ ID NO: 416 - DNA, 73 bp, Homo sapiens
  source 1..73: mol_type = genomic DNA
aggcgcggtggccaagtggtaaggcgtcggtctcgtaaaccgaagatcacgggttcgaaccccgtccgtgcct

SEQ ID NO: 417 - DNA, 83 bp, Homo sapiens
  source 1..83: mol_type = genomic DNA
ggtagcgtggccgagtggtctaaggcgctggatttaggctccagtcatttcgatggcgtgggttcgaatcccaccgctgccac

SEQ ID NO: 418 - DNA, 83 bp, Homo sapiens
  source 1..83: mol_type = genomic DNA
gggtagcgtggccgagcggtctaaggcgctggattaaggctccagtctcttcgggggcgtgggttcgaatcccaccgctgcca

SEQ ID NO: 419 - DNA, 84 bp, Homo sapiens
  source 1..84: mol_type = genomic DNA
agtcaggatggccgagcggtctaaggcgctgcgttcaggtcgcagtctcccctggaggcgtgggttcgaatcccacttctgaca

SEQ ID NO: 420 - DNA, 84 bp, Homo sapiens
  source 1..84: mol_type = genomic DNA
gtcaggatggccgagcggtctaaggcgctgcgttcaggtcgcagtctcccctggaggcgtgggttcgaatcccacttctgacag

SEQ ID NO: 421 - DNA, 74 bp, Homo sapiens
  source 1..74: mol_type = genomic DNA
gcctcgttagcgcagtaggcagcgcgtcagtctcataatctgaaggtcgtgagttcgagcctcacacggggcag

SEQ ID NO: 422 - DNA, 83 bp, Homo sapiens
  source 1..83: mol_type = genomic DNA
ggtagcgtggccgagcggtctaaggcgctggatttaggctccagtctcttcggaggcgtgggttcgaatcccaccgctgccag

SEQ ID NO: 423 - DNA, 89 bp, Homo sapiens
  source 1..89: mol_type = genomic DNA
tggctctgtggcgcaatggatagcgcattggacttctagtgacgaatagagcaattcaaaggttgtgggttcgaatcccaccagagtcg

SEQ ID NO: 424 - DNA, 72 bp, Homo sapiens
  source 1..72: mol_type = genomic DNA
cgcattggtggttcagtggtagaattctcgcctgccacgcgggaggcccgggttcgattcccggccaatgca

SEQ ID NO: 425 - DNA, 83 bp, Homo sapiens
  source 1..83: mol_type = genomic DNA
gctgtgatggccgagtggttaaggcgttggactcgaaatccaatggggtctccccgcgcaggttcgaatcctgctcacagcgt

SEQ ID NO: 426 - DNA, 75 bp, Homo sapiens
  source 1..75: mol_type = genomic DNA
ggcgccgtggcttagctggttaaagcgcctgtctagtaaacaggagatcctgggttcgaatcccagcggtgcctg

SEQ ID NO: 427 - DNA, 73 bp, Homo sapiens
  source 1..73: mol_type = genomic DNA
cgacctcgtggcgcaacggtagcgcgtctgactccagatcagaaggttgcgtgttcaaatcacgtcggggtca

SEQ ID NO: 428 - DNA, 83 bp, Homo sapiens
  source 1..83: mol_type = genomic DNA
agacgaggtggccgagtggttaaggcgatggactgctaatccattgtgctctgcacgcgtgggttcgaatcccatcctcgtcg

SEQ ID NO: 429 - DNA, 75 bp, Homo sapiens
  source 1..75: mol_type = genomic DNA
cggcgccgtggcttagttggttaaagcgcctgtctagtaaacaggagatcctgggttcgaatcccagcggtgcct

SEQ ID NO: 430 - DNA, 73 bp, Homo sapiens
  source 1..73: mol_type = genomic DNA
ggcctcgtggcgcaacggtagcgcgtctgactccagatcagaaggttgcgtgttcaaatcacgtcggggtcaa

SEQ ID NO: 431 - DNA, 73 bp, Homo sapiens
  source 1..73: mol_type = genomic DNA
agcgttggtggtatagtggtaagcatagctgccttccaagcagttgacccgggttcgattcccggccaacgca

SEQ ID NO: 432 - DNA, 73 bp, Homo sapiens
  source 1..73: mol_type = genomic DNA
tcctcgttagtatagtggtgagtatccccgcctgtcacgcgggagaccggggttcgattccccgacggggaga

SEQ ID NO: 433 - DNA, 73 bp, Homo sapiens
  source 1..73: mol_type = genomic DNA
ggctcgttggtctaggggtatgattctcgcttcgggtgcgagaggtcccgggttcaaatcccggacgagccct

SEQ ID NO: 434 - DNA, 75 bp, Homo sapiens
  source 1..75: mol_type = genomic DNA
ggcgccgtggcttagttggttaaagcgcctgtctagtaaacaggagatcctgggttcgaatcccagcggtgcctt

SEQ ID NO: 435 - DNA, 83 bp, Homo sapiens
  source 1..83: mol_type = genomic DNA
gtagtcgtggccgagtggttaaggcgatggactagaaatccattggggtctccccgcgcaggttcgaatcctgccgactacgt

SEQ ID NO: 436 - DNA, 73 bp, Homo sapiens
  source 1..73: mol_type = genomic DNA
tgacctcgtggcgcaatggtagcgcgtctgactccagatcagaaggttgcgtgttcaagtcacgtcggggtca

SEQ ID NO: 437 - DNA, 73 bp, Homo sapiens
  source 1..73: mol_type = genomic DNA
aggcgcggtggccaagtggtaaggcgtcggtctcgtaaaccgaagatcgcgggttcgaaccccgtccgtgcct

SEQ ID NO: 438 - DNA, 73 bp, Homo sapiens
  source 1..73: mol_type = genomic DNA
agggggtatagctcagtggtagagcatttgactgcagatcaagaggtccccggttcaaatccgggtgccccct

SEQ ID NO: 439 - DNA, 73 bp, Homo sapiens
  source 1..73: mol_type = genomic DNA
gggggtatagctcagtggtagagcatttgactgcagatcaagaggtccctggttcaaatccgggtgccccctc

SEQ ID NO: 440 - DNA, 73 bp, Homo sapiens
  source 1..73: mol_type = genomic DNA
gggggtatagctcagtggtagagcatttgactgcagatcaagaggtccccggttcaaatccgggtgccccctc

SEQ ID NO: 441 - DNA, 73 bp, Homo sapiens
  source 1..73: mol_type = genomic DNA
aggtcccatggtgtaatggttagcactctggactttgaatccagcgatccgagttcaaatctcggtgggacct

SEQ ID NO: 442 - DNA, 74 bp, Homo sapiens
  source 1..74: mol_type = genomic DNA
gacccagtggcctaatggataaggcatcagcctccggagctggggattgtgggttcgagtcccatctgggtcgc

SEQ ID NO: 443 - DNA, 74 bp, Homo sapiens
  source 1..74: mol_type = genomic DNA
agccccagtggcctaatggataaggcactggcctcctaagccagggattgtgggttcgagtcccacctggggta

SEQ ID NO: 444 - DNA, 74 bp, Homo sapiens
  source 1..74: mol_type = genomic DNA
gccccagtggcctaatggataaggcactggcctcctaagccagggattgtgggttcgagtcccacctggggtgt

SEQ ID NO: 445 - DNA, 74 bp, Homo sapiens
  source 1..74: mol_type = genomic DNA
agaccgcgtggcctaatggataaggcgtctgacttcggatcagaagattgagggttcgagtcccttcgtggtcg

SEQ ID NO: 446 - DNA, 75 bp, Homo sapiens
  source 1..75: mol_type = genomic DNA
cgtctctgtggcgcaatcggttagcgcgttcggctgttaaccgaaaggttggtggttcgagcccacccagggacg

SEQ ID NO: 447 - DNA, 73 bp, Homo sapiens
  source 1..73: mol_type = genomic DNA
ggcgttggtggtatagtggttagcatagctgccttccaagcagttgacccgggttcgattcccggccaacgca

SEQ ID NO: 448 - DNA, 74 bp, Homo sapiens
  source 1..74: mol_type = genomic DNA
gtttccgtagtgtagcggttatcacattcgcctcacacgcgaaaggtccccggttcgatcccgggcggaaacag

SEQ ID NO: 449 - DNA, 75 bp, Homo sapiens
  source 1..75: mol_type = genomic DNA
tggcgccgtggcttagttggttaaagcgcctgtctagtaaacaggagatcctgggttcgaatcccagcggtgcct

SEQ ID NO: 450 - DNA, 94 bp, Homo sapiens
  source 1..94: mol_type = genomic DNA
gctccagtggcgcaatcggttagcgcgcggtacttatatgacagtgcgagcggagcaatgccgaggttgtgagttcgatcctcacctggagcac

SEQ ID NO: 451 - DNA, 72 bp, Homo sapiens
  source 1..72: mol_type = genomic DNA
gcatgggtggttcagtggtagaattctcgcctgccacgcgggaggcccgggttcgattcccggcccatgcag

SEQ ID NO: 452 - DNA, 18 bp, synthetic construct
  misc_feature 1..18: note = source = /note="Description of Sequence: Synthetic oligonucleotide"
  source 1..18: mol_type = other DNA
aaaatataaatatatttc

SEQ ID NO: 453-454 - skipped (2 entries)

SEQ ID NO: 455 - DNA, 15 bp, synthetic construct
  misc_feature 1..15: note = source = /note="Description of Sequence: Synthetic oligonucleotide"
  source 1..15: mol_type = other DNA
aattcttcggaatgt

SEQ ID NO: 456-460 - skipped (5 entries)

SEQ ID NO: 461 - DNA, 20 bp, synthetic construct
  misc_feature 1..20: note = source = /note="Description of Sequence: Synthetic oligonucleotide"
  source 1..20: mol_type = other DNA
caggcgggttctgcccgcgc

SEQ ID NO: 462 - DNA, 18 bp, synthetic construct
  misc_feature 1..18: note = source = /note="Description of Sequence: Synthetic oligonucleotide"
  source 1..18: mol_type = other DNA
catacctgcaagggtatc

SEQ ID NO: 463 - DNA, 15 bp, synthetic construct
  misc_feature 1..15: note = source = /note="Description of Sequence: Synthetic oligonucleotide"
  source 1..15: mol_type = other DNA
cgaccgcaaggttgt

SEQ ID NO: 464 - DNA, 15 bp, synthetic construct
  misc_feature 1..15: note = source = /note="Description of Sequence: Synthetic oligonucleotide"
  source 1..15: mol_type = other DNA
cgaccttgcggtcat

SEQ ID NO: 465 - DNA, 18 bp, synthetic construct
  misc_feature 1..18: note = source = /note="Description of Sequence: Synthetic oligonucleotide"
  source 1..18: mol_type = other DNA
cgatgctaatcacatcgt

SEQ ID NO: 466 - DNA, 15 bp, synthetic construct
  misc_feature 1..15: note = source = /note="Description of Sequence: Synthetic oligonucleotide"
  source 1..15: mol_type = other DNA
cgatggtgacatcat

SEQ ID NO: 467 - DNA, 16 bp, synthetic construct
  misc_feature 1..16: note = source = /note="Description of Sequence: Synthetic oligonucleotide"
  source 1..16: mol_type = other DNA
cgatggtttacatcgt

SEQ ID NO: 468 - DNA, 13 bp, synthetic construct
  misc_feature 1..13: note = source = /note="Description of Sequence: Synthetic oligonucleotide"
  source 1..13: mol_type = other DNA
cgccgtaaggtgt

SEQ ID NO: 469 - DNA, 12 bp, synthetic construct
  misc_feature 1..12: note = source = /note="Description of Sequence: Synthetic oligonucleotide"
  source 1..12: mol_type = other DNA
cgccttaggtgt

SEQ ID NO: 470 - DNA, 15 bp, synthetic construct
  misc_feature 1..15: note = source = /note="Description of Sequence: Synthetic oligonucleotide"
  source 1..15: mol_type = other DNA
cgcctttcgacgcgt

SEQ ID NO: 471 - DNA, 13 bp, synthetic construct
  misc_feature 1..13: note = source = /note="Description of Sequence: Synthetic oligonucleotide"
  source 1..13: mol_type = other DNA
cgcttcacggcgt

SEQ ID NO: 472 - DNA, 15 bp, synthetic construct
  misc_feature 1..15: note = source = /note="Description of Sequence: Synthetic oligonucleotide"
  source 1..15: mol_type = other DNA
cggcagcaatgctgt

SEQ ID NO: 473 - DNA, 13 bp, synthetic construct
  misc_feature 1..13: note = source = /note="Description of Sequence: Synthetic oligonucleotide"
  source 1..13: mol_type = other DNA
cggctccgccttc

SEQ ID NO: 474 - DNA, 16 bp, synthetic construct
  misc_feature 1..16: note = source = /note="Description of Sequence: Synthetic oligonucleotide"
  source 1..16: mol_type = other DNA
cgggtatcacagggtc

SEQ ID NO: 475 - DNA, 17 bp, synthetic construct
  misc_feature 1..17: note = source = /note="Description of Sequence: Synthetic oligonucleotide"
  source 1..17: mol_type = other DNA
cggtgcgcaagcgctgt

SEQ ID NO: 476 - DNA, 18 bp, synthetic construct
  misc_feature 1..18: note = source = /note="Description of Sequence: Synthetic oligonucleotide"
  source 1..18: mol_type = other DNA
cgtacgggtgaccgtacc

SEQ ID NO: 477 - DNA, 13 bp, synthetic construct
  misc_feature 1..13: note = source = /note="Description of Sequence: Synthetic oligonucleotide"
  source 1..13: mol_type = other DNA
cgtcaaagacttc

SEQ ID NO: 478 - DNA, 13 bp, synthetic construct
  misc_feature 1..13: note = source = /note="Description of Sequence: Synthetic oligonucleotide"
  source 1..13: mol_type = other DNA
cgtcgtaagactt

SEQ ID NO: 479 - DNA, 14 bp, synthetic construct
  misc_feature 1..14: note = source = /note="Description of Sequence: Synthetic oligonucleotide"
  source 1..14: mol_type = other DNA
cgttgaataaacgt

SEQ ID NO: 480-485 - skipped (6 entries)

SEQ ID NO: 486 - DNA, 19 bp, synthetic construct
  misc_feature 1..19: note = source = /note="Description of Sequence: Synthetic oligonucleotide"
  source 1..19: mol_type = other DNA
taactagatactttcagat

SEQ ID NO: 487 - DNA, 15 bp, synthetic construct
  misc_feature 1..15: note = source = /note="Description of Sequence: Synthetic oligonucleotide"
  source 1..15: mol_type = other DNA
tactcgtatgggtgc

SEQ ID NO: 488 - DNA, 13 bp, synthetic construct
  misc_feature 1..13: note = source = /note="Description of Sequence: Synthetic oligonucleotide"
  source 1..13: mol_type = other DNA
tactttgcggtgt

SEQ ID NO: 489 - DNA, 19 bp, synthetic construct
  misc_feature 1..19: note = source = /note="Description of Sequence: Synthetic oligonucleotide"
  source 1..19: mol_type = other DNA
taggcgagtaacatcgtgc

SEQ ID NO: 490 - DNA, 19 bp, synthetic construct
  misc_feature 1..19: note = source = /note="Description of Sequence: Synthetic oligonucleotide"
  source 1..19: mol_type = other DNA
taggcgtgaatagcgcctc

SEQ ID NO: 491 - DNA, 19 bp, synthetic construct
  misc_feature 1..19: note = source = /note="Description of Sequence: Synthetic oligonucleotide"
  source 1..19: mol_type = other DNA
taggtcgcgagagcggcgc

SEQ ID NO: 492 - DNA, 19 bp, synthetic construct
  misc_feature 1..19: note = source = /note="Description of Sequence: Synthetic oligonucleotide"
  source 1..19: mol_type = other DNA
taggtcgcgtaagcggcgc

SEQ ID NO: 493 - DNA, 17 bp, synthetic construct
  misc_feature 1..17: note = source = /note="Description of Sequence: Synthetic oligonucleotide"
  source 1..17: mol_type = other DNA
taggtggttatccacgc

SEQ ID NO: 494-495 - skipped (2 entries)

SEQ ID NO: 496 - DNA, 18 bp, synthetic construct
  misc_feature 1..18: note = source = /note="Description of Sequence: Synthetic oligonucleotide"
  source 1..18: mol_type = other DNA
tatacgtgaaagcgtatc

SEQ ID NO: 497 - DNA, 21 bp, synthetic construct
  misc_feature 1..21: note = source = /note="Description of Sequence: Synthetic oligonucleotide"
  source 1..21: mol_type = other DNA
tatagggtcaaaaactctatc

SEQ ID NO: 498 - DNA, 20 bp, synthetic construct
  misc_feature 1..20: note = source = /note="Description of Sequence: Synthetic oligonucleotide"
  source 1..20: mol_type = other DNA
tatgcagaaatacctgcatc

SEQ ID NO: 499 - DNA, 15 bp, synthetic construct
  misc_feature 1..15: note = source = /note="Description of Sequence: Synthetic oligonucleotide"
  source 1..15: mol_type = other DNA
tccccatacgggggc

SEQ ID NO: 500 - DNA, 14 bp, synthetic construct
  misc_feature 1..14: note = source = /note="Description of Sequence: Synthetic oligonucleotide"
  source 1..14: mol_type = other DNA
tcccgaaggggttc

SEQ ID NO: 501 - DNA, 15 bp, synthetic construct
  misc_feature 1..15: note = source = /note="Description of Sequence: Synthetic oligonucleotide"
  source 1..15: mol_type = other DNA
tctacgtatgtgggc

SEQ ID NO: 502 - DNA, 14 bp, synthetic construct
  misc_feature 1..14: note = source = /note="Description of Sequence: Synthetic oligonucleotide"
  source 1..14: mol_type = other DNA
tctcataggagttc

SEQ ID NO: 503 - DNA, 14 bp, synthetic construct
  misc_feature 1..14: note = source = /note="Description of Sequence: Synthetic oligonucleotide"
  source 1..14: mol_type = other DNA
tctcctctggaggc

SEQ ID NO: 504 - DNA, 15 bp, synthetic construct
  misc_feature 1..15: note = source = /note="Description of Sequence: Synthetic oligonucleotide"
  source 1..15: mol_type = other DNA
tcttagcaataaggt

SEQ ID NO: 505 - DNA, 14 bp, synthetic construct
  misc_feature 1..14: note = source = /note="Description of Sequence: Synthetic oligonucleotide"
  source 1..14: mol_type = other DNA
tcttgtaggagttc

SEQ ID NO: 506 - DNA, 15 bp, synthetic construct
  misc_feature 1..15: note = source = /note="Description of Sequence: Synthetic oligonucleotide"
  source 1..15: mol_type = other DNA
tgaacgtaagttcgc

SEQ ID NO: 507 - DNA, 16 bp, synthetic construct
  misc_feature 1..16: note = source = /note="Description of Sequence: Synthetic oligonucleotide"
  source 1..16: mol_type = other DNA
tgaactgcgaggttcc

SEQ ID NO: 508 - skipped

SEQ ID NO: 509 - DNA, 15 bp, synthetic construct
  misc_feature 1..15: note = source = /note="Description of Sequence: Synthetic oligonucleotide"
  source 1..15: mol_type = other DNA
tgaccgaaaggtcgt

SEQ ID NO: 510 - DNA, 15 bp, synthetic construct
  misc_feature 1..15: note = source = /note="Description of Sequence: Synthetic oligonucleotide"
  source 1..15: mol_type = other DNA
tgaccgcaaggtcgt

SEQ ID NO: 511 - DNA, 14 bp, synthetic construct
  misc_feature 1..14: note = source = /note="Description of Sequence: Synthetic oligonucleotide"
  source 1..14: mol_type = other DNA
tgagctctgctctc

SEQ ID NO: 512 - DNA, 18 bp, synthetic construct
  misc_feature 1..18: note = source = /note="Description of Sequence: Synthetic oligonucleotide"
  source 1..18: mol_type = other DNA
tgaggcctcacggcctac

SEQ ID NO: 513 - DNA, 15 bp, synthetic construct
  misc_feature 1..15: note = source = /note="Description of Sequence: Synthetic oligonucleotide"
  source 1..15: mol_type = other DNA
tgagggcaacttcgt

SEQ ID NO: 514 - DNA, 16 bp, synthetic construct
  misc_feature 1..16: note = source = /note="Description of Sequence: Synthetic oligonucleotide"
  source 1..16: mol_type = other DNA
tgagggtcatacctcc

SEQ ID NO: 515 - DNA, 18 bp, synthetic construct
  misc_feature 1..18: note = source = /note="Description of Sequence: Synthetic oligonucleotide"
  source 1..18: mol_type = other DNA
tgagggtgcaaatcctcc

SEQ ID NO: 516 - DNA, 13 bp, synthetic construct
  misc_feature 1..13: note = source = /note="Description of Sequence: Synthetic oligonucleotide"
  source 1..13: mol_type = other DNA
tgccgaaaggcgt

SEQ ID NO: 517 - DNA, 13 bp, synthetic construct
  misc_feature 1..13: note = source = /note="Description of Sequence: Synthetic oligonucleotide"
  source 1..13: mol_type = other DNA
tgccgtaaggcgt

SEQ ID NO: 518 - DNA, 14 bp, synthetic construct
  misc_feature 1..14: note = source = /note="Description of Sequence: Synthetic oligonucleotide"
  source 1..14: mol_type = other DNA
tgcggtctccgcgc

SEQ ID NO: 519 - DNA, 11 bp, synthetic construct
  misc_feature 1..11: note = source = /note="Description of Sequence: Synthetic oligonucleotide"
  source 1..11: mol_type = other DNA
tgctagagcat

SEQ ID NO: 520 - DNA, 16 bp, synthetic construct
  misc_feature 1..16: note = source = /note="Description of Sequence: Synthetic oligonucleotide"
  source 1..16: mol_type = other DNA
tgctcgtatagagctc

SEQ ID NO: 521 - DNA, 15 bp, synthetic construct
  misc_feature 1..15: note = source = /note="Description of Sequence: Synthetic oligonucleotide"
  source 1..15: mol_type = other DNA
tggacaattgtctgc

SEQ ID NO: 522 - DNA, 15 bp, synthetic construct
  misc_feature 1..15: note = source = /note="Description of Sequence: Synthetic oligonucleotide"
  source 1..15: mol_type = other DNA
tggacagatgtccgt

SEQ ID NO: 523 - DNA, 15 bp, synthetic construct
  misc_feature 1..15: note = source = /note="Description of Sequence: Synthetic oligonucleotide"
  source 1..15: mol_type = other DNA
tggacaggtgtccgc

SEQ ID NO: 524 - DNA, 15 bp, synthetic construct
  misc_feature 1..15: note = source = /note="Description of Sequence: Synthetic oligonucleotide"
  source 1..15: mol_type = other DNA
tggacggttgtccgc

SEQ ID NO: 525 - DNA, 13 bp, synthetic construct
  misc_feature 1..13: note = source = /note="Description of Sequence: Synthetic oligonucleotide"
  source 1..13: mol_type = other DNA
tggacttgtggtc

SEQ ID NO: 526 - DNA, 16 bp, synthetic construct
  misc_feature 1..16: note = source = /note="Description of Sequence: Synthetic oligonucleotide"
  source 1..16: mol_type = other DNA
tggagattctctccgc

SEQ ID NO: 527 - DNA, 14 bp, synthetic construct
  misc_feature 1..14: note = source = /note="Description of Sequence: Synthetic oligonucleotide"
  source 1..14: mol_type = other DNA
tggcataggcctgc

SEQ ID NO: 528 - DNA, 14 bp, synthetic construct
  misc_feature 1..14: note = source = /note="Description of Sequence: Synthetic oligonucleotide"
  source 1..14: mol_type = other DNA
tggcttatgtctac

SEQ ID NO: 529 - DNA, 16 bp, synthetic construct
  misc_feature 1..16: note = source = /note="Description of Sequence: Synthetic oligonucleotide"
  source 1..16: mol_type = other DNA
tgggagttaatcccgt

SEQ ID NO: 530 - DNA, 14 bp, synthetic construct
  misc_feature 1..14: note = source = /note="Description of Sequence: Synthetic oligonucleotide"
  source 1..14: mol_type = other DNA
tgggatcttcccgc

SEQ ID NO: 531 - DNA, 16 bp, synthetic construct
  misc_feature 1..16: note = source = /note="Description of Sequence: Synthetic oligonucleotide"
  source 1..16: mol_type = other DNA
tgggcagaaatgtctc

SEQ ID NO: 532 - DNA, 15 bp, synthetic construct
  misc_feature 1..15: note = source = /note="Description of Sequence: Synthetic oligonucleotide"
  source 1..15: mol_type = other DNA
tgggcgttcgcccgc

SEQ ID NO: 533 - DNA, 14 bp, synthetic construct
  misc_feature 1..14: note = source = /note="Description of Sequence: Synthetic oligonucleotide"
  source 1..14: mol_type = other DNA
tgggcttcgcccgc

SEQ ID NO: 534 - DNA, 16 bp, synthetic construct
  misc_feature 1..16: note = source = /note="Description of Sequence: Synthetic oligonucleotide"
  source 1..16: mol_type = other DNA
tgggggataaccccgt

SEQ ID NO: 535 - DNA, 15 bp, synthetic construct
  misc_feature 1..15: note = source = /note="Description of Sequence: Synthetic oligonucleotide"
  source 1..15: mol_type = other DNA
tgggggtttccccgt

SEQ ID NO: 536 - skipped

SEQ ID NO: 537 - DNA, 15 bp, synthetic construct
  misc_feature 1..15: note = source = /note="Description of Sequence: Synthetic oligonucleotide"
  source 1..15: mol_type = other DNA
tggtggcaacaccgt

SEQ ID NO: 538 - DNA, 14 bp, synthetic construct
  misc_feature 1..14: note = source = /note="Description of Sequence: Synthetic oligonucleotide"
  source 1..14: mol_type = other DNA
tggtttatagccgt

SEQ ID NO: 539 - DNA, 19 bp, synthetic construct
  misc_feature 1..19: note = source = /note="Description of Sequence: Synthetic oligonucleotide"
  source 1..19: mol_type = other DNA
tgtacggtaataccgtacc

SEQ ID NO: 540 - DNA, 15 bp, synthetic construct
  misc_feature 1..15: note = source = /note="Description of Sequence: Synthetic oligonucleotide"
  source 1..15: mol_type = other DNA
tgtccgcaaggacgt

SEQ ID NO: 541 - DNA, 15 bp, synthetic construct
  misc_feature 1..15: note = source = /note="Description of Sequence: Synthetic oligonucleotide"
  source 1..15: mol_type = other DNA
tgtcctaacggacgt

SEQ ID NO: 542 - DNA, 18 bp, synthetic construct
  misc_feature 1..18: note = source = /note="Description of Sequence: Synthetic oligonucleotide"
  source 1..18: mol_type = other DNA
tgtcctattaacggacgt

SEQ ID NO: 543 - DNA, 16 bp, synthetic construct
  misc_feature 1..16: note = source = /note="Description of Sequence: Synthetic oligonucleotide"
  source 1..16: mol_type = other DNA
tgtccttcacgggcgt

SEQ ID NO: 544 - DNA, 13 bp, synthetic construct
  misc_feature 1..13: note = source = /note="Description of Sequence: Synthetic oligonucleotide"
  source 1..13: mol_type = other DNA
tgtcttaggacgt

SEQ ID NO: 545 - DNA, 18 bp, synthetic construct
  misc_feature 1..18: note = source = /note="Description of Sequence: Synthetic oligonucleotide"
  source 1..18: mol_type = other DNA
tgtgcgttaacgcgtacc

SEQ ID NO: 546 - DNA, 16 bp, synthetic construct
  misc_feature 1..16: note = source = /note="Description of Sequence: Synthetic oligonucleotide"
  source 1..16: mol_type = other DNA
tgtgtcgcaaggcacc

SEQ ID NO: 547 - DNA, 15 bp, synthetic construct
  misc_feature 1..15: note = source = /note="Description of Sequence: Synthetic oligonucleotide"
  source 1..15: mol_type = other DNA
tgttcgtaaggactt

SEQ ID NO: 548 - DNA, 16 bp, synthetic construct
  misc_feature 1..16: note = source = /note="Description of Sequence: Synthetic oligonucleotide"
  source 1..16: mol_type = other DNA
ttcacagaaatgtgtc

SEQ ID NO: 549 - DNA, 14 bp, synthetic construct
  misc_feature 1..14: note = source = /note="Description of Sequence: Synthetic oligonucleotide"
  source 1..14: mol_type = other DNA
ttccctcgtggagt

SEQ ID NO: 550 - DNA, 14 bp, synthetic construct
  misc_feature 1..14: note = source = /note="Description of Sequence: Synthetic oligonucleotide"
  source 1..14: mol_type = other DNA
ttccctctgggagc

SEQ ID NO: 551 - DNA, 14 bp, synthetic construct
  misc_feature 1..14: note = source = /note="Description of Sequence: Synthetic oligonucleotide"
  source 1..14: mol_type = other DNA
ttcccttgtggatc

SEQ ID NO: 552 - DNA, 13 bp, synthetic construct
  misc_feature 1..13: note = source = /note="Description of Sequence: Synthetic oligonucleotide"
  source 1..13: mol_type = other DNA
ttccttcgggagc

SEQ ID NO: 553 - DNA, 15 bp, synthetic construct
  misc_feature 1..15: note = source = /note="Description of Sequence: Synthetic oligonucleotide"
  source 1..15: mol_type = other DNA
ttctagcaatagagt

SEQ ID NO: 554 - DNA, 16 bp, synthetic construct
  misc_feature 1..16: note = source = /note="Description of Sequence: Synthetic oligonucleotide"
  source 1..16: mol_type = other DNA
ttctccactggggagc

SEQ ID NO: 555 - DNA, 15 bp, synthetic construct
  misc_feature 1..15: note = source = /note="Description of Sequence: Synthetic oligonucleotide"
  source 1..15: mol_type = other DNA
ttctcgagagggagc

SEQ ID NO: 556 - DNA, 15 bp, synthetic construct
  misc_feature 1..15: note = source = /note="Description of Sequence: Synthetic oligonucleotide"
  source 1..15: mol_type = other DNA
ttctcgtatgagagc

SEQ ID NO: 557 - DNA, 19 bp, synthetic construct
  misc_feature 1..19: note = source = /note="Description of Sequence: Synthetic oligonucleotide"
  source 1..19: mol_type = other DNA
tttaaggttttcccttaac

SEQ ID NO: 558 - DNA, 14 bp, synthetic construct
  misc_feature 1..14: note = source = /note="Description of Sequence: Synthetic oligonucleotide"
  source 1..14: mol_type = other DNA
tttcattgtggagt

SEQ ID NO: 559 - DNA, 14 bp, synthetic construct
  misc_feature 1..14: note = source = /note="Description of Sequence: Synthetic oligonucleotide"
  source 1..14: mol_type = other DNA
tttcgaaggaatcc

SEQ ID NO: 560 - DNA, 13 bp, synthetic construct
  misc_feature 1..13: note = source = /note="Description of Sequence: Synthetic oligonucleotide"
  source 1..13: mol_type = other DNA
tttcttcggaagc

SEQ ID NO: 561 - DNA, 16 bp, synthetic construct
  misc_feature 1..16: note = source = /note="Description of Sequence: Synthetic oligonucleotide"
  source 1..16: mol_type = other DNA
tttggggcaactcaac

SEQ ID NO: 562 - RNA, 342 nt, synthetic construct
  variation 5..6: note = a, c, u, or g
  variation 15..16: note = a, c, u, or g
  variation 21: note = a, c, u, or g
  variation 30..32: note = a, c, u, or g
  variation 34: note = a, c, u, or g
  variation 37: note = a, c, u, or g
  variation 41..45: note = a, c, u, or g
  variation 47..317: note = /note="This region may encompass 1-271 nucleotides"
  variation 47..320: note = a, c, u, or g
  variation 328..329: note = a, c, u, or g
  variation 333..334: note = a, c, u, or g
  variation 336..337: note = a, c, u, or g
  misc_feature 1..342: note = source = /note="Description of Sequence: Synthetic polynucleotide"
  source 1..342: mol_type = other RNA
rkssnndtrghbyannytgrndgwdvdydnnnbnhbnryrnnnnndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrrdtyrannyybnnhnnbwccd

SEQ ID NO: 563 - RNA, 341 nt, synthetic construct
  variation 16: note = a, c, u, or g
  variation 30: note = a, c, u, or g
  variation 46..316: note = a, c, u, or g
  variation 46..316: note = /note="This region may encompass 1-271 nucleotides"
  variation 319: note = a, c, u, or g
  variation 328: note = a, c, u, or g
  misc_feature 1..341: note = source = /note="Description of Sequence: Synthetic polynucleotide"
  source 1..341: mol_type = other RNA
rksgrwdkrghbyavnyggtdgarvrydynhkywbhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgdtcrayncyydvhhyywccd

SEQ ID NO: 564 - RNA, 341 nt, synthetic construct
  variation 16: note = a, c, u, or g
  variation 30: note = a, c, u, or g
  variation 46..316: note = a, c, u, or g
  variation 46..316: note = /note="This region may encompass 1-271 nucleotides"
  variation 319: note = a, c, u, or g
  misc_feature 1..341: note = source = /note="Description of Sequence: Synthetic polynucleotide"
  source 1..341: mol_type = other RNA
ggggrwdtrghbyavnyggtdgarvrydynhkywkhryrhdhdhrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbhnrgdtcraybccydvhhyytcca

SEQ ID NO: 565 - RNA, 343 nt, synthetic construct
  variation 2..8: note = a, c, u, or g
  variation 12..13: note = a, c, u, or g
  variation 17: note = a, c, u, or g
  variation 22..24: note = a, c, u, or g
  variation 26..27: note = a, c, u, or g
  variation 30..35: note = a, c, u, or g
  variation 38: note = a, c, u, or g
  variation 43: note = a, c, u, or g
  variation 45..322: note = a, c, u, or g
  variation 48..318: note = /note="This region may encompass 1-271 nucleotides"
  variation 329: note = a, c, u, or g
  variation 333..342: note = a, c, u, or g
  misc_feature 1..343: note = source = /note="Description of Sequence: Synthetic polynucleotide"
  source 1..343: mol_type = other RNA
gnnnnnnnkvgnnhddnhwrrnnnrnnvynnnnnnbtnckrhnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttyranyybnnnnnnnnnnd

SEQ ID NO: 566 - RNA, 342 nt, synthetic construct
  variation 3: note = a, c, u, or g
  variation 6..7: note = a, c, u, or g
  variation 13: note = a, c, u, or g
  variation 26: note = a, c, u, or g
  variation 32: note = a, c, u, or g
  variation 37: note = a, c, u, or g
  variation 44: note = a, c, u, or g
  variation 47..317: note = a, c, u, or g
  variation 47..317: note = /note="This region may encompass 1-271 nucleotides"
  variation 328: note = a, c, u, or g
  variation 336..338: note = a, c, u, or g
  variation 340: note = a, c, u, or g
  misc_feature 1..342: note = source = /note="Description of Sequence: Synthetic polynucleotide"
  source 1..342: mol_type = other RNA
grnvbnnvkbgbnydrwtrgwygarnrydyynsvytnckrmkbnrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshvggttyrantycbdbynnnbnhr

SEQ ID NO: 567 - RNA, 342 nt, synthetic construct
  variation 3: note = a, c, u, or g
  variation 7: note = a, c, u, or g
  variation 47..317: note = a, c, u, or g
  variation 47..317: note = /note="This region may encompass 1-271 nucleotides"
  variation 336: note = a, c, u, or g
  variation 340: note = a, c, u, or g
  misc_feature 1..342: note = source = /note="Description of Sequence: Synthetic polynucleotide"
  source 1..342: mol_type = other RNA
grnvyvnskkgssydawtggaygarsgyryykgmythckrakymrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnshrggttygavtycydbynbdbnyg

SEQ ID NO: 568 - RNA, 341 nt, synthetic construct
  variation 2: note = a, c, u, or g
  variation 4..6: note = a, c, u, or g
  variation 12: note = a, c, u, or g
  variation 17: note = a, c, u, or g
  variation 25: note = a, c, u, or g
  variation 28..30: note = a, c, u, or g
  variation 43..316: note = a, c, u, or g
  variation 46..316: note = /note="This region may encompass 1-271 nucleotides"
  variation 318..319: note = a, c, u, or g
  variation 327: note = a, c, u, or g
  variation 331..332: note = a, c, u, or g
  variation 334..337: note = a, c, u, or g
  variation 339..340: note = a, c, u, or g
  misc_feature 1..341: note = source = /note="Description of Sequence: Synthetic polynucleotide"
  source 1..341: mol_type = other RNA
dnynnndtrghnhvrynggbhtrdnrynnnbsryyrttwahbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgtkhvwnhcynnbnnnndnnr

SEQ ID NO: 569 - RNA, 341 nt, synthetic construct
  variation 17: note = a, c, u, or g
  variation 28: note = a, c, u, or g
  variation 46..316: note = a, c, u, or g
  variation 46..316: note = /note="This region may encompass 1-271 nucleotides"
  misc_feature 1..341: note = source = /note="Description of Sequence: Synthetic polynucleotide"
  source 1..341: mol_type = other RNA
rtytcygtrghryvrtnggbytarhgcnttysryyrttwahbddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgtrgtkhvwdmcyayysvrrrrbr

SEQ ID NO: 570 - RNA, 341 nt, synthetic construct
  variation 46..316: note = a, c, u, or g
  variation 46..316: note = /note="This region may encompass 1-271 nucleotides"
  misc_feature 1..341: note = source = /note="Description of Sequence: Synthetic polynucleotide"
  source 1..341: mol_type = other RNA
rtytcygtrghryvrtbggbytaghgcbttysryyrttaahyddannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgtrgtkhvwdmcyayysvrrrryg

SEQ ID NO: 571 - RNA, 342 nt, synthetic construct
  variation 2..6: note = a, c, u, or g
  variation 12: note = a, c, u, or g
  variation 16: note = a, c, u, or g
  variation 22: note = a, c, u, or g
  variation 26: note = a, c, u, or g
  variation 29: note = a, c, u, or g
  variation 31..32: note = a, c, u, or g
  variation 44: note = a, c, u, or g
  variation 47..317: note = /note="This region may encompass 1-271 nucleotides"
  variation 47..319: note = a, c, u, or g
  variation 328: note = a, c, u, or g
  variation 333..334: note = a, c, u, or g
  variation 336..339: note = a, c, u, or g
  misc_feature 1..342: note = source = /note="Description of Sequence: Synthetic polynucleotide"
  source 1..342: mol_type = other RNA
knnnnndyrrynhrrnyygrhntmdnrynbnnvhytrtcrhdbnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsrgytbrhnyyysnnhnnnnbmr

SEQ ID NO: 572 - RNA, 339 nt, synthetic construct
  variation 2: note = a, c, u, or g
  variation 6: note = a, c, u, or g
  variation 44..314: note = a, c, u, or g
  variation 44..314: note = /note="This region may encompass 1-271 nucleotides"
  variation 333: note = a, c, u, or g
  misc_feature 1..339: note = source = /note="Description of Sequence: Synthetic polynucleotide"
  source 1..339: mol_type = other RNA
knsbbnwyarywyrgtggtkmgwrtbysyryytrtcaygbrsrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnykgrgytbrhkyycchrwnvvksmr

SEQ ID NO: 573 - RNA, 339 nt, synthetic construct
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA
tmctyrtyagtatrgtggtkmgtrtyycygyctgtcaygyggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyggrgttyrmkyyccyrasrrggag

SEQ ID NO: 574 - RNA, 342 nt, synthetic construct
  variation 3..6: note = a, c, u, or g
  variation 12..13: note = a, c, u, or g
  variation 16: note = a, c, u, or g
  variation 24: note = a, c, u, or g
  variation 26: note = a, c, u, or g
  variation 29..32: note = a, c, u, or g
  variation 34: note = a, c, u, or g
  variation 42: note = a, c, u, or g
  variation 44..319: note = a, c, u, or g
  variation 47..317: note = /note="This region may encompass 1-271 nucleotides"
  variation 328: note = a, c, u, or g
  variation 333..334: note = a, c, u, or g
  variation 336..340: note = a, c, u, or g
  misc_feature 1..342: note = source = /note="Description of Sequence: Synthetic polynucleotide"
  source 1..342: mol_type = other RNA
gsnnnndtrvynnavnkcgdbyynrnvbnnnnvnyyrcarmnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdrttcranyyyvnnhnnnnnyy

SEQ ID NO: 575 - RNA, 340 nt, synthetic construct
  variation 16: note = a, c, u, or g
  variation 45..315: note = a, c, u, or g
  variation 45..315: note = /note="This region may encompass 1-271 nucleotides"
  misc_feature 1..340: note = source = /note="Description of Sequence: Synthetic polynucleotide"
  source 1..340: mol_type = other RNA
gsgsryrkrgytbasnkgdktaravyahttgrcyrcaratcmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyydrttcrartyyvgktryychyy

SEQ ID NO: 576 - RNA, 340 nt, synthetic construct
  variation 16: note = a, c, u, or g
  variation 45..315: note = a, c, u, or g
  variation 45..315: note = /note="This region may encompass 1-271 nucleotides"
  misc_feature 1..340: note = source = /note="Description of Sequence: Synthetic polynucleotide"
  source 1..340: mol_type = other RNA
gggsryrkrgytyasntgrktagagcayttgactgcaratcmarnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyyyrrttcraatyyvgktrcycmyy

SEQ ID NO: 577 - RNA, 341 nt, synthetic construct
  variation 2: note = a, c, u, or g
  variation 4..6: note = a, c, u, or g
  variation 12..13: note = a, c, u, or g
  variation 16: note = a, c, u, or g
  variation 20..21: note = a, c, u, or g
  variation 24: note = a, c, u, or g
  variation 28..31: note = a, c, u, or g
  variation 33: note = a, c, u, or g
  variation 40..41: note = a, c, u, or g
  variation 43..319: note = a, c, u, or g
  variation 46..316: note = /note="This region may encompass 1-271 nucleotides"
  variation 327: note = a, c, u, or g
  variation 331..332: note = a, c, u, or g
  variation 335..339: note = a, c, u, or g
  misc_feature 1..341: note = source = /note="Description of Sequence: Synthetic polynucleotide"
  source 1..341: mol_type = other RNA
dnbnnndtvgbnnarnhggnnhanvvynnnnvnytbtkrnnhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrrttyvanybynnbhnnnnnhd

SEQ ID NO: 578 - RNA, 340 nt, synthetic construct
  variation 4..5: note = a, c, u, or g
  variation 12: note = a, c, u, or g
  variation 21: note = a, c, u, or g
  variation 27..28: note = a, c, u, or g
  variation 45..315: note = /note="This region may encompass 1-271 nucleotides"
  variation 45..317: note = a, c, u, or g
  variation 331: note = a, c, u, or g
  variation 335..336: note = a, c, u, or g
  misc_feature 1..340: note = source = /note="Description of Sequence: Synthetic polynucleotide"
  source 1..340: mol_type = other RNA
kdbnnydtggynbarkmggdnavvrynnhbvrytbtkrrbhvdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrrttyvadybyhnbhdnnvhmd

SEQ ID NO: 579 - RNA, 340 nt, synthetic construct
  variation 4: note = a, c, u, or g
  variation 21: note = a, c, u, or g
  variation 27..28: note = a, c, u, or g
  variation 45..315: note = /note="This region may encompass 1-271 nucleotides"
  variation 45..317: note = a, c, u, or g
  variation 331: note = a, c, u, or g
  variation 336: note = a, c, u, or g
  misc_feature 1..340: note = source = /note="Description of Sequence: Synthetic polynucleotide"
  source 1..340: mol_type = other RNA
kdbnvydtggydbartmggknavvrynnwsrrytbtkrabhsdvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrrttyvadysyhnbhdbnvhmk

SEQ ID NO: 580 - RNA, 342 nt, synthetic construct
  variation 1..2: note = a, c, u, or g
  variation 5..6: note = a, c, u, or g
  variation 9: note = a, c, u, or g
  variation 12: note = a, c, u, or g
  variation 16: note = a, c, u, or g
  variation 20..21: note = a, c, u, or g
  variation 26..27: note = a, c, u, or g
  variation 29..33: note = a, c, u, or g
  variation 41: note = a, c, u, or g
  variation 44..318: note = a, c, u, or g
  variation 47..317: note = /note="This region may encompass 1-271 nucleotides"
  variation 320..321: note = a, c, u, or g
  variation 328: note = a, c, u, or g
  variation 333..336: note = a, c, u, or g
  variation 340..341: note = a, c, u, or g
  misc_feature 1..342: note = source = /note="Description of Sequence: Synthetic polynucleotide"
  source 1..342: mol_type = other RNA
nnbbnndtnrwnhrdnhygnnbhrdnnynnnnnvytytsrnbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnrytbrwnyyhbnnnndvvnnd

SEQ ID NO: 581 - RNA, 340 nt, synthetic construct
  variation 1..2: note = a, c, u, or g
  variation 5: note = a, c, u, or g
  variation 12: note = a, c, u, or g
  variation 20: note = a, c, u, or g
  variation 29: note = a, c, u, or g
  variation 31: note = a, c, u, or g
  variation 39: note = a, c, u, or g
  variation 43: note = a, c, u, or g
  variation 45..315: note = /note="This region may encompass 1-271 nucleotides"
  variation 45..316: note = a, c, u, or g
  variation 319: note = a, c, u, or g
  variation 326: note = a, c, u, or g
  variation 334: note = a, c, u, or g
  variation 338: note = a, c, u, or g
  misc_feature 1..340: note = source = /note="Description of Sequence: Synthetic polynucleotide"
  source 1..340: mol_type = other RNA
nnbynbdyrgtntrdyagbnkrrvvywbnbnvytytsanbvhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnydnrytbrwnychbrkbndvvnvd

SEQ ID NO: 582 - RNA, 339 nt, synthetic construct
  variation 1: note = a, c, u, or g
  variation 28: note = a, c, u, or g
  variation 30: note = a, c, u, or g
  variation 42: note = a, c, u, or g
  variation 44..314: note = a, c, u, or g
  variation 44..314: note = /note="This region may encompass 1-271 nucleotides"
  variation 318: note = a, c, u, or g
  variation 333: note = a, c, u, or g
  misc_feature 1..339: note = source = /note="Description of Sequence: Synthetic polynucleotide"
  source 1..339: mol_type = other RNA
ncsydbrtggtstakygshkrrsaywbnknsytytcahbvmndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbyrnrytyrwytcyyrgbnwrsraw

SEQ ID NO: 583 - RNA, 342 nt, synthetic construct
  variation 4..7: note = a, c, u, or g
  variation 12: note = a, c, u, or g
  variation 16: note = a, c, u, or g
  variation 21..22: note = a, c, u, or g
  variation 26: note = a, c, u, or g
  variation 29..34: note = a, c, u, or g
  variation 41..319: note = a, c, u, or g
  variation 47..317: note = /note="This region may encompass 1-271 nucleotides"
  variation 328: note = a, c, u, or g
  variation 333..338: note = a, c, u, or g
  misc_feature 1..342: note = source = /note="Description of Sequence: Synthetic polynucleotide"
  source 1..342: mol_type = other RNA
rbvnnnntvrynyrrnytbdnntadnrynnnnnnyybccvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvvstbbrwnhcbbnnnnnnbbyw

SEQ ID NO: 584 - RNA, 340 nt, synthetic construct
  variation 5: note = a, c, u, or g
  variation 28: note = a, c, u, or g
  variation 39..40: note = a, c, u, or g
  variation 42: note = a, c, u, or g
  variation 45..315: note = a, c, u, or g
  variation 45..315: note = /note="This region may encompass 1-271 nucleotides"
  variation 317: note = a, c, u, or g
  variation 335: note = a, c, u, or g
  misc_feature 1..340: note = source = /note="Description of Sequence: Synthetic polynucleotide"
  source 1..340: mol_type = other RNA
gbvbnsvtrrtdyrrdcggkdadmatdnhhrhytbccannynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnrgsttbrwttccyksbsnvygca

SEQ ID NO: 585 - RNA, 340 nt, synthetic construct
  variation 5: note = a, c, u, or g
  variation 28: note = a, c, u, or g
  variation 39: note = a, c, u, or g
  variation 42: note = a, c, u, or g
  variation 45..315: note = a, c, u, or g
  variation 45..315: note = /note="This region may encompass 1-271 nucleotides"
  variation 335: note = a, c, u, or g
  misc_feature 1..340: note = source = /note="Description of Sequence: Synthetic polynucleotide"
  source 1..340: mol_type = other RNA
gyvynsvtrrtdyrrwcggkkadmatdnhhrhytbccandynkdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbvrgsttyrwttccyksbsnvygca

SEQ ID NO: 586 - RNA, 342 nt, synthetic construct
  variation 4..7: note = a, c, u, or g
  variation 13: note = a, c, u, or g
  variation 25..26: note = a, c, u, or g
  variation 29..31: note = a, c, u, or g
  variation 34: note = a, c, u, or g
  variation 42: note = a, c, u, or g
  variation 45..317: note = a, c, u, or g
  variation 47..317: note = /note="This region may encompass 1-271 nucleotides"
  variation 319..320: note = a, c, u, or g
  variation 328: note = a, c, u, or g
  variation 332..333: note = a, c, u, or g
  variation 336..338: note = a, c, u, or g
  misc_feature 1..342: note = source = /note="Description of Sequence: Synthetic polynucleotide"
  source 1..342: mol_type = other RNA
kgynnnndtvgbnhagbytgghyannvynnndbnytgtgrhnvhnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgytcranycynnbhnnnvrym

SEQ ID NO: 587 - RNA, 339 nt, synthetic construct
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA
gchrtgaybgtayagkggttasyactctgygytgtggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwyggytcraatcygagtcaygrca

SEQ ID NO: 588 - RNA, 339 nt, synthetic construct
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA
gcmrtgayygtatagtggttagyactctgygytgtggccrcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncwyggttcraatcygagtcaygrca

SEQ ID NO: 589 - RNA, 342 nt, synthetic construct
  variation 1: note = a, c, u, or g
  variation 4..5: note = a, c, u, or g
  variation 7: note = a, c, u, or g
  variation 17: note = a, c, u, or g
  variation 31..32: note = a, c, u, or g
  variation 34: note = a, c, u, or g
  variation 37: note = a, c, u, or g
  variation 42..320: note = a, c, u, or g
  variation 47..317: note = /note="This region may encompass 1-271 nucleotides"
  variation 328..329: note = a, c, u, or g
  variation 332..334: note = a, c, u, or g
  variation 336..339: note = a, c, u, or g
  misc_feature 1..342: note = source = /note="Description of Sequence: Synthetic polynucleotide"
  source 1..342: mol_type = other RNA
nvsnnbndtrgybyrrnywggbhrdvrydbnnbnytnatrannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndsywydannchnnnhnnnnsba

SEQ ID NO: 590 - RNA, 340 nt, synthetic construct
  variation 45..315: note = a, c, u, or g
  variation 45..315: note = /note="This region may encompass 1-271 nucleotides"
  variation 326..327: note = a, c, u, or g
  variation 335: note = a, c, u, or g
  misc_feature 1..340: note = source = /note="Description of Sequence: Synthetic polynucleotide"
  source 1..340: mol_type = other RNA
gsyybrktrgykyrrtyggyhagmgcrygktrctdataayryyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnryrrsywydanncycrymyngrsca

SEQ ID NO: 591 - RNA, 340 nt, synthetic construct
  variation 45..315: note = a, c, u, or g
  variation 45..315: note = /note="This region may encompass 1-271 nucleotides"
  misc_feature 1..340: note = source = /note="Description of Sequence: Synthetic polynucleotide"
  source 1..340: mol_type = other RNA
gsyysrktrgykcartyggyhagmgcgyggtrctdataayrcyrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngygrgytcrabhcycrymybgrsca

SEQ ID NO: 592 - RNA, 341 nt, synthetic construct
  variation 1: note = a, c, u, or g
  variation 3..6: note = a, c, u, or g
  variation 11..12: note = a, c, u, or g
  variation 16..17: note = a, c, u, or g
  variation 21..22: note = a, c, u, or g
  variation 25..26: note = a, c, u, or g
  variation 28..31: note = a, c, u, or g
  variation 41: note = a, c, u, or g
  variation 43..316: note = a, c, u, or g
  variation 46..316: note = /note="This region may encompass 1-271 nucleotides"
  variation 318..319: note = a, c, u, or g
  variation 327: note = a, c, u, or g
  variation 331..332: note = a, c, u, or g
  variation 335..338: note = a, c, u, or g
  variation 340: note = a, c, u, or g
  misc_feature 1..341: note = source = /note="Description of Sequence: Synthetic polynucleotide"
  source 1..341: mol_type = other RNA
nbnnnndtrsnnbarnnhggnnddnnbnnnnvdhycatahnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnrgdtsdanhmynnbhnnnnvnw

SEQ ID NO: 593 - RNA, 339 nt, synthetic construct
  variation 4: note = a, c, u, or g
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA
rbcnbvktrgygcagydgghagyryryyrgkyycataahyyvrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsdkrgdtsdadmmymwsmbvbgsya

SEQ ID NO: 594 - RNA, 339 nt, synthetic construct
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA
rschbvktrgygcagydgghagcryryyrgkyycataaycyrrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnswkrgwtsdadmcymwsmbvkgsya

SEQ ID NO: 595 - RNA, 342 nt, synthetic construct
  variation 2..7: note = a, c, u, or g
  variation 9..12: note = a, c, u, or g
  variation 16: note = a, c, u, or g
  variation 23: note = a, c, u, or g
  variation 26: note = a, c, u, or g
  variation 28..34: note = a, c, u, or g
  variation 37: note = a, c, u, or g
  variation 41..317: note = a, c, u, or g
  variation 47..317: note = /note="This region may encompass 1-271 nucleotides"
  variation 319..320: note = a, c, u, or g
  variation 328: note = a, c, u, or g
  variation 332..333: note = a, c, u, or g
  variation 335..340: note = a, c, u, or g
  misc_feature 1..342: note = source = /note="Description of Sequence: Synthetic polynucleotide"
  source 1..342: mol_type = other RNA
vnnnnnntnnnnvrrnhhgkhhndhnvnnnnnnnhynardnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvbythvanymbnnbnnnnnnbr

SEQ ID NO: 596 - RNA, 342 nt, synthetic construct
  variation 6: note = a, c, u, or g
  variation 33: note = a, c, u, or g
  variation 41: note = a, c, u, or g
  variation 43: note = a, c, u, or g
  variation 46..317: note = a, c, u, or g
  variation 47..317: note = /note="This region may encompass 1-271 nucleotides"
  variation 319: note = a, c, u, or g
  variation 328: note = a, c, u, or g
  variation 333: note = a, c, u, or g
  variation 336: note = a, c, u, or g
  variation 340: note = a, c, u, or g
  misc_feature 1..342: note = source = /note="Description of Sequence: Synthetic polynucleotide"
  source 1..342: mol_type = other RNA
rbbvvndtrkhhsrrbhygkhhtrwbrhdbhdnrythardnynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbythvanymybnbbnbbvnya

SEQ ID NO: 597 - RNA, 342 nt, synthetic construct
  variation 6: note = a, c, u, or g
  variation 33: note = a, c, u, or g
  variation 43: note = a, c, u, or g
  variation 46..317: note = a, c, u, or g
  variation 47..317: note = /note="This region may encompass 1-271 nucleotides"
  variation 319: note = a, c, u, or g
  variation 333: note = a, c, u, or g
  variation 336: note = a, c, u, or g
  variation 340: note = a, c, u, or g
  misc_feature 1..342: note = source = /note="Description of Sequence: Synthetic polynucleotide"
  source 1..342: mol_type = other RNA
rbbvvndtrkhhsrrbhygkhhtrwkrhdbhdnrythardhynhdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnvgbythvavymybnbynbbvnya

SEQ ID NO: 598 - RNA, 342 nt, synthetic construct
  variation 1..7: note = a, c, u, or g
  variation 11..12: note = a, c, u, or g
  variation 16: note = a, c, u, or g
  variation 21: note = a, c, u, or g
  variation 26: note = a, c, u, or g
  variation 30..32: note = a, c, u, or g
  variation 44..320: note = a, c, u, or g
  variation 47..317: note = /note="This region may encompass 1-271 nucleotides"
  variation 328: note = a, c, u, or g
  variation 332..333: note = a, c, u, or g
  variation 335..340: note = a, c, u, or g
  misc_feature 1..342: note = source = /note="Description of Sequence: Synthetic polynucleotide"
  source 1..342: mol_type = other RNA
nnnnnnntrsnnharnhwrrnttdrnrydnnnsvyyyttmmbvnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvrytyrwnhybnnbnnnnnnhd

SEQ ID NO: 599 - RNA, 340 nt, synthetic construct
  variation 1: note = a, c, u, or g
  variation 6: note = a, c, u, or g
  variation 11: note = a, c, u, or g
  variation 29: note = a, c, u, or g
  variation 43: note = a, c, u, or g
  variation 45..315: note = /note="This region may encompass 1-271 nucleotides"
  variation 45..317: note = a, c, u, or g
  variation 331: note = a, c, u, or g
  variation 333..334: note = a, c, u, or g
  variation 336: note = a, c, u, or g
  misc_feature 1..340: note = source = /note="Description of Sequence: Synthetic polynucleotide"
  source 1..340: mol_type = other RNA
nhvbdnvkrsnhharbhrrbtdrdrydbndgryyyttmmymhndnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrgrytyrwbyysynbnnhndrhr

SEQ ID NO: 600 - RNA, 340 nt, synthetic construct
  variation 45..315: note = /note="This region may encompass 1-271 nucleotides"
  variation 45..316: note = a, c, u, or g
  variation 333: note = a, c, u, or g
  variation 336: note = a, c, u, or g
  misc_feature 1..340: note = source = /note="Description of Sequence: Synthetic polynucleotide"
  source 1..340: mol_type = other RNA
vhvbrdvkascwharthrrbtdrwrcrkvdgactyttmatchswnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrgrytyrwkyysyhbnhynkryr

SEQ ID NO: 601 - RNA, 341 nt, synthetic construct
  variation 4..6: note = a, c, u, or g
  variation 29..31: note = a, c, u, or g
  variation 33: note = a, c, u, or g
  variation 41: note = a, c, u, or g
  variation 43..318: note = a, c, u, or g
  variation 46..316: note = /note="This region may encompass 1-271 nucleotides"
  variation 327: note = a, c, u, or g
  variation 331..332: note = a, c, u, or g
  variation 335..338: note = a, c, u, or g
  misc_feature 1..341: note = source = /note="Description of Sequence: Synthetic polynucleotide"
  source 1..341: mol_type = other RNA
kbbnnndtagyyyarhytggbwwgrryrnnnbnyygaarvnbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbdgttcranychnnbhnnnnvma

SEQ ID NO: 602 - RNA, 340 nt, synthetic construct
  variation 45..315: note = a, c, u, or g
  variation 45..315: note = /note="This region may encompass 1-271 nucleotides"
  misc_feature 1..340: note = source = /note="Description of Sequence: Synthetic polynucleotide"
  source 1..340: mol_type = other RNA
kbyvwrrtwgyyyarttggstagrrydykrbrcygaarrysyhmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyckggttcrayycysggtywbrvma

SEQ ID NO: 603 - RNA, 339 nt, synthetic construct
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA
gcyraratwgctcarttgggagagygttasacygaagatctwmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnyctggttcrayycygggtttcrvca

SEQ ID NO: 604 - RNA, 342 nt, synthetic construct
  variation 4..6: note = a, c, u, or g
  variation 16: note = a, c, u, or g
  variation 21: note = a, c, u, or g
  variation 29..34: note = a, c, u, or g
  variation 37: note = a, c, u, or g
  variation 41..320: note = a, c, u, or g
  variation 47..317: note = /note="This region may encompass 1-271 nucleotides"
  variation 328: note = a, c, u, or g
  variation 331..334: note = a, c, u, or g
  variation 336..338: note = a, c, u, or g
  misc_feature 1..342: note = source = /note="Description of Sequence: Synthetic polynucleotide"
  source 1..342: mol_type = other RNA
sksnnndtvgbbyagnyygbnyyrdvvynnnnnnhtnggrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndgttcranycnnnnhnnnbmsm

SEQ ID NO: 605 - RNA, 338 nt, synthetic construct
  variation 39: note = a, c, u, or g
  variation 43..313: note = a, c, u, or g
  variation 43..313: note = /note="This region may encompass 1-271 nucleotides"
  misc_feature 1..338: note = source = /note="Description of Sequence: Synthetic polynucleotide"
  source 1..338: mol_type = other RNA
ggctsgttggkctagkgbtrtgrttctcrsythggrysnragnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncygggttcaaatcvyrgasgagccc

SEQ ID NO: 606 - RNA, 338 nt, synthetic construct
  variation 43..313: note = a, c, u, or g
  variation 43..313: note = /note="This region may encompass 1-271 nucleotides"
  misc_feature 1..338: note = source = /note="Description of Sequence: Synthetic polynucleotide"
  source 1..338: mol_type = other RNA
ggctcgttggtctagkggtrtgrttctcgstthggrysbgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccgggttcaaatcccggacgagccc

SEQ ID NO: 607 - RNA, 342 nt, synthetic construct
  variation 2..7: note = a, c, u, or g
  variation 9..13: note = a, c, u, or g
  variation 16: note = a, c, u, or g
  variation 21: note = a, c, u, or g
  variation 25..28: note = a, c, u, or g
  variation 30..34: note = a, c, u, or g
  variation 37: note = a, c, u, or g
  variation 42..320: note = a, c, u, or g
  variation 47..317: note = /note="This region may encompass 1-271 nucleotides"
  variation 332..340: note = a, c, u, or g
  misc_feature 1..342: note = source = /note="Description of Sequence: Synthetic polynucleotide"
  source 1..342: mol_type = other RNA
dnnnnnnynnnnnarnbhggnbbannnndnnnnnyynswrmnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnrkttyradycynnnnnnnnnbd

SEQ ID NO: 608 - RNA, 341 nt, synthetic construct
  variation 2..5: note = a, c, u, or g
  variation 36: note = a, c, u, or g
  variation 46..316: note = a, c, u, or g
  variation 46..316: note = /note="This region may encompass 1-271 nucleotides"
  variation 319: note = a, c, u, or g
  variation 331: note = a, c, u, or g
  variation 335..336: note = a, c, u, or g
  variation 338..339: note = a, c, u, or g
  misc_feature 1..341: note = source = /note="Description of Sequence: Synthetic polynucleotide"
  source 1..341: mol_type = other RNA
knnnnbdtrgchsarkbtggttavdghrdbkdrcynswraybmvdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvyngkttyradyccnrbhnnbnnyd

SEQ ID NO: 609 - RNA, 341 nt, synthetic construct
  variation 2..3: note = a, c, u, or g
  variation 5: note = a, c, u, or g
  variation 36: note = a, c, u, or g
  variation 46..316: note = a, c, u, or g
  variation 46..316: note = /note="This region may encompass 1-271 nucleotides"
  variation 335..336: note = a, c, u, or g
  variation 338..339: note = a, c, u, or g
  misc_feature 1..341: note = source = /note="Description of Sequence: Synthetic polynucleotide"
  source 1..341: mol_type = other RNA
knndnbrtrgchsarkctggttavdghrwykrrctnswrayymrdnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvydggttyraaycchrbynnynnyk

SEQ ID NO: 610 - RNA, 341 nt, synthetic construct
  variation 4..7: note = a, c, u, or g
  variation 12: note = a, c, u, or g
  variation 16: note = a, c, u, or g
  variation 25: note = a, c, u, or g
  variation 29..31: note = a, c, u, or g
  variation 33: note = a, c, u, or g
  variation 36: note = a, c, u, or g
  variation 41: note = a, c, u, or g
  variation 43..319: note = a, c, u, or g
  variation 46..316: note = /note="This region may encompass 1-271 nucleotides"
  variation 327: note = a, c, u, or g
  variation 331..337: note = a, c, u, or g
  variation 341: note = a, c, u, or g
  misc_feature 1..341: note = source = /note="Description of Sequence: Synthetic polynucleotide"
  source 1..341: mol_type = other RNA
rbbnnnntrgynhadnhykghyrdnryrnnnsnhyngtwansnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndgwyyvanbyhnnnnnnnrvyn

SEQ ID NO: 611 - RNA, 340 nt, synthetic construct
  variation 4: note = a, c, u, or g
  variation 6: note = a, c, u, or g
  variation 12: note = a, c, u, or g
  variation 24: note = a, c, u, or g
  variation 45..315: note = a, c, u, or g
  variation 45..315: note = /note="This region may encompass 1-271 nucleotides"
  variation 317: note = a, c, u, or g
  variation 326: note = a, c, u, or g
  variation 331..332: note = a, c, u, or g
  variation 334: note = a, c, u, or g
  variation 336: note = a, c, u, or g
  misc_feature 1..340: note = source = /note="Description of Sequence: Synthetic polynucleotide"
  source 1..340: mol_type = other RNA
rbbnbndtvgynharbygghyrdnryryhbsbcyhgtwavsvdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvndrgwtyvanbyyhnnbndnrvyb

SEQ ID NO: 612 - RNA, 340 nt, synthetic construct
  variation 4: note = a, c, u, or g
  variation 6: note = a, c, u, or g
  variation 12: note = a, c, u, or g
  variation 24: note = a, c, u, or g
  variation 45..315: note = a, c, u, or g
  variation 45..315: note = /note="This region may encompass 1-271 nucleotides"
  variation 326: note = a, c, u, or g
  variation 332: note = a, c, u, or g
  variation 334: note = a, c, u, or g
  variation 336: note = a, c, u, or g
  misc_feature 1..340: note = source = /note="Description of Sequence: Synthetic polynucleotide"
  source 1..340: mol_type = other RNA
rsynbndtvgynharbtgghyrdnryryhksbcyhgtaavsmdrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvhdrgwtyvanbyyhdnbndnrsyt

SEQ ID NO: 613 - RNA, 342 nt, synthetic construct
  variation 4..6: note = a, c, u, or g
  variation 29..32: note = a, c, u, or g
  variation 34: note = a, c, u, or g
  variation 42: note = a, c, u, or g
  variation 44..317: note = a, c, u, or g
  variation 47..317: note = /note="This region may encompass 1-271 nucleotides"
  variation 319: note = a, c, u, or g
  variation 321: note = a, c, u, or g
  variation 328: note = a, c, u, or g
  variation 333: note = a, c, u, or g
  variation 336..338: note = a, c, u, or g
  misc_feature 1..342: note = source = /note="Description of Sequence: Synthetic polynucleotide"
  source 1..342: mol_type = other RNA
rvvnnndtrrybhhrhyhgghytadvvynnnnrnbtccavanbnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnrngttcranychynbhnnnbbhd

SEQ ID NO: 614 - RNA, 339 nt, synthetic construct
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA
grmmkvrkggysmarytggharssykkybrrstccasakbvrwnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsygkgttcradycmcrkyksbgyma

SEQ ID NO: 615 - RNA, 339 nt, synthetic construct; identical to SEQ ID NO: 614
  variation 44..314: note = a, c, u, or g
  variation 44..314: note = /note="This region may encompass 1-271 nucleotides"
  misc_feature 1..339: note = source = /note="Description of Sequence: Synthetic polynucleotide"
  source 1..339: mol_type = other RNA

SEQ ID NO: 616 - RNA, 342 nt, synthetic construct
  variation 3..6: note = a, c, u, or g
  variation 9..10: note = a, c, u, or g
  variation 12..13: note = a, c, u, or g
  variation 16: note = a, c, u, or g
  variation 25..26: note = a, c, u, or g
  variation 30..32: note = a, c, u, or g
  variation 42: note = a, c, u, or g
  variation 44..317: note = a, c, u, or g
  variation 47..317: note = /note="This region may encompass 1-271 nucleotides"
  variation 319..320: note = a, c, u, or g
  variation 328: note = a, c, u, or g
  variation 332..333: note = a, c, u, or g
  variation 336..340: note = a, c, u, or g
  misc_feature 1..342: note = source = /note="Description of Sequence: Synthetic polynucleotide"
  source 1..342: mol_type = other RNA
bbnnnndtnnynnarnytgsyhwrnnrbdnnnrdctrtarvnynnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnvnnvgwtyrantcbnnbhnnnnnvd

SEQ ID NO: 617 - RNA, 340 nt, synthetic construct
  variation 45..315: note = a, c, u, or g
  variation 45..315: note = /note="This region may encompass 1-271 nucleotides"
  variation 336: note = a, c, u, or g
  misc_feature 1..340: note = source = /note="Description of Sequence: Synthetic polynucleotide"
  source 1..340: mol_type = other RNA
bbkkbdatagytcagbtgsytagagydkwkractrtagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsctggwtyrahtcyrrstbsnmsvd

SEQ ID NO: 618 - RNA, 340 nt, synthetic construct
  variation 45..315: note = a, c, u, or g
  variation 45..315: note = /note="This region may encompass 1-271 nucleotides"
  misc_feature 1..340: note = source = /note="Description of Sequence: Synthetic polynucleotide"
  source 1..340: mol_type = other RNA
bbkksdatagytcagytggytagagcdkwkgactrtagrkymwknnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnsctggttyrawtccrrstysdmsva

SEQ ID NO: 619 - RNA, 341 nt, synthetic construct
  variation 2: note = a, c, u, or g
  variation 4..7: note = a, c, u, or g
  variation 12: note = a, c, u, or g
  variation 15..16: note = a, c, u, or g
  variation 21: note = a, c, u, or g
  variation 24..25: note = a, c, u, or g
  variation 28: note = a, c, u, or g
  variation 30..33: note = a, c, u, or g
  variation 36: note = a, c, u, or g
  variation 40..319: note = a, c, u, or g
  variation 46..316: note = /note="This region may encompass 1-271 nucleotides"
  variation 327: note = a, c, u, or g
  variation 330..339: note = a, c, u, or g
  misc_feature 1..341: note = source = /note="Description of Sequence: Synthetic polynucleotide"
  source 1..341: mol_type = other RNA
dndnnnntvrynbrnnhtgknyannrynbnnnnhtnacrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnndrdtbranhynnnnnnnnnnyv

SEQ ID NO: 620 - RNA, 340 nt, synthetic construct
  variation 15..16: note = a, c, u, or g
  variation 24: note = a, c, u, or g
  variation 27: note = a, c, u, or g
  variation 29..30: note = a, c, u, or g
  variation 41..43: note = a, c, u, or g
  variation 45..315: note = a, c, u, or g
  variation 45..315: note = /note="This region may encompass 1-271 nucleotides"
  variation 317..318: note = a, c, u, or g
  variation 326: note = a, c, u, or g
  variation 330: note = a, c, u, or g
  variation 333: note = a, c, u, or g
  misc_feature 1..340: note = source = /note="Description of Sequence: Synthetic polynucleotide"
  source 1..340: mol_type = other RNA
kbkbshdtrrydbrnnhgktyadnrynynndhythacrhknnnrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnnrrdtbranhybndvndsdwvyv

SEQ ID NO: 621 - RNA, 340 nt, synthetic construct
  variation 45..315: note = a, c, u, or g
  variation 45..315: note = /note="This region may encompass 1-271 nucleotides"
  variation 317..318: note = a, c, u, or g
  variation 330: note = a, c, u, or g
  misc_feature 1..340: note = source = /note="Description of Sequence: Synthetic polynucleotide"
  source 1..340: mol_type = other RNA
kbkbsydtrgydbrbhtgktyadhryvyhhdyythacahkhddrnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnbnngrdtbradhyyndvhrsdwvya

SEQ ID NO: 622 - DNA, 72 bp, synthetic construct; identical to SEQ ID NO: 262
  misc_feature 1..72: note = source = /note="Description of Sequence: Synthetic oligonucleotide"
  source 1..72: mol_type = other DNA

SEQ ID NO: 623 - DNA, 20 bp, synthetic construct
  misc_feature 1..20: note = source = /note="Description of Sequence: Synthetic primer"
  source 1..20: mol_type = other DNA
tgagttggcaacctgtggta

SEQ ID NO: 624 - DNA, 20 bp, synthetic construct
  misc_feature 1..20: note = source = /note="Description of Sequence: Synthetic primer"
  source 1..20: mol_type = other DNA
ttgggtgtccatgaaaatca

SEQ ID NO: 625 - DNA, 26 bp, synthetic construct
  misc_feature 1..26: note = source = /note="Description of Sequence: Synthetic probe"
  source 1..26: mol_type = other DNA
tagcagaggatggtttcgatccatca